(19) United States
(12) Patent Application Publication
(10) Pub. No.: US 2023/0253066 A1
(43) Pub. Date: Aug. 10, 2023

(54) DESIGNED PROTEINS FOR LIGAND BINDING

(71) Applicant: The Regents of the University of California, Oakland, CA (US)

(72) Inventors: William F. DeGrado, San Francisco, CA (US); Nicholas Polizzi, San Francisco, CA (US)

(21) Appl. No.: 18/015,582

(22) PCT Filed: Jul. 21, 2021
(86) PCT No.: PCT/US2021/042647

§ 371 Date: Jan. 11, 2023

Related U.S. Application Data
(60) Provisional application No. 63/054,585, filed on Jul. 21, 2020.

(51) Int. Cl. G16B 15/30

(52) U.S. Cl. CPC G16B 15/30

(57) ABSTRACT

Disclosed herein, inter alia, are methods and systems for optimizing protein ligand interactions.

### CROSS-REFERENCES TO RELATED APPLICATIONS

This application claims priority to U.S. Provisional Application No. 63/054,585, entitled “DESIGNED PROTEINS FOR LIGAND BINDING” and filed on Jul. 21, 2020, the disclosure of which is incorporated herein by reference in its entirety.

### STATEMENT AS TO RIGHTS TO INVENTIONS MADE UNDER FEDERALLY SPONSORED RESEARCH AND DEVELOPMENT

This invention was made with Government support under grant numbers R35 GM122603, awarded by The National Institutes of Health, and 1709506, awarded by the National Science Foundation, and FA9550-19-1-0331, awarded by the Air Force Office of Scientific Research. The Government has certain rights in the invention.

### BACKGROUND

The Anfinsen hypothesis states that a protein's sequence encodes its tertiary structure and underlying function (1). Conversely, a protein's tertiary structure encodes the possible sequences compatible with a particular function. De novo protein design has succeeded in the creation of proteins that fold to various targeted tertiary structures (structure to sequence) (2, 3). Nevertheless, it has been extremely challenging to design proteins that not only fold but also bind to complex small molecules (function/structure to sequence) (2-4). Algorithms optimized for packing apolar protein cores struggle to design polar cavities required for binding hydrophilic molecules (5). Consequently, design of small-molecule-binding proteins has generally required recursive experimental screening and large libraries to engender function, mostly starting with natural proteins rather than de novo structures (FIG. 1A) (3, 4, 6-9). Disclosed herein, inter alia, are solutions to these and other problems in the art.

### BRIEF SUMMARY

In one aspect, there is provided a system that includes at least one data processor and at least one memory storing instructions. The instructions may cause operations when executed by the at least one data processor. The operations may include: querying a van der Mer (vdM) database to identify a first van der Mer known to interact with a first portion of a first compound, the first van der Mer corresponding to an in silico unit of protein structure that defines, based at least on a statistically preferred orientation of the first portion of the first compound relative to a backbone structure of a protein, a binding site for the first compound relative to the backbone structure of the protein; and generating, based at least on the first van der Mer, a sequence for the protein such that the protein exhibits a binding affinity for the first compound.

In some variations, one or more features disclosed herein including the following features can optionally be included in any feasible combination. The van der Mer database may include a plurality of van der Mers. Each of the plurality of van der Mers may be associated with a portion of a compound and a backbone structure.

In some variations, the plurality of van der Mers may be organized into one or more clusters of van der Mers exhibiting a same or similar interaction with the portion of the compound.

In some variations, the plurality of van der Mers may be clustered based at least on a first set of atomic protein coordinates associated with the portion of the compound and a second set of atomic protein coordinates associated with the backbone structure.

In some variations, the plurality of van der Mers included in the van der Mer database may be identified by searching a database of known protein structures for one or more units of protein structure exhibiting a van der Waals (vdW) contact with the portion of the compound.

In some variations, the one or more units of protein structure exhibiting the van der Waals (vdW) contact with the portion of the compound may be identified as van der Mers based at least on a nature of contact with the portion of the compound.

In some variations, the nature of contact may be one of a hydrogen bond, a close van der Waals contact, and a wide van der Waals contact.

In some variations, the operations may further include: generating a first set of coordinates corresponding to the backbone structure of the protein; generating a second set of coordinates corresponding to the compound or the portion of the compound; and querying, based at least on the first set of coordinates and the second set of coordinates, the van der Mer database.

In some variations, the operations may further include: querying the van der Mer database to identify a second van der Mer known to interact with the first portion of the first compound or a second portion of the first compound; and generating, based at least on the second van der Mer, the sequence for the protein.

In some variations, the backbone structure of the protein may include one of a plurality of backbone structures with a geometry consistent with a known plasticity of a selected protein fold.

In some variations, the sequence of the protein may be further generated by packing additional residues in the binding site.

In some variations, the sequence of the protein may be further generated by packing a core of the protein.

In some variations, the portion of the compound may include a chemical group.

In some variations, the compound may include a ligand.

In some variations, the ligand may include a peptide, a protein, a small molecule, or a small molecule-metal-ion complex.

In some variations, the first van der Mer may be selected instead of a second van der Mer based at least on the first van der Mer being observed in more experimentally determined protein structures than the second van der Mer.

In some variations, the operations may further include: optimizing the sequence of the protein by at least identifying a location of the binding site relative to the backbone structure of the protein associated with a minimum energy function.

In some variations, the optimizing may be performed by applying one or more of an interative algorithm, a heurisitic algorithm, a Monte Carlo sampling algorithm, a dead-end elimination algorithm, a branch and bound algorithm, a pruning algorithm, a simplex algorithm, a memetic algorithm, a differential evolution algorithm, an evolutionary algorithm, a genetic algorithm, a tabu algorithm, a particle swarm algorithm, and a simulated annealing algorithm.

In some variations, the energy function may include a molecular mechanics function, a structural bioinformatics function, an amino acid sidechain packing function, and/or protein radius of gyration function.

In some variations, the sequence of the protein is further generated such that the protein exhibits a desired tertiary structure including one or more folds.

In another aspect, there is provided a computer-implemented method that includes: querying a van der Mer (vdM) database to identify a first van der Mer known to interact with a first portion of a first compound, the first van der Mer corresponding to an in silico unit of protein structure that defines, based at least on a statistically preferred orientation of the first portion of the first compound relative to a backbone structure of a protein, a binding site for the first compound relative to the backbone structure of the protein; and generating, based at least on the first van der Mer, a sequence for the protein such that the protein exhibits a binding affinity for the first compound.

In some variations, one or more features disclosed herein including the following features can optionally be included in any feasible combination. The van der Mer database may include a plurality of van der Mers. Each of the plurality of van der Mers may be associated with a portion of a compound and a backbone structure.

In some variations, the plurality of van der Mers may be organized into one or more clusters of van der Mers exhibiting a same or similar interaction with the portion of the compound.

In some variations, the plurality of van der Mers may be clustered based at least on a first set of atomic protein coordinates associated with the portion of the compound and a second set of atomic protein coordinates associated with the backbone structure.

In some variations, the plurality of van der Mers included in the van der Mer database may be identified by searching a database of known protein structures for one or more units of protein structure exhibiting a van der Waals (vdW) contact with the portion of the compound.

In some variations, the one or more units of protein structure exhibiting the van der Waals (vdW) contact with the portion of the compound may be identified as van der Mers based at least on a nature of contact with the portion of the compound.

In some variations, the nature of contact may be one of a hydrogen bond, a close van der Waals contact, and a wide van der Waals contact.

In some variations, the method may further include: generating a first set of coordinates corresponding to the backbone structure of the protein; generating a second set of coordinates corresponding to the compound or the portion of the compound; and querying, based at least on the first set of coordinates and the second set of coordinates, the van der Mer database.

In some variations, the method may further include: querying the van der Mer database to identify a second van der Mer known to interact with the first portion of the first compound or a second portion of the first compound; and generating, based at least on the second van der Mer, the sequence for the protein.

In some variations, the backbone structure of the protein may include one of a plurality of backbone structures with a geometry consistent with a known plasticity of a selected protein fold.

In some variations, the sequence of the protein may be further generated by packing additional residues in the binding site.

In some variations, the sequence of the protein may be further generated by packing a core of the protein.

In some variations, the portion of the compound may include a chemical group.

In some variations, the compound may include a ligand.

In some variations, the ligand may include a peptide, a protein, a small molecule, or a small molecule-metal-ion complex.

In some variations, the first van der Mer may be selected instead of a second van der Mer based at least on the first van der Mer being observed in more experimentally determined protein structures than the second van der Mer.

In some variations, the method may further include: optimizing the sequence of the protein by at least identifying a location of the binding site relative to the backbone structure of the protein associated with a minimum energy function.

In some variations, the optimizing may be performed by applying one or more of an interative algorithm, a heurisitic algorithm, a Monte Carlo sampling algorithm, a dead-end elimination algorithm, a branch and bound algorithm, a pruning algorithm, a simplex algorithm, a memetic algorithm, a differential evolution algorithm, an evolutionary algorithm, a genetic algorithm, a tabu algorithm, a particle swarm algorithm, and a simulated annealing algorithm.

In some variations, the energy function may include a molecular mechanics function, a structural bioinformatics function, an amino acid sidechain packing function, and/or protein radius of gyration function.

In some variations, the sequence of the protein is further generated such that the protein exhibits a desired tertiary structure including one or more folds.

In another aspect, there is provided a non-transitory computer readable medium storing instructions that cause operations when executed by at least one data processor. The operations may include: querying a van der Mer (vdM) database to identify a first van der Mer known to interact with a first portion of a first compound, the first van der Mer corresponding to an in silico unit of protein structure that defines, based at least on a statistically preferred orientation of the first portion of the first compound relative to a backbone structure of a protein, a binding site for the first compound relative to the backbone structure of the protein; and generating, based at least on the first van der Mer, a sequence for the protein such that the protein exhibits a binding affinity for the first compound.

In another aspect, there is provided an apparatus that includes: means for querying a van der Mer (vdM) database to identify a first van der Mer known to interact with a first portion of a first compound, the first van der Mer corresponding to an in silico unit of protein structure that defines, based at least on a statistically preferred orientation of the first portion of the first compound relative to a backbone structure of a protein, a binding site for the first compound relative to the backbone structure of the protein; and means for generating, based at least on the first van der Mer, a sequence for the protein such that the protein exhibits a binding affinity for the first compound.

In another aspect is provided a computer-implemented method for identifying a protein capable of binding a compound, including identifying van der Mers representing the chemical groups of the compound and amino acid residues of the protein capable of interacting with the chemical groups of the compound in silico, and wherein the protein has secondary and tertiary protein structure when bound to the compound.

In an aspect is provided a computer-implemented method for identifying a protein capable of binding a compound, including:
(a) generating a first set of atomic protein coordinates representing a backbone structure of the protein;
(b) identifying a first van der Mer from a van der Mer database including a first set of atomic van der Mer coordinates representing a first chemical group of the compound, a first amino acid side chain and a first portion of a protein backbone bound to the first amino acid side chain, wherein the first chemical group interacts in silico with the first portion of a protein backbone or the first amino acid side chain;
(c) identifying portions of the backbone structure of the protein having atomic protein coordinates that are capable of overlapping with the atomic van der Mer coordinates of the first portion of a protein backbone of the first van der Mer;
(d) repeating steps (b) and (c) for at least one additional van der Mer independently representing an optionally different chemical group of the compound, an additional independent amino acid side chain and an additional independent portion of a protein backbone bound to the independent additional amino acid side chain;
(e) generating a set of atomic chemical coordinates representing the compound;
(f) generating at least one set of atomic coordinates of an in silico complex of the protein capable of binding the compound bound to the compound; wherein the in silico complex optimizes the overlap between the atomic chemical coordinates of the compound chemical groups and the atomic van der Mer coordinates representing the independent chemical groups of the compound identified in steps (b) to (d);
(g) generating a set of atomic amino acid coordinates for amino acid side chains and portions of protein backbone independently representing those portions of the backbone structure of the protein that are not overlapping with atomic van der Mer coordinates of the van der Mer including atomic van der Mer coordinates that are overlapping with the atomic chemical coordinates representing the compound of the in silico complex of step (f);
(h) based at least in part on steps (a) to (g), optimizing atomic coordinates of the compound and protein thereby identifying a protein capable of binding the compound.

In an aspect is provided a computer-implemented method for identifying a complex of a protein bound to a compound, including:
(a) generating a first set of atomic protein coordinates representing the side chain and backbone structure of the protein;
(b) identifying a first van der Mer from a van der Mer database including a first set of atomic van der Mer coordinates representing a first chemical group of the compound, a first amino acid side chain and a first portion of a protein backbone bound to the first amino acid side chain, wherein the first chemical group interacts in silico with the first portion of a protein backbone or the first amino acid side chain;
(c) identifying portions of the backbone structure of the protein having atomic protein coordinates that are capable of overlapping with the atomic van der Mer coordinates of the first portion of a protein backbone of the first van der Mer wherein the amino acid side chain of the van der Mer and the amino acid side chain directly attached to the overlapping portions of the backbone structure of the protein are the same side chain;
(d) repeating steps (b) and (c) for at least one additional van der Mer independently representing an optionally different chemical group of the compound, an additional independent amino acid side chain and an additional independent portion of a protein backbone bound to the independent additional amino acid side chain;
(e) generating a set of atomic chemical coordinates representing the compound;
(f) generating at least one set of atomic coordinates of an in silico complex of the protein capable of binding the compound bound to the compound; wherein the in silico complex optimizes the overlap between the atomic chemical coordinates of the compound chemical groups and the atomic van der Mer coordinates representing the independent chemical groups of the compound identified in steps (b) to (d);
(g) based at least in part on steps (a) to (f), optimizing atomic coordinates of the compound and protein thereby identifying a complex of a protein bound to a compound.

In an aspect is provided a computer-implemented method for identifying a protein capable of binding a compound, including:
(a) generating a first set of atomic protein coordinates representing a protein backbone structure;
(b) generating a first set of atomic chemical coordinates representing a first chemical group of the compound;
(c) identifying a first van der Mer from a van der Mer database including a first set of atomic van der Mer coordinates representing the first chemical group of the compound, a first amino acid side chain and a first portion of a protein backbone bound to the first amino acid side chain, wherein the first chemical group interacts in silico with the first portion of a protein backbone or the first amino acid side chain;
(d) generating a second set of atomic chemical coordinates representing a second chemical group of the compound;
(e) identifying a second van der Mer from the van der Mer database including a second set of atomic van der Mer coordinates representing the second chemical group of the compound, a second amino acid side chain and a second portion of a protein backbone bound to the second amino acid side chain, wherein the second chemical group interacts in silico with the second portion of a protein backbone or the second amino acid side chain;
(f) calculating an energetic stability of the protein backbone structure bound to the compound using the first set of atomic van der Mer coordinates and the second set of atomic van der Mer coordinates in silico;
(g) repeating steps (a) to (f) for additional van der Mers representing the first chemical group of the compound, a first amino acid side chain and a first portion of a protein backbone bound to the first amino acid side chain and additional van der Mers representing the second chemical group of the compound, a second amino acid side chain and a second portion of a protein backbone bound to the second amino acid side chain;
(h) generating a set of atomic amino acid coordinates for amino acid side chains and portions of protein backbone independently representing those portions of the protein backbone structure not represented by a van der Mer of steps (a) to (g);
(i) based at least in part on steps (a) to (h), optimizing atomic coordinates of the compound and protein thereby identifying a protein capable of binding the compound.

In an aspect is provided a computer-implemented method for identifying a protein capable of binding a compound, including:
(a) identifying a first van der Mer from a van der Mer database including atomic van der Mer coordinates of a chemical group of the compound, wherein the atomic van der Mer coordinates of the chemical group in the first van der Mer overlap with the atomic chemical coordinates of the chemical group of the compound;
(b) identifying a protein backbone for the protein wherein the atoms of the protein backbone are associated with a set of atomic protein coordinates;
(c) identifying an overlap between the atomic van der Mer coordinates of the amino acid backbone of the first van der Mer identified in step (a) and the atomic protein coordinates of an amino acid residue of the protein backbone identified in step (b);
(d) optionally repeating steps (a) to (c) for a different chemical group of the compound;
(e) identifying independent sets of van der Mer identified in steps (a) to (d) wherein all van der Mer of each independent set include atomic van der Mer coordinates that collectively simultaneously overlap atomic protein coordinates of the protein backbone identified in step (b);
(f) identifying at least one independent set of van der Mer identified in step (e) with a cluster score above a threshold;
(g) identifying an amino acid residue for each amino acid of the protein backbone identified in step (b) having atomic amino acid coordinates that are not overlapping with the atomic van der Mer coordinates of the set of van der Mer identified in step (f);
(h) optimizing atomic coordinates of the compound and protein;
(i) wherein the optimization is performed using at least an energy minimization calculation, and wherein the optimization is performed to energetically stabilize a complex of the compound and protein.

In an aspect is provided a computer-implemented method for identifying a protein capable of binding a compound, including:
(a) identifying covalently bonded amino acid backbone residues of the protein wherein each amino acid backbone residue atom is associated with a set of atomic protein coordinates;
(b) identifying an independent set of van der Mer associated with an amino acid backbone residue and a chemical group of the compound, wherein each van der Mer is associated with a set of atomic van der Mer coordinates for an amino acid and chemical group of the compound and the atomic van der Mer coordinates for the van der Mer amino acid backbone atoms of each independent set of van der Mer overlap with amino acid backbone residue atomic protein coordinates of the protein;
(c) identifying and removing from each independent set of van der Mer, any van der Mer wherein atomic van der Mer coordinates of a sidechain or chemical group of the van der Mer overlap with atomic protein coordinates of the covalently bonded amino acid backbone residues of the protein;
(d) identifying and removing any van der Mer wherein atomic van der Mer coordinates of the chemical group of the van der Mer is characterized as exposed to bulk solvent;
(e) identifying independent sets of atomic chemical coordinates of the compound wherein, the atomic chemical coordinates of the compound chemical group atoms of each independent set overlap with atomic van der Mer coordinates of chemical group atoms of van der Mer identified in steps (b) to (d) and atomic van der Mer coordinates of the van der Mer further include atomic van der Mer coordinates for amino acid backbone atoms that overlap with atomic protein coordinates of amino acid backbone atoms of the protein;
(f) identifying and sorting independent sets of atomic chemical coordinates of the compound of step (e) based on the value of the compound van der Mer cluster score;
(g) identifying a preferred amino acid for an amino acid residue position of the protein when the amino acid residue position of the protein has amino acid backbone atom atomic protein coordinates that overlap with the amino acid backbone atomic van der Mer coordinates of a van der Mer identified in step (f) and the preferred amino acid is the amino acid associated with the van der Mer;
(h) optimizing atomic coordinates of the compound and amino acid residues of the protein;
(i) wherein the optimization is performed using at least an energy minimization calculation, and wherein the optimization is performed to energetically stabilize the protein.

### BRIEF DESCRIPTION OF THE DRAWINGS

FIGS. 1A-1E. van der Mers (vdMs) are a structural unit relating chemical-group position to the protein backbone. A) Workflow of a traditional protein design strategy vs Convergent Motifs for Binding Sites (COMBS). B) Definition of a vdM. A chemical group is interacting if it is in van der Waals contact with the protein sidechain or mainchain. Like rotamers, vdMs are derived from a large set of high-quality protein crystal structures. A vdM of aspartic acid (Asp) and carboxamide (CONH₂) is shown. C) vdMs are φ/ψ- and rotamer-dependent, illustrated by the top vdMs of the m-30 rotamer of Asp, clustered by location of CONH₂ after exact superposition of mainchain N, Cα, and C atoms. D, E) We rank vdMs by prevalence in the PDB, quantified via a cluster score C (the natural logarithm of the ratio of the number of members in a cluster (N_{vdM}) to the average number of members in a cluster ((N_{vdM})). The seventh-largest cluster of Asp/CONH₂ vdMs is shown as an example in (D).

FIGS. 2A-2D. Prevalent vdMs describe the binding site of biotin in streptavidin. A, B) We construct vdMs of the polar chemical groups of biotin by searching the PDB for protein interactions with 1) backbone amide nitrogen (N—H); 2) backbone carbonyl or carbonyl from Asn/Gln sidechain (C═O), and 3) carboxylate of Asp/Glu sidechain (COO—). C) vdMs are sampled on the streptavidin backbone to generate possible locations for productive interactions with the chemical groups. Here, Asn and Ser vdMs of COO— are sampled at two positions of the backbone. D) vdMs with chemical groups that are nearest-neighbors (0.6 Å RMSD) to those of biotin in its binding site are overlaid on top of biotin.

FIGS. 3A-3H. Apixaban-binding helical bundle (ABLE) design strategy. A-F) Steps of the design process. A) We target simultaneous engagement of two carbonyls (C═O) and the carboxamide (CONH₂) of apixaban. B) We computationally generated a set of 32 designable 4-helix bundle folds based on a mathematical parameterization. C) vdM sampling of CONH₂ and C═O enumerates statistically preferred locations of these chemical groups relative to the backbone. D) We use a precomputed set of vdMs with apixaban superimposed by one of its chemical groups to position apixaban within the bundle, such that it is guaranteed to have at least one vdM that accommodates its position. Chemical groups of vdMs that overlap with those of apixaban are found by a nearest-neighbors look-up. Multiple vdMs contributing from one residue position are possible, e.g. His/C═O and Trp/C═O vdMs, and can be used in separate designs. E) Specific choices of vdMs for each chemical group of the ligand are made by maximizing the use of highly enriched vdMs in the binding site (high C score), see FIG. 1D-1E. Final ligand positions and interactions for the six experimentally characterized designs were chosen by maximizing both C and the burial of apolar surface area of apixaban. The vdMs chosen to comprise the binding site of ABLE are shown along with their cluster scores. F) The location of apixaban and its vdM-derived interactions with the protein are constrained in a subsequent flexible-backbone sequence design protocol. G) The electronic absorbance spectrum of apixaban is red-shifted upon binding to ABLE. The left spectrum shows apixaban (4 μM) in 50 mM NaPi, 100 mM NaCl, pH 7.4 buffer. The right spectrum is the difference of the absorbance spectrum of ABLE alone (20 μM) and the spectrum of ABLE (20 μM) with apixaban (4 μM). The spectra were normalized to the peak maximum for comparison. These experiments were facilitated by the high extinction coefficient of apixaban, and the lack of Trp in ABLE. H) Global fit of a single-site binding model to the absorbance changes at 305 nm upon titration of apixaban into 5, 10, and 20 μM solutions of ABLE. The dissociation constant (KD) from the fit is 5 (±1) μM, which was confirmed by fluorescence polarization competition experiments (see examples and additional figures).

FIGS. 4A-4D. The structure of apixaban-bound ABLE agrees with the design. A) Superposition of backbone Ca atoms of structure and design (gray, 0.7 Å RMSD), showing sidechains of amino acids in the protein core. B) ABLE's binding site from the structure (1.3 Å resolution), showing vdM-derived interactions with apixaban. The 2mFo-DFc composite omit map is contoured at 1.5-σ. The map was generated from a model that omitted coordinates of apixaban. Protein backbone of these residues is shown in cartoon format. C) Overlay of designed interactions, after the designed model was superimposed onto the Cα atoms of the structure. D) Fluorescence anisotropy competition experiments (485 nm excitation, 528 nm emission) show that ABLE binds apixaban specifically. The bound fluorophore (apixaban-peg-FITC, see examples and FIG. S9), is dislodged by addition of competing ligand. Anisotropy was converted to fraction bound via a one-site binding model (Examples). ABLE concentration was 20 μM and apixaban-peg-FITC concentration was 25 nM in 50 mM NaPi, 100 mM NaCl, pH 7.4 buffer. ApixabanCOO⁻ is identical to apixaban except that it contains a carboxylate instead of a carboxamide (circled). Rivaroxaban is another inhibitor that also binds tightly to factor Xa via the same binding mode as apixaban, but shows only very weak binding to ABLE. Fits to a competitive binding model are shown as smooth lines. K_{D} of rivaroxaban is 130 (±10) μM; apixbanCOO⁻ is 50 (±5) μM; apixaban is 7 (±2) μM.

FIGS. 5A-5I. Drug-free ABLE has a preorganized structure with an open binding site competent for binding. A) Slice through a surface representation of the 1.3 Å resolution structure of unliganded ABLE shows an open binding cavity. B) Same slice shown for the structure of apixaban-bound ABLE. C) The Cα atom backbone superposition of unliganded- and liganded ABLE. Colored squares surrounding the structure correspond to panels in G, H, and I, looking down from the top. D) The binding site of drug-free ABLE shows 9 buried, crystallographic waters (spheres, occupancy >0.9) involved in an extensive H-bonded network with binding site residues Tyr6, Gln14, Tyr46, and His49. 2mFo-DFc electron density map of drug-free ABLE is contoured at 1 σ. An acetate (Act) group from the crystallization condition H-bonds with His49. His49 and Tyr46 are observed with alternate rotamers. E) Same view as in D but with the addition of the corresponding residues from the apixaban-bound structure, after an all-Ca-atom backbone superposition. The 1-σ 2mFo-DFc electron density of apixaban from the drug-bound structure shows where the crystallographic waters bind in the ligand-free structure relative to the bound structure. A water (shown as a sphere) mediates the H-bond between Tyr46 and apixaban. This water is not observed in the unliganded structure. F) Binding of apixaban in the drug-bound structure displaces all of the 9 buried waters in the drug-free structure. Stick renderings, as well as the surface background, shows the binding site of the ABLE-apixaban complex. G, H) Binding site overlay of liganded and unliganded ABLE shows preorganized rotamers. I) The remote folding core contains identical rotamers in drug-free and drug-bound ABLE, predisposing the drug-free protein for binding.

FIGS. 6A-6D. Design inferences from the structure and function of ABLE. A) Exact sidechain positioning is not necessary for precise placement of ligand chemical groups relative to the mainchain. The placement of the C═O chemical group of apixaban relative to the backbone of residue 49 is exact (0.25 Å RMSD). The His49/C═O vdM from the design (also see FIG. 3E) was superimposed onto His49 of the drug-bound ABLE structure via backbone atoms (N, Cα, C atoms, spheres). This backbone superposition places the C═O group of the original vdM precisely (0.25 Å RMSD) onto that of apixaban in the structure. The cluster describing the His/C═O vdM, shown beneath, contains multiple rotamers of His that achieve the same placement of C═O relative to the position of the backbone. The rotamers of His49 in the structure and His from the original vdM are both observed in the cluster. B) Flexible-backbone sequence design (see FIG. 3F) resulted in recruitment of two additional polar interactions with apixaban from Tyr6 and Tyr46. A Tyr6/CONH₂ vdM is prevalent in the PDB whereas the Tyr/C═O interaction is not found in the database. C) A water mediates an H-bond between Tyr46 and the C═O group of apixaban. Thr122 H-bonds the C═O of the helix backbone at residue 108. D) Relative binding affinities of ABLE mutants with apixaban-peg-FITC fluorophore via fluorescence anisotropy experiments (see Examples and FIG. S18).

FIG. 7 depicts a system diagram illustrating an example of a protein design system, in accordance with some example embodiments.

FIG. 8 depicts a flowchart illustrating an example of a process for protein design, in accordance with some example embodiments.

FIG. 9 depicts a block diagram illustrating an example of a computing system, in accordance with some example embodiments.

FIGS. S1A-S1D. van der Mers (vdMs) have similar sidechain dihedral-angle distributions as traditional rotamers. A) The five most prevalent His/C═O vdMs, after superimposing and clustering by N, Cα, C atoms of His mainchain and C═O atoms of the chemical group. The top vdM (cluster 1) makes predominantly mainchain H-bonds to C═O, with some multivalent His sidechain H-bonds. Clusters 2, 3, 4, and 5 H-bond to C═O predominantly via His sidechain. The dihedral angles of the His sidechain for clusters 2 through 5 are plotted in B, and are color-coded according to the cluster label in A. C) Sidechain dihedral angles of each His/C═O vdM with His sidechain H-bonding to C═O. A total of 2,692 His/C═O vdMs are plotted. D) Sidechain dihedral angles of each His in the COMBS protein database. A total of 51,033 His rotamers are plotted. The X1 and X2 distributions are similar between C and D.

FIGS. S2A-S2D. Using vdMs for sampling and scoring. A, B) Representative vdMs are made for sampling onto backbone coordinates by aligning all vdMs by mainchain atoms and then tightly clustering (0.1 Å heavy atom RMSD) by coordinates of the sidechain and chemical group. C, D) vdMs are scored by grouping them based on a different criterion than what is used for sampling. We score vdMs by pairwise superimposing them via mainchain and chemical group coordinates (sidechain coordinates are not considered) and clustering by heavy atom RMSD (0.5 Å) of these coordinates. This reduces lever-arm effects that occur when small movements of backbone sweep out a large solid angle causing large movement of a distant chemical group. For example, (A) and (C) show the same vdMs but superimposed only by mainchain in (A) while superimposed by both mainchain and chemical group in (C). D) The cluster score C quantifies the prevalence of a vdM in the PDB. For example, cluster 7 with 164 members has a higher C score than cluster 60 with 40 members. We use representatives in (A, B) for sampling vdMs on a protein backbone but keep track of their C scores from the alternative clustering performed in (C, D) for ranking binding sites later in the design process.

FIG. S3. The conformational space of observed interaction geometries is largely captured by a small number of vdM clusters. H-bonding vdMs of Asp with carboxamide (CONH₂) were clustered by root mean squared deviation (R.M.S.D. less than 0.5 Å) of coordinates of backbone (N, Cα, C) and chemical-group heavy atoms, after all-by-all pairwise superposition of vdMs by these same coordinates. The plot shows the number of clusters needed to account for the total percent of observed H-bonded Asp/CONH₂ vdMs. Blue line indicates the point at which half of the vdMs are found in clusters. The inset shows the 7th largest vdM cluster, which is mainly comprised of alpha-helical residues.

FIG. S4. The conformational space of observed interaction geometries is largely captured by a small number of vdM clusters. H-bonding vdMs of CONH₂ for each amino acid type were clustered by root mean squared deviation (R.M.S.D. less than 0.5 Å) of coordinates of backbone (N, Cα, C) and chemical-group heavy atoms, after all-by-all pairwise superposition of vdMs by these same coordinates. The plots show the number of clusters needed to account for the total percent of observed H-bonded amino acid/carboxamide (CONH₂) vdMs. Left rectangular lines indicate the point at which half of the vdMs are found in clusters. Right rectangular lines indicate the point at which the clusters to the right are singletons. Insets list the total number of H-bonded vdM members (v) and the total number of clusters (c) for that amino acid/CONH₂ vdM type. Hydrophobic amino acids H-bond with CONH₂ groups primarily through main-chain interactions, but aromatic H-bonds (e.g. N—H of CONH₂ with phenyl of Phe) are also included.

FIG. S5. Convergent Motifs for Binding Sites (COMBS) design protocol.

FIGS. S6A-S6B. Apixaban-bound factor Xa structure. A) Overview of the factor Xa structure, with apixaban bound in the active site. B) View of the inhibitor binding site. Apixaban makes H-bonds (dashes) with backbone amides in loops, as well as a water-mediated H-bond to a backbone amide.

FIGS. S7A-S7C. Apixaban conformers. A) Conformers searched with COMBS for design of Apixaban-binding proteins. Binding sites of conformers 2, 3, and 4 scored lower (less favorably) than binding sites using the apixaban conformer from the factor Xa complex, so designs of these conformers were not tested experimentally. B) Small-molecule crystal structures of apixaban from the Cambridge Structural Database show conformers similar to that in factor Xa and ABLE. C) DFT (B3LYP/6-31G*)-optimized geometry of apixaban and two apixaban conformers found in ABLE crystal structure.

FIG. S8. Computational models of designed proteins. Overview and close-up of binding sites of the 6 proteins designed via the COMBS strategy. Apixaban is shown. All sidechains within an 8 Å radius of apixaban are shown. Note the different topologies, bundle lengths, binding modes and binding residues. ABLE and LABLE share most of the same vdM-derived binding residues and the same binding position of apixaban, but differ in topology, size, and overall sequence (22% sequence homology).

FIG. S9. Circular dichroism spectra of the designed proteins show that all are helical. Spectra were collected at room temperature in 50 mM NaPi, 100 mM NaCl, pH 7.4 buffer.

FIG. S10. Nuclear magnetic resonance spectroscopy shows ABLE and LABLE bind apixaban. 1-dimensional ¹H spectra of LABLE, ABLE, and other designs (150 μM) with (dark gray) and without (light gray) 1 equivalent of apixaban. The 1D-spectra of LABLE and ABLE are well-dispersed and show clear differences in chemical shift upon addition of apixaban. The remainder of the designs showed no change of chemical shift upon addition of apixaban or did not display well-dispersed chemical shifts. Design 4 showed broad peaks in the methyl region, indicative of a molten globule, and was not tested for binding. 1-dimensional ¹H spectra were recorded on Bruker 800 MHz spectrometer. Buffer for all experiments was 50 mM NaPi, 100 mM NaCl, pH 7.4 with 5% d₆-dimethylsulfoxide.

FIGS. S11A-S11D. Spectral titration of apixaban into a solution of ABLE shows low-μM binding. A-C) Absorbance at 305 nm was monitored for several concentrations of ABLE ([ABLE]_{T}=20, 10, and 5 uM) as a function of total apixaban concentration ([Apx]_{T}). The smooth solid line without circles is an extrapolation of a linear fit to the first 3 datapoints for low [Apx]_{T}. Deviation from the line is evidence of binding (approaching saturation). The data in A-C (circles) were globally fit to a single-site binding model (see Examples), and the results of the fit are shown with lines (A-C). D) The electronic absorbance spectrum of apixaban is red-shifted upon binding to ABLE. The left spectrum shows apixaban (4 μM) in 50 mM NaPi, 100 mM NaCl, pH 7.4 buffer. The right spectrum is the difference of the absorbance spectrum of ABLE alone (20 μM) and the spectrum of ABLE (20 μM) with apixaban (4 μM). The spectra were normalized to the peak maximum for comparison. E) Global fit of a single-site binding model to the absorbance changes at 305 nm upon titration of apixaban into 5, 10, and 20 μM solutions of ABLE. The data points shown are transformations of the raw data in A-C. The first two terms in Equation 1 were subtracted from the raw data to isolate the contribution from the bound complex. Solid lines show the result of the global fit. The parameters from the fit (see Equation 1) were Δε (305 nm)=3900 (±420) M⁻¹ cm⁻¹, K_{D}=5 (±1) μM, N=1.4 (±0.07), and ε (305 nm)=9570 (±28) M⁻¹ cm⁻¹ [20 μM ABLE], 9530 (±53) M⁻¹ cm⁻¹ [10 μM ABLE], 9680 (±93) M⁻¹ cm⁻¹ [5 μM ABLE]. Errors are the standard deviation of the fitted parameters. The experiment was repeated three times with new samples.

FIGS. S12A-S12D. Fluorescence anisotropy binding experiments of ABLE and LABLE with apixaban. A) Apx-peg-FITC was used for fluorescence anisotropy experiments to assess binding of apixaban to ABLE and LABLE. B) Anisotropy data was converted to fraction bound (circles) of Apx-peg-FITC to ABLE or LABLE and fit to a single-site binding model (solid lines). K_{D} of LABLE to Apx-peg-FITC was found to be 5.3 (±0.2) μM and that of ABLE to Apx-peg-FITC was 18.8 (±1.3) μM. C and D) Competition experiments with apixaban show that ABLE and LABLE bind apixaban with K_{D} of 8.1 (±1.2) μM and 0.57 (±0.07) μM, respectively. Errors are the standard deviation of the fitted parameters. The experiments were repeated at least twice.

FIG. S13. ¹H-¹⁵N HSQC spectrum of 400 μM LABLE with (dark gray) and without (light gray) 1 equivalent of apixaban. The 2D-spectrum of LABLE is well-dispersed and show clear differences in chemical shift upon additional of apixaban. 2-dimensional ¹H-¹⁵N HSQC spectra were recorded on Bruker 800 MHz spectrometer. Buffer was 50 mM NaPi, 100 mM NaCl, pH 7.4 with 5% d₆-dimethylsulfoxide.

FIG. S14. Analytical gel filtration analysis of apo- and holo-ABLE shows a monomeric protein. Samples were run on a Superdex 75 5/150 column concentrations of 140 μM and 75 μM for apo and holo, respectively, in 50 mM NaPi, 100 mM NaCl, pH 7.4 buffer. The peak near 2.7 ml elution volume in holo-ABLE is attributed to both DMSO and unbound (excess) apixaban, which was added in excess (200 uM). The 6×His-tag and TEV cleavage linker on the N-terminus add to the MW of the protein.

FIGS. S15A-S15B. Apo- and holo-ABLE are thermostable. Circular dichroism signal at 222 nm for various temperatures shows that apo- and holo-ABLE have melting temperatures >95° C. Markers show the average CD signal with error bars denoting the standard deviation of two sequential experiments. B) is the same as (A) but scaled to a smaller region of the plot.

FIGS. S16A-S16B. Crystallographic asymmetric unit of apixaban-bound ABLE. A) Two highly similar monomers were found in the asymmetric unit (0.64 Å Cα RMSD). B) Superimposed conformations of apixaban in the two subunits largely agree, differing by rotation around the methoxy-phenyl bond and the terminal oxopiperidine.

FIG. S17. DFT-optimized structure of apixaban (light gray) and structure from drug-bound ABLE (dark gray). The conformation of apixaban relaxes slightly in drug-bound ABLE relative to its initial starting geometry, which was taken from the co-crystal structure of factor Xa (PDB 2p16).

FIG. S18. Anisotropy data of single-site mutants of ABLE. Anisotropy data was converted to fraction bound (markers) of Apx-peg-FITC to ABLE or LABLE and fit to a single-site binding model (solid lines). Dissociation constants (K_{D}) to Apx-peg-FITC are: 17.0 (±0.6) μM for T112A, 36.5 (±1.3) μM for Y46F, 60 (±3) μM for H49A, 76 (±3) μM for Q14A, 102 (±4) μM for Y46A, 137 (±6) μM for Y6F, 230 (±10) μM for Y6A. Errors are the standard deviation of the fitted parameters.

FIGS. S19A-S19C. Structure of H49A mutant of ABLE. A) The 1.6 Å resolution structure of the H49A mutant of ABLE is superimposed on that of apo-ABLE via Cα coordinates of residues 1-110 (0.9 Å Cα RMSD). B) The drug-free binding site of the H49A mutant shows a water-filled pocket with several relaxed rotamers, e.g. L53 and M72. C) Comparison of H49A mutant with apo-ABLE shows that the absence of H49A allows L53 to adopt its preferred rotamer, which in turn allows methionine M72 to adopts its preferred rotamer. Phenylalanine F75 allows for space of the L53 rotamer by abutting the C-terminal helix, which kinks near residue 113 to avoid sterically clashing with F75. Waters from unliganded ABLE are shown as dark gray spheres, and waters from H49A mutant are shown as light gray spheres.

FIGS. S20A-S20C. Ab initio folding of designed sequences. A and B) The ABLE and LABLE design models are accurately predicted by the lowest energy, lowest RMSD (<2 Å) ab initio models. C) Other designs were predicted either not to fold or showed collapsed binding sites with higher RMSD (>2 Å).

TABLE S1 lists crick parameters for generation of parametric 4-helix bundle ensemble.

TABLE S2 lists PDB accession codes and Cα RMSD values of matches to a 4-helix query (10-residues each helix) of the initial parameterized backbone of ABLE.

TABLE S3 lists data collection and refinement statistics of drug-free- and drug-bound ABLE.

TABLE S4 lists data collection and refinement statistics of H49A mutant of unliganded ABLE.

### DETAILED DESCRIPTION

### I. Definitions

The terms “a” or “an,” as used in herein means one or more. In addition, the phrase “substituted with a[n]” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

An amino acid residue in a protein “corresponds” to a given residue when it occupies the same essential structural position within the protein as the given residue.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid. The terms “non-naturally occurring amino acid” and “unnatural amino acid” refer to amino acid analogs, synthetic amino acids, and amino acid mimetics, which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety. In embodiments, the protein includes at least 30 amino acid residues. A protein may be characterized as having a protein backbone. A “protein backbone” is used herein in accordance with its ordinary meaning and refers to the polymer of amino acid residues that create a continuous chain. For example, a protein backbone may refer to the series of amino acid residues covalently linked together, e.g.,

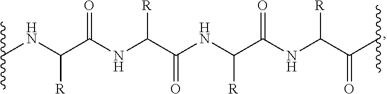

wherein each R independently represents optionally different amino acid side chains. In embodiments, the protein backbone includes core amino acid residues and ligand binding amino acid residues. In embodiments, the protein backbone includes core amino acid residues. In embodiments, the protein backbone includes ligand binding amino acid residues.

The term “amino acid side chain” refers to the functional substituent contained on amino acids. For example, an amino acid side chain may be the side chain of a naturally occurring amino acid. Naturally occurring amino acids are those encoded by the genetic code (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine), as well as those amino acids that are later modified, e.g., hydroxyproline, γ-carboxyglutamate, and O-phosphoserine. In embodiments, the amino acid side chain may be a non-natural amino acid side chain. In embodiments, the amino acid side chain is

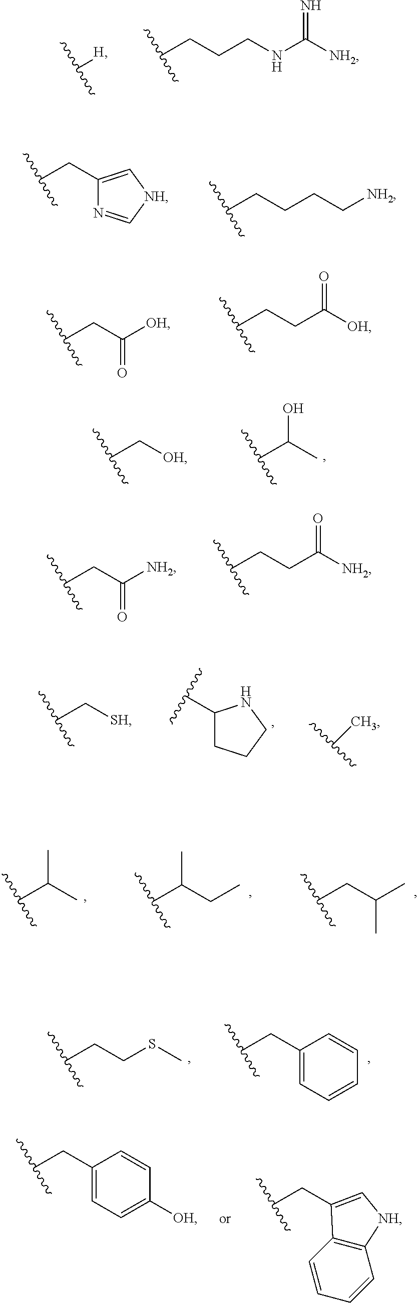

wherein the symbol “ [symbol] ” corresponds to the attachment of a chemical moiety (e.g., side chain) to the remainder of a molecule or chemical formula (e.g., the amino acid core, or

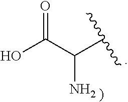 [SMILES: *C(N)C(=O)O]

The term “non-natural amino acid side chain” refers to the functional substituent of compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium, allylalanine, 2-aminoisobutryric acid. Non-natural amino acids are non-proteinogenic amino acids that either occur naturally or are chemically synthesized. Such analogs have modified R groups (e.g., norleucine) or modified peptide backbones, but retain the same basic chemical structure as a naturally occurring amino acid. Non-limiting examples include exo-cis-3-aminobicyclo[2.2.1]hept-5-ene-2-carboxylic acid hydrochloride, cis-2-aminocycloheptanecarboxylic acid hydrochloride,cis-6-amino-3-cyclohexene-1-carboxylic acid hydrochloride, cis-2-amino-2-methylcyclohexanecarboxylic acid hydrochloride, cis-2-amino-2-methylcyclopentanecarboxylic acid hydrochloride, 2-(Boc-aminomethyl)benzoic acid, 2-(Boc-amino)octanedioic acid, Boc-4,5-dehydro-Leu-OH (dicyclohexylammonium), Boc-4-(Fmoc-amino)-L-phenylalanine, Boc-β-Homopyr-OH, Boc-(2-indanyl)-Gly-OH, 4-Boc-3-morpholineacetic acid, 4-Boc-3-morpholineacetic acid, Boc-pentafluoro-D-phenylalanine, Boc-pentafluoro-L-phenylalanine, Boc-Phe(2-Br)—OH, Boc-Phe(4-Br)—OH, Boc-D-Phe(4-Br)—OH, Boc-D-Phe(3-Cl)—OH, Boc-Phe(4-NH2)-OH, Boc-Phe(3-NO2)-OH, Boc-Phe(3,5-F2)-OH, 2-(4-Boc-piperazino)-2-(3,4-dimethoxyphenyl)acetic acid purum, 2-(4-Boc-piperazino)-2-(2-fluorophenyl)acetic acid purum, 2-(4-Boc-piperazino)-2-(3-fluorophenyl)acetic acid purum, 2-(4-Boc-piperazino)-2-(4-fluorophenyl)acetic acid purum, 2-(4-Boc-piperazino)-2-(4-methoxyphenyl)acetic acid purum, 2-(4-Boc-piperazino)-2-phenylacetic acid purum, 2-(4-Boc-piperazino)-2-(3-pyridyl)acetic acid purum, 2-(4-Boc-piperazino)-2-[4-(trifluoromethyl)phenyl]acetic acid purum, Boc-β-(2-quinolyl)-Ala-OH, N—Boc-1,2,3,6-tetrahydro-2-pyridinecarboxylic acid, Boc-β-(4-thiazolyl)-Ala-OH, Boc-β-(2-thienyl)-D-Ala-OH, Fmoc-N-(4-Boc-aminobutyl)-Gly-OH, Fmoc-N-(2-Boc-aminoethyl)-Gly-OH, Fmoc-N-(2,4-dimethoxybenzyl)-Gly-OH, Fmoc-(2-indanyl)-Gly-OH, Fmoc-pentafluoro-L-phenylalanine, Fmoc-Pen(Trt)-OH, Fmoc-Phe(2-Br)—OH, Fmoc-Phe(4-Br)—OH, Fmoc-Phe(3,5-F2)-OH, Fmoc-β-(4-thiazolyl)-Ala-OH, Fmoc-β-(2-thienyl)-Ala-OH, 4-(hydroxymethyl)-D-phenylalanine.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about means the specified value.

The terms “bind” and “bound” as used herein is used in accordance with its plain and ordinary meaning and refers to the association between atoms or molecules. The association can be direct or indirect. For example, bound atoms or molecules may be direct, e.g., by covalent bond or linker (e.g. a first linker or second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi or hydrophobic effects), hydrophobic interactions and the like).

The term “compound” refers to a substance formed when two or more chemical elements are chemically bonded (e.g., covalent, ionic, etc.) together (e.g., small molecule, biomolecule, agonist, antagonist, protein). In embodiments, the compound is capable of binding to a protein (e.g., a protein described herein). In embodiments, a compound binds (e.g., covalently or non-covalently) to a protein. Typically, upon binding the compound has an effect on the protein (e.g., structural change of the protein, modulation of signaling pathways). A compound is associated with a set of compound atomic coordinates (e.g., Cartesian coordinates, internal coordinates, polar coordinates, spherical coordinates) which define the compound in space (e.g., Euclidean space). The compound may be endogenous or exogenous. Non-limiting examples of compounds include a catalyst, detectable agent, therapeutic agent, biological agent, cytotoxic agent, diagnostic agent, theranostic (e.g., a combined therapeutic and diagnostic agent), photodynamic therapy (PDT) agent, porphyrin, porphycene, rubyrin, rosarin, hexaphyrin, sapphyrin, chlorophyll, chlorin, phthalocyanine, porphyrazine, corrole, N-confused porphyrin, bacteriochlorophyll, pheophytin, texaphyrin, or related macrocyclic-based component that is capable of binding a metal ion. In embodiments, the compound is a peptide (e.g., 2 to 30 amino acid residues), a protein (e.g., greater than 30 amino acid residues), a small molecule (e.g., a compound with a molecular weight of less than 2000 Daltons), or a small molecule-metal-ion complex (e.g., a metalloporphyrin). In embodiments, the compound is endogenous. In embodiments, the compound is exogenous. In embodiments, the compound is a chemical molecule having a molecular weight of less than 10000 Daltons (e.g., less than 9000, 8000, 7000, 6000, 5000, 4000, 3000, 2000, 1000, 900, 800, 700, 600, 500, 400, 300, 200, or 100).

The term “ligand” refers to an agent (e.g., compound, metal, ion, biomolecule, agonist, antagonist) which is capable of binding to a protein (e.g., a protein described herein). In embodiments, a ligand refers to an agent (e.g., compound, metal, ion, biomolecule) which binds (e.g., covalently or non-covalently) to a protein. Typically, upon binding the ligand has an effect on the protein (e.g., structural change of the protein, modulation of signaling pathways). A ligand is associated with a set of ligand atomic coordinates (e.g., Cartesian coordinates, internal coordinates, polar coordinates, spherical coordinates) which define the ligand in space (e.g., Euclidean space). The ligand may be endogenous or exogenous. Non-limiting examples of ligands include a catalyst, detectable agent, therapeutic agent, biological agent, cytotoxic agent, magnetic resonance imaging (MRI) agent, positron emission tomography (PET) agent, radiological imaging agent, diagnostic agent, theranostic (e.g., a combined therapeutic and diagnostic agent), photodynamic therapy (PDT) agent, porphyrin, porphycene, rubyrin, rosarin, hexaphyrin, sapphyrin, chlorophyll, chlorin, phthalocyanine, porphyrazine, corrole, N-confused porphyrin, bacteriochlorophyll, pheophytin, texaphyrin, or related macrocyclic-based component that is capable of binding a metal ion. In embodiments, the ligand is a peptide (e.g., 2 to 30 amino acid residues), a protein (e.g., greater than 30 amino acid residues), a small molecule (e.g., a compound with a molecular weight of less than 2000 Daltons), or a small molecule-metal-ion complex (e.g., a metalloporphyrin). In embodiments, the ligand is endogenous. In embodiments, the ligand is exogenous. In embodiments, the ligand is a chemical molecule having a molecular weight of less than 10000 Daltons (e.g., less than 9000, 8000, 7000, 6000, 5000, 4000, 3000, 2000, 1000, 900, 800, 700, 600, 500, 400, 300, 200, or 100). In embodiments, the ligand is a compound.

The terms “optimizing” and “optimization” are used in accordance with their ordinary meaning in mathematics and computer science and refers to identifying a favorable outcome subject to certain criteria (e.g., constraints) from a set of available possibilities. Optimizing may employ iterative or heuristic algorithms, such as simplex algorithm, memetic algorithm, differential evolution algorithm, evolutionary algorithm, genetic algorithm, tabu algorithm, particle swarm algorithm, stimulated annealing algorithm, Monte Carlo sampling algorithm, dead-end elimination algorithm, branch and bound algorithm, or a pruning algorithm. For example, optimizing typically includes evaluating an energy function (e.g., force field model) and finding the minimum (e.g., global minimum or local minimum). Optimizing may include repeated evaluations of the energy function and may include fixing an atomic coordinate (e.g., fixing an atomic coordinate of at least one ligand binding amino acid residue atomic coordinate), introducing additional amino acid residues into the set of amino acid residues (e.g., the set of ligand binding amino acid residues), restricting the introduction of additional amino acid residues into the set of amino acid residues (e.g., the set of ligand binding amino acid residues), or a geometric transformation (e.g., translation or rotation) of an amino acid residue atomic coordinate (e.g., the atomic coordinate of the ligand binding amino acid residue atomic coordinates). The output of an optimization process may provide a set of compound binding amino acid residues and a corresponding set of compound binding amino acid residue atomic coordinates, and a set of core amino acid residues and a corresponding set of core amino acid residue atomic coordinates, which corresponds to an energetically stabilized protein. In embodiments the outcome of the optimization is the global minimum (e.g., the most energetically stabilized protein). In embodiments the outcome of the optimization is a local minimum (e.g., a minimum energy given the domain). In embodiments the optimization is complete when the derivative of the energy with respect to the position of the atoms, ∂E/∂r, is zero and the Hessian matrix has positive eigenvalues. In embodiments, optimizing includes a plurality of minimization calculations. In embodiments the optimization is a finite number of iterations.

An energy minimization calculation refers to the process of evaluating the energy as a function of the atomic coordinates, V(r). The energy function may include intra- and intermolecular energy terms within the system (e.g., protein) which may be written as Vₜₒₜₐₗ(r)=V_{bonds}(r)+V_{angles}(r) V_{dihedral}(r) Vᵢₘₚᵣₒₚₑᵣ(r) V_{nonbonding}(r) V_{electrostatics}(r); where Vₜₒₜₐₗ(r) corresponds to the total energy as a function of the atomic positions; V_{bonds}(r) corresponds to the energy contribution from bonded atoms, V_{angles}(r) corresponds to the energy contribution from angles; V_{dihedral}(r) corresponds to the energy contribution from dihedral torsions; Vᵢₘₚᵣₒₚₑᵣ(r) corresponds to the energy contribution from out-of-plane torsions; V_{nonbonding}(r) corresponds to the energy contribution from nonbonding interactions; and V_{electrostatics}(r) corresponds to the energy contribution from electrostatic interactions. Additional energy function terms may also be included in the total energy function, Vₜₒₜₐₗ(r), for example additional functions from molecular mechanics, functions from structural bioinformatics (log-odds scores), amino acid sidechain packing functions (e.g., functions and algorithms which vary the identity and rotamer of an amino acid side chain), protein radius of gyration functions, or a penalty function.

The term biomolecule as used herein refers to a molecule present in living organisms (e.g., proteins, carbohydrates, lipids, and nucleic acids, metabolites) and may be endogenous or exogenous in origin.

The term “energetically stabilized protein” is used in accordance with its ordinary meaning in the art, and is understood to refer to a protein which is structurally and thermodynamically stable relative to the protein that has not been energetically stabilized. For example, an energetically stabilized protein is determined to be energetically stabilized by determining the difference in the Gibbs free energy between the folded and unfolded states of the protein, also referred to herein as ΔG_{folding}. An energetically stabilized protein may be characterized by a well-dispersed NMR spectrum and/or the presence of a significantly folded core. In embodiments, the energetically stabilized protein is an enzyme. In embodiments, the energetically stabilized protein is an apo protein (e.g., a protein that is not bound to a ligand). In embodiments, the energetically stabilized protein is a holo protein (e.g., a protein that is bound to a ligand). In embodiments, the energetically stabilized protein is an apo protein which is capable of becoming a holo protein upon ligand binding. In embodiments, an energetically stabilized protein refers to a protein which is capable of performing a function (e.g., modulating a signal pathway). In embodiments, the energetically stabilized protein resists side-reactions such as aggregation and proteolysis. In embodiments, the energetically stabilized protein has a ΔG_{folding} of about −5 to about −40 kcal/mol in standard physiological conditions (e.g., temperature range of 20-40 degrees Celsius, atmospheric pressure of 1, pH of 6-8, glucose concentration of 1-20 mM, atmospheric oxygen concentration).

The term “small molecule” or the like as used herein refers, unless indicated otherwise, to a molecule having a molecular weight of less than about 700 Dalton, e.g., less than about 700, 650, 600, 550, 500, 450, 400, 350, 300, 250, 200, 100, or 50 Dalton.

In this disclosure, “comprises,” “comprising,” “containing” and “having” and the like can have the meaning ascribed to them in U.S. Patent law and can mean “includes,” “including,” and the like. “Consisting essentially of or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

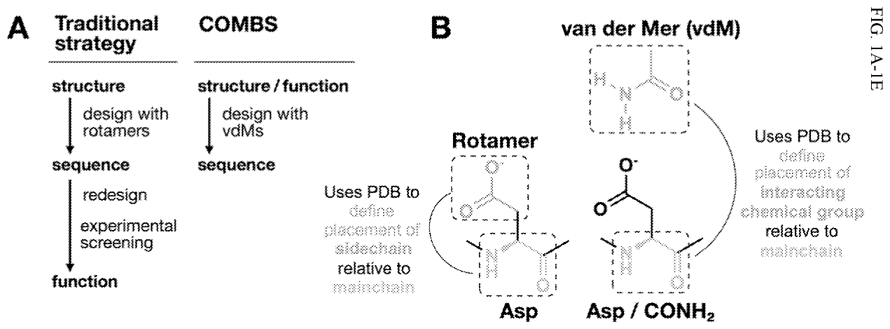

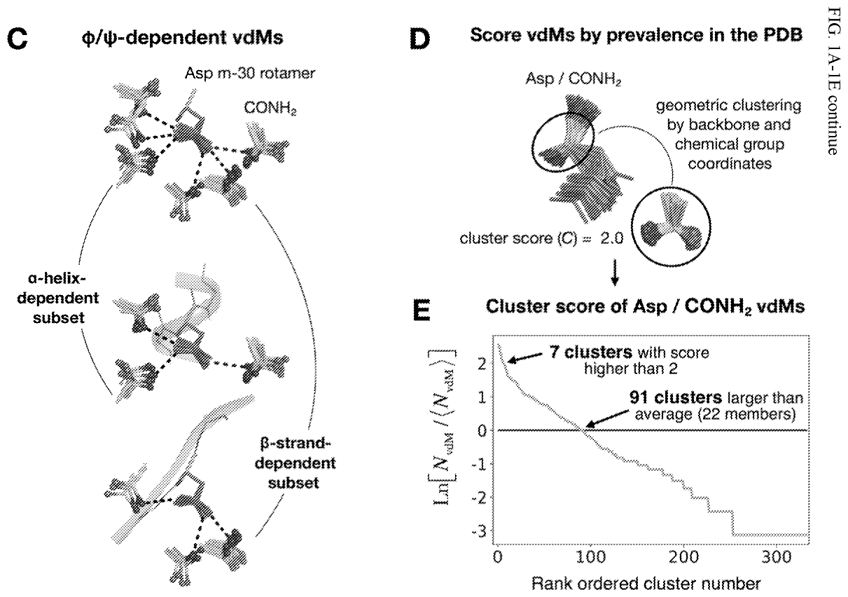

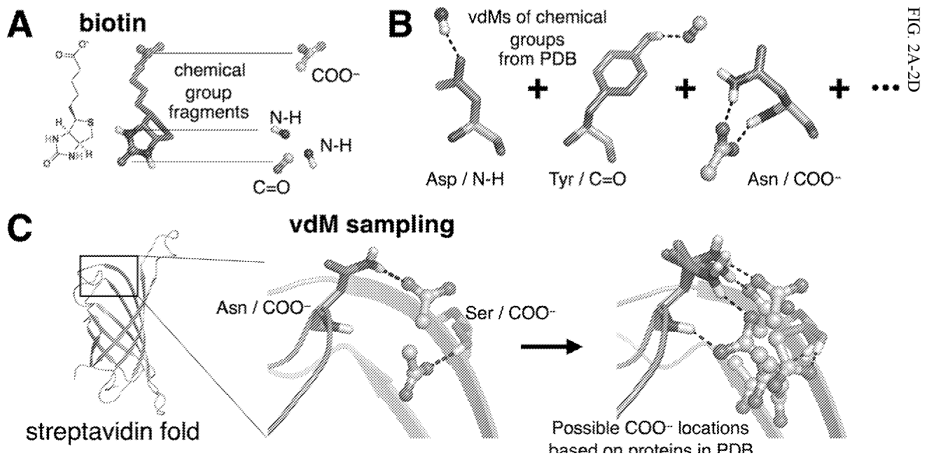

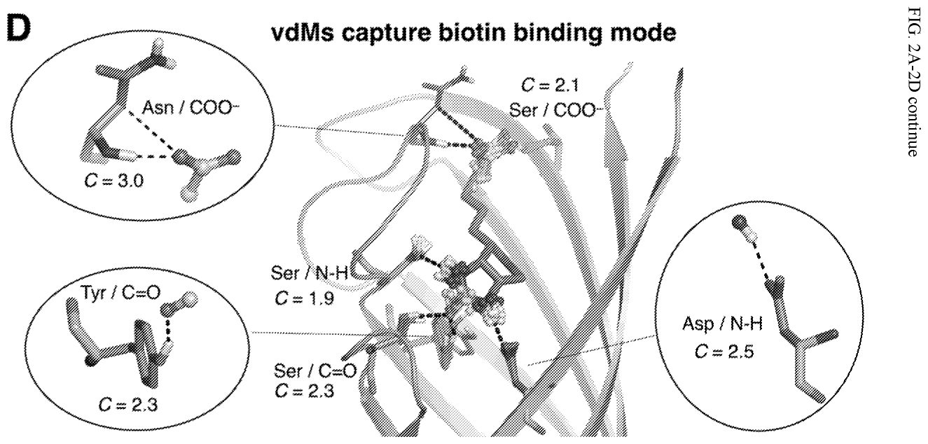

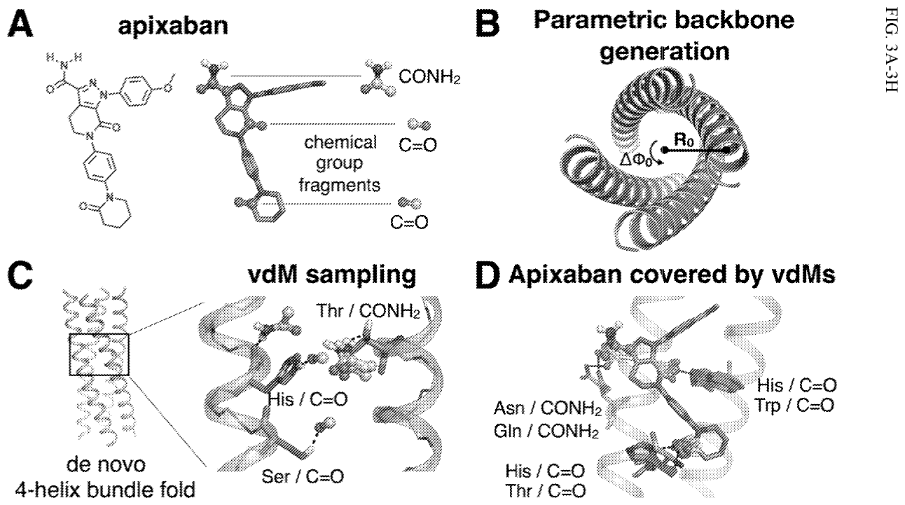

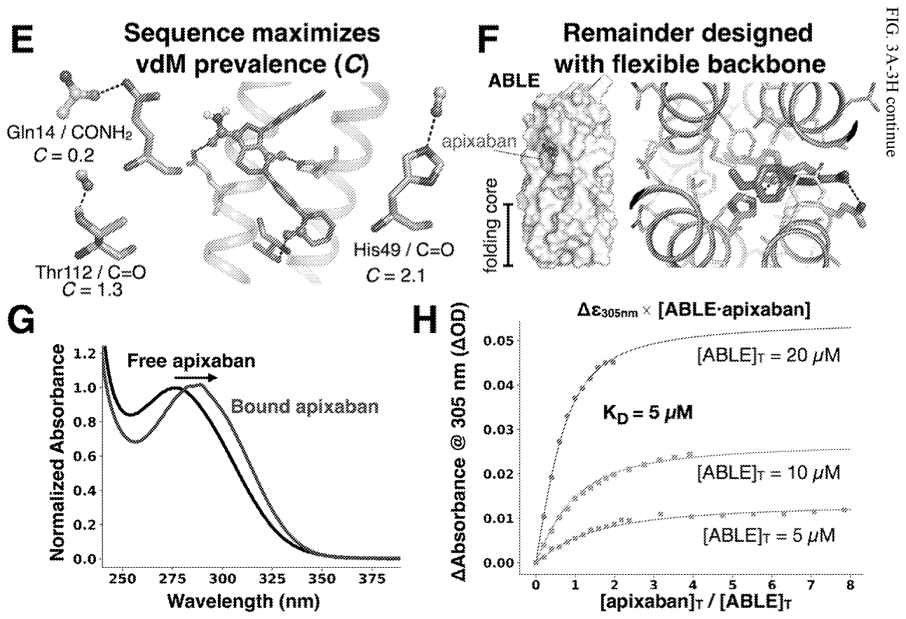

The term “van der Mer” as used herein refers to an in silico unit of protein structure interacting with a portion of a compound. In embodiments, the van der Mer may be used to map the backbone of an amino acid type to a statistically preferred position when interacting with specific chemical groups. A van der Mer may include a unit of local protein structure that directly links a tertiary structure to key interactions that engender tight and specific binding and defines the placement of key chemical groups in the ligand (compound) relative to the backbone atoms of the contacting amino acid residue (see for example FIG. 1B). In embodiments, van der Mers (vdMs) are culled from a non-redundant set of protein structures by 1) identifying all residues of a certain type that interact with a particular chemical group, 2) performing an all-by-all pairwise superposition of only the backbone and chemical-group atomic coordinates (sidechains are not considered in the superposition, allowing variation in their conformation), and 3) geometric clustering (e.g., with a tight RMSD cutoff (0.5 Å)). In embodiments, single clusters may contain multiple rotamers (see for example FIGS. 1D, 6A, and FIGS. S1A-S1D), because sidechain coordinates are not explicitly considered in clustering. In embodiments, vdMs sample locations of chemical groups relative to the backbone that have been experimentally vetted to achieve binding, regardless of ideality of the interaction. In embodiments, vdMs also implicitly consider interactions with ordered or bulk water, which might influence their interaction geometries. In embodiments, vdMs may derive from contacts with either mainchain, sidechain or both in a multivalent interaction. In embodiments, a vdM is a cluster of interactions of a specific chemical group and a specific amino acid having a 0.5 Å RMSD cutoff. In embodiments, the individual members of the vdM is called a van der Mer cluster member. In embodiments, a vdM representative is a sub-cluster of a van der Mer having a tighter RMSD cutoff than the van der Mer from which it derives (e.g., 0.1 Å). In embodiments, vdM representatives are used for sampling. In embodiments, vdM representatives are identified by aligning the vdMs exactly by backbone atoms (N, Cα, C), and tightly clustered them (e.g., using a greedy clustering algorithm) by sidechain and CG coordinates (all-heavy-atom RMSD of 0.1 Å). In embodiments, the centroids of each cluster were used directly in sampling of vdMs on protein backbones. In embodiments, each vdM can be divided to a smaller number of vdM representatives.

The term “atomic coordinates” as used herein refers to a set of numbers that define the location of an atom or group of atoms (e.g., covalently bonded atoms in a compound or amino acid backbone, residue, or sidechain) in space (e.g., Euclidean space) in silico. Atomic coordinates may be, for example, Cartesian coordinates, internal coordinates, polar coordinates, spherical coordinates). In embodiments, the atomic coordinates of an atom will be understood to describe the location of all portions of the atom as understood by a person having ordinary skill in the art. For example, the atomic coordinates of an atom may be numbers describing a single point in space, however, it will be understood that the atomic coordinates further include the three dimensional space around the single point that would be occupied by the atom based on the radius of the atom as understood by a person having ordinary skill in the art. For example, when atomic coordinates described covalently attached atoms of a chemical group or amino acid (e.g., portion of backbone or sidechain), it will be understood that the atomic coordinates may explicitly describe single points in space for each atom but the atomic coordinates will be understood to also include the three dimensional space occupied by the atoms and the space occupied by the bonds between the atoms. The term “atomic protein coordinates” refers to atomic coordinates representing the atom(s) of a protein (e.g., backbone atom(s) of a protein, a protein capable of binding a compound). The term “atomic van der Mer coordinates” refers to atomic coordinates representing the atom(s) of a van der Mer, for example the atom(s) of a chemical group of a van der Mer or the atom(s) of an amino acid side chain of a van der Mer or the atom(s) of a portion of a protein backbone of a van der Mer bound to the atom(s) of a side chain of a van der Mer. The term “atomic chemical coordinates” refers to the atomic coordinates representing the atom(s) of a compound (e.g., a compound a protein is capable of binding to) or ligand (e.g., a ligand a protein is capable of binding to). The term “atomic amino acid coordinates” refers to the atomic coordinates representing the atom(s) of an amino acid residue (e.g., portion of the protein backbone and attached sidechain) of a protein in a complex of a protein bound to a compound, wherein the amino acid is not represented (e.g., overlap) by a van der Mer or wherein the amino acid does not interact with a chemical group of the compound bound to the protein.

The term “overlapping” when referring to juxtaposition of atomic coordinates (e.g., atomic van der Mer coordinates and atomic protein coordinates or atomic van der Mer coordinates and atomic chemical coordinates or atomic amino acid coordinates and atomic protein coordinates) refers to the situation wherein atoms (or bonds) represented by atomic coordinates of two different sources (e.g., atomic van der Mer coordinates and atomic protein coordinates or atomic van der Mer coordinates and atomic chemical coordinates or atomic amino acid coordinates and atomic protein coordinates) occupy the same space in three dimensions. It will be understood that the atomic coordinates may provide the location of a single point or multiple single points in space however, overlap will be determined by comparing the locations of the space around such single point(s) that is understood to be occupied by the atom represented by the atomic coordinates or by the atoms and bonds represented by the atomic coordinates of covalently bonded atoms. It will be understood that overlapping may be partial and complete overlap of all portions of an atom or bond are not necessary for overlap to occur.

### II. Methods

In an aspect is provided a computer-implemented method for identifying a protein capable of binding a compound, including identifying van der Mers representing the chemical groups of the compound and amino acid residues of the protein capable of interacting with the chemical groups of the compound in silico, and wherein the protein has secondary and tertiary protein structure when bound to the compound.

In an aspect is provided a computer-implemented method for identifying a protein capable of binding a compound, including:
(a) generating a first set of atomic protein coordinates representing a backbone structure of the protein;
(b) identifying a first van der Mer from a van der Mer database including a first set of atomic van der Mer coordinates representing a first chemical group of the compound, a first amino acid side chain and a first portion of a protein backbone bound to the first amino acid side chain, wherein the first chemical group interacts in silico with the first portion of a protein backbone or the first amino acid side chain;
(c) identifying portions of the backbone structure of the protein having atomic protein coordinates that are capable of overlapping with the atomic van der Mer coordinates of the first portion of a protein backbone of the first van der Mer;
(d) repeating steps (b) and (c) for at least one additional van der Mer independently representing an optionally different chemical group of the compound, an additional independent amino acid side chain and an additional independent portion of a protein backbone bound to the independent additional amino acid side chain;
(e) generating a set of atomic chemical coordinates representing the compound;
(f) generating at least one set of atomic coordinates of an in silico complex of the protein capable of binding the compound bound to the compound; wherein the in silico complex optimizes the overlap between the atomic chemical coordinates of the compound chemical groups and the atomic van der Mer coordinates representing the independent chemical groups of the compound identified in steps (b) to (d);
(g) generating a set of atomic amino acid coordinates for amino acid side chains and portions of protein backbone independently representing those portions of the backbone structure of the protein that are not overlapping with atomic van der Mer coordinates of the van der Mer including atomic van der Mer coordinates that are overlapping with the atomic chemical coordinates representing the compound of the in silico complex of step (f);
(h) based at least in part on steps (a) to (g), optimizing atomic coordinates of the compound and protein thereby identifying a protein capable of binding the compound.

In embodiments, the method includes generating a plurality of sets of atomic coordinates of an in silico complex of the protein capable of binding the compound bound to the compound in step (f). In embodiments, the method includes generating all possible sets of atomic coordinates of an in silico complex of the protein capable of binding the compound bound to the compound in step (f). In embodiments, the plurality of sets of atomic coordinates of an in silico complex of the protein capable of binding the compound bound to the compound are independently different from each other and optimization of the overlap between the atomic chemical coordinates of the compound chemical groups and the atomic van der Mer coordinates representing the independent chemical groups of the compound identified in steps (b) to (d) is performed without duplication of the sets. In embodiments, the plurality of independent sets of atomic coordinates of an in silico complex of the protein capable of binding the compound bound to the compound are independently different from each other and are scored. In embodiments, the scoring includes calculating a cluster score for each of the plurality of sets of atomic coordinates of an in silico complex of the protein capable of binding the compound bound to the compound. In embodiments, the cluster score is a function (e.g., including but not limited to a natural log or logistical function) of the ratio of 1) the number of members in an independent set of geometrically overlapping van der Mer of one chemical group and one amino acid to 2) the average number of members in all independent sets van der Mer of the chemical group and the amino acid. In embodiments, the members in an independent set of geometrically overlapping compound van der Mer of one chemical group are identified by having an RMSD below a threshold, wherein the RMSD is calculated using the atomic coordinates of the chemical group and the backbone atoms of the amino acid residue of a van der Mer. In embodiments, the RMSD threshold is 0.5 angstrom. In embodiments, step (a) includes generating a plurality of independent sets of atomic protein coordinates representing independent backbone structures of the protein. In embodiments, the plurality of independent backbone structures of the protein have a similar overall three dimensional fold. In embodiments, the plurality of independent backbone structures of the protein have an RMSD of less than 3 angstrom. In embodiments, the compound chemical groups and van der Mer chemical groups are polar groups. In embodiments, steps (g) and (h) include use of a method described in international application no. WO2019/023644. In embodiments, step (c) includes identifying all portions of the backbone structure of the protein having atomic protein coordinates that are capable of overlapping with the atomic van der Mer coordinates of the first portion of a protein backbone of the first van der Mer. In embodiments, step (d) includes repeating steps (b) and (c) for all van der Mer in the van der Mer database independently representing all chemical groups of the compound, an additional independent amino acid side chain and an additional independent portion of a protein backbone bound to said independent additional amino acid side chain. In embodiments, the overlap of the van der Mers and the compound chemical groups are not selected one at a time, but rather in pairs, triplets or higher order combinations. In embodiments, van der Mers at multiple sites are selected and the RMSD between these van der Mer chemical groups and the compound chemical groups is computed. In embodiments, the RMSD between van der Mer chemical groups and the compound chemical groups is precomputed and saved in lookup tables. In embodiments, the overlap of the van der Mers and the compound chemical groups are selected in pairs. In embodiments, the overlap of the van der Mers and the compound chemical groups are selected in triplets. In embodiments, the overlap of the van der Mers and the compound chemical groups are selected in combinations greater than three. In embodiments an identified van der Mer is a cluster having a 0.5 angstrom RMSD between members. In embodiments an identified van der Mer is a sub-cluster of a van der Mer. In embodiments an identified van der Mer is a van der Mer cluster member. In embodiments an identified van der Mer is a van der Mer representative. In embodiments an identified van der Mer is a cluster having a 0.1 angstrom RMSD between members.

In an aspect is provided a computer-implemented method for identifying a complex of a protein bound to a compound, including:
(a) generating a first set of atomic protein coordinates representing the side chain and backbone structure of the protein;
(b) identifying a first van der Mer from a van der Mer database including a first set of atomic van der Mer coordinates representing a first chemical group of the compound, a first amino acid side chain and a first portion of a protein backbone bound to the first amino acid side chain, wherein the first chemical group interacts in silico with the first portion of a protein backbone or the first amino acid side chain;
(c) identifying portions of the backbone structure of the protein having atomic protein coordinates that are capable of overlapping with the atomic van der Mer coordinates of the first portion of a protein backbone of the first van der Mer wherein the amino acid side chain of the van der Mer and the amino acid side chain directly attached to the overlapping portions of the backbone structure of the protein are the same side chain;
(d) repeating steps (b) and (c) for at least one additional van der Mer independently representing an optionally different chemical group of the compound, an additional independent amino acid side chain and an additional independent portion of a protein backbone bound to the independent additional amino acid side chain;
(e) generating a set of atomic chemical coordinates representing the compound;
(f) generating at least one set of atomic coordinates of an in silico complex of the protein capable of binding the compound bound to the compound; wherein the in silico complex optimizes the overlap between the atomic chemical coordinates of the compound chemical groups and the atomic van der Mer coordinates representing the independent chemical groups of the compound identified in steps (b) to (d);
(g) based at least in part on steps (a) to (f), optimizing atomic coordinates of the compound and protein thereby identifying a complex of a protein bound to a compound.

In an aspect is provided a computer-implemented method for identifying a protein capable of binding a compound, including:
(a) generating a first set of atomic protein coordinates representing a protein backbone structure;
(b) generating a first set of atomic chemical coordinates representing a first chemical group of the compound;
(c) identifying a first van der Mer from a van der Mer database including a first set of atomic van der Mer coordinates representing the first chemical group of the compound, a first amino acid side chain and a first portion of a protein backbone bound to the first amino acid side chain, wherein the first chemical group interacts in silico with the first portion of a protein backbone or the first amino acid side chain;
(d) generating a second set of atomic chemical coordinates representing a second chemical group of the compound;
(e) identifying a second van der Mer from the van der Mer database including a second set of atomic van der Mer coordinates representing the second chemical group of the compound, a second amino acid side chain and a second portion of a protein backbone bound to the second amino acid side chain, wherein the second chemical group interacts in silico with the second portion of a protein backbone or the second amino acid side chain;
(f) calculating an energetic stability of the protein backbone structure bound to the compound using the first set of atomic van der Mer coordinates and the second set of atomic van der Mer coordinates in silico;
(g) repeating steps (a) to (f) for additional van der Mers representing the first chemical group of the compound, a first amino acid side chain and a first portion of a protein backbone bound to the first amino acid side chain and additional van der Mers representing the second chemical group of the compound, a second amino acid side chain and a second portion of a protein backbone bound to the second amino acid side chain;
(h) generating a set of atomic amino acid coordinates for amino acid side chains and portions of protein backbone independently representing those portions of the protein backbone structure not represented by a van der Mer of steps (a) to (g);
(i) based at least in part on steps (a) to (h), optimizing atomic coordinates of the compound and protein thereby identifying a protein capable of binding the compound.

In an aspect is provided a computer-implemented method for identifying a protein capable of binding a compound, including:
(a) identifying a first van der Mer from a van der Mer database including atomic van der Mer coordinates of a chemical group of the compound, wherein the atomic van der Mer coordinates of the chemical group in the first van der Mer overlap with the atomic chemical coordinates of the chemical group of the compound;
(b) identifying a protein backbone for the protein wherein the atoms of the protein backbone are associated with a set of atomic protein coordinates;
(c) identifying an overlap between the atomic van der Mer coordinates of the amino acid backbone of the first van der Mer identified in step (a) and the atomic protein coordinates of an amino acid residue of the protein backbone identified in step (b);
(d) optionally repeating steps (a) to (c) for a different chemical group of the compound;
(e) identifying independent sets of van der Mer identified in steps (a) to (d) wherein all van der Mer of each independent set include atomic van der Mer coordinates that collectively simultaneously overlap atomic protein coordinates of the protein backbone identified in step (b);
(f) identifying at least one independent set of van der Mer identified in step (e) with a cluster score above a threshold;
(g) identifying an amino acid residue for each amino acid of the protein backbone identified in step (b) having atomic amino acid coordinates that are not overlapping with the atomic van der Mer coordinates of the set of van der Mer identified in step (f);
(h) optimizing atomic coordinates of the compound and protein;
(i) wherein the optimization is performed using at least an energy minimization calculation, and wherein the optimization is performed to energetically stabilize a complex of the compound and protein.

In an aspect is provided a computer-implemented method for identifying a protein capable of binding a compound, including:
(a) identifying covalently bonded amino acid backbone residues of the protein wherein each amino acid backbone residue atom is associated with a set of atomic protein coordinates;
(b) identifying an independent set of van der Mer associated with an amino acid backbone residue and a chemical group of the compound, wherein each van der Mer is associated with a set of atomic van der Mer coordinates for an amino acid and chemical group of the compound and the atomic van der Mer coordinates for the van der Mer amino acid backbone atoms of each independent set of van der Mer overlap with amino acid backbone residue atomic protein coordinates of the protein;
(c) identifying and removing from each independent set of van der Mer, any van der Mer wherein atomic van der Mer coordinates of a sidechain or chemical group of the van der Mer overlap with atomic protein coordinates of the covalently bonded amino acid backbone residues of the protein;
(d) identifying and removing any van der Mer wherein atomic van der Mer coordinates of the chemical group of the van der Mer is characterized as exposed to bulk solvent;
(e) identifying independent sets of atomic chemical coordinates of the compound wherein, the atomic chemical coordinates of the compound chemical group atoms of each independent set overlap with atomic van der Mer coordinates of chemical group atoms of van der Mer identified in steps (b) to (d) and atomic van der Mer coordinates of the van der Mer further include atomic van der Mer coordinates for amino acid backbone atoms that overlap with atomic protein coordinates of amino acid backbone atoms of the protein;
(f) identifying and sorting independent sets of atomic chemical coordinates of the compound of step (e) based on the value of the compound van der Mer cluster score;
(g) identifying a preferred amino acid for an amino acid residue position of the protein when the amino acid residue position of the protein has amino acid backbone atom atomic protein coordinates that overlap with the amino acid backbone atomic van der Mer coordinates of a van der Mer identified in step (f) and the preferred amino acid is the amino acid associated with the van der Mer;
(h) optimizing atomic coordinates of the compound and amino acid residues of the protein;
(i) wherein the optimization is performed using at least an energy minimization calculation, and wherein the optimization is performed to energetically stabilize the protein.

In embodiments, the optimizing includes an iterative or heuristic algorithm. In embodiments, the optimizing includes a simplex algorithm, memetic algorithm, differential evolution algorithm, evolutionary algorithm, genetic algorithm, tabu algorithm, particle swarm algorithm, or stimulated annealing algorithm. In embodiments, the optimizing includes a Monte Carlo sampling algorithm, dead-end elimination algorithm, branch and bound algorithm, or a pruning algorithm. In embodiments, the optimizing includes an energy minimization calculation. In embodiments, the energy minimization calculation includes a molecular mechanics function, a structural bioinformatics function, an amino acid sidechain packing function, a protein radius of gyration function, or a combination thereof. In embodiments, identifying atomic van der Mer coordinates of a chemical group of a van der Mer as exposed to bulk solvent is performed using a convex hull algorithm. In embodiments, the cluster score is a function (e.g., including but not limited to a natural log or logistical function) of the ratio of 1) the number of members in an independent set of geometrically overlapping van der Mer of one chemical group and one amino acid to 2) the average number of members in all independent sets van der Mer of the chemical group and the amino acid. In embodiments, the members in an independent set of geometrically overlapping compound van der Mer of one chemical group are identified by having an RMSD below a threshold, wherein the RMSD is calculated using the atomic coordinates of the chemical group and the backbone atoms of the amino acid residue of a van der Mer. In embodiments, the RMSD threshold is 0.5 angstrom. In embodiments, the preferred amino acid of step (g) is an amino acid in a van der Mer having a cluster score greater than 2. In embodiments, identifying an amino acid residue for each protein backbone residue having atomic amino acid coordinates that are not overlapping with the atomic van der Mer coordinates of a van der Mer is performed using The Rosetta Software. In embodiments, the van der Mer database is a collection of independent van der Mer each including a unique set of atomic van der Mer coordinates describing the three dimensional positions of a chemical group interacting in silico with an amino acid residue, further wherein the interacting was identified in an empirically determined protein and chemical group complex. In embodiments, the protein is a 4-helix bundle protein. In embodiments, the compound includes a charged chemical group at physiological pH. In embodiments, the compound includes a polar chemical group at physiological pH. In embodiments, the method further includes making the protein. In embodiments, the method further includes making the protein using molecular biology techniques. In embodiments, the method further includes making the protein using peptide synthesis. In embodiments, the method further includes making the protein by expressing the protein from an exogenous nucleic acid. In embodiments, the method includes use of a method described in international application no. WO2019/023644. In embodiments, the overlap of the van der Mers and the compound chemical groups are not selected one at a time, but rather in pairs, triplets or higher order combinations. In embodiments, van der Mers at multiple sites are selected and the RMSD between these van der Mer chemical groups and the compound chemical groups is computed. In embodiments, the RMSD between van der Mer chemical groups and the compound chemical groups is precomputed and saved in lookup tables. In embodiments, the overlap of the van der Mers and the compound chemical groups are selected in pairs. In embodiments, the overlap of the van der Mers and the compound chemical groups are selected in triplets. In embodiments, the overlap of the van der Mers and the compound chemical groups are selected in combinations greater than three. In embodiments an identified van der Mer is a cluster having a 0.5 angstrom RMSD between members. In embodiments an identified van der Mer is a sub-cluster of a van der Mer. In embodiments an identified van der Mer is a van der Mer cluster member. In embodiments an identified van der Mer is a van der Mer representative. In embodiments an identified van der Mer is a cluster having a 0.1 angstrom RMSD between members.

In embodiments, the optimizing includes an iterative or heuristic algorithm. In embodiments, the optimizing includes an iterative algorithm. In embodiments, the optimizing includes a heuristic algorithm. In embodiments, the optimizing includes a simplex algorithm, memetic algorithm, differential evolution algorithm, evolutionary algorithm, genetic algorithm, tabu algorithm, particle swarm algorithm, or stimulated annealing algorithm. In embodiments, the optimizing includes a Monte Carlo sampling algorithm, dead-end elimination algorithm, branch and bound algorithm, or a pruning algorithm. In embodiments, the optimizing includes knobs-into-holes side chain packing. In embodiments, the optimization may begin with an idealized, parameterized backbone. In embodiments, optimization may relax the backbone structure of the protein, for example, by using gradient descent algorithms, while optimizing the protein sequence via rotamer sampling and minimization.

In embodiments, the optimizing includes introducing an additional compound binding amino acid residue into the set of compound binding amino acid residues, deleting a compound binding amino acid residue from the set of compound binding amino acid residues, a geometric transformation of at least one atomic coordinate of the compound binding amino acid residue atomic coordinates.

In embodiments, the optimizing includes introducing an additional compound binding amino acid residue into the set of compound binding amino acid residues (e.g., designating an amino acid residue previously not designated as a compound binding amino acid residue to a compound binding amino acid residue). In embodiments, the optimizing includes replacing a compound binding amino acid residue within the set of compound binding amino acid residues. In embodiments, the optimizing includes deleting a compound binding amino acid residue from the set of compound binding amino acid residues. In embodiments, the optimizing includes a geometric transformation of at least one atomic coordinate of the compound binding amino acid residue atomic coordinates. In embodiments, the optimizing includes a geometric transformation of the atomic coordinates of at least one of the compound binding amino acid residue atomic coordinates. In embodiments, the optimizing includes a geometric transformation of the atomic coordinates of the compound binding amino acid residue atomic coordinates.

In embodiments, the geometric transformation includes a translation (i.e., a geometric transformation that moves a coordinate by the same distance in a given direction) or a rotation of at least one atomic coordinate of the compound binding amino acid residue atomic coordinates. In embodiments, the geometric transformation includes a translation (e.g., displacing the x coordinate) of at least one atomic coordinate of the compound binding amino acid residue atomic coordinates. In embodiments, the geometric transformation includes a translation of at least two atomic coordinates of the compound binding amino acid residue atomic coordinates. In embodiments, the geometric transformation includes a translation of all atomic coordinates (e.g., x, y, and z coordinates in Cartesian space) of the compound binding amino acid residue atomic coordinates. In embodiments, the geometric transformation includes a rotation of at least one atomic coordinate of the compound binding amino acid residue atomic coordinates. In embodiments, the geometric transformation includes a rotation of at least two atomic coordinates of the compound binding amino acid residue atomic coordinates. In embodiments, the geometric transformation includes a rotation of at least three atomic coordinates of the compound binding amino acid residue atomic coordinates. In embodiments, the geometric transformation includes a rotation of all atomic coordinates of the compound binding amino acid residue atomic coordinates.

In embodiments, the optimizing includes a geometric transformation of at least one atomic coordinate of the non-compound binding amino acid residue atomic coordinates. In embodiments, the geometric transformation includes a translation or a rotation of at least one atomic coordinate of the non-compound binding amino acid residue atomic coordinates. In embodiments, the geometric transformation includes a translation of at least one atomic coordinate of the non-compound binding amino acid residue atomic coordinates. In embodiments, the geometric transformation includes a translation of at least two atomic coordinates of the non-compound binding amino acid residue atomic coordinates. In embodiments, the geometric transformation includes a translation of all atomic coordinates of the non-compound binding amino acid residue atomic coordinates. In embodiments, the geometric transformation includes a rotation of at least one atomic coordinate of the non-compound binding amino acid residue atomic coordinates. In embodiments, the geometric transformation includes a rotation of at least two atomic coordinates of the non-compound binding amino acid residue atomic coordinates. In embodiments, the geometric transformation includes a rotation of at least three atomic coordinates of the non-compound binding amino acid residue atomic coordinates. In embodiments, the geometric transformation includes a rotation of all atomic coordinates of the non-compound binding amino acid residue atomic coordinates.

In embodiments, the optimizing includes 1a) calculating the force on each atom in the protein (e.g., the set of compound binding amino acid residues; the set of non-compound binding amino acid residues; and the compound); 2a) evaluating the calculation to determine if it is the minimum or below an acceptable threshold; 3a) if the force is less than a threshold, the optimization is finished, otherwise perform a geometric transformation (e.g., translation) of at least one atomic coordinate on the atoms in the protein; and 4a) repeat.

In embodiments, the geometric transformation of at least one atomic coordinate includes no greater than a 6 Å displacement of any atomic coordinate. In embodiments, the geometric transformation of at least one atomic coordinate includes no greater than a 3 Å displacement of any atomic coordinate. In embodiments, the displacement is no greater than 0.1, 0.2, 0.3, 0.4, 0.5, 1, 2, 3, 4, 5, 6, 7, 8, 9, or 10 Å displacement of any atomic coordinate. In embodiments, the displacement is no greater than 0.01, 0.02, 0.03, 0.04, 0.05, 0.1, 0.2, 0.3, 0.4, 0.5, 0.6, 0.7, 0.8, 0.9, or 1.0 Å displacement of any atomic coordinate.

In embodiments, the set of compound binding amino acids includes at least 50 amino acid residues. In embodiments, the set of compound binding amino acids includes at least 40 amino acid residues. In embodiments, the set of compound binding amino acids includes at least 30 amino acid residues. In embodiments, the set of compound binding amino acids includes at least 20 amino acid residues. In embodiments, the set of compound binding amino acids includes at least 12 amino acid residues. In embodiments, the set of compound binding amino acids includes at least 10 amino acid residues. In embodiments, the set of compound binding amino acids includes at least 8 amino acid residues. In embodiments, the set of compound binding amino acids includes at least 6 amino acid residues. In embodiments, the set of compound binding amino acids includes at least 5 amino acid residues. In embodiments, the set of compound binding amino acids includes at least 4 amino acid residues. In embodiments, the set of compound binding amino acids includes at least 3 amino acid residues. In embodiments, the set of compound binding amino acids includes at least 2 amino acid residues. In embodiments the compound binding amino acids are apolar. In embodiments the compound binding amino acids are hydrophilic.

In embodiments, the set of compound binding amino acids includes 50 amino acid residues. In embodiments, the set of compound binding amino acids includes 40 amino acid residues. In embodiments, the set of compound binding amino acids includes 30 amino acid residues. In embodiments, the set of compound binding amino acids includes 20 amino acid residues. In embodiments, the set of compound binding amino acids includes 12 amino acid residues. In embodiments, the set of compound binding amino acids includes 10 amino acid residues. In embodiments, the set of compound binding amino acids includes 8 amino acid residues. In embodiments, the set of compound binding amino acids includes 6 amino acid residues. In embodiments, the set of compound binding amino acids includes 5 amino acid residues. In embodiments, the set of compound binding amino acids includes 4 amino acid residues. In embodiments, the set of compound binding amino acids includes 3 amino acid residues. In embodiments, the set of compound binding amino acids includes 2 amino acid residues. In embodiments the compound binding amino acids are polar. In embodiments the compound binding amino acids are hydrophilic.

In embodiments, the energy minimization calculation includes a molecular mechanics function, a structural bioinformatics function, an amino acid sidechain packing function, a protein radius of gyration function, or a combination thereof. In embodiments, the energy minimization calculation includes a penalty function.

In embodiments, the compound is a porphyrin, porphycene, rubyrin, rosarin, hexaphyrin, sapphyrin, chlorophyll, chlorin, phthalocyanine, porphyrazine, corrole, N-confused porphyrin, bacteriochlorophyll, pheophytin, texaphyrin, or related macrocyclic-based component, that is capable of binding a metal ion. In embodiments, the compound is a detectable agent. In embodiments, the compound is a therapeutic agent, biological agent, cytotoxic agent, magnetic resonance imaging (MRI) agent, positron emission tomography (PET) agent, radiological imaging agent, diagnostic agent, theragnostic, or a photodynamic therapy (PDT) agent. In embodiments, the compound is a therapeutic agent. In embodiments, the compound is a biological agent. In embodiments, the compound is a cytotoxic agent (e.g., an anticancer agent). In embodiments, the compound is a magnetic resonance imaging (MRI) agent. In embodiments, the compound is a positron emission tomography (PET) agent. In embodiments, the compound is a radiological imaging agent. In embodiments, the compound is a diagnostic agent. In embodiments, the compound is a theragnostic agent. In embodiments, the compound is a photodynamic therapy (PDT) agent. In embodiments, the compound is a small molecule.

In embodiments, the compound is a catalyst. In embodiments, the catalyst catalyzes an abiological or bio-orthogonal reaction. In embodiments, the compound is a molecule that exists within a living system (e.g., within an organism or a cell). In embodiments, the compound atomic coordinates are optimized using known methods in the art (e.g., density functional theory using the B3-LYP functional).

In embodiments, the method further includes synthesizing the protein (e.g., utilizing the expression vectors such as the plasmid method described in the Example, such as cloning into the IPTG-inducible pET-11a plasmid). In embodiments, the method further includes expressing the protein.

These compound binding amino acid residues can form the backbone of a protein. Each compound binding amino acid residue within the protein can be associated with a set of compound binding amino acid residue atomic coordinates, which can define the compound binding amino acid residue in space. Furthermore, each atom of the compound can be associated with a set of ligand atomic coordinates, which can define the compound in space. As noted herein, these coordinates can be Cartesian coordinates, internal coordinates, polar coordinates, spherical coordinates, and/or the like.

The set of compound binding amino acid residues, the set of compound binding amino acid residue atomic coordinates, the set of non-compound binding amino acid residues, and the set of non-compound binding amino acid residue atomic coordinates can be optimized. For example, the optimization can be performed using an energy minimization calculation including, for example, a molecular mechanics function, a structural bioinformatics function, an amino acid sidechain packing function, a protein radius of gyration function, and/or the like. Optimizing the set of compound binding amino acid residues, the set of compound binding amino acid residue atomic coordinates, the set of non-compound binding amino acid residues, and the set of non-compound binding amino acid residue atomic coordinates can generate an energetically stabilized protein.

In an aspect is provided a computer-implemented method for identifying a protein capable of binding a ligand (e.g., compound), including identifying van der Mers representing the chemical groups of the ligand (e.g., compound) and amino acid residues of the protein capable of interacting with the chemical groups of the ligand (e.g., compound) in silico, and wherein the protein has secondary and tertiary protein structure when bound to the ligand (e.g., compound).

In an aspect is provided a computer-implemented method for identifying a protein capable of binding a ligand (e.g., compound), including:
(a) generating a first set of atomic protein coordinates representing a backbone structure of the protein;
(b) identifying a first van der Mer from a van der Mer database including a first set of atomic van der Mer coordinates representing a first chemical group of the ligand (e.g., compound), a first amino acid side chain and a first portion of a protein backbone bound to the first amino acid side chain, wherein the first chemical group interacts in silico with the first portion of a protein backbone or the first amino acid side chain;
(c) identifying portions of the backbone structure of the protein having atomic protein coordinates that are capable of overlapping with the atomic van der Mer coordinates of the first portion of a protein backbone of the first van der Mer;
(d) repeating steps (b) and (c) for at least one additional van der Mer independently representing an optionally different chemical group of the ligand (e.g., compound), an additional independent amino acid side chain and an additional independent portion of a protein backbone bound to the independent additional amino acid side chain;
(e) generating a set of atomic chemical coordinates representing the ligand (e.g., compound);
(f) generating at least one set of atomic coordinates of an in silico complex of the protein capable of binding the ligand (e.g., compound) bound to the ligand (e.g., compound); wherein the in silico complex optimizes the overlap between the atomic chemical coordinates of the ligand (e.g., compound) chemical groups and the atomic van der Mer coordinates representing the independent chemical groups of the ligand (e.g., compound) identified in steps (b) to (d);
(g) generating a set of atomic amino acid coordinates for amino acid side chains and portions of protein backbone independently representing those portions of the backbone structure of the protein that are not overlapping with atomic van der Mer coordinates of the van der Mer including atomic van der Mer coordinates that are overlapping with the atomic chemical coordinates representing the ligand (e.g., compound) of the in silico complex of step (f);
(h) based at least in part on steps (a) to (g), optimizing atomic coordinates of the ligand (e.g., compound) and protein thereby identifying a protein capable of binding the ligand (e.g., compound).

In an aspect is provided a computer-implemented method for identifying a complex of a protein bound to a ligand (e.g., compound), including:
(a) generating a first set of atomic protein coordinates representing the side chain and backbone structure of the protein;
(b) identifying a first van der Mer from a van der Mer database including a first set of atomic van der Mer coordinates representing a first chemical group of the ligand (e.g., compound), a first amino acid side chain and a first portion of a protein backbone bound to the first amino acid side chain, wherein the first chemical group interacts in silico with the first portion of a protein backbone or the first amino acid side chain;
(c) identifying portions of the backbone structure of the protein having atomic protein coordinates that are capable of overlapping with the atomic van der Mer coordinates of the first portion of a protein backbone of the first van der Mer wherein the amino acid side chain of the van der Mer and the amino acid side chain directly attached to the overlapping portions of the backbone structure of the protein are the same side chain;
(d) repeating steps (b) and (c) for at least one additional van der Mer independently representing an optionally different chemical group of the ligand (e.g., compound), an additional independent amino acid side chain and an additional independent portion of a protein backbone bound to the independent additional amino acid side chain;
(e) generating a set of atomic chemical coordinates representing the ligand (e.g., compound);
(f) generating at least one set of atomic coordinates of an in silico complex of the protein capable of binding the ligand (e.g., compound) bound to the ligand (e.g., compound); wherein the in silico complex optimizes the overlap between the atomic chemical coordinates of the ligand (e.g., compound) chemical groups and the atomic van der Mer coordinates representing the independent chemical groups of the ligand (e.g., compound) identified in steps (b) to (d);
(g) based at least in part on steps (a) to (f), optimizing atomic coordinates of the ligand (e.g., compound) and protein thereby identifying a complex of a protein bound to a ligand (e.g., compound).

In an aspect is provided a computer-implemented method for identifying a protein capable of binding a ligand (e.g., compound), including:
(a) generating a first set of atomic protein coordinates representing a protein backbone structure;
(b) generating a first set of atomic chemical coordinates representing a first chemical group of the ligand (e.g., compound);
(c) identifying a first van der Mer from a van der Mer database including a first set of atomic van der Mer coordinates representing the first chemical group of the ligand (e.g., compound), a first amino acid side chain and a first portion of a protein backbone bound to the first amino acid side chain, wherein the first chemical group interacts in silico with the first portion of a protein backbone or the first amino acid side chain;
(d) generating a second set of atomic chemical coordinates representing a second chemical group of the ligand (e.g., compound);
(e) identifying a second van der Mer from the van der Mer database including a second set of atomic van der Mer coordinates representing the second chemical group of the ligand (e.g., compound), a second amino acid side chain and a second portion of a protein backbone bound to the second amino acid side chain, wherein the second chemical group interacts in silico with the second portion of a protein backbone or the second amino acid side chain;
(f) calculating an energetic stability of the protein backbone structure bound to the ligand (e.g., compound) using the first set of atomic van der Mer coordinates and the second set of atomic van der Mer coordinates in silico;
(g) repeating steps (a) to (f) for additional van der Mers representing the first chemical group of the ligand (e.g., compound), a first amino acid side chain and a first portion of a protein backbone bound to the first amino acid side chain and additional van der Mers representing the second chemical group of the ligand (e.g., compound), a second amino acid side chain and a second portion of a protein backbone bound to the second amino acid side chain;
(h) generating a set of atomic amino acid coordinates for amino acid side chains and portions of protein backbone independently representing those portions of the protein backbone structure not represented by a van der Mer of steps (a) to (g);
(i) based at least in part on steps (a) to (h), optimizing atomic coordinates of the ligand (e.g., compound) and protein thereby identifying a protein capable of binding the ligand (e.g., compound).

In an aspect is provided a computer-implemented method for identifying a protein capable of binding a ligand (e.g., compound), including:
(a) identifying a first van der Mer from a van der Mer database including atomic van der Mer coordinates of a chemical group of the ligand (e.g., compound), wherein the atomic van der Mer coordinates of the chemical group in the first van der Mer overlap with the atomic chemical coordinates of the chemical group of the ligand (e.g., compound);
(b) identifying a protein backbone for the protein wherein the atoms of the protein backbone are associated with a set of atomic protein coordinates;
(c) identifying an overlap between the atomic van der Mer coordinates of the amino acid backbone of the first van der Mer identified in step (a) and the atomic protein coordinates of an amino acid residue of the protein backbone identified in step (b);
(d) optionally repeating steps (a) to (c) for a different chemical group of the ligand (e.g., compound);
(e) identifying independent sets of van der Mer identified in steps (a) to (d) wherein all van der Mer of each independent set include atomic van der Mer coordinates that collectively simultaneously overlap atomic protein coordinates of the protein backbone identified in step (b);
(f) identifying at least one independent set of van der Mer identified in step (e) with a cluster score above a threshold;
(g) identifying an amino acid residue for each amino acid of the protein backbone identified in step (b) having atomic amino acid coordinates that are not overlapping with the atomic van der Mer coordinates of the set of van der Mer identified in step (f);
(h) optimizing atomic coordinates of the ligand (e.g., compound) and protein;
(i) wherein the optimization is performed using at least an energy minimization calculation, and wherein the optimization is performed to energetically stabilize a complex of the ligand (e.g., compound) and protein.

In an aspect is provided a computer-implemented method for identifying a protein capable of binding a ligand (e.g., compound), including:
(a) identifying covalently bonded amino acid backbone residues of the protein wherein each amino acid backbone residue atom is associated with a set of atomic protein coordinates;
(b) identifying an independent set of van der Mer associated with an amino acid backbone residue and a chemical group of the ligand (e.g., compound), wherein each van der Mer is associated with a set of atomic van der Mer coordinates for an amino acid and chemical group of the ligand (e.g., compound) and the atomic van der Mer coordinates for the van der Mer amino acid backbone atoms of each independent set of van der Mer overlap with amino acid backbone residue atomic protein coordinates of the protein;
(c) identifying and removing from each independent set of van der Mer, any van der Mer wherein atomic van der Mer coordinates of a sidechain or chemical group of the van der Mer overlap with atomic protein coordinates of the covalently bonded amino acid backbone residues of the protein;
(d) identifying and removing any van der Mer wherein atomic van der Mer coordinates of the chemical group of the van der Mer is characterized as exposed to bulk solvent;
(e) identifying independent sets of atomic chemical coordinates of the ligand (e.g., compound) wherein, the atomic chemical coordinates of the ligand (e.g., compound) chemical group atoms of each independent set overlap with atomic van der Mer coordinates of chemical group atoms of van der Mer identified in steps (b) to (d) and atomic van der Mer coordinates of the van der Mer further include atomic van der Mer coordinates for amino acid backbone atoms that overlap with atomic protein coordinates of amino acid backbone atoms of the protein;
(f) identifying and sorting independent sets of atomic chemical coordinates of the ligand (e.g., compound) of step (e) based on the value of the ligand (e.g., compound) van der Mer cluster score;
(g) identifying a preferred amino acid for an amino acid residue position of the protein when the amino acid residue position of the protein has amino acid backbone atom atomic protein coordinates that overlap with the amino acid backbone atomic van der Mer coordinates of a van der Mer identified in step (f) and the preferred amino acid is the amino acid associated with the van der Mer;
(h) optimizing atomic coordinates of the ligand (e.g., compound) and amino acid residues of the protein;
(i) wherein the optimization is performed using at least an energy minimization calculation, and wherein the optimization is performed to energetically stabilize the protein.

In embodiments, the ligand is a compound. In embodiments, the compound is a chemical molecule having molecular weight of less than 10000 Daltons (e.g., less than 9000, 8000, 7000, 6000, 5000, 4000, 3000, 2000, 1000, 900, 800, 700, 600, 500, 400, 300, 200, or 100).

In embodiments, the method includes generating a plurality of sets of atomic coordinates of an in silico complex of the protein capable of binding the ligand bound to the ligand in step (f). In embodiments, the method includes generating all possible sets of atomic coordinates of an in silico complex of the protein capable of binding the ligand bound to the ligand in step (f). In embodiments, the plurality of sets of atomic coordinates of an in silico complex of the protein capable of binding the ligand bound to the ligand are independently different from each other and optimization of the overlap between the atomic chemical coordinates of the ligand chemical groups and the atomic van der Mer coordinates representing the independent chemical groups of the ligand identified in steps (b) to (d) is performed without duplication of the sets. In embodiments, the plurality of independent sets of atomic coordinates of an in silico complex of the protein capable of binding the ligand bound to the ligand are independently different from each other and are scored. In embodiments, the scoring includes calculating a cluster score for each of the plurality of sets of atomic coordinates of an in silico complex of the protein capable of binding the ligand bound to the ligand. In embodiments, the cluster score is a function (e.g., including but not limited to a natural log or logistical function) of the ratio of 1) the number of members in an independent set of geometrically overlapping van der Mer of one chemical group and one amino acid to 2) the average number of members in all independent sets van der Mer of the chemical group and the amino acid. In embodiments, the members in an independent set of geometrically overlapping ligand van der Mer of one chemical group are identified by having an RMSD below a threshold, wherein the RMSD is calculated using the atomic coordinates of the chemical group and the backbone atoms of the amino acid residue of a van der Mer. In embodiments, the RMSD threshold is 0.5 angstrom. In embodiments, step (a) includes generating a plurality of independent sets of atomic protein coordinates representing independent backbone structures of the protein. In embodiments, the plurality of independent backbone structures of the protein have a similar overall three dimensional fold. In embodiments, the plurality of independent backbone structures of the protein have an RMSD of less than 3 angstrom. In embodiments, the ligand chemical groups and van der Mer chemical groups are polar groups. In embodiments, steps (g) and (h) include use of a method described in international application no. WO2019/023644. In embodiments, step (c) includes identifying all portions of the backbone structure of the protein having atomic protein coordinates that are capable of overlapping with the atomic van der Mer coordinates of the first portion of a protein backbone of the first van der Mer. In embodiments, step (d) includes repeating steps (b) and (c) for all van der Mer in the van der Mer database independently representing all chemical groups of the ligand, an additional independent amino acid side chain and an additional independent portion of a protein backbone bound to said independent additional amino acid side chain. In embodiments, the overlap of the van der Mers and the ligand chemical groups are not selected one at a time, but rather in pairs, triplets or higher order combinations. In embodiments, van der Mers at multiple sites are selected and the RMSD between these van der Mer chemical groups and the ligand chemical groups is computed. In embodiments, the RMSD between van der Mer chemical groups and the ligand chemical groups is precomputed and saved in lookup tables. In embodiments, the overlap of the van der Mers and the ligand chemical groups are selected in pairs. In embodiments, the overlap of the van der Mers and the ligand chemical groups are selected in triplets. In embodiments, the overlap of the van der Mers and the ligand chemical groups are selected in combinations greater than three. In embodiments an identified van der Mer is a cluster having a 0.5 angstrom RMSD between members. In embodiments an identified van der Mer is a sub-cluster of a van der Mer. In embodiments an identified van der Mer is a van der Mer cluster member. In embodiments an identified van der Mer is a van der Mer representative. In embodiments an identified van der Mer is a cluster having a 0.1 angstrom RMSD between members.

In embodiments, the members in an independent set of geometrically overlapping ligand van der Mer of one chemical group are identified by having an RMSD below a threshold, wherein the RMSD is calculated using the atomic coordinates of the chemical group and the backbone atoms of the amino acid residue of a van der Mer. In embodiments, the RMSD threshold is 0.5 angstrom. In embodiments, the preferred amino acid of step (g) is an amino acid in a van der Mer having a cluster score greater than 2. In embodiments, identifying an amino acid residue for each protein backbone residue having atomic amino acid coordinates that are not overlapping with the atomic van der Mer coordinates of a van der Mer is performed using The Rosetta Software. In embodiments, the van der Mer database is a collection of independent van der Mer each including a unique set of atomic van der Mer coordinates describing the three dimensional positions of a chemical group interacting in silico with an amino acid residue, further wherein the interacting was identified in an empirically determined protein and chemical group complex. In embodiments, the protein is a 4-helix bundle protein. In embodiments, the ligand includes a charged chemical group at physiological pH. In embodiments, the ligand includes a polar chemical group at physiological pH. In embodiments, the method further includes making the protein. In embodiments, the method further includes making the protein using molecular biology techniques. In embodiments, the method further includes making the protein using peptide synthesis. In embodiments, the method further includes making the protein by expressing the protein from an exogenous nucleic acid. In embodiments, the method includes use of a method described in international application no. WO2019/023644.

In embodiments, the optimizing includes introducing an additional ligand binding amino acid residue into the set of ligand binding amino acid residues, deleting a ligand binding amino acid residue from the set of ligand binding amino acid residues, a geometric transformation of at least one atomic coordinate of the ligand binding amino acid residue atomic coordinates.

In embodiments, the optimizing includes introducing an additional ligand binding amino acid residue into the set of ligand binding amino acid residues (e.g., designating an amino acid residue previously not designated as a ligand binding amino acid residue to a ligand binding amino acid residue). In embodiments, the optimizing includes replacing a ligand binding amino acid residue within the set of ligand binding amino acid residues. In embodiments, the optimizing includes deleting a ligand binding amino acid residue from the set of ligand binding amino acid residues. In embodiments, the optimizing includes a geometric transformation of at least one atomic coordinate of the ligand binding amino acid residue atomic coordinates. In embodiments, the optimizing includes a geometric transformation of the atomic coordinates of at least one of the ligand binding amino acid residue atomic coordinates. In embodiments, the optimizing includes a geometric transformation of the atomic coordinates of the ligand binding amino acid residue atomic coordinates.

In embodiments, the geometric transformation includes a translation (i.e., a geometric transformation that moves a coordinate by the same distance in a given direction) or a rotation of at least one atomic coordinate of the ligand binding amino acid residue atomic coordinates. In embodiments, the geometric transformation includes a translation (e.g., displacing the x coordinate) of at least one atomic coordinate of the ligand binding amino acid residue atomic coordinates. In embodiments, the geometric transformation includes a translation of at least two atomic coordinates of the ligand binding amino acid residue atomic coordinates. In embodiments, the geometric transformation includes a translation of all atomic coordinates (e.g., x, y, and z coordinates in Cartesian space) of the ligand binding amino acid residue atomic coordinates. In embodiments, the geometric transformation includes a rotation of at least one atomic coordinate of the ligand binding amino acid residue atomic coordinates. In embodiments, the geometric transformation includes a rotation of at least two atomic coordinates of the ligand binding amino acid residue atomic coordinates. In embodiments, the geometric transformation includes a rotation of at least three atomic coordinates of the ligand binding amino acid residue atomic coordinates. In embodiments, the geometric transformation includes a rotation of all atomic coordinates of the ligand binding amino acid residue atomic coordinates.

In embodiments, the optimizing includes a geometric transformation of at least one atomic coordinate of the non-ligand binding amino acid residue atomic coordinates. In embodiments, the geometric transformation includes a translation or a rotation of at least one atomic coordinate of the non-ligand binding amino acid residue atomic coordinates. In embodiments, the geometric transformation includes a translation of at least one atomic coordinate of the non-ligand binding amino acid residue atomic coordinates. In embodiments, the geometric transformation includes a translation of at least two atomic coordinates of the non-ligand binding amino acid residue atomic coordinates. In embodiments, the geometric transformation includes a translation of all atomic coordinates of the non-ligand binding amino acid residue atomic coordinates. In embodiments, the geometric transformation includes a rotation of at least one atomic coordinate of the non-ligand binding amino acid residue atomic coordinates. In embodiments, the geometric transformation includes a rotation of at least two atomic coordinates of the non-ligand binding amino acid residue atomic coordinates. In embodiments, the geometric transformation includes a rotation of at least three atomic coordinates of the non-ligand binding amino acid residue atomic coordinates. In embodiments, the geometric transformation includes a rotation of all atomic coordinates of the non-ligand binding amino acid residue atomic coordinates.

In embodiments, the optimizing includes 1a) calculating the force on each atom in the protein (e.g., the set of ligand binding amino acid residues; the set of non-ligand binding amino acid residues; and the ligand); 2a) evaluating the calculation to determine if it is the minimum or below an acceptable threshold; 3a) if the force is less than a threshold, the optimization is finished, otherwise perform a geometric transformation (e.g., translation) of at least one atomic coordinate on the atoms in the protein; and 4a) repeat.

In embodiments, the geometric transformation of at least one atomic coordinate includes no greater than a 6 Å displacement of any atomic coordinate. In embodiments, the geometric transformation of at least one atomic coordinate includes no greater than a 3 Å displacement of any atomic coordinate. In embodiments, the displacement is no greater than 0.1, 0.2, 0.3, 0.4, 0.5, 1, 2, 3, 4, 5, 6, 7, 8, 9, or 10 Å displacement of any atomic coordinate. In embodiments, the displacement is no greater than 0.01, 0.02, 0.03, 0.04, 0.05, 0.1, 0.2, 0.3, 0.4, 0.5, 0.6, 0.7, 0.8, 0.9, or 1.0 Å displacement of any atomic coordinate.

In embodiments, the set of ligand binding amino acids includes at least 50 amino acid residues. In embodiments, the set of ligand binding amino acids includes at least 40 amino acid residues. In embodiments, the set of ligand binding amino acids includes at least 30 amino acid residues. In embodiments, the set of ligand binding amino acids includes at least 20 amino acid residues. In embodiments, the set of ligand binding amino acids includes at least 12 amino acid residues. In embodiments, the set of ligand binding amino acids includes at least 10 amino acid residues. In embodiments, the set of ligand binding amino acids includes at least 8 amino acid residues. In embodiments, the set of ligand binding amino acids includes at least 6 amino acid residues. In embodiments, the set of ligand binding amino acids includes at least 5 amino acid residues. In embodiments, the set of ligand binding amino acids includes at least 4 amino acid residues. In embodiments, the set of ligand binding amino acids includes at least 3 amino acid residues. In embodiments, the set of ligand binding amino acids includes at least 2 amino acid residues. In embodiments the ligand binding amino acids are apolar. In embodiments the ligand binding amino acids are hydrophilic.

In embodiments, the set of ligand binding amino acids includes 50 amino acid residues. In embodiments, the set of ligand binding amino acids includes 40 amino acid residues. In embodiments, the set of ligand binding amino acids includes 30 amino acid residues. In embodiments, the set of ligand binding amino acids includes 20 amino acid residues. In embodiments, the set of ligand binding amino acids includes 12 amino acid residues. In embodiments, the set of ligand binding amino acids includes 10 amino acid residues. In embodiments, the set of ligand binding amino acids includes 8 amino acid residues. In embodiments, the set of ligand binding amino acids includes 6 amino acid residues. In embodiments, the set of ligand binding amino acids includes 5 amino acid residues. In embodiments, the set of ligand binding amino acids includes 4 amino acid residues. In embodiments, the set of ligand binding amino acids includes 3 amino acid residues. In embodiments, the set of ligand binding amino acids includes 2 amino acid residues. In embodiments the ligand binding amino acids are polar. In embodiments the ligand binding amino acids are hydrophilic.

In embodiments, the energy minimization calculation includes a molecular mechanics function, a structural bioinformatics function, an amino acid sidechain packing function, a protein radius of gyration function, or a combination thereof. In embodiments, the energy minimization calculation includes a penalty function.

In embodiments, the ligand is a porphyrin, porphycene, rubyrin, rosarin, hexaphyrin, sapphyrin, chlorophyll, chlorin, phthalocyanine, porphyrazine, corrole, N-confused porphyrin, bacteriochlorophyll, pheophytin, texaphyrin, or related macrocyclic-based component, that is capable of binding a metal ion. In embodiments, the ligand is a detectable agent. In embodiments, the ligand is a therapeutic agent, biological agent, cytotoxic agent, magnetic resonance imaging (MRI) agent, positron emission tomography (PET) agent, radiological imaging agent, diagnostic agent, theragnostic, or a photodynamic therapy (PDT) agent. In embodiments, the ligand is a therapeutic agent. In embodiments, the ligand is a biological agent. In embodiments, the ligand is a cytotoxic agent (e.g., an anticancer agent). In embodiments, the ligand is a magnetic resonance imaging (MRI) agent. In embodiments, the ligand is a positron emission tomography (PET) agent. In embodiments, the ligand is a radiological imaging agent.

In embodiments, the ligand is a diagnostic agent. In embodiments, the ligand is a theragnostic agent. In embodiments, the ligand is a photodynamic therapy (PDT) agent. In embodiments, the ligand is a small molecule.

In embodiments, the ligand is a catalyst. In embodiments, the catalyst catalyzes an abiological or bio-orthogonal reaction. In embodiments, the ligand is a molecule that exists within a living system (e.g., within an organism or a cell). In embodiments, the ligand atomic coordinates are optimized using known methods in the art (e.g., density functional theory using the B3-LYP functional). In embodiments, the ligand is a small molecule. In embodiments, the ligand is a metal cofactor. In embodiments, the ligand is a metal ion. In embodiments, the ligand is a protein. In embodiments, the ligand is a compound.

In embodiments, the method further includes synthesizing the protein (e.g., utilizing the expression vectors such as the plasmid method described in the Example, such as cloning into the IPTG-inducible pET-11a plasmid). In embodiments, the method further includes expressing the protein.

These ligand binding amino acid residues can form the backbone of a protein. Each ligand binding amino acid residue within the protein can be associated with a set of ligand binding amino acid residue atomic coordinates, which can define the ligand binding amino acid residue in space. Furthermore, each atom of the ligand can be associated with a set of ligand atomic coordinates, which can define the ligand in space. As noted herein, these coordinates can be Cartesian coordinates, internal coordinates, polar coordinates, spherical coordinates, and/or the like.

The set of ligand binding amino acid residues, the set of ligand binding amino acid residue atomic coordinates, the set of non-ligand binding amino acid residues, and the set of non-ligand binding amino acid residue atomic coordinates can be optimized. For example, the optimization can be performed using an energy minimization calculation including, for example, a molecular mechanics function, a structural bioinformatics function, an amino acid sidechain packing function, a protein radius of gyration function, and/or the like. Optimizing the set of ligand binding amino acid residues, the set of ligand binding amino acid residue atomic coordinates, the set of non-ligand binding amino acid residues, and the set of non-ligand binding amino acid residue atomic coordinates can generate an energetically stabilized protein.

### III. Systems and Mediums

In some example embodiments, challenges associated with designing de novo a protein capable of binding to a small molecule (or another ligand) are addressed by mapping the tertiary structure of a protein directly to the sequences of amino acids that encode the folding and the binding exhibited by the protein. For example, a protein may be designed de novo by creating an ensemble of backbones with geometries consistent with the known plasticity of a selected protein fold. For each backbone, one or more van der Mers (vdMs) that interact with a portion of the ligand, such as one or more targeted chemical groups within the small molecule, may be identified. As each van der Mer is a structural unit occupying a specific residue position on the backbone of a protein, the identification of van der Mers may also determine the binding sites of the small molecule (or other ligand). Thus, the backbone geometry of the protein may be dictated by a maximum binding affinity to the desired small molecule (or other ligand). Once the binding sites are identified, additional residues within the binding sites and the protein core may be packed. The resulting protein may therefore exhibit a tertiary structure and sequence that support the desired function of binding to the small molecule (or other ligand).

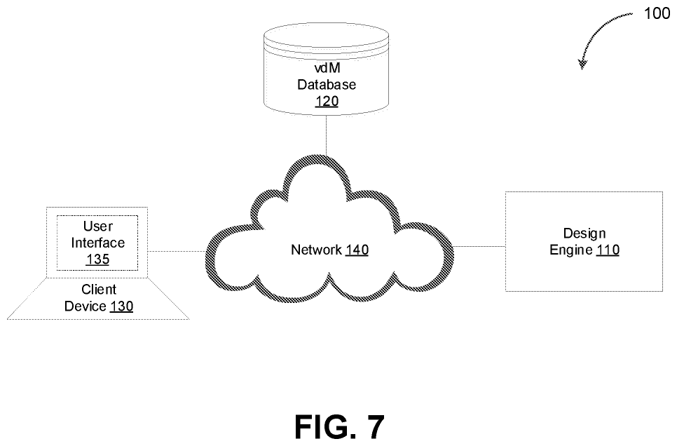

FIG. 7 depicts a system diagram illustrating an example of a protein design system 100, in accordance with some example embodiments. The example of the protein design system 100 shown in FIG. 7 may include a design engine 110, a van der Mer (vdM) database 120, and a client device 130. As shown in FIG. 7, the design engine 110, the van der Mer database 120, and the client device 130 may be communicatively coupled via a network 140. The network 140 may be a wired network and/or a wireless network including, for example, a local area network (LAN), a virtual local area network (VLAN), a wide area network (WAN), a public land mobile network (PLMN), the Internet, and/or the like.

In some example embodiments, the design engine 110 may be configured to support the de novo design of a protein exhibiting a binding affinity for a ligand including, for example, a peptide (e.g., 2 to 30 amino acid residues), a protein (e.g., greater than 30 amino acid residues), a small molecule (e.g., a compound with a molecular weight of less than 2000 Daltons), a small molecule-metal-ion complex (e.g., a metalloporphyrin), and/or the like. The design engine 110 may design the protein by creating an ensemble of backbones with geometries consistent with the known plasticity of a selected protein fold. In the example of the protein design system 100 shown in FIG. 7, the design engine 110 may receive, from the client device 130, one or more user inputs selecting a protein fold. The selection of the protein fold may be made, for example, via a user interface 135 at the client device 130.

For each backbone, the design engine 110 may identify one or more van der Mers (vdMs) that interact with a portion of the ligand, such as one or more targeted chemical groups within the small molecule. Each van der Mer may be an in silico unit of local protein structure known to interact with a portion of the ligand. Moreover, each van der Mer may occupy a specific residue position (e.g., a statistically preferred position) on the backbone structure of the protein when the van der Mer is interacting with the portion of the ligand (e.g., the targeted chemical groups of the small molecule). Thus, the van der Mers identified by the design engine 110 may provide a direct link between the tertiary structures of the protein to a desired function, such as a binding affinity for the ligand. For example, the van der Mers that are identified by the design engine 110 may define the binding sites of the ligand with additional residues within the binding sites and the protein core being packed accordingly.

In some example embodiments, the one or more van der Mers known to interact with the portion of the ligand, such as the targeted chemical groups of a small molecule, may be identified by the design engine 110 querying the van der Mer database 120. The van der Mer database 120 may include a selection of van der Mers, each of which known to exhibit an interaction with a portion of a ligand (e.g., a targeted chemical group such as aspartic acid (Asp), carboxamide (CONH₂), and/or the like). The selection of van der Mers may be curated by searching a database of known protein structures (e.g., from the Protein Data Bank (PDB) and/or the like). For example, a unit of a protein structure may be identified as a van der Mer of a chemical group if the amino-acid residues contained therein are in van der Waals (vdW) contact with the given chemical group. Furthermore, a unit of a protein structure may be identified as the van der Mer of a chemical group based on the nature of the contact (e.g., H-bond, close vdW contact, wide vdW contact). For instance, van der Mers of the chemical group carboxamide may be identified by iterating through Asn and Gln residues in each unique known protein chain (e.g., in the Portein Data Bank (PDB) and/or the like). Van der Mers of the chemical group carboxamide may be those residues that are within van der Waals contact with the sidechain's carboxamide (e.g., CB, CG, OD1, ND2, HD21, HD22 atoms of Asn) by forming H-bonded interactions.

In some example embodiments, the van der Mers included in the van der Mer database 120 may be organized into clusters of related van der Mers that exhibit similar observed interactions with a chemical group. For example, the van der Mers of a chemical group may be clustered based on the coordinates of the protein backbone and the coordinates of the chemical group bound to the protein. This clustering may facilitate subsequent searches through the van der Mer database 120. For instance, only 31 clusters of Asp/carboxamide vdMs are needed to capture half of the observed interactions. Each van der Mer cluster may be associated with a cluster score (C), which provides a quantitative measure for how representative that cluster's interaction geometry is for that residue type across the known protein structures (e.g, in the Protein Data Bank (PDB) and/or the like). The score may be determined based on the placement of a chemical group relative to a protein backbone, since the coordinates of the backbone and chemical group are the only coordinates involved in the clustering. A positive cluster score may indicate that the location of the chemical group relative to the backbone, represented by the cluster, is enriched relative to other locations of the cluster group. Thus, in some example embodiments, the design engine 110 may select van der Mers having a positive cluster score when identifying van der Mers for the de novo design of a protein.

As noted, the identification of one or more van der Mers exhibiting an interaction with a portion of a ligand (e.g., a targeted chemical group of a small molecule) may determine the binding sites for the ligand on the backbone of a protein being designed to exhibit a binding affinity for the ligand. This is because each van der Mer may occupy a specific residue position on the backbone structure of the protein when the van der Mer is interacting with the portion of the ligand (e.g., the targeted chemical groups of the small molecule). The position of each van der Mer may correspond to the statistically preferred orientation of the portion of the ligand relative to the backbone structure of the protein when the van der Mer is interacting with the portion of the ligand. The remainder of the protein may be designed by the design engine 110 packing additional residues within the binding sites and packing the protein core. The resulting protein may exhibit a tertiary structure and amino acid sequence that supports the desired binding affinity to a particular ligand.

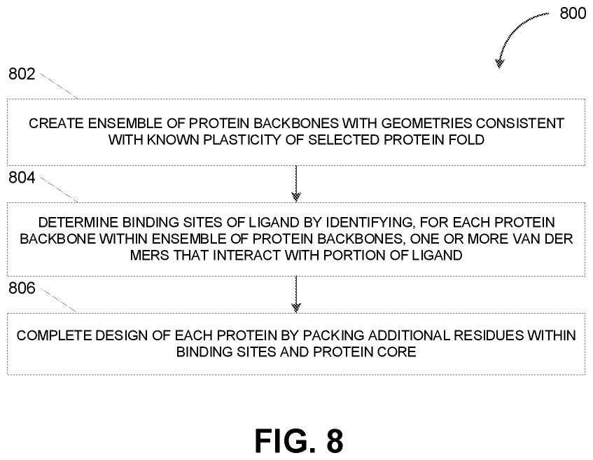

FIG. 8 depicts a flowchart illustrating an example of a process 800 for protein design, in accordance with some example embodiments. Referring to FIGS. 7-8, the process 800 may be performed by the design engine 110 to design a protein exhibits a binding affinity for a ligand such as, for example, a peptide (e.g., 2 to 30 amino acid residues), a protein (e.g., greater than 30 amino acid residues), a small molecule (e.g., a compound with a molecular weight of less than 2000 Daltons), a small molecule-metal-ion complex (e.g., a metalloporphyrin), and/or the like.

The design engine 110 may creating an ensemble of protein backbones with geometries consistent with the known plasticity of a selected protein fold (802). For example, the design engine 110 may receive, from the client device 130, one or more user inputs identifying a designable protein fold. The design engine 110 may create an ensemble of protein backbones with geometries that are consistent with the known plasticity of the designable protein fold.

The design engine 110 may determine the binding sites for a ligand by identifying, for each protein backbone from the ensemble of protein backbones, one or more van der Mers that interact with a portion of the ligand (804). In some example embodiments, the design engine 110 may identify one or more van der Mers known to interact with the portion of the ligand, such as the targeted chemical groups of a small molecule, by querying the van der Mer database 120.

The van der Mer database 120 may include a selection of van der Mers, each of which known to exhibit an interaction with a portion of a ligand (e.g., a targeted chemical group such as aspartic acid (Asp), carboxamide (CONH₂), and/or the like). The van der Mers included in the van der Mer database 120 may be organized into clusters of related van der Mers that exhibit similar observed interactions with a chemical group. For example, the van der Mers of a chemical group may be clustered based on the coordinates of the protein backbone and the coordinates of the chemical group bound to the protein. By organizing van der Mers into clusters, queries to the van der Mer database 120 may be executed faster and with less computational resources.

The design engine 110 may complete the design for each protein by packing additional residues within the binding site and/or the protein core (806). In some example embodiments, upon identifying the binding sites for the ligand, the remainder of the protein structure may be designed by the design engine 110 packing additional residues within the binding sites and packing the protein core. The design engine 110 may apply a variety of algorithms to pack each protein structure. For example, the protein structure may be packed with hydrophobic amino acid residues and/or hydrophilic amino acid residues.

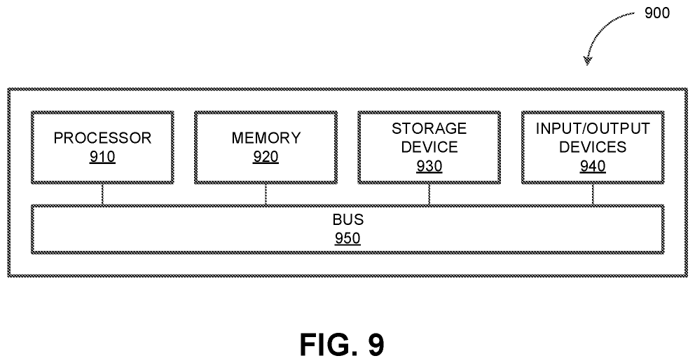

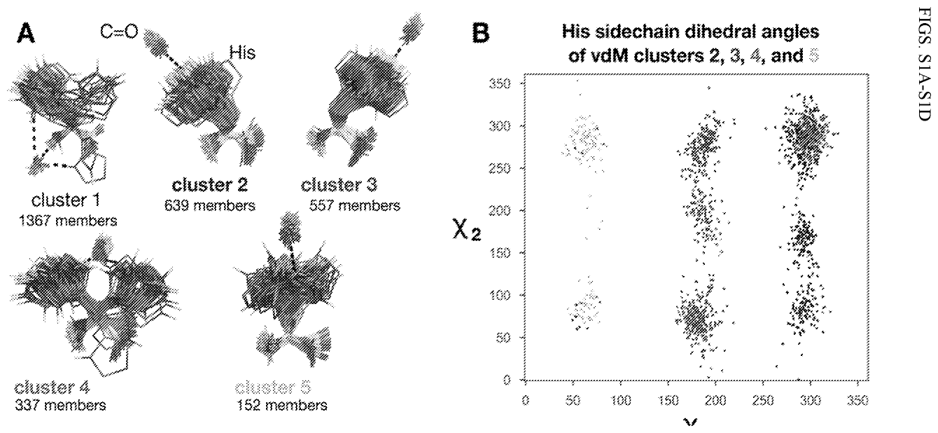

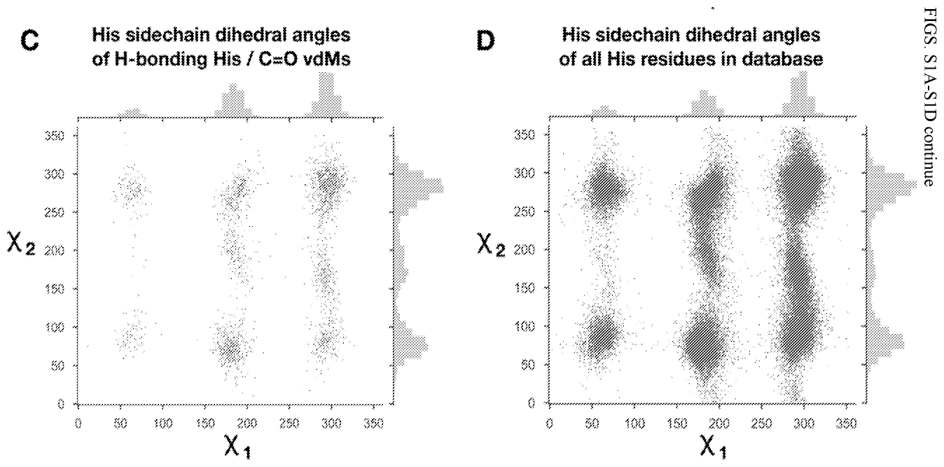

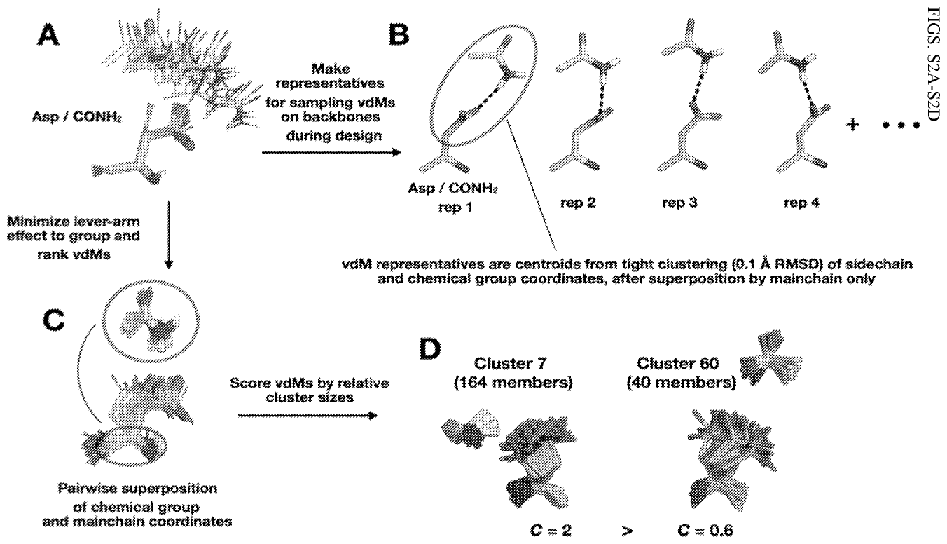

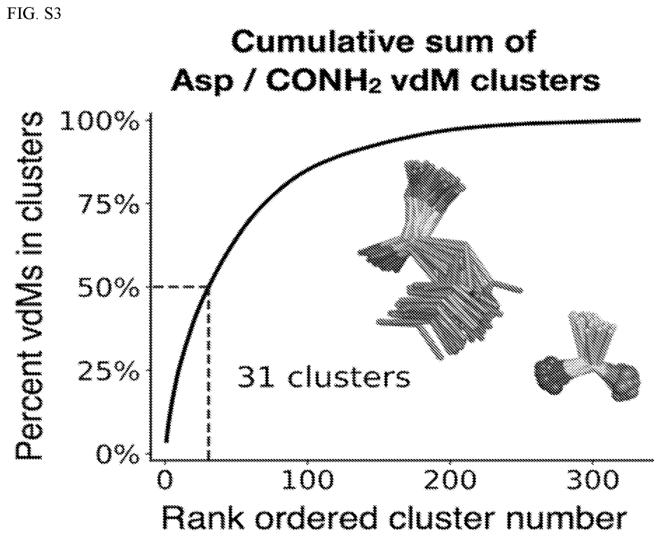

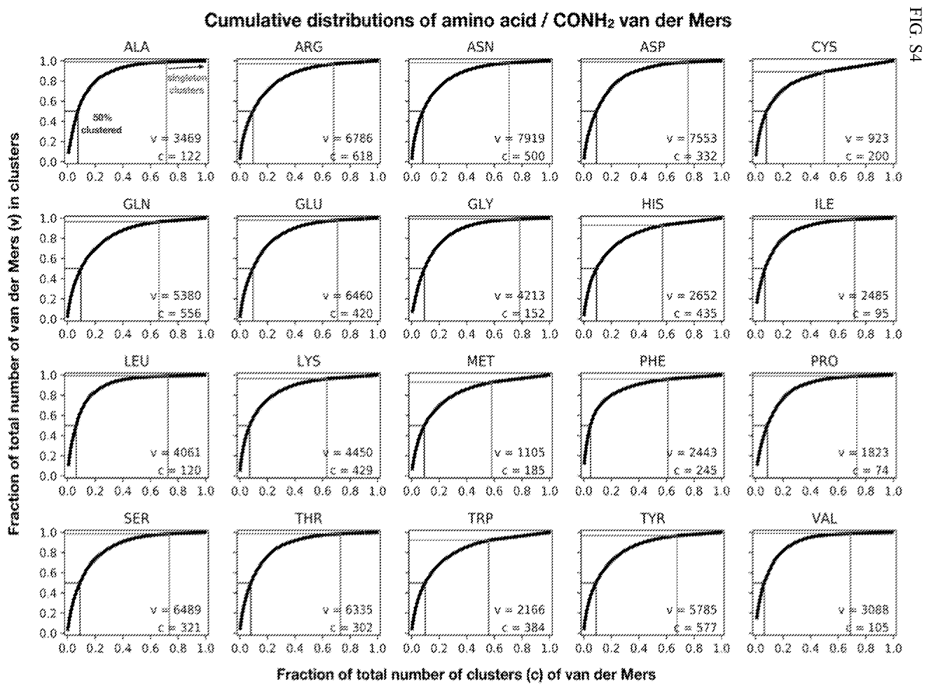

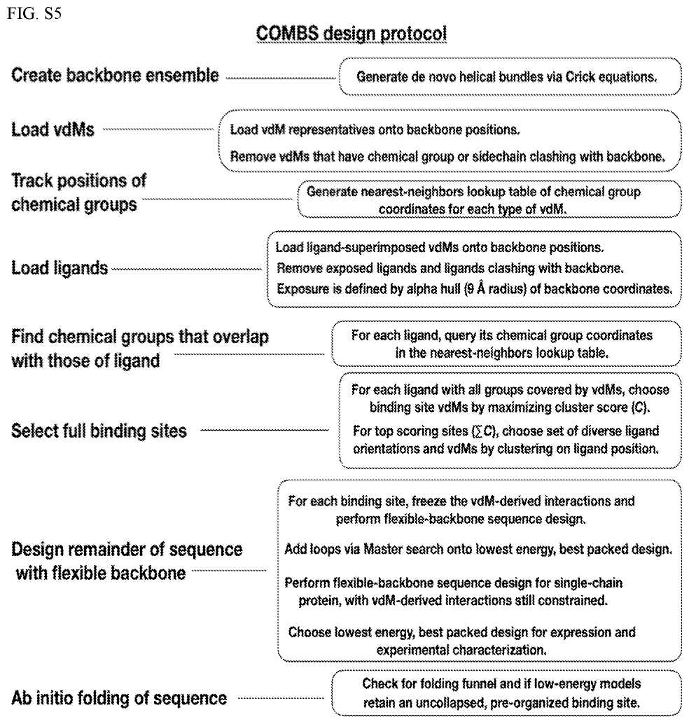

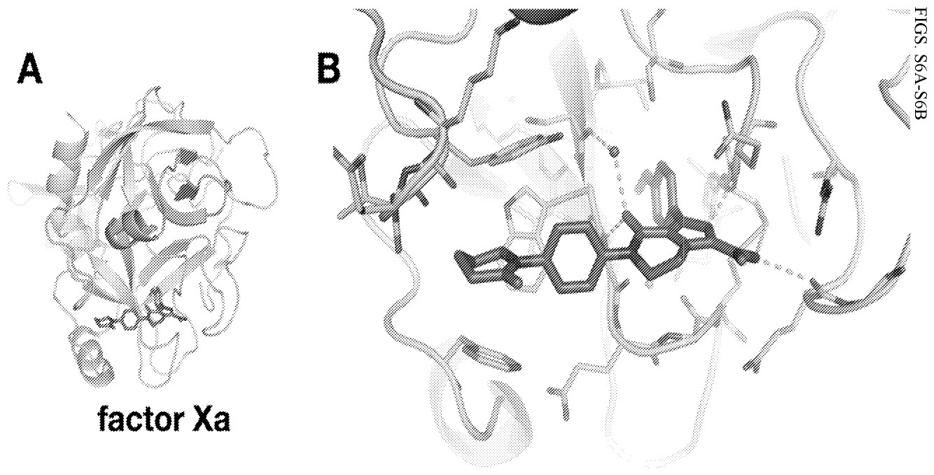

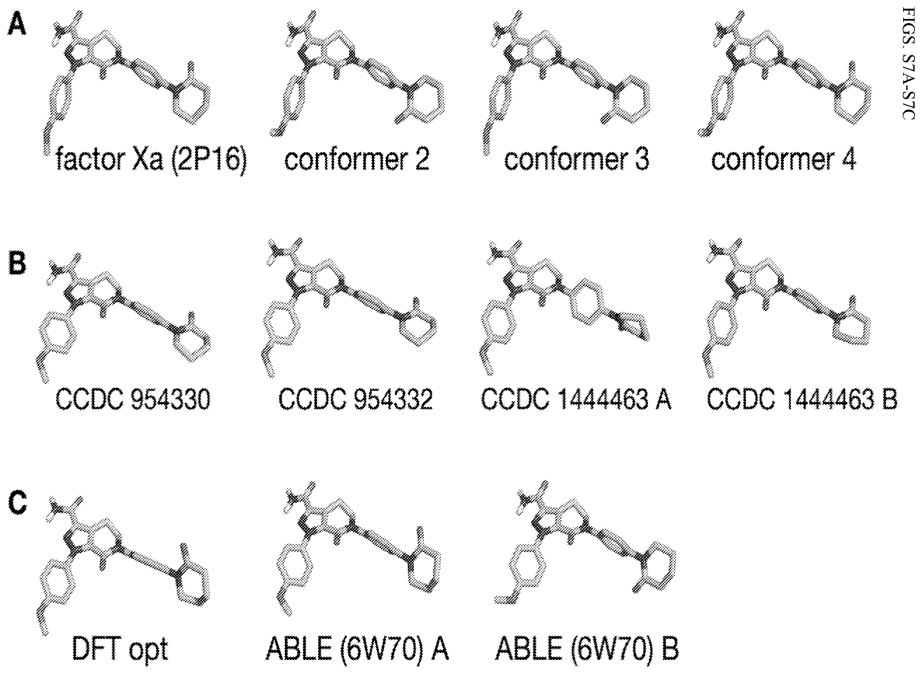

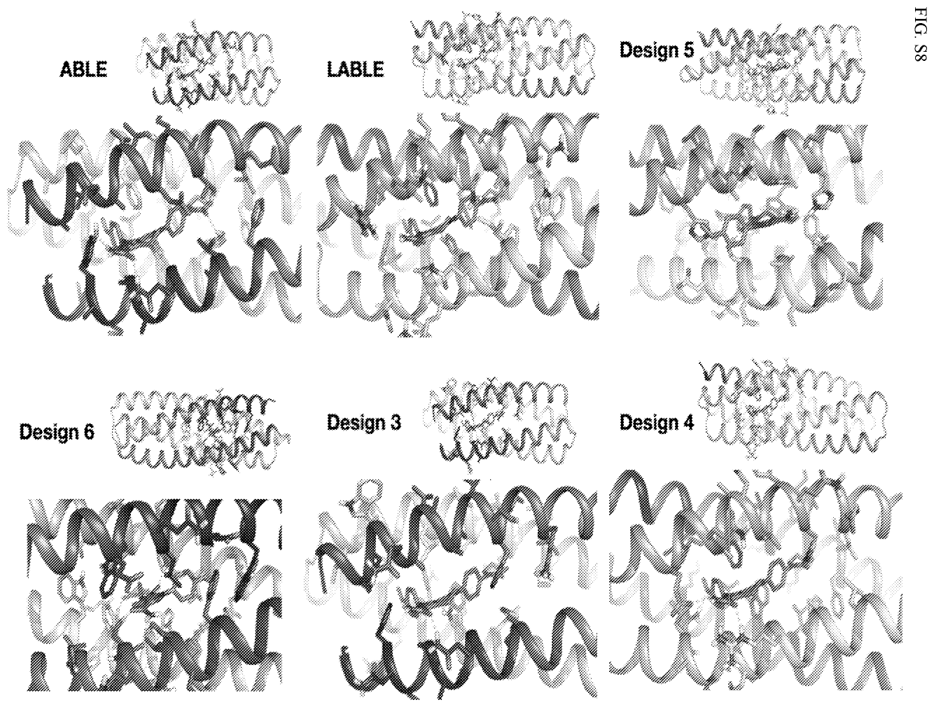

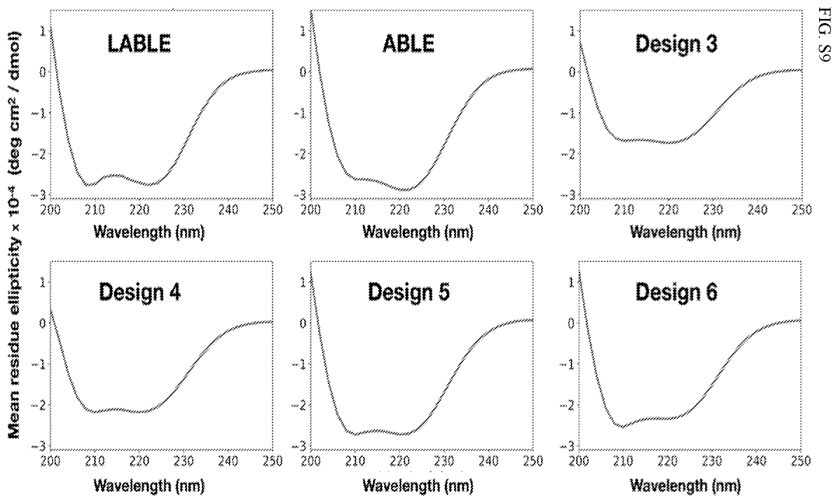

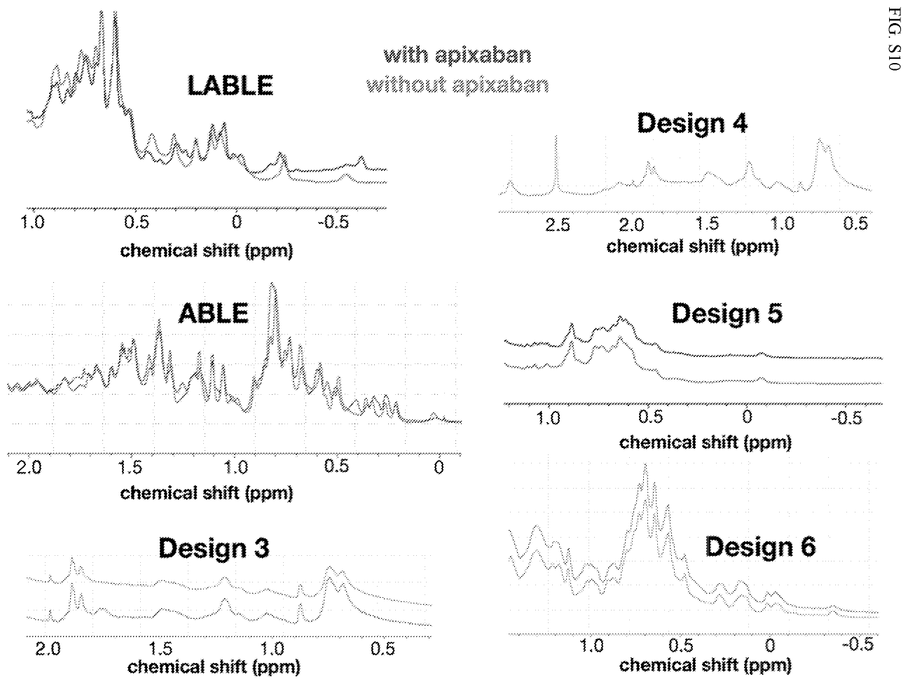

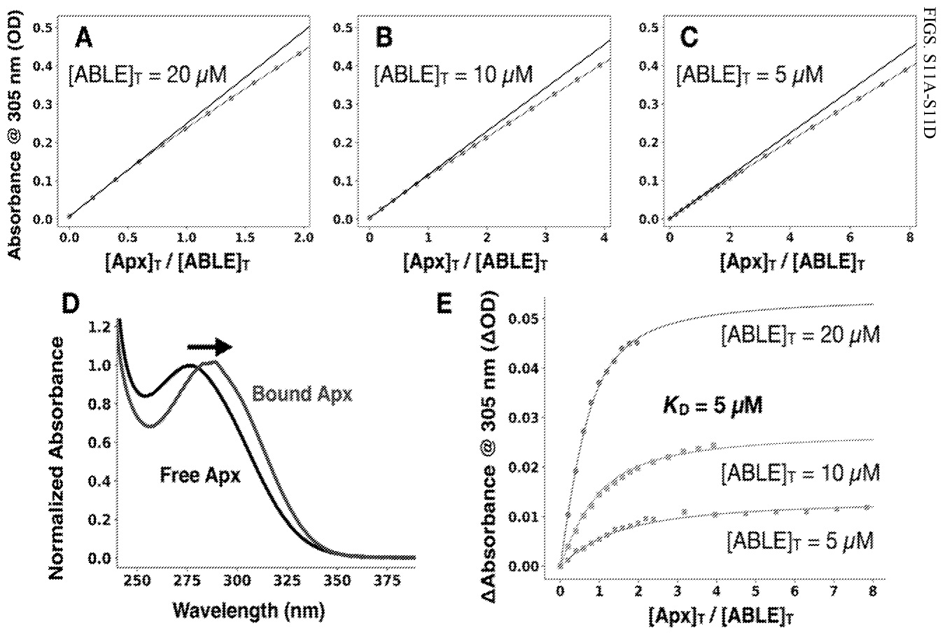

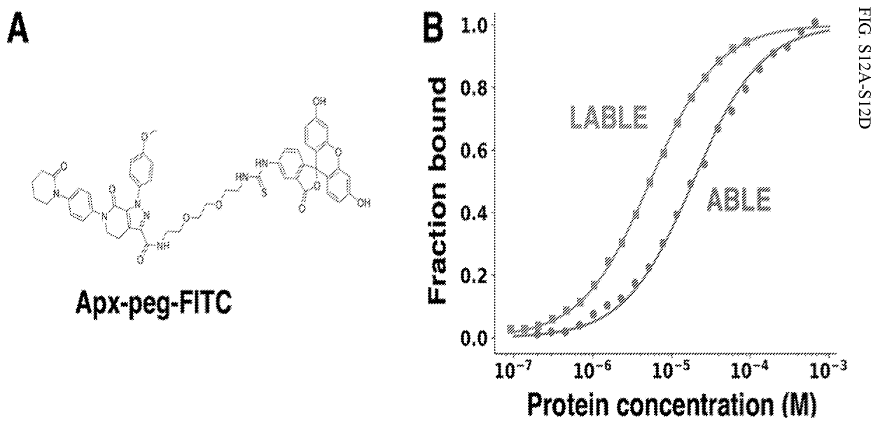

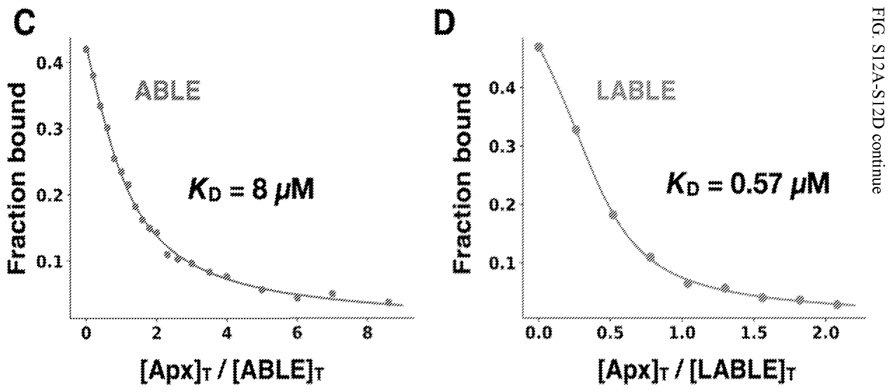

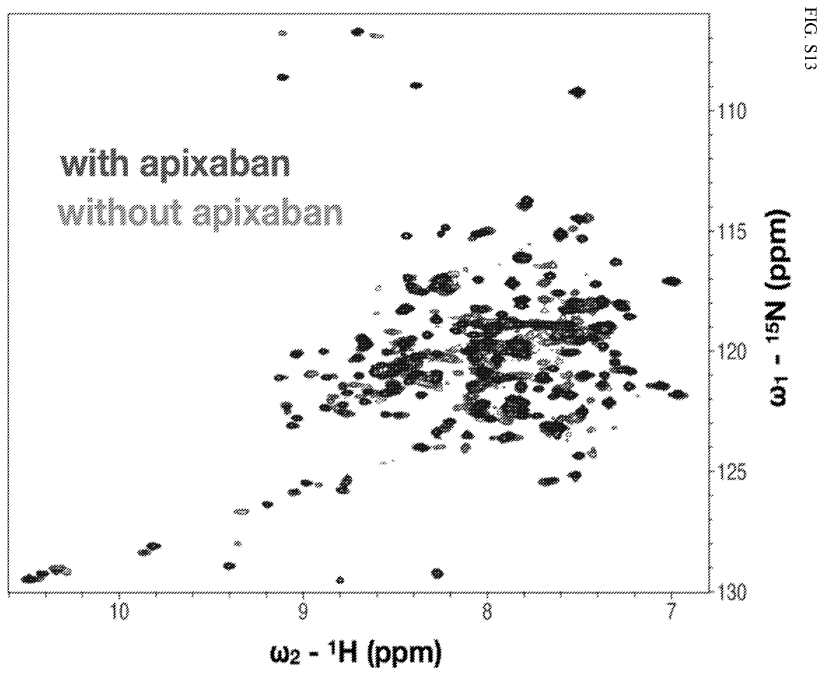

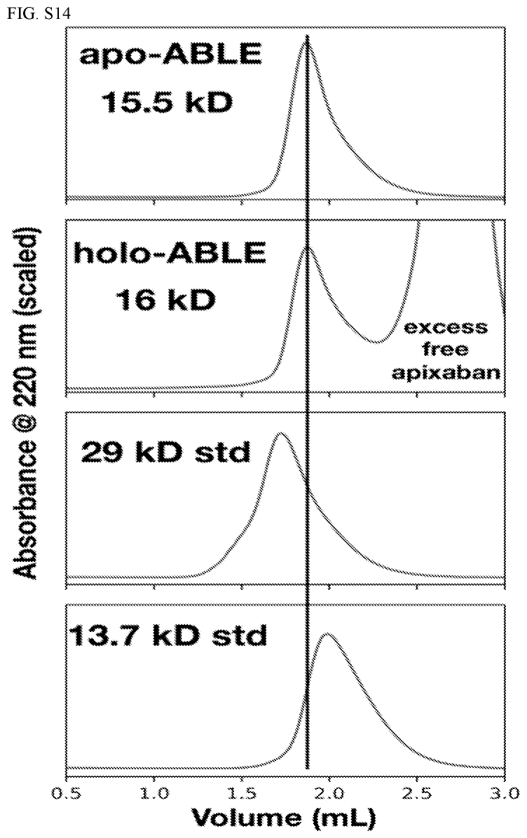

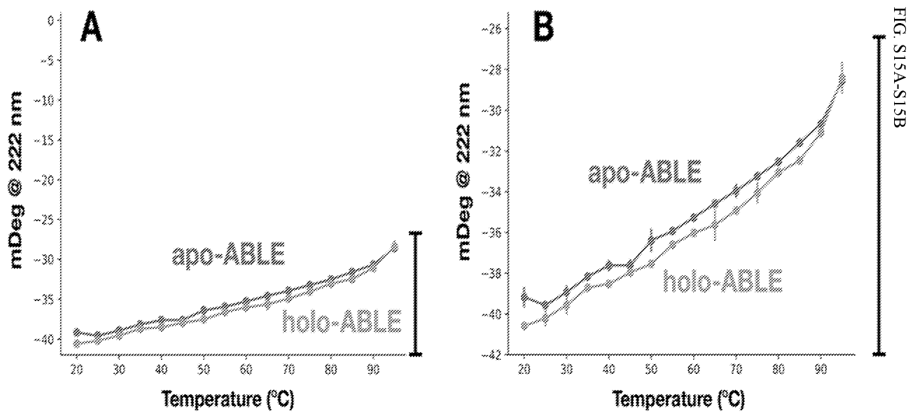

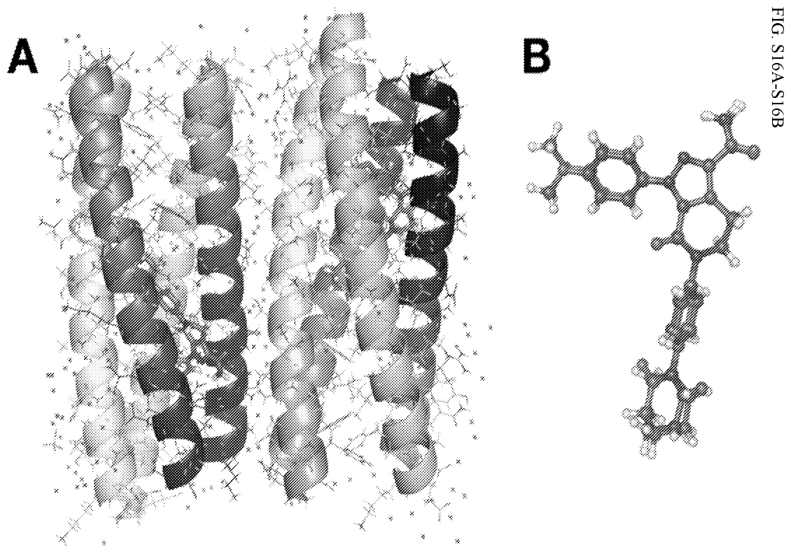

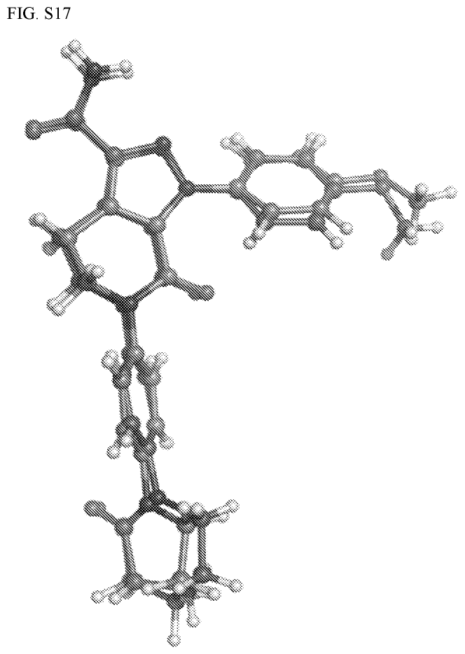

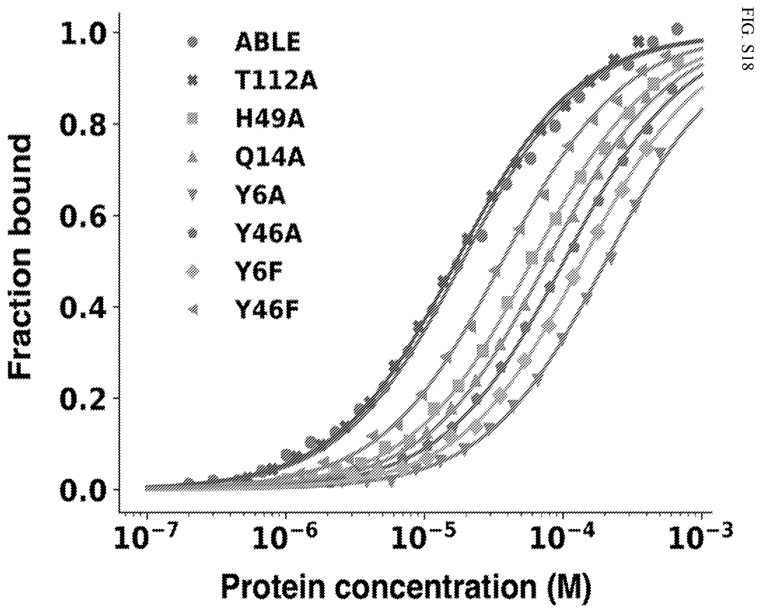

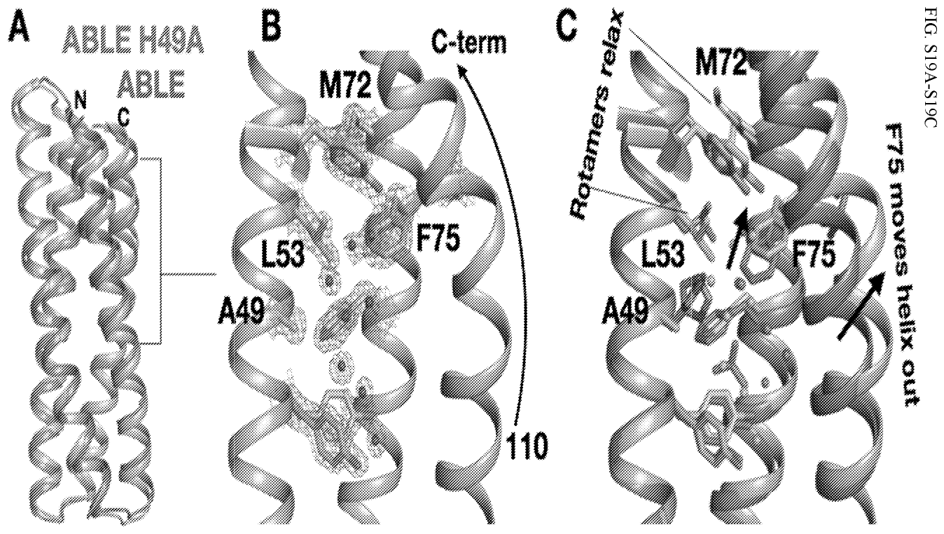

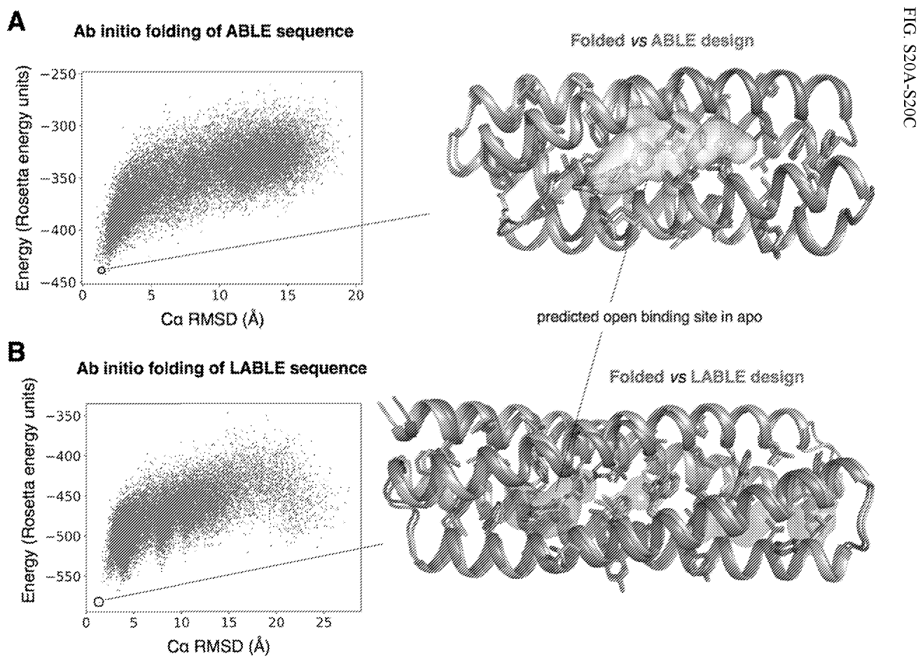

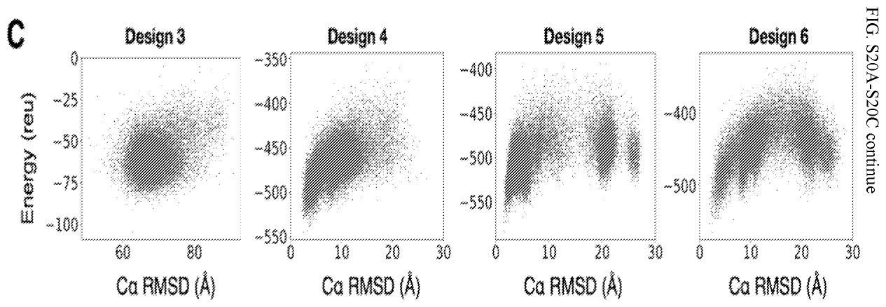

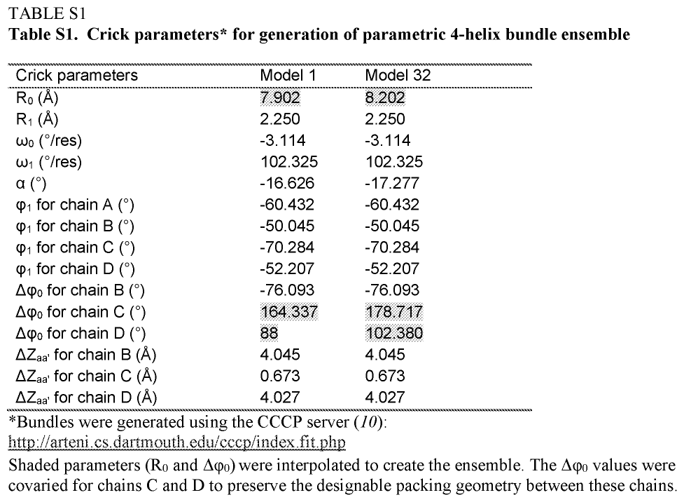

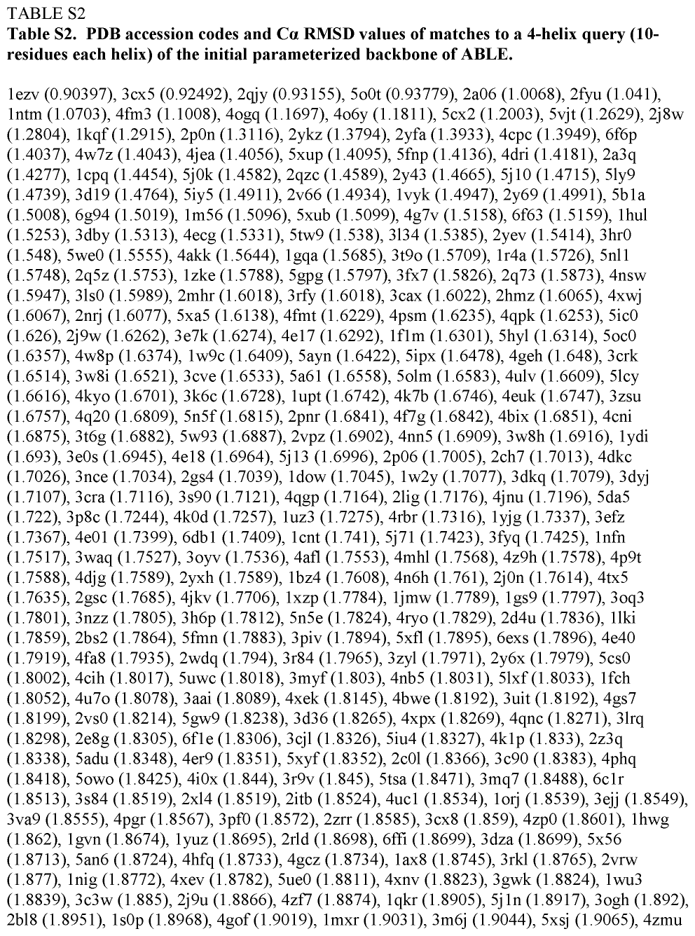

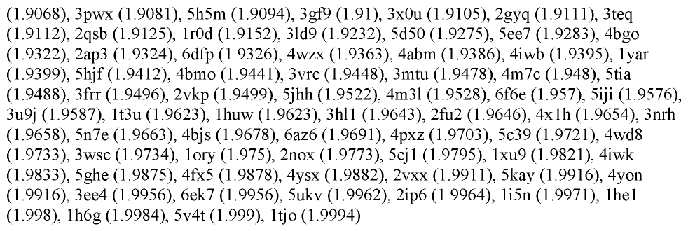

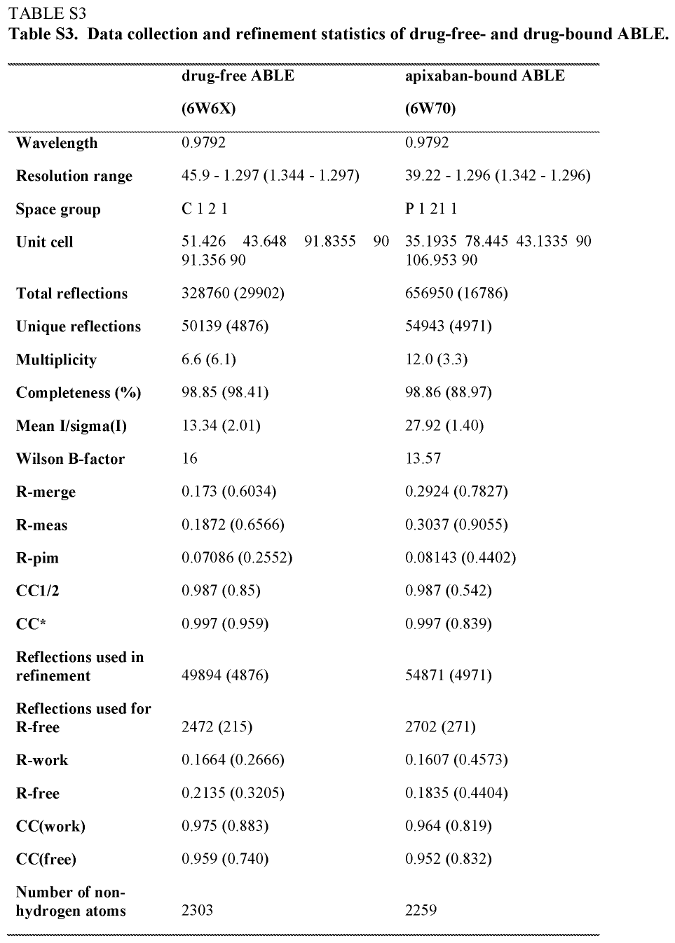

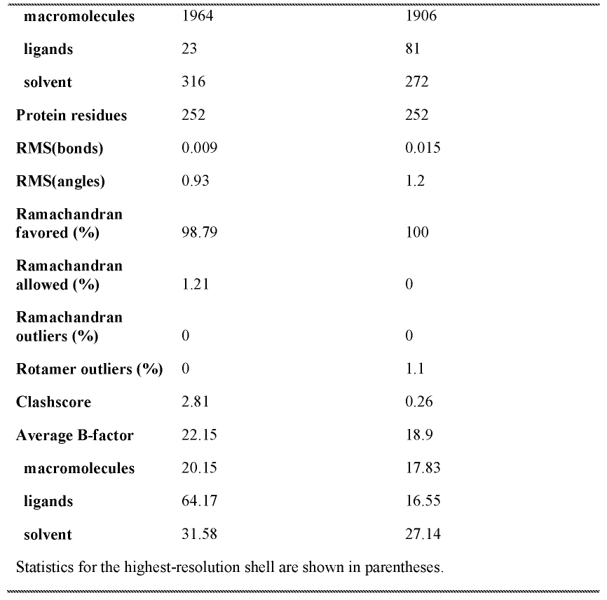

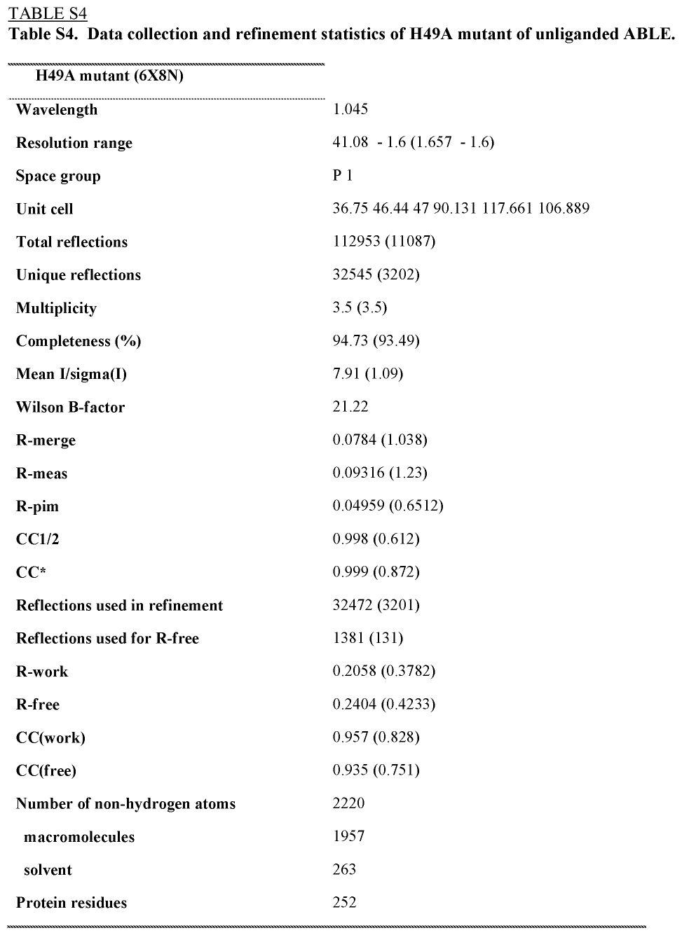

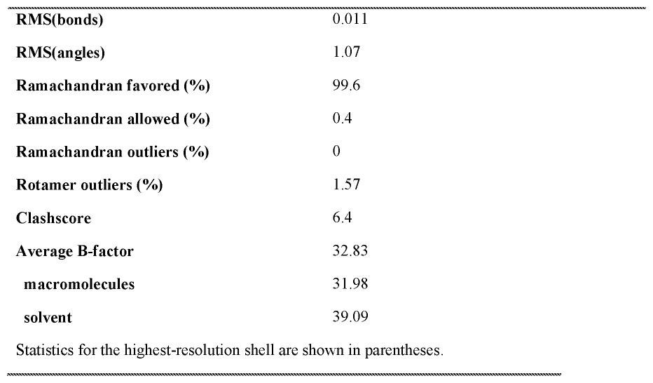

FIG. 9 depicts a block diagram illustrating an example of computing system 900, in accordance with some example embodiments. Referring to FIGS. 7-9, the computing system 900 may be used to implement the design engine 110, the client device 130, and/or any components therein.

As shown in FIG. 9, the computing system 900 can include a processor 910, a memory 920, a storage device 930, and input/output devices 940. The processor 910, the memory 920, the storage device 930, and the input/output devices 940 can be interconnected via a system bus 950. The processor 910 is capable of processing instructions for execution within the computing system 900. Such executed instructions can implement one or more components of, for example, the design engine 110, the client device 130, and/or the like. In some example embodiments, the processor 910 can be a single-threaded processor. Alternately, the processor 910 can be a multi-threaded processor. The processor 910 is capable of processing instructions stored in the memory 920 and/or on the storage device 930 to display graphical information for a user interface provided via the input/output device 940.

The memory 920 is a computer readable medium such as volatile or non-volatile that stores information within the computing system 900. The memory 920 can store data structures representing configuration object databases, for example. The storage device 930 is capable of providing persistent storage for the computing system 900. The storage device 930 can be a floppy disk device, a hard disk device, an optical disk device, or a tape device, or other suitable persistent storage means. The input/output device 940 provides input/output operations for the computing system 900. In some example embodiments, the input/output device 940 includes a keyboard and/or pointing device. In various implementations, the input/output device 940 includes a display unit for displaying graphical user interfaces.

The memory 920 is a computer readable medium such as volatile or non-volatile that stores information within the computing system 900. The memory 920 can store data structures representing configuration object databases, for example. The storage device 930 is capable of providing persistent storage for the computing system 900. The storage device 930 can be a floppy disk device, a hard disk device, an optical disk device, or a tape device, or other suitable persistent storage means. The input/output device 940 provides input/output operations for the computing system 900. In some example embodiments, the input/output device 940 includes a keyboard and/or pointing device. In various implementations, the input/output device 940 includes a display unit for displaying graphical user interfaces.

In some example embodiments, the computing system 900 can be used to execute various interactive computer software applications that can be used for organization, analysis and/or storage of data in various formats. Alternatively, the computing system 900 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 940. The user interface can be generated and presented to a user by the computing system 900 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

In an aspect is provided a system for identifying a protein capable of binding a compound including at least one data processor; and at least one memory storing instructions which, when executed by the at least one data processor, result in operations including identifying van der Mers representing the chemical groups of the compound and amino acid residues of the protein capable of interacting with the chemical groups of the compound in silico, and wherein the protein has secondary and tertiary protein structure when bound to the compound.

In an aspect is provided a system for identifying a protein capable of binding a compound, including: at least one data processor; and at least one memory storing instructions which, when executed by the at least one data processor, result in operations including:
(a) generating a first set of atomic protein coordinates representing a backbone structure of the protein;
(b) identifying a first van der Mer from a van der Mer database including a first set of atomic van der Mer coordinates representing a first chemical group of the compound, a first amino acid side chain and a first portion of a protein backbone bound to the first amino acid side chain, wherein the first chemical group interacts in silico with the first portion of a protein backbone or the first amino acid side chain;
(c) identifying portions of the backbone structure of the protein having atomic protein coordinates that are capable of overlapping with the atomic van der Mer coordinates of the first portion of a protein backbone of the first van der Mer;
(d) repeating steps (b) and (c) for at least one additional van der Mer independently representing an optionally different chemical group of the compound, an additional independent amino acid side chain and an additional independent portion of a protein backbone bound to the independent additional amino acid side chain;
(e) generating a set of atomic chemical coordinates representing the compound;
(f) generating at least one set of atomic coordinates of an in silico complex of the protein capable of binding the compound bound to the compound; wherein the in silico complex optimizes the overlap between the atomic chemical coordinates of the compound chemical groups and the atomic van der Mer coordinates representing the independent chemical groups of the compound identified in steps (b) to (d);
(g) generating a set of atomic amino acid coordinates for amino acid side chains and portions of protein backbone independently representing those portions of the backbone structure of the protein that are not overlapping with atomic van der Mer coordinates of the van der Mer including atomic van der Mer coordinates that are overlapping with the atomic chemical coordinates representing the compound of the in silico complex of step (f);
(h) based at least in part on steps (a) to (g), optimizing atomic coordinates of the compound and protein thereby identifying a protein capable of binding the compound.

In embodiments, the system includes generating a plurality of sets of atomic coordinates of an in silico complex of the protein capable of binding the compound bound to the compound in step (f). In embodiments, the system includes generating all possible sets of atomic coordinates of an in silico complex of the protein capable of binding the compound bound to the compound in step (f). In embodiments, the plurality of sets of atomic coordinates of an in silico complex of the protein capable of binding the compound bound to the compound are independently different from each other and optimization of the overlap between the atomic chemical coordinates of the compound chemical groups and the atomic van der Mer coordinates representing the independent chemical groups of the compound identified in steps (b) to (d) is performed without duplication of the sets. In embodiments, the plurality of independent sets of atomic coordinates of an in silico complex of the protein capable of binding the compound bound to the compound are independently different from each other and are scored. In embodiments, the scoring includes calculating a cluster score for each of the plurality of sets of atomic coordinates of an in silico complex of the protein capable of binding the compound bound to the compound. In embodiments, the cluster score is a function (e.g., including but not limited to a natural log or logistical function) of the ratio of 1) the number of members in an independent set of geometrically overlapping van der Mer of one chemical group and one amino acid to 2) the average number of members in all independent sets van der Mer of the chemical group and the amino acid. In embodiments, the members in an independent set of geometrically overlapping compound van der Mer of one chemical group are identified by having an RMSD below a threshold, wherein the RMSD is calculated using the atomic coordinates of the chemical group and the backbone atoms of the amino acid residue of a van der Mer. In embodiments, the RMSD threshold is 0.5 angstrom. In embodiments, step (a) includes generating a plurality of independent sets of atomic protein coordinates representing independent backbone structures of the protein. In embodiments, the plurality of independent backbone structures of the protein have a similar overall three dimensional fold. In embodiments, the plurality of independent backbone structures of the protein have an RMSD of less than 3 angstrom. In embodiments, the compound chemical groups and van der Mer chemical groups are polar groups. In embodiments, steps (g) and (h) include use of a method described in international application no. WO2019/023644. In embodiments, step (c) includes identifying all portions of the backbone structure of the protein having atomic protein coordinates that are capable of overlapping with the atomic van der Mer coordinates of the first portion of a protein backbone of the first van der Mer. In embodiments, step (d) includes repeating steps (b) and (c) for all van der Mer in the van der Mer database independently representing all chemical groups of the compound, an additional independent amino acid side chain and an additional independent portion of a protein backbone bound to said independent additional amino acid side chain. In embodiments, the overlap of the van der Mers and the compound chemical groups are not selected one at a time, but rather in pairs, triplets or higher order combinations. In embodiments, van der Mers at multiple sites are selected and the RMSD between these van der Mer chemical groups and the compound chemical groups is computed. In embodiments, the RMSD between van der Mer chemical groups and the compound chemical groups is precomputed and saved in lookup tables. In embodiments, the overlap of the van der Mers and the compound chemical groups are selected in pairs. In embodiments, the overlap of the van der Mers and the compound chemical groups are selected in triplets. In embodiments, the overlap of the van der Mers and the compound chemical groups are selected in combinations greater than three. In embodiments an identified van der Mer is a cluster having a 0.5 angstrom RMSD between members. In embodiments an identified van der Mer is a sub-cluster of a van der Mer. In embodiments an identified van der Mer is a van der Mer cluster member. In embodiments an identified van der Mer is a van der Mer representative. In embodiments an identified van der Mer is a cluster having a 0.1 angstrom RMSD between members.

In an aspect is provided a system for identifying a complex of a protein bound to a compound, including: at least one data processor; and at least one memory storing instructions which, when executed by the at least one data processor, result in operations including:
(a) generating a first set of atomic protein coordinates representing the side chain and backbone structure of the protein;
(b) identifying a first van der Mer from a van der Mer database including a first set of atomic van der Mer coordinates representing a first chemical group of the compound, a first amino acid side chain and a first portion of a protein backbone bound to the first amino acid side chain, wherein the first chemical group interacts in silico with the first portion of a protein backbone or the first amino acid side chain;
(c) identifying portions of the backbone structure of the protein having atomic protein coordinates that are capable of overlapping with the atomic van der Mer coordinates of the first portion of a protein backbone of the first van der Mer wherein the amino acid side chain of the van der Mer and the amino acid side chain directly attached to the overlapping portions of the backbone structure of the protein are the same side chain;
(d) repeating steps (b) and (c) for at least one additional van der Mer independently representing an optionally different chemical group of the compound, an additional independent amino acid side chain and an additional independent portion of a protein backbone bound to the independent additional amino acid side chain;
(e) generating a set of atomic chemical coordinates representing the compound;
(f) generating at least one set of atomic coordinates of an in silico complex of the protein capable of binding the compound bound to the compound; wherein the in silico complex optimizes the overlap between the atomic chemical coordinates of the compound chemical groups and the atomic van der Mer coordinates representing the independent chemical groups of the compound identified in steps (b) to (d);
(g) based at least in part on steps (a) to (f), optimizing atomic coordinates of the compound and protein thereby identifying a complex of a protein bound to a compound.

In an aspect is provided a system for identifying a protein capable of binding a compound, including: at least one data processor; and at least one memory storing instructions which, when executed by the at least one data processor, result in operations including:
(a) generating a first set of atomic protein coordinates representing a protein backbone structure;
(b) generating a first set of atomic chemical coordinates representing a first chemical group of the compound;
(c) identifying a first van der Mer from a van der Mer database including a first set of atomic van der Mer coordinates representing the first chemical group of the compound, a first amino acid side chain and a first portion of a protein backbone bound to the first amino acid side chain, wherein the first chemical group interacts in silico with the first portion of a protein backbone or the first amino acid side chain;
(d) generating a second set of atomic chemical coordinates representing a second chemical group of the compound;
(e) identifying a second van der Mer from the van der Mer database including a second set of atomic van der Mer coordinates representing the second chemical group of the compound, a second amino acid side chain and a second portion of a protein backbone bound to the second amino acid side chain, wherein the second chemical group interacts in silico with the second portion of a protein backbone or the second amino acid side chain;
(f) calculating an energetic stability of the protein backbone structure bound to the compound using the first set of atomic van der Mer coordinates and the second set of atomic van der Mer coordinates in silico;
(g) repeating steps (a) to (f) for additional van der Mers representing the first chemical group of the compound, a first amino acid side chain and a first portion of a protein backbone bound to the first amino acid side chain and additional van der Mers representing the second chemical group of the compound, a second amino acid side chain and a second portion of a protein backbone bound to the second amino acid side chain;
(h) generating a set of atomic amino acid coordinates for amino acid side chains and portions of protein backbone independently representing those portions of the protein backbone structure not represented by a van der Mer of steps (a) to (g);
(i) based at least in part on steps (a) to (h), optimizing atomic coordinates of the compound and protein thereby identifying a protein capable of binding the compound.

In an aspect is provided a system for identifying a protein capable of binding a compound, including: at least one data processor; and at least one memory storing instructions which, when executed by the at least one data processor, result in operations including:
(a) identifying a first van der Mer from a van der Mer database including atomic van der Mer coordinates of a chemical group of the compound, wherein the atomic van der Mer coordinates of the chemical group in the first van der Mer overlap with the atomic chemical coordinates of the chemical group of the compound;
(b) identifying a protein backbone for the protein wherein the atoms of the protein backbone are associated with a set of atomic protein coordinates;
(c) identifying an overlap between the atomic van der Mer coordinates of the amino acid backbone of the first van der Mer identified in step (a) and the atomic protein coordinates of an amino acid residue of the protein backbone identified in step (b);
(d) optionally repeating steps (a) to (c) for a different chemical group of the compound;
(e) identifying independent sets of van der Mer identified in steps (a) to (d) wherein all van der Mer of each independent set include atomic van der Mer coordinates that collectively simultaneously overlap atomic protein coordinates of the protein backbone identified in step (b);
(f) identifying at least one independent set of van der Mer identified in step (e) with a cluster score above a threshold;
(g) identifying an amino acid residue for each amino acid of the protein backbone identified in step (b) having atomic amino acid coordinates that are not overlapping with the atomic van der Mer coordinates of the set of van der Mer identified in step (f);
(h) optimizing atomic coordinates of the compound and protein;
(i) wherein the optimization is performed using at least an energy minimization calculation, and wherein the optimization is performed to energetically stabilize a complex of the compound and protein.

In an aspect is provided a system for identifying a protein capable of binding a compound, including: at least one data processor; and at least one memory storing instructions which, when executed by the at least one data processor, result in operations including:
(a) identifying covalently bonded amino acid backbone residues of the protein wherein each amino acid backbone residue atom is associated with a set of atomic protein coordinates;
(b) identifying an independent set of van der Mer associated with an amino acid backbone residue and a chemical group of the compound, wherein each van der Mer is associated with a set of atomic van der Mer coordinates for an amino acid and chemical group of the compound and the atomic van der Mer coordinates for the van der Mer amino acid backbone atoms of each independent set of van der Mer overlap with amino acid backbone residue atomic protein coordinates of the protein;
(c) identifying and removing from each independent set of van der Mer, any van der Mer wherein atomic van der Mer coordinates of a sidechain or chemical group of the van der Mer overlap with atomic protein coordinates of the covalently bonded amino acid backbone residues of the protein;
(d) identifying and removing any van der Mer wherein atomic van der Mer coordinates of the chemical group of the van der Mer is characterized as exposed to bulk solvent;
(e) identifying independent sets of atomic chemical coordinates of the compound wherein, the atomic chemical coordinates of the compound chemical group atoms of each independent set overlap with atomic van der Mer coordinates of chemical group atoms of van der Mer identified in steps (b) to (d) and atomic van der Mer coordinates of the van der Mer further include atomic van der Mer coordinates for amino acid backbone atoms that overlap with atomic protein coordinates of amino acid backbone atoms of the protein;
(f) identifying and sorting independent sets of atomic chemical coordinates of the compound of step (e) based on the value of the compound van der Mer cluster score;
(g) identifying a preferred amino acid for an amino acid residue position of the protein when the amino acid residue position of the protein has amino acid backbone atom atomic protein coordinates that overlap with the amino acid backbone atomic van der Mer coordinates of a van der Mer identified in step (f) and the preferred amino acid is the amino acid associated with the van der Mer;
(h) optimizing atomic coordinates of the compound and amino acid residues of the protein;
(i) wherein the optimization is performed using at least an energy minimization calculation, and wherein the optimization is performed to energetically stabilize the protein.

In embodiments, the optimizing includes an iterative or heuristic algorithm. In embodiments, the optimizing includes a simplex algorithm, memetic algorithm, differential evolution algorithm, evolutionary algorithm, genetic algorithm, tabu algorithm, particle swarm algorithm, or stimulated annealing algorithm. In embodiments, the optimizing includes a Monte Carlo sampling algorithm, dead-end elimination algorithm, branch and bound algorithm, or a pruning algorithm. In embodiments, the energy minimization calculation includes a molecular mechanics function, a structural bioinformatics function, an amino acid sidechain packing function, a protein radius of gyration function, or a combination thereof. In embodiments, identifying atomic van der Mer coordinates of a chemical group of a van der Mer as exposed to bulk solvent is performed using a convex hull algorithm. In embodiments, the cluster score is a function (e.g., including but not limited to a natural log or logistical function) of the ratio of 1) the number of members in an independent set of geometrically overlapping van der Mer of one chemical group and one amino acid to 2) the average number of members in all independent sets van der Mer of the chemical group and the amino acid. In embodiments, the members in an independent set of geometrically overlapping compound van der Mer of one chemical group are identified by having an RMSD below a threshold, wherein the RMSD is calculated using the atomic coordinates of the chemical group and the backbone atoms of the amino acid residue of a van der Mer. In embodiments, the RMSD threshold is 0.5 angstrom. In embodiments, the preferred amino acid of step (g) is an amino acid in a van der Mer having a cluster score greater than 2. In embodiments, identifying an amino acid residue for each protein backbone residue having atomic amino acid coordinates that are not overlapping with the atomic van der Mer coordinates of a van der Mer is performed using The Rosetta Software. In embodiments, the van der Mer database is a collection of independent van der Mer each including a unique set of atomic van der Mer coordinates describing the three dimensional positions of a chemical group interacting in silico with an amino acid residue, further wherein the interacting was identified in an empirically determined protein and chemical group complex. In embodiments, the protein is a 4-helix bundle protein. In embodiments, the compound includes a charged chemical group at physiological pH. In embodiments, the compound includes a polar chemical group at physiological pH. In embodiments, the system includes use of a system described in international application no. WO2019/023644. In embodiments, the overlap of the van der Mers and the compound chemical groups are not selected one at a time, but rather in pairs, triplets or higher order combinations. In embodiments, van der Mers at multiple sites are selected and the RMSD between these van der Mer chemical groups and the compound chemical groups is computed. In embodiments, the RMSD between van der Mer chemical groups and the compound chemical groups is precomputed and saved in lookup tables. In embodiments, the overlap of the van der Mers and the compound chemical groups are selected in pairs. In embodiments, the overlap of the van der Mers and the compound chemical groups are selected in triplets. In embodiments, the overlap of the van der Mers and the compound chemical groups are selected in combinations greater than three. In embodiments an identified van der Mer is a cluster having a 0.5 angstrom RMSD between members. In embodiments an identified van der Mer is a sub-cluster of a van der Mer. In embodiments an identified van der Mer is a van der Mer cluster member. In embodiments an identified van der Mer is a van der Mer representative. In embodiments an identified van der Mer is a cluster having a 0.1 angstrom RMSD between members.

In an aspect is provided a non-transitory computer-readable storage medium including program code for identifying a protein capable of binding a compound including when executed by at least one data processor, causes operations including identifying van der Mers representing the chemical groups of the compound and amino acid residues of the protein capable of interacting with the chemical groups of the compound in silico, and wherein the protein has secondary and tertiary protein structure when bound to the compound.

In an aspect is provided a non-transitory computer-readable storage medium including program code for identifying a protein capable of binding a compound, which when executed by at least one data processor, causes operations including:
(a) generating a first set of atomic protein coordinates representing a backbone structure of the protein;
(b) identifying a first van der Mer from a van der Mer database including a first set of atomic van der Mer coordinates representing a first chemical group of the compound, a first amino acid side chain and a first portion of a protein backbone bound to the first amino acid side chain, wherein the first chemical group interacts in silico with the first portion of a protein backbone or the first amino acid side chain;
(c) identifying portions of the backbone structure of the protein having atomic protein coordinates that are capable of overlapping with the atomic van der Mer coordinates of the first portion of a protein backbone of the first van der Mer;
(d) repeating steps (b) and (c) for at least one additional van der Mer independently representing an optionally different chemical group of the compound, an additional independent amino acid side chain and an additional independent portion of a protein backbone bound to the independent additional amino acid side chain;
(e) generating a set of atomic chemical coordinates representing the compound;
(f) generating at least one set of atomic coordinates of an in silico complex of the protein capable of binding the compound bound to the compound; wherein the in silico complex optimizes the overlap between the atomic chemical coordinates of the compound chemical groups and the atomic van der Mer coordinates representing the independent chemical groups of the compound identified in steps (b) to (d);
(g) generating a set of atomic amino acid coordinates for amino acid side chains and portions of protein backbone independently representing those portions of the backbone structure of the protein that are not overlapping with atomic van der Mer coordinates of the van der Mer including atomic van der Mer coordinates that are overlapping with the atomic chemical coordinates representing the compound of the in silico complex of step (f);
(h) based at least in part on steps (a) to (g), optimizing atomic coordinates of the compound and protein thereby identifying a protein capable of binding the compound.

In embodiments, the non-transitory computer-readable storage medium including program code includes generating a plurality of sets of atomic coordinates of an in silico complex of the protein capable of binding the compound bound to the compound in step (f). In embodiments, the non-transitory computer-readable storage medium including program code includes generating all possible sets of atomic coordinates of an in silico complex of the protein capable of binding the compound bound to the compound in step (f). In embodiments, the plurality of sets of atomic coordinates of an in silico complex of the protein capable of binding the compound bound to the compound are independently different from each other and optimization of the overlap between the atomic chemical coordinates of the compound chemical groups and the atomic van der Mer coordinates representing the independent chemical groups of the compound identified in steps (b) to (d) is performed without duplication of the sets. In embodiments, the plurality of independent sets of atomic coordinates of an in silico complex of the protein capable of binding the compound bound to the compound are independently different from each other and are scored. In embodiments, the scoring includes calculating a cluster score for each of the plurality of sets of atomic coordinates of an in silico complex of the protein capable of binding the compound bound to the compound. In embodiments, the cluster score is the natural logarithm of the ratio of 1) the number of members in an independent set of geometrically overlapping van der Mer of one chemical group and one amino acid to 2) the average number of members in all independent sets van der Mer of the chemical group and the amino acid. In embodiments, the members in an independent set of geometrically overlapping compound van der Mer of one chemical group are identified by having an RMSD below a threshold, wherein the RMSD is calculated using the atomic coordinates of the chemical group and the backbone atoms of the amino acid residue of a van der Mer. In embodiments, the RMSD threshold is 0.5 angstrom. In embodiments, step (a) includes generating a plurality of independent sets of atomic protein coordinates representing independent backbone structures of the protein. In embodiments, the plurality of independent backbone structures of the protein have a similar overall three dimensional fold. In embodiments, the plurality of independent backbone structures of the protein have an RMSD of less than 3 angstrom. In embodiments, the compound chemical groups and van der Mer chemical groups are polar groups. In embodiments, steps (g) and (h) include use of a method described in international application no. WO2019/023644. In embodiments, step (c) includes identifying all portions of the backbone structure of the protein having atomic protein coordinates that are capable of overlapping with the atomic van der Mer coordinates of the first portion of a protein backbone of the first van der Mer. In embodiments, step (d) includes repeating steps (b) and (c) for all van der Mer in the van der Mer database independently representing all chemical groups of the compound, an additional independent amino acid side chain and an additional independent portion of a protein backbone bound to said independent additional amino acid side chain. In embodiments, the overlap of the van der Mers and the compound chemical groups are not selected one at a time, but rather in pairs, triplets or higher order combinations. In embodiments, van der Mers at multiple sites are selected and the RMSD between these van der Mer chemical groups and the compound chemical groups is computed. In embodiments, the RMSD between van der Mer chemical groups and the compound chemical groups is precomputed and saved in lookup tables. In embodiments, the overlap of the van der Mers and the compound chemical groups are selected in pairs. In embodiments, the overlap of the van der Mers and the compound chemical groups are selected in triplets. In embodiments, the overlap of the van der Mers and the compound chemical groups are selected in combinations greater than three. In embodiments an identified van der Mer is a cluster having a 0.5 angstrom RMSD between members. In embodiments an identified van der Mer is a sub-cluster of a van der Mer. In embodiments an identified van der Mer is a van der Mer cluster member. In embodiments an identified van der Mer is a van der Mer representative. In embodiments an identified van der Mer is a cluster having a 0.1 angstrom RMSD between members.

In an aspect is provided a non-transitory computer-readable storage medium including program code for identifying a complex of a protein bound to a compound, which, when executed by at least one data processor, causes operations including:
(a) generating a first set of atomic protein coordinates representing the side chain and backbone structure of the protein;
(b) identifying a first van der Mer from a van der Mer database including a first set of atomic van der Mer coordinates representing a first chemical group of the compound, a first amino acid side chain and a first portion of a protein backbone bound to the first amino acid side chain, wherein the first chemical group interacts in silico with the first portion of a protein backbone or the first amino acid side chain;
(c) identifying portions of the backbone structure of the protein having atomic protein coordinates that are capable of overlapping with the atomic van der Mer coordinates of the first portion of a protein backbone of the first van der Mer wherein the amino acid side chain of the van der Mer and the amino acid side chain directly attached to the overlapping portions of the backbone structure of the protein are the same side chain;
(d) repeating steps (b) and (c) for at least one additional van der Mer independently representing an optionally different chemical group of the compound, an additional independent amino acid side chain and an additional independent portion of a protein backbone bound to the independent additional amino acid side chain;
(e) generating a set of atomic chemical coordinates representing the compound;
(f) generating at least one set of atomic coordinates of an in silico complex of the protein capable of binding the compound bound to the compound; wherein the in silico complex optimizes the overlap between the atomic chemical coordinates of the compound chemical groups and the atomic van der Mer coordinates representing the independent chemical groups of the compound identified in steps (b) to (d);
(g) based at least in part on steps (a) to (f), optimizing atomic coordinates of the compound and protein thereby identifying a complex of a protein bound to a compound.

In an aspect is provided a non-transitory computer-readable storage medium including program code for identifying a protein capable of binding a compound, which, when executed by at least one data processor, causes operations including:
(a) generating a first set of atomic protein coordinates representing a protein backbone structure;
(b) generating a first set of atomic chemical coordinates representing a first chemical group of the compound;
(c) identifying a first van der Mer from a van der Mer database including a first set of atomic van der Mer coordinates representing the first chemical group of the compound, a first amino acid side chain and a first portion of a protein backbone bound to the first amino acid side chain, wherein the first chemical group interacts in silico with the first portion of a protein backbone or the first amino acid side chain;
(d) generating a second set of atomic chemical coordinates representing a second chemical group of the compound;
(e) identifying a second van der Mer from the van der Mer database including a second set of atomic van der Mer coordinates representing the second chemical group of the compound, a second amino acid side chain and a second portion of a protein backbone bound to the second amino acid side chain, wherein the second chemical group interacts in silico with the second portion of a protein backbone or the second amino acid side chain;
(f) calculating an energetic stability of the protein backbone structure bound to the compound using the first set of atomic van der Mer coordinates and the second set of atomic van der Mer coordinates in silico;
(g) repeating steps (a) to (f) for additional van der Mers representing the first chemical group of the compound, a first amino acid side chain and a first portion of a protein backbone bound to the first amino acid side chain and additional van der Mers representing the second chemical group of the compound, a second amino acid side chain and a second portion of a protein backbone bound to the second amino acid side chain;
(h) generating a set of atomic amino acid coordinates for amino acid side chains and portions of protein backbone independently representing those portions of the protein backbone structure not represented by a van der Mer of steps (a) to (g);
(i) based at least in part on steps (a) to (h), optimizing atomic coordinates of the compound and protein thereby identifying a protein capable of binding the compound.

In an aspect is provided a non-transitory computer-readable storage medium including program code for identifying a protein capable of binding a compound, which, when executed by at least one data processor, causes operations including:
(a) identifying a first van der Mer from a van der Mer database including atomic van der Mer coordinates of a chemical group of the compound, wherein the atomic van der Mer coordinates of the chemical group in the first van der Mer overlap with the atomic chemical coordinates of the chemical group of the compound;
(b) identifying a protein backbone for the protein wherein the atoms of the protein backbone are associated with a set of atomic protein coordinates;
(c) identifying an overlap between the atomic van der Mer coordinates of the amino acid backbone of the first van der Mer identified in step (a) and the atomic protein coordinates of an amino acid residue of the protein backbone identified in step (b);
(d) optionally repeating steps (a) to (c) for a different chemical group of the compound;
(e) identifying independent sets of van der Mer identified in steps (a) to (d) wherein all van der Mer of each independent set include atomic van der Mer coordinates that collectively simultaneously overlap atomic protein coordinates of the protein backbone identified in step (b);
(f) identifying at least one independent set of van der Mer identified in step (e) with a cluster score above a threshold;
(g) identifying an amino acid residue for each amino acid of the protein backbone identified in step (b) having atomic amino acid coordinates that are not overlapping with the atomic van der Mer coordinates of the set of van der Mer identified in step (f);
(h) optimizing atomic coordinates of the compound and protein;
(i) wherein the optimization is performed using at least an energy minimization calculation, and wherein the optimization is performed to energetically stabilize a complex of the compound and protein.

In an aspect is provided a non-transitory computer-readable storage medium including program code for identifying a protein capable of binding a compound, which, when executed by at least one data processor, causes operations including:
(a) identifying covalently bonded amino acid backbone residues of the protein wherein each amino acid backbone residue atom is associated with a set of atomic protein coordinates;
(b) identifying an independent set of van der Mer associated with an amino acid backbone residue and a chemical group of the compound, wherein each van der Mer is associated with a set of atomic van der Mer coordinates for an amino acid and chemical group of the compound and the atomic van der Mer coordinates for the van der Mer amino acid backbone atoms of each independent set of van der Mer overlap with amino acid backbone residue atomic protein coordinates of the protein;
(c) identifying and removing from each independent set of van der Mer, any van der Mer wherein atomic van der Mer coordinates of a sidechain or chemical group of the van der Mer overlap with atomic protein coordinates of the covalently bonded amino acid backbone residues of the protein;
(d) identifying and removing any van der Mer wherein atomic van der Mer coordinates of the chemical group of the van der Mer is characterized as exposed to bulk solvent;
(e) identifying independent sets of atomic chemical coordinates of the compound wherein, the atomic chemical coordinates of the compound chemical group atoms of each independent set overlap with atomic van der Mer coordinates of chemical group atoms of van der Mer identified in steps (b) to (d) and atomic van der Mer coordinates of the van der Mer further include atomic van der Mer coordinates for amino acid backbone atoms that overlap with atomic protein coordinates of amino acid backbone atoms of the protein;
(f) identifying and sorting independent sets of atomic chemical coordinates of the compound of step (e) based on the value of the compound van der Mer cluster score;
(g) identifying a preferred amino acid for an amino acid residue position of the protein when the amino acid residue position of the protein has amino acid backbone atom atomic protein coordinates that overlap with the amino acid backbone atomic van der Mer coordinates of a van der Mer identified in step (f) and the preferred amino acid is the amino acid associated with the van der Mer;
(h) optimizing atomic coordinates of the compound and amino acid residues of the protein;
(i) wherein the optimization is performed using at least an energy minimization calculation, and wherein the optimization is performed to energetically stabilize the protein.

In embodiments, the optimizing includes an iterative or heuristic algorithm. In embodiments, the optimizing includes a simplex algorithm, memetic algorithm, differential evolution algorithm, evolutionary algorithm, genetic algorithm, tabu algorithm, particle swarm algorithm, or stimulated annealing algorithm. In embodiments, the optimizing includes a Monte Carlo sampling algorithm, dead-end elimination algorithm, branch and bound algorithm, or a pruning algorithm. In embodiments, the energy minimization calculation includes a molecular mechanics function, a structural bioinformatics function, an amino acid sidechain packing function, a protein radius of gyration function, or a combination thereof. In embodiments, identifying atomic van der Mer coordinates of a chemical group of a van der Mer as exposed to bulk solvent is performed using a convex hull algorithm. In embodiments, the cluster score is the natural logarithm of the ratio of 1) the number of members in an independent set of geometrically overlapping van der Mer of one chemical group and one amino acid to 2) the average number of members in all independent sets van der Mer of the chemical group and the amino acid. In embodiments, the members in an independent set of geometrically overlapping compound van der Mer of one chemical group are identified by having an RMSD below a threshold, wherein the RMSD is calculated using the atomic coordinates of the chemical group and the backbone atoms of the amino acid residue of a van der Mer. In embodiments, the RMSD threshold is 0.5 angstrom. In embodiments, the preferred amino acid of step (g) is an amino acid in a van der Mer having a cluster score greater than 2. In embodiments, identifying an amino acid residue for each protein backbone residue having atomic amino acid coordinates that are not overlapping with the atomic van der Mer coordinates of a van der Mer is performed using The Rosetta Software. In embodiments, the van der Mer database is a collection of independent van der Mer each including a unique set of atomic van der Mer coordinates describing the three dimensional positions of a chemical group interacting in silico with an amino acid residue, further wherein the interacting was identified in an empirically determined protein and chemical group complex. In embodiments, the protein is a 4-helix bundle protein. In embodiments, the compound includes a charged chemical group at physiological pH. In embodiments, the compound includes a polar chemical group at physiological pH. In embodiments, the non-transitory computer-readable storage medium including program code includes use of a method described in international application no. WO2019/023644. In embodiments, the overlap of the van der Mers and the compound chemical groups are not selected one at a time, but rather in pairs, triplets or higher order combinations. In embodiments, van der Mers at multiple sites are selected and the RMSD between these van der Mer chemical groups and the compound chemical groups is computed. In embodiments, the RMSD between van der Mer chemical groups and the compound chemical groups is precomputed and saved in lookup tables. In embodiments, the overlap of the van der Mers and the compound chemical groups are selected in pairs. In embodiments, the overlap of the van der Mers and the compound chemical groups are selected in triplets. In embodiments, the overlap of the van der Mers and the compound chemical groups are selected in combinations greater than three. In embodiments an identified van der Mer is a cluster having a 0.5 angstrom RMSD between members. In embodiments an identified van der Mer is a sub-cluster of a van der Mer. In embodiments an identified van der Mer is a van der Mer cluster member. In embodiments an identified van der Mer is a van der Mer representative. In embodiments an identified van der Mer is a cluster having a 0.1 angstrom RMSD between members.

In an aspect is provided a system for identifying a protein capable of binding a ligand (e.g., compound) including at least one data processor; and at least one memory storing instructions which, when executed by the at least one data processor, result in operations including identifying van der Mers representing the chemical groups of the ligand (e.g., compound) and amino acid residues of the protein capable of interacting with the chemical groups of the ligand (e.g., compound) in silico, and wherein the protein has secondary and tertiary protein structure when bound to the ligand (e.g., compound).

In an aspect is provided a system for identifying a protein capable of binding a ligand (e.g., compound), including: at least one data processor; and at least one memory storing instructions which, when executed by the at least one data processor, result in operations including:
(a) generating a first set of atomic protein coordinates representing a backbone structure of the protein;
(b) identifying a first van der Mer from a van der Mer database including a first set of atomic van der Mer coordinates representing a first chemical group of the ligand (e.g., compound), a first amino acid side chain and a first portion of a protein backbone bound to the first amino acid side chain, wherein the first chemical group interacts in silico with the first portion of a protein backbone or the first amino acid side chain;
(c) identifying portions of the backbone structure of the protein having atomic protein coordinates that are capable of overlapping with the atomic van der Mer coordinates of the first portion of a protein backbone of the first van der Mer;
(d) repeating steps (b) and (c) for at least one additional van der Mer independently representing an optionally different chemical group of the ligand (e.g., compound), an additional independent amino acid side chain and an additional independent portion of a protein backbone bound to the independent additional amino acid side chain;
(e) generating a set of atomic chemical coordinates representing the ligand (e.g., compound);
(f) generating at least one set of atomic coordinates of an in silico complex of the protein capable of binding the ligand (e.g., compound) bound to the ligand (e.g., compound); wherein the in silico complex optimizes the overlap between the atomic chemical coordinates of the ligand (e.g., compound) chemical groups and the atomic van der Mer coordinates representing the independent chemical groups of the ligand (e.g., compound) identified in steps (b) to (d);
(g) generating a set of atomic amino acid coordinates for amino acid side chains and portions of protein backbone independently representing those portions of the backbone structure of the protein that are not overlapping with atomic van der Mer coordinates of the van der Mer including atomic van der Mer coordinates that are overlapping with the atomic chemical coordinates representing the ligand (e.g., compound) of the in silico complex of step (f);
(h) based at least in part on steps (a) to (g), optimizing atomic coordinates of the ligand (e.g., compound) and protein thereby identifying a protein capable of binding the ligand (e.g., compound).

In an aspect is provided a system for identifying a complex of a protein bound to a ligand (e.g., compound), including: at least one data processor; and at least one memory storing instructions which, when executed by the at least one data processor, result in operations including:
(a) generating a first set of atomic protein coordinates representing the side chain and backbone structure of the protein;
(b) identifying a first van der Mer from a van der Mer database including a first set of atomic van der Mer coordinates representing a first chemical group of the ligand (e.g., compound), a first amino acid side chain and a first portion of a protein backbone bound to the first amino acid side chain, wherein the first chemical group interacts in silico with the first portion of a protein backbone or the first amino acid side chain;
(c) identifying portions of the backbone structure of the protein having atomic protein coordinates that are capable of overlapping with the atomic van der Mer coordinates of the first portion of a protein backbone of the first van der Mer wherein the amino acid side chain of the van der Mer and the amino acid side chain directly attached to the overlapping portions of the backbone structure of the protein are the same side chain;
(d) repeating steps (b) and (c) for at least one additional van der Mer independently representing an optionally different chemical group of the ligand (e.g., compound), an additional independent amino acid side chain and an additional independent portion of a protein backbone bound to the independent additional amino acid side chain;
(e) generating a set of atomic chemical coordinates representing the ligand (e.g., compound);
(f) generating at least one set of atomic coordinates of an in silico complex of the protein capable of binding the ligand (e.g., compound) bound to the ligand (e.g., compound); wherein the in silico complex optimizes the overlap between the atomic chemical coordinates of the ligand (e.g., compound) chemical groups and the atomic van der Mer coordinates representing the independent chemical groups of the ligand (e.g., compound) identified in steps (b) to (d);
(g) based at least in part on steps (a) to (f), optimizing atomic coordinates of the ligand (e.g., compound) and protein thereby identifying a complex of a protein bound to a ligand (e.g., compound).

In an aspect is provided a system for identifying a protein capable of binding a ligand (e.g., compound), including: at least one data processor; and at least one memory storing instructions which, when executed by the at least one data processor, result in operations including:
(a) generating a first set of atomic protein coordinates representing a protein backbone structure;
(b) generating a first set of atomic chemical coordinates representing a first chemical group of the ligand (e.g., compound);
(c) identifying a first van der Mer from a van der Mer database including a first set of atomic van der Mer coordinates representing the first chemical group of the ligand (e.g., compound), a first amino acid side chain and a first portion of a protein backbone bound to the first amino acid side chain, wherein the first chemical group interacts in silico with the first portion of a protein backbone or the first amino acid side chain;
(d) generating a second set of atomic chemical coordinates representing a second chemical group of the ligand (e.g., compound);
(e) identifying a second van der Mer from the van der Mer database including a second set of atomic van der Mer coordinates representing the second chemical group of the ligand (e.g., compound), a second amino acid side chain and a second portion of a protein backbone bound to the second amino acid side chain, wherein the second chemical group interacts in silico with the second portion of a protein backbone or the second amino acid side chain;
(f) calculating an energetic stability of the protein backbone structure bound to the ligand (e.g., compound) using the first set of atomic van der Mer coordinates and the second set of atomic van der Mer coordinates in silico;
(g) repeating steps (a) to (f) for additional van der Mers representing the first chemical group of the ligand (e.g., compound), a first amino acid side chain and a first portion of a protein backbone bound to the first amino acid side chain and additional van der Mers representing the second chemical group of the ligand (e.g., compound), a second amino acid side chain and a second portion of a protein backbone bound to the second amino acid side chain;
(h) generating a set of atomic amino acid coordinates for amino acid side chains and portions of protein backbone independently representing those portions of the protein backbone structure not represented by a van der Mer of steps (a) to (g);
(i) based at least in part on steps (a) to (h), optimizing atomic coordinates of the ligand (e.g., compound) and protein thereby identifying a protein capable of binding the ligand (e.g., compound).

In an aspect is provided a system for identifying a protein capable of binding a ligand (e.g., compound), including: at least one data processor; and at least one memory storing instructions which, when executed by the at least one data processor, result in operations including:
(a) identifying a first van der Mer from a van der Mer database including atomic van der Mer coordinates of a chemical group of the ligand (e.g., compound), wherein the atomic van der Mer coordinates of the chemical group in the first van der Mer overlap with the atomic chemical coordinates of the chemical group of the ligand (e.g., compound);
(b) identifying a protein backbone for the protein wherein the atoms of the protein backbone are associated with a set of atomic protein coordinates;
(c) identifying an overlap between the atomic van der Mer coordinates of the amino acid backbone of the first van der Mer identified in step (a) and the atomic protein coordinates of an amino acid residue of the protein backbone identified in step (b);
(d) optionally repeating steps (a) to (c) for a different chemical group of the ligand (e.g., compound);
(e) identifying independent sets of van der Mer identified in steps (a) to (d) wherein all van der Mer of each independent set include atomic van der Mer coordinates that collectively simultaneously overlap atomic protein coordinates of the protein backbone identified in step (b);
(f) identifying at least one independent set of van der Mer identified in step (e) with a cluster score above a threshold;
(g) identifying an amino acid residue for each amino acid of the protein backbone identified in step (b) having atomic amino acid coordinates that are not overlapping with the atomic van der Mer coordinates of the set of van der Mer identified in step (f);
(h) optimizing atomic coordinates of the ligand (e.g., compound) and protein;
(i) wherein the optimization is performed using at least an energy minimization calculation, and wherein the optimization is performed to energetically stabilize a complex of the ligand (e.g., compound) and protein.

In an aspect is provided a system for identifying a protein capable of binding a ligand (e.g., compound), including: at least one data processor; and at least one memory storing instructions which, when executed by the at least one data processor, result in operations including:
(a) identifying covalently bonded amino acid backbone residues of the protein wherein each amino acid backbone residue atom is associated with a set of atomic protein coordinates;
(b) identifying an independent set of van der Mer associated with an amino acid backbone residue and a chemical group of the ligand (e.g., compound), wherein each van der Mer is associated with a set of atomic van der Mer coordinates for an amino acid and chemical group of the ligand (e.g., compound) and the atomic van der Mer coordinates for the van der Mer amino acid backbone atoms of each independent set of van der Mer overlap with amino acid backbone residue atomic protein coordinates of the protein;
(c) identifying and removing from each independent set of van der Mer, any van der Mer wherein atomic van der Mer coordinates of a sidechain or chemical group of the van der Mer overlap with atomic protein coordinates of the covalently bonded amino acid backbone residues of the protein;
(d) identifying and removing any van der Mer wherein atomic van der Mer coordinates of the chemical group of the van der Mer is characterized as exposed to bulk solvent;
(e) identifying independent sets of atomic chemical coordinates of the ligand (e.g., compound) wherein, the atomic chemical coordinates of the ligand (e.g., compound) chemical group atoms of each independent set overlap with atomic van der Mer coordinates of chemical group atoms of van der Mer identified in steps (b) to (d) and atomic van der Mer coordinates of the van der Mer further include atomic van der Mer coordinates for amino acid backbone atoms that overlap with atomic protein coordinates of amino acid backbone atoms of the protein;
(f) identifying and sorting independent sets of atomic chemical coordinates of the ligand (e.g., compound) of step (e) based on the value of the ligand (e.g., compound) van der Mer cluster score;
(g) identifying a preferred amino acid for an amino acid residue position of the protein when the amino acid residue position of the protein has amino acid backbone atom atomic protein coordinates that overlap with the amino acid backbone atomic van der Mer coordinates of a van der Mer identified in step (f) and the preferred amino acid is the amino acid associated with the van der Mer;
(h) optimizing atomic coordinates of the ligand (e.g., compound) and amino acid residues of the protein;
(i) wherein the optimization is performed using at least an energy minimization calculation, and wherein the optimization is performed to energetically stabilize the protein.

In an aspect is provided a non-transitory computer-readable storage medium including program code for identifying a protein capable of binding a ligand (e.g., compound) including when executed by at least one data processor, causes operations including identifying van der Mers representing the chemical groups of the ligand (e.g., compound) and amino acid residues of the protein capable of interacting with the chemical groups of the ligand (e.g., compound) in silico, and wherein the protein has secondary and tertiary protein structure when bound to the ligand (e.g., compound).

In an aspect is provided a non-transitory computer-readable storage medium including program code for identifying a protein capable of binding a ligand (e.g., compound), which when executed by at least one data processor, causes operations including:
(a) generating a first set of atomic protein coordinates representing a backbone structure of the protein;
(b) identifying a first van der Mer from a van der Mer database including a first set of atomic van der Mer coordinates representing a first chemical group of the ligand (e.g., compound), a first amino acid side chain and a first portion of a protein backbone bound to the first amino acid side chain, wherein the first chemical group interacts in silico with the first portion of a protein backbone or the first amino acid side chain;
(c) identifying portions of the backbone structure of the protein having atomic protein coordinates that are capable of overlapping with the atomic van der Mer coordinates of the first portion of a protein backbone of the first van der Mer;
(d) repeating steps (b) and (c) for at least one additional van der Mer independently representing an optionally different chemical group of the ligand (e.g., compound), an additional independent amino acid side chain and an additional independent portion of a protein backbone bound to the independent additional amino acid side chain;
(e) generating a set of atomic chemical coordinates representing the ligand (e.g., compound);
(f) generating at least one set of atomic coordinates of an in silico complex of the protein capable of binding the ligand (e.g., compound) bound to the ligand (e.g., compound); wherein the in silico complex optimizes the overlap between the atomic chemical coordinates of the ligand (e.g., compound) chemical groups and the atomic van der Mer coordinates representing the independent chemical groups of the ligand (e.g., compound) identified in steps (b) to (d);
(g) generating a set of atomic amino acid coordinates for amino acid side chains and portions of protein backbone independently representing those portions of the backbone structure of the protein that are not overlapping with atomic van der Mer coordinates of the van der Mer including atomic van der Mer coordinates that are overlapping with the atomic chemical coordinates representing the ligand (e.g., compound) of the in silico complex of step (f);
(h) based at least in part on steps (a) to (g), optimizing atomic coordinates of the ligand (e.g., compound) and protein thereby identifying a protein capable of binding the ligand (e.g., compound).

In an aspect is provided a non-transitory computer-readable storage medium including program code for identifying a complex of a protein bound to a ligand (e.g., compound), which, when executed by at least one data processor, causes operations including:
(a) generating a first set of atomic protein coordinates representing the side chain and backbone structure of the protein;
(b) identifying a first van der Mer from a van der Mer database including a first set of atomic van der Mer coordinates representing a first chemical group of the ligand (e.g., compound), a first amino acid side chain and a first portion of a protein backbone bound to the first amino acid side chain, wherein the first chemical group interacts in silico with the first portion of a protein backbone or the first amino acid side chain;
(c) identifying portions of the backbone structure of the protein having atomic protein coordinates that are capable of overlapping with the atomic van der Mer coordinates of the first portion of a protein backbone of the first van der Mer wherein the amino acid side chain of the van der Mer and the amino acid side chain directly attached to the overlapping portions of the backbone structure of the protein are the same side chain;
(d) repeating steps (b) and (c) for at least one additional van der Mer independently representing an optionally different chemical group of the ligand (e.g., compound), an additional independent amino acid side chain and an additional independent portion of a protein backbone bound to the independent additional amino acid side chain;
(e) generating a set of atomic chemical coordinates representing the ligand (e.g., compound);
(f) generating at least one set of atomic coordinates of an in silico complex of the protein capable of binding the ligand (e.g., compound) bound to the ligand (e.g., compound); wherein the in silico complex optimizes the overlap between the atomic chemical coordinates of the ligand (e.g., compound) chemical groups and the atomic van der Mer coordinates representing the independent chemical groups of the ligand (e.g., compound) identified in steps (b) to (d);
(g) based at least in part on steps (a) to (f), optimizing atomic coordinates of the ligand (e.g., compound) and protein thereby identifying a complex of a protein bound to a ligand (e.g., compound).

In an aspect is provided a non-transitory computer-readable storage medium including program code for identifying a protein capable of binding a ligand (e.g., compound), which, when executed by at least one data processor, causes operations including:
(a) generating a first set of atomic protein coordinates representing a protein backbone structure;
(b) generating a first set of atomic chemical coordinates representing a first chemical group of the ligand (e.g., compound);
(c) identifying a first van der Mer from a van der Mer database including a first set of atomic van der Mer coordinates representing the first chemical group of the ligand (e.g., compound), a first amino acid side chain and a first portion of a protein backbone bound to the first amino acid side chain, wherein the first chemical group interacts in silico with the first portion of a protein backbone or the first amino acid side chain;
(d) generating a second set of atomic chemical coordinates representing a second chemical group of the ligand (e.g., compound);
(e) identifying a second van der Mer from the van der Mer database including a second set of atomic van der Mer coordinates representing the second chemical group of the ligand (e.g., compound), a second amino acid side chain and a second portion of a protein backbone bound to the second amino acid side chain, wherein the second chemical group interacts in silico with the second portion of a protein backbone or the second amino acid side chain;
(f) calculating an energetic stability of the protein backbone structure bound to the ligand (e.g., compound) using the first set of atomic van der Mer coordinates and the second set of atomic van der Mer coordinates in silico;
(g) repeating steps (a) to (f) for additional van der Mers representing the first chemical group of the ligand (e.g., compound), a first amino acid side chain and a first portion of a protein backbone bound to the first amino acid side chain and additional van der Mers representing the second chemical group of the ligand (e.g., compound), a second amino acid side chain and a second portion of a protein backbone bound to the second amino acid side chain;
(h) generating a set of atomic amino acid coordinates for amino acid side chains and portions of protein backbone independently representing those portions of the protein backbone structure not represented by a van der Mer of steps (a) to (g);
(i) based at least in part on steps (a) to (h), optimizing atomic coordinates of the ligand (e.g., compound) and protein thereby identifying a protein capable of binding the ligand (e.g., compound).

In an aspect is provided a non-transitory computer-readable storage medium including program code for identifying a protein capable of binding a ligand (e.g., compound), which, when executed by at least one data processor, causes operations including:
(a) identifying a first van der Mer from a van der Mer database including atomic van der Mer coordinates of a chemical group of the ligand (e.g., compound), wherein the atomic van der Mer coordinates of the chemical group in the first van der Mer overlap with the atomic chemical coordinates of the chemical group of the ligand (e.g., compound);
(b) identifying a protein backbone for the protein wherein the atoms of the protein backbone are associated with a set of atomic protein coordinates;
(c) identifying an overlap between the atomic van der Mer coordinates of the amino acid backbone of the first van der Mer identified in step (a) and the atomic protein coordinates of an amino acid residue of the protein backbone identified in step (b);
(d) optionally repeating steps (a) to (c) for a different chemical group of the ligand (e.g., compound);
(e) identifying independent sets of van der Mer identified in steps (a) to (d) wherein all van der Mer of each independent set include atomic van der Mer coordinates that collectively simultaneously overlap atomic protein coordinates of the protein backbone identified in step (b);
(f) identifying at least one independent set of van der Mer identified in step (e) with a cluster score above a threshold;
(g) identifying an amino acid residue for each amino acid of the protein backbone identified in step (b) having atomic amino acid coordinates that are not overlapping with the atomic van der Mer coordinates of the set of van der Mer identified in step (f);
(h) optimizing atomic coordinates of the ligand (e.g., compound) and protein;
(i) wherein the optimization is performed using at least an energy minimization calculation, and wherein the optimization is performed to energetically stabilize a complex of the ligand (e.g., compound) and protein.

In an aspect is provided a non-transitory computer-readable storage medium including program code for identifying a protein capable of binding a ligand (e.g., compound), which, when executed by at least one data processor, causes operations including:
(a) identifying covalently bonded amino acid backbone residues of the protein wherein each amino acid backbone residue atom is associated with a set of atomic protein coordinates;
(b) identifying an independent set of van der Mer associated with an amino acid backbone residue and a chemical group of the ligand (e.g., compound), wherein each van der Mer is associated with a set of atomic van der Mer coordinates for an amino acid and chemical group of the ligand (e.g., compound) and the atomic van der Mer coordinates for the van der Mer amino acid backbone atoms of each independent set of van der Mer overlap with amino acid backbone residue atomic protein coordinates of the protein;
(c) identifying and removing from each independent set of van der Mer, any van der Mer wherein atomic van der Mer coordinates of a sidechain or chemical group of the van der Mer overlap with atomic protein coordinates of the covalently bonded amino acid backbone residues of the protein;
(d) identifying and removing any van der Mer wherein atomic van der Mer coordinates of the chemical group of the van der Mer is characterized as exposed to bulk solvent;
(e) identifying independent sets of atomic chemical coordinates of the ligand (e.g., compound) wherein, the atomic chemical coordinates of the ligand (e.g., compound) chemical group atoms of each independent set overlap with atomic van der Mer coordinates of chemical group atoms of van der Mer identified in steps (b) to (d) and atomic van der Mer coordinates of the van der Mer further include atomic van der Mer coordinates for amino acid backbone atoms that overlap with atomic protein coordinates of amino acid backbone atoms of the protein;
(f) identifying and sorting independent sets of atomic chemical coordinates of the ligand (e.g., compound) of step (e) based on the value of the ligand (e.g., compound) van der Mer cluster score;
(g) identifying a preferred amino acid for an amino acid residue position of the protein when the amino acid residue position of the protein has amino acid backbone atom atomic protein coordinates that overlap with the amino acid backbone atomic van der Mer coordinates of a van der Mer identified in step (f) and the preferred amino acid is the amino acid associated with the van der Mer;
(h) optimizing atomic coordinates of the ligand (e.g., compound) and amino acid residues of the protein;
(i) wherein the optimization is performed using at least an energy minimization calculation, and wherein the optimization is performed to energetically stabilize the protein.

In embodiments, the ligand is a compound. In embodiments, the compound is a chemical molecule having molecular weight of less than 10000 Daltons (e.g., less than 9000, 8000, 7000, 6000, 5000, 4000, 3000, 2000, 1000, 900, 800, 700, 600, 500, 400, 300, 200, or 100). In embodiments, the ligand is a small molecule. In embodiments, the ligand is a metal cofactor. In embodiments, the ligand is a metal ion. In embodiments, the ligand is a protein. In embodiments, the ligand is a compound.

In embodiments, the system includes generating a plurality of sets of atomic coordinates of an in silico complex of the protein capable of binding the ligand bound to the ligand in step (f). In embodiments, the system includes generating all possible sets of atomic coordinates of an in silico complex of the protein capable of binding the ligand bound to the ligand in step (f). In embodiments, the plurality of sets of atomic coordinates of an in silico complex of the protein capable of binding the ligand bound to the ligand are independently different from each other and optimization of the overlap between the atomic chemical coordinates of the ligand chemical groups and the atomic van der Mer coordinates representing the independent chemical groups of the ligand identified in steps (b) to (d) is performed without duplication of the sets. In embodiments, the plurality of independent sets of atomic coordinates of an in silico complex of the protein capable of binding the ligand bound to the ligand are independently different from each other and are scored. In embodiments, the scoring includes calculating a cluster score for each of the plurality of sets of atomic coordinates of an in silico complex of the protein capable of binding the ligand bound to the ligand. In embodiments, the cluster score is a function (e.g., including but not limited to a natural log or logistical function) of the ratio of 1) the number of members in an independent set of geometrically overlapping van der Mer of one chemical group and one amino acid to 2) the average number of members in all independent sets van der Mer of the chemical group and the amino acid. In embodiments, the members in an independent set of geometrically overlapping ligand van der Mer of one chemical group are identified by having an RMSD below a threshold, wherein the RMSD is calculated using the atomic coordinates of the chemical group and the backbone atoms of the amino acid residue of a van der Mer. In embodiments, the RMSD threshold is 0.5 angstrom. In embodiments, step (a) includes generating a plurality of independent sets of atomic protein coordinates representing independent backbone structures of the protein. In embodiments, the plurality of independent backbone structures of the protein have a similar overall three dimensional fold. In embodiments, the plurality of independent backbone structures of the protein have an RMSD of less than 3 angstrom. In embodiments, the ligand chemical groups and van der Mer chemical groups are polar groups. In embodiments, steps (g) and (h) include use of a method described in international application no. WO2019/023644. In embodiments, step (c) includes identifying all portions of the backbone structure of the protein having atomic protein coordinates that are capable of overlapping with the atomic van der Mer coordinates of the first portion of a protein backbone of the first van der Mer. In embodiments, step (d) includes repeating steps (b) and (c) for all van der Mer in the van der Mer database independently representing all chemical groups of the ligand, an additional independent amino acid side chain and an additional independent portion of a protein backbone bound to said independent additional amino acid side chain. In embodiments, the overlap of the van der Mers and the ligand chemical groups are not selected one at a time, but rather in pairs, triplets or higher order combinations. In embodiments, van der Mers at multiple sites are selected and the RMSD between these van der Mer chemical groups and the ligand chemical groups is computed. In embodiments, the RMSD between van der Mer chemical groups and the ligand chemical groups is precomputed and saved in lookup tables. In embodiments, the overlap of the van der Mers and the ligand chemical groups are selected in pairs. In embodiments, the overlap of the van der Mers and the ligand chemical groups are selected in triplets. In embodiments, the overlap of the van der Mers and the ligand chemical groups are selected in combinations greater than three. In embodiments an identified van der Mer is a cluster having a 0.5 angstrom RMSD between members. In embodiments an identified van der Mer is a sub-cluster of a van der Mer. In embodiments an identified van der Mer is a van der Mer cluster member. In embodiments an identified van der Mer is a van der Mer representative. In embodiments an identified van der Mer is a cluster having a 0.1 angstrom RMSD between members.

In embodiments, the optimizing includes an iterative or heuristic algorithm. In embodiments, the optimizing includes a simplex algorithm, memetic algorithm, differential evolution algorithm, evolutionary algorithm, genetic algorithm, tabu algorithm, particle swarm algorithm, or stimulated annealing algorithm. In embodiments, the optimizing includes a Monte Carlo sampling algorithm, dead-end elimination algorithm, branch and bound algorithm, or a pruning algorithm. In embodiments, the energy minimization calculation includes a molecular mechanics function, a structural bioinformatics function, an amino acid sidechain packing function, a protein radius of gyration function, or a combination thereof. In embodiments, identifying atomic van der Mer coordinates of a chemical group of a van der Mer as exposed to bulk solvent is performed using a convex hull algorithm. In embodiments, the cluster score is a function (e.g., including but not limited to a natural log or logistical function) of the ratio of 1) the number of members in an independent set of geometrically overlapping van der Mer of one chemical group and one amino acid to 2) the average number of members in all independent sets van der Mer of the chemical group and the amino acid. In embodiments, the members in an independent set of geometrically overlapping ligand van der Mer of one chemical group are identified by having an RMSD below a threshold, wherein the RMSD is calculated using the atomic coordinates of the chemical group and the backbone atoms of the amino acid residue of a van der Mer. In embodiments, the RMSD threshold is 0.5 angstrom. In embodiments, the preferred amino acid of step (g) is an amino acid in a van der Mer having a cluster score greater than 2. In embodiments, identifying an amino acid residue for each protein backbone residue having atomic amino acid coordinates that are not overlapping with the atomic van der Mer coordinates of a van der Mer is performed using The Rosetta Software. In embodiments, the van der Mer database is a collection of independent van der Mer each including a unique set of atomic van der Mer coordinates describing the three dimensional positions of a chemical group interacting in silico with an amino acid residue, further wherein the interacting was identified in an empirically determined protein and chemical group complex. In embodiments, the protein is a 4-helix bundle protein. In embodiments, the ligand includes a charged chemical group at physiological pH. In embodiments, the ligand includes a polar chemical group at physiological pH. In embodiments, the system includes use of a system described in international application no. WO2019/023644. In embodiments, the overlap of the van der Mers and the ligand chemical groups are not selected one at a time, but rather in pairs, triplets or higher order combinations. In embodiments, van der Mers at multiple sites are selected and the RMSD between these van der Mer chemical groups and the ligand chemical groups is computed. In embodiments, the RMSD between van der Mer chemical groups and the ligand chemical groups is precomputed and saved in lookup tables. In embodiments, the overlap of the van der Mers and the ligand chemical groups are selected in pairs. In embodiments, the overlap of the van der Mers and the ligand chemical groups are selected in triplets. In embodiments, the overlap of the van der Mers and the ligand chemical groups are selected in combinations greater than three. In embodiments an identified van der Mer is a cluster having a 0.5 angstrom RMSD between members. In embodiments an identified van der Mer is a sub-cluster of a van der Mer. In embodiments an identified van der Mer is a van der Mer cluster member. In embodiments an identified van der Mer is a van der Mer representative. In embodiments an identified van der Mer is a cluster having a 0.1 angstrom RMSD between members.

In embodiments, the non-transitory computer-readable storage medium including program code includes generating a plurality of sets of atomic coordinates of an in silico complex of the protein capable of binding the ligand bound to the ligand in step (f). In embodiments, the non-transitory computer-readable storage medium including program code includes generating all possible sets of atomic coordinates of an in silico complex of the protein capable of binding the ligand bound to the ligand in step (f). In embodiments, the plurality of sets of atomic coordinates of an in silico complex of the protein capable of binding the ligand bound to the ligand are independently different from each other and optimization of the overlap between the atomic chemical coordinates of the ligand chemical groups and the atomic van der Mer coordinates representing the independent chemical groups of the ligand identified in steps (b) to (d) is performed without duplication of the sets. In embodiments, the plurality of independent sets of atomic coordinates of an in silico complex of the protein capable of binding the ligand bound to the ligand are independently different from each other and are scored. In embodiments, the scoring includes calculating a cluster score for each of the plurality of sets of atomic coordinates of an in silico complex of the protein capable of binding the ligand bound to the ligand. In embodiments, the cluster score is the natural logarithm of the ratio of 1) the number of members in an independent set of geometrically overlapping van der Mer of one chemical group and one amino acid to 2) the average number of members in all independent sets van der Mer of the chemical group and the amino acid. In embodiments, the members in an independent set of geometrically overlapping ligand van der Mer of one chemical group are identified by having an RMSD below a threshold, wherein the RMSD is calculated using the atomic coordinates of the chemical group and the backbone atoms of the amino acid residue of a van der Mer. In embodiments, the RMSD threshold is 0.5 angstrom. In embodiments, step (a) includes generating a plurality of independent sets of atomic protein coordinates representing independent backbone structures of the protein. In embodiments, the plurality of independent backbone structures of the protein have a similar overall three dimensional fold. In embodiments, the plurality of independent backbone structures of the protein have an RMSD of less than 3 angstrom. In embodiments, the ligand chemical groups and van der Mer chemical groups are polar groups. In embodiments, steps (g) and (h) include use of a method described in international application no. WO2019/023644. In embodiments, step (c) includes identifying all portions of the backbone structure of the protein having atomic protein coordinates that are capable of overlapping with the atomic van der Mer coordinates of the first portion of a protein backbone of the first van der Mer. In embodiments, step (d) includes repeating steps (b) and (c) for all van der Mer in the van der Mer database independently representing all chemical groups of the ligand, an additional independent amino acid side chain and an additional independent portion of a protein backbone bound to said independent additional amino acid side chain. In embodiments, the overlap of the van der Mers and the ligand chemical groups are not selected one at a time, but rather in pairs, triplets or higher order combinations. In embodiments, van der Mers at multiple sites are selected and the RMSD between these van der Mer chemical groups and the ligand chemical groups is computed. In embodiments, the RMSD between van der Mer chemical groups and the ligand chemical groups is precomputed and saved in lookup tables. In embodiments, the overlap of the van der Mers and the ligand chemical groups are selected in pairs. In embodiments, the overlap of the van der Mers and the ligand chemical groups are selected in triplets. In embodiments, the overlap of the van der Mers and the ligand chemical groups are selected in combinations greater than three. In embodiments an identified van der Mer is a cluster having a 0.5 angstrom RMSD between members. In embodiments an identified van der Mer is a sub-cluster of a van der Mer. In embodiments an identified van der Mer is a van der Mer cluster member. In embodiments an identified van der Mer is a van der Mer representative. In embodiments an identified van der Mer is a cluster having a 0.1 angstrom RMSD between members.

In embodiments, the optimizing includes an iterative or heuristic algorithm. In embodiments, the optimizing includes a simplex algorithm, memetic algorithm, differential evolution algorithm, evolutionary algorithm, genetic algorithm, tabu algorithm, particle swarm algorithm, or stimulated annealing algorithm. In embodiments, the optimizing includes a Monte Carlo sampling algorithm, dead-end elimination algorithm, branch and bound algorithm, or a pruning algorithm. In embodiments, the energy minimization calculation includes a molecular mechanics function, a structural bioinformatics function, an amino acid sidechain packing function, a protein radius of gyration function, or a combination thereof. In embodiments, identifying atomic van der Mer coordinates of a chemical group of a van der Mer as exposed to bulk solvent is performed using a convex hull algorithm. In embodiments, the cluster score is the natural logarithm of the ratio of 1) the number of members in an independent set of geometrically overlapping van der Mer of one chemical group and one amino acid to 2) the average number of members in all independent sets van der Mer of the chemical group and the amino acid. In embodiments, the members in an independent set of geometrically overlapping ligand van der Mer of one chemical group are identified by having an RMSD below a threshold, wherein the RMSD is calculated using the atomic coordinates of the chemical group and the backbone atoms of the amino acid residue of a van der Mer. In embodiments, the RMSD threshold is 0.5 angstrom. In embodiments, the preferred amino acid of step (g) is an amino acid in a van der Mer having a cluster score greater than 2. In embodiments, identifying an amino acid residue for each protein backbone residue having atomic amino acid coordinates that are not overlapping with the atomic van der Mer coordinates of a van der Mer is performed using The Rosetta Software. In embodiments, the van der Mer database is a collection of independent van der Mer each including a unique set of atomic van der Mer coordinates describing the three dimensional positions of a chemical group interacting in silico with an amino acid residue, further wherein the interacting was identified in an empirically determined protein and chemical group complex. In embodiments, the protein is a 4-helix bundle protein. In embodiments, the ligand includes a charged chemical group at physiological pH. In embodiments, the ligand includes a polar chemical group at physiological pH. In embodiments, the non-transitory computer-readable storage medium including program code includes use of a method described in international application no. WO2019/023644. In embodiments, the overlap of the van der Mers and the ligand chemical groups are not selected one at a time, but rather in pairs, triplets or higher order combinations. In embodiments, van der Mers at multiple sites are selected and the RMSD between these van der Mer chemical groups and the ligand chemical groups is computed. In embodiments, the RMSD between van der Mer chemical groups and the ligand chemical groups is precomputed and saved in lookup tables. In embodiments, the overlap of the van der Mers and the ligand chemical groups are selected in pairs. In embodiments, the overlap of the van der Mers and the ligand chemical groups are selected in triplets. In embodiments, the overlap of the van der Mers and the ligand chemical groups are selected in combinations greater than three. In embodiments an identified van der Mer is a cluster having a 0.5 angstrom RMSD between members. In embodiments an identified van der Mer is a sub-cluster of a van der Mer. In embodiments an identified van der Mer is a van der Mer cluster member. In embodiments an identified van der Mer is a van der Mer representative. In embodiments an identified van der Mer is a cluster having a 0.1 angstrom RMSD between members.

### EXAMPLES

### Example 1—Strategy for Designing Hyperstable, Non-Natural Protein-Cofactor Complexes with Sub-Å Accuracy

A defined structural unit enables de novo design of small-molecule-binding proteins. A new representation of protein—chemical-group interactions enables design of proteins that bind the drug, apixaban. Computational code and design scripts are available in the supplement and github. Coordinates and data files of ABLE structures have been deposited to the PDB with accession codes: 6W6X (drug-free ABLE), 6W70 (apixaban-bound ABLE), 6X8N (H49A ABLE mutant).

The de novo design of proteins that bind highly functionalized small molecules represents a great challenge. To enable computational design of binders, we developed a unit of protein structure—a van der Mer (vdM)— that maps the backbone of each amino acid to statistically preferred positions of interacting chemical groups. Using vdMs, we designed six de novo proteins to bind the drug apixaban; two bound with low and sub-micromolar affinity. X-ray crystallography and mutagenesis confirmed a structure with a precisely designed cavity that forms favorable interactions in the drug-protein complex. vdMs may enable design of functional proteins for applications in sensing, medicine, and catalysis.

Here, we accomplish the reverse of the Anfinsen hypothesis by simultaneously designing structure and binding function from scratch, targeting a small-molecule drug with significant polarity and structural complexity. To do this, we developed a unit of local protein structure that directly links a tertiary structure to key interactions that engender tight and specific binding. These findings illuminate the principles underlying the emergence and evolution of complex function in proteins, and provide a methodology for designing useful proteins.

### Targeted Function and Fold

We targeted the factor Xa inhibitor, apixaban, an organic compound with five rotatable bonds and eight heteroatoms. Our first objective was to compute a tertiary structure capable of cooperatively binding the polar groups of apixaban. Instead of repurposing natural binding proteins or folds that have been shown to bind a similar ligand, in this work we use de novo 4-helix bundles because they are mathematically parameterized (10, 11), designable (12), and share no similarity to the fold of factor Xa. 4-helix bundles generally do not bind small molecules and instead bind metal ions or metalloporphyrins by strong coordinate bonds (10, 13-16). However, 4-helix bundles are tubular and can be designed to have high thermodynamic stability (11, 13) to compensate for the energetically demanding process of building binding cavities replete with buried polar functionality (17). Thus, the design of a de novo helical bundle that binds the drug apixaban critically tests the design method.

### The Van Der Mer Structural Unit

The design of proteins relies on optimal packing of interior sidechains in discrete conformations called rotamers (2, 3, 18-22). However, the design of ligand-binding proteins additionally requires sidechains that interact favorably with the target small molecule. Previous design strategies have approached this problem by computationally appending the target ligand to rotamers with idealized interaction geometries that—although comprised of billions of conformations—sample only a small fraction of the possible conformational space (6, 8, 23). These strategies rarely deliver sub-millimolar binders from the initial computational design so subsequent steps rely on experimental random mutagenesis and screening of libraries.

We wondered how much of the vast, possible conformational space of protein-chemical-group interactions is actually sampled in observed protein structures, and if sampling interactions directly from this distribution might aid the design of high-affinity binders. While previous analyses have focused on local sidechain contacts with chemical groups (24), we sought a structural unit that directly maps backbone coordinates to chemical-group locations, the link between the protein fold and binding function. We developed a unit of protein structure analogous to rotamers—van der Mers—that defines the placement of key chemical groups in the ligand relative to the backbone atoms of the contacting residue (FIG. 1B). van der Mers (vdMs) are culled from a non-redundant set of protein structures by 1) identifying all residues of a certain type that interact with a particular chemical group, 2) performing an all-by-all pairwise superposition of only the backbone and chemical-group coordinates (sidechains are not considered in the superposition, allowing some variation in their conformation), and 3) geometric clustering with a tight RMSD cutoff (0.5 Å). The resulting vdMs show backbone Φ/ψ-dependence (FIG. 1C) and capture compensatory effects of backbone and chemical-group placement. Furthermore, single clusters may contain multiple rotamers (FIG. 1D, FIG. 6A, and FIGS. S1A-S1D), given that sidechain coordinates are not explicitly considered in clustering.

The use of vdMs contrasts with procedures that place ligands at idealized locations relative to the terminal atoms of a sidechain (6, 8, 23, 25), which results in vast numbers of ligand—rotamer combinations that might never occur in proteins. Instead, vdMs sample locations of chemical groups relative to the backbone that have been experimentally vetted to achieve binding, regardless of ideality of the interaction. They also implicitly consider interactions with ordered or bulk water, which might influence their interaction geometries. Moreover, unlike ligand-appended and inverse rotamers used in earlier approaches (6, 8, 23, 25), vdMs may derive from contacts with either mainchain, sidechain or both in a multivalent interaction. Finally, the prevalence of a given vdM in the Protein Data Bank (PDB) can be used in scoring functions, similarly to scoring rotamers, which may assist automated selection of binding-site residues for design.

To maximize the number of observed protein—chemical-group contacts, we created vdMs using the chemical groups of amino acids that comprise the protein (e.g., CONH₂ of Gln and Asn, N—H and C═O of backbone amide). To avoid bias from local structure, we counted only the interactions that were distant in the linear polypeptide chain, as described in the supplement. The set of chemical groups can also be expanded to include those from small-molecule drugs, metal ions, and cofactors, although these are not as pervasive in crystal structures.

We rank vdMs by their prevalence in the PDB using a log-odds score, C (FIG. 1D, FIG. 1E, FIGS. S2A-S2D, and supplement). Although there are hundreds of vdMs associated with a given residue/chemical-group combination (FIGS. S3 and S4), only a small fraction of vdMs are highly enriched in protein structures (C>0). For example, only 91 Asp/CONH₂ vdMs have C>0; these top vdMs map the locations of CONH₂, relative to the backbone of an Asp residue, that are statistically preferred by proteins in the PDB (FIG. 1E and FIG. S2D). This is on the order of the number of rotamers used for an amino acid during a typical protein design packing calculation (26). Thus, when combined with an efficient search algorithm, sampling protein—chemical-group interactions with vdMs to design ligand-binding sites might be as expedient as sampling rotamers to pack a protein core. Furthermore, functionally relevant lower-probability rotamers may be included if contained in a high-scoring vdM.

Proteins use the same set of 20 amino acids to fold as well as to recognize a vast array of highly functionalized ligands. We therefore hypothesized that the interaction modes used by amino acids to stabilize their tertiary structures would also be used to achieve tight binding of ligands, even those containing structurally distinct heterocyclic chemical groups. To test this hypothesis, we examined the streptavidin—biotin complex (FIGS. 2A-2D). Using the natural sequence of streptavidin, we examined the positions of vdMs of N—H, C═O and COO⁻, where these groups were derived from protein mainchain and sidechain. In each case, we observed that the sidechain interactions with biotin's polar groups involved highly favorable vdMs, with enrichment scores of approximately 8-fold or greater (C>2). The streptavidin sequence/fold pairing cooperatively positions highly favorable vdMs to cover each polar chemical group of biotin simultaneously.

Our analysis of the streptavidin—biotin complex suggests that binding sites can be designed by considering folds that position vdMs to collectively bind the distinct chemical groups found in a target small-molecule ligand. Moreover, the vdMs of the binding site should be maximally prevalent in the PDB. We developed a search algorithm, called Convergent Motifs for Binding Sites (COMBS), to discover favorable poses of a ligand that satisfy these criteria.

### De Novo Design Strategy

Our design strategy consists of several hierarchic steps, which prioritize the most essential and difficult features to avoid sampling regions in sequence/structure space with little chance of success (FIG. S5). First, we define the chemical groups within the small molecule that will be targeted. We initially focus on polar chemical groups, which are the most challenging to dehydrate but must be satisfied with H-bonds to achieve high affinity and specificity (27). Secondly, we choose a designable protein fold, and create an ensemble of backbones with geometries that are consistent with the known plasticity of the fold. Next, for each backbone we use COMBS to identify members of the backbone ensemble that can position vdMs to collectively engage each of the targeted chemical groups of the small molecule. In this way, the binding of the desired ligand dictates the precise backbone geometry. Having discovered candidate backbones and binding sites, the design is completed by engineering a tightly packed folding core that supports the vdM-derived keystone interactions in the binding site (13). In this step, we constrain the keystone interactions and use flexible backbone design (13, 26) to pack additional residues within the binding site while simultaneously packing the protein core.

We focused on apixaban's carboxamide (both the C═O and —NH₂), as well as two additional carbonyls (FIG. 3A, Other groups that were internally H-bonded or easily dehydrated were not initially targeted). We created a set of vdMs of carboxamide (CONH₂ from Asn and Gln sidechains) and carbonyl (C═O from protein backbone, see supplement) and used these vdMs to discover preferred CONH₂ and C═O binding locations within a set of 32 mathematically generated de novo poly-glycine backbones (10, 28) (FIG. 3B, FIG. 3C, FIGS. S2A-S2D, and Table S1). For each of the mathematically generated backbones, we placed apixaban in the protein interior by using a separate set of vdMs with apixaban superimposed onto the chemical group of the vdM. For example, the CONH₂ of apixaban can be superimposed on the CONH₂ of a vdM, uniquely defining the position of apixaban in the binding site. Apixaban's conformation in this step was fixed in a low-energy conformer found in its co-crystal structure with factor Xa (PDB code 2p16; FIG. 3A and FIGS. S6A-S6B; extension to multiple conformers is discussed in the supplement and FIGS. S7A-S7C). vdMs that cover the remaining C═O groups of the placed ligand, as well as additional vdMs to the carboxamide, were then queried in the nearby space (FIG. 3D). We chose binding poses by maximizing the PDB-prevalence of sterically compatible vdMs (ΣC, FIG. 3E).

Sidechains from vdMs in six selected binding poses were fixed, and their H-bonding interactions with apixaban were constrained in all subsequent steps of sequence design using Rosetta. After insertion of interhelical loops, we used a flexible-backbone design protocol (13) (FIG. 3F) to compute the hydrophobic core while simultaneously completing the packing of the binding site. For some designs, new polar interactions were recruited during this step, as well as Gly residues, which are known to interact favorably with aromatic groups (27). The use of small residues to make hydrophobic contacts minimizes the number of large, apolar sidechains that might lead to non-specific binding or hydrophobic collapse in the absence of ligand.

### Description of Designs and Biophysical Characterization

We designed six proteins of varying length, topology, ligand position, ligand burial, and keystone interactions (FIG. S8). Contrary to factor Xa, which engages polar groups of apixaban via main-chain amides in loops (FIGS. S6A-S6B), the designs interact with apixaban using predominantly sidechains in helices. The six designs expressed well in bacteria, and each was helical based on far UV circular dichroism spectroscopy (FIG. S9). Proton NMR showed that two designs, ABLE (apixaban-binding helical bundle) and LABLE (Longer ABLE), bound apixaban (FIG. S10). These two designs had the same orientation of apixaban within the bundle and shared the same vdM-derived keystone interactions (FIG. 3E and FIG. S8). For example, they shared a buried, high-scoring His/C═O vdM (8-fold enrichment, C=2.1, FIG. 3E). However, ABLE and LABLE differed in length (125 vs. 165 residues), topology, loop geometry, and shared only 22% sequence homology.

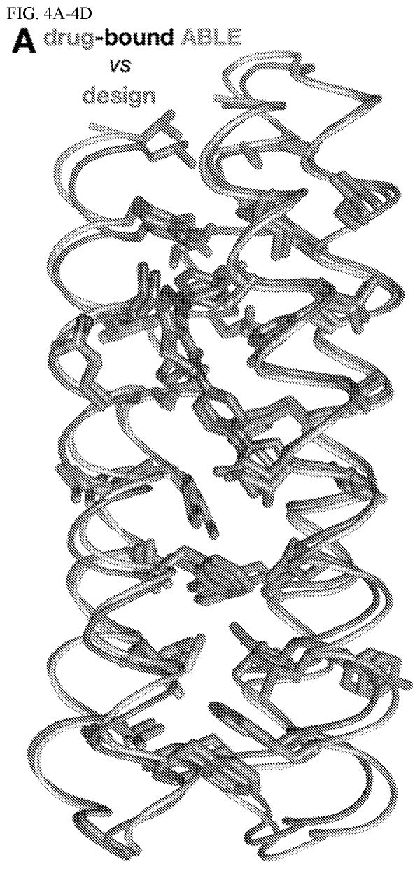

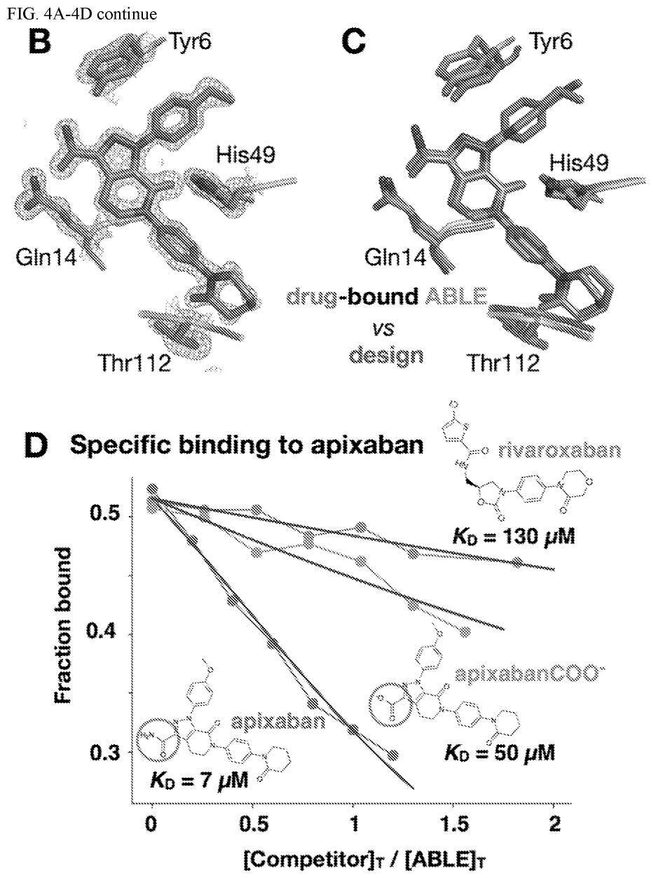

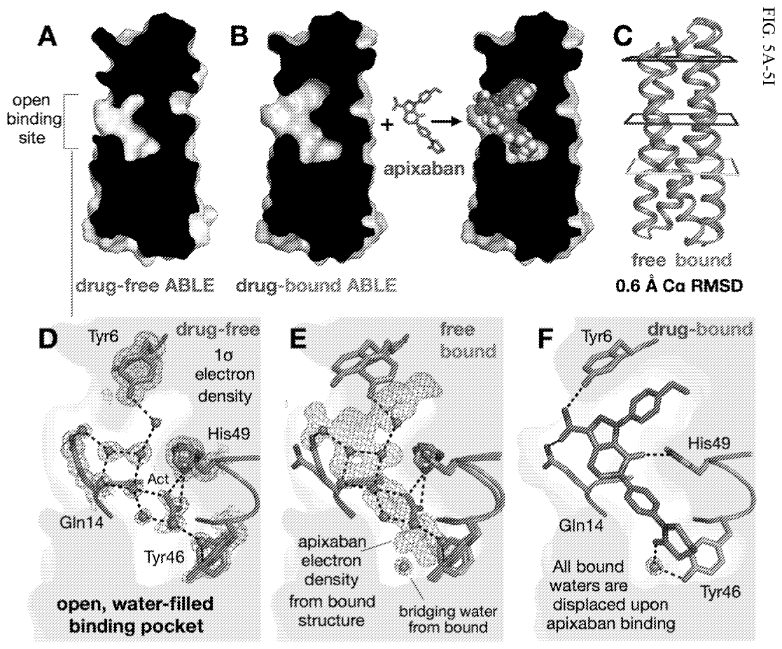

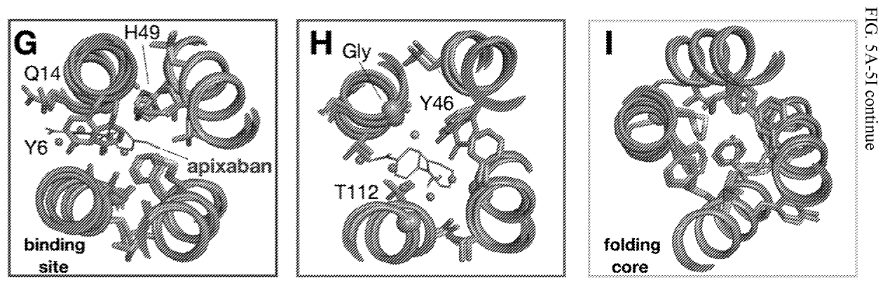

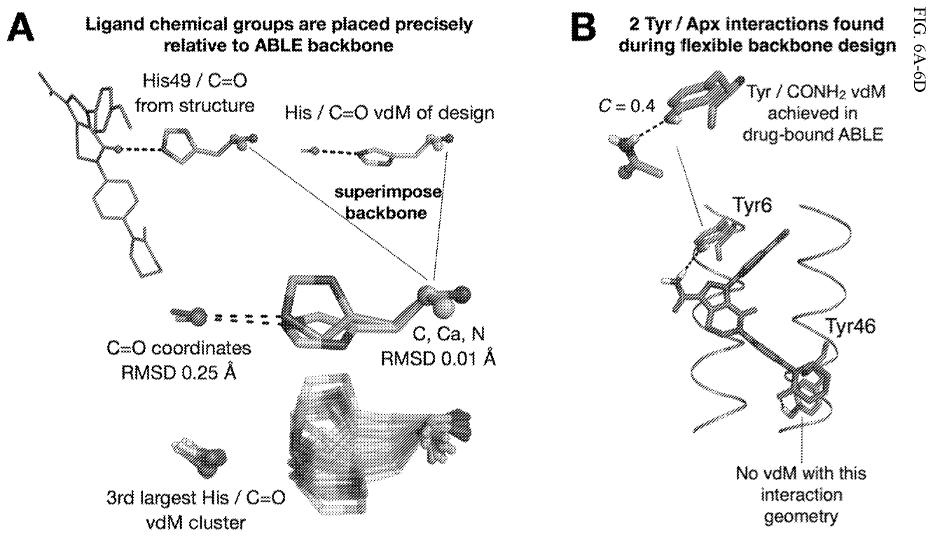

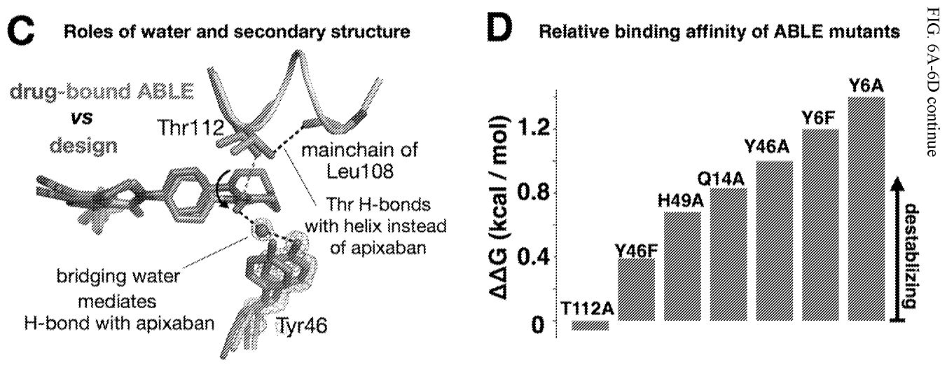

Binding of apixaban to ABLE restricts the drug's conformation, resulting in a redshift of its electronic absorbance spectrum (FIG. 3G). Spectral titrations and fluorescence polarization competition experiments showed that ABLE and LABLE bind apixaban with a dissociation constant (K_{D}) of 5 (±1) μM and 0.6 (±0.1) μM, respectively (FIG. 3H, FIG. 4D, FIGS. S11A-S11D and FIGS. S12A-S12D). Although LABLE showed a dispersed 2-dimensional ¹H-¹⁵N HSQC spectrum by NMR (FIG. S13), indicative of a well-structured protein, it failed to crystallize in a sparse matrix screen; so we focused our attention on characterization of ABLE. ABLE is monomeric in solution (FIG. S14) and highly stable to heat denaturation (melting temperature >95° C.), despite the inclusion of three Gly and a polar His within its core (FIGS. S15A-S15B).

### Structures of Apixaban-Bound and Drug-Free ABLE

ABLE readily crystalized with apixaban and diffracted to 1.3 Å resolution. Two very closely related monomers are observed in the asymmetric unit (FIGS. 516A-516B); apixaban is bound to both monomers, as expected for a specific, high-affinity complex. The structure of the drug-bound protein is in excellent agreement with the design (Cα RMSD of 0.7 Å, FIGS. 4A-4D). The rotamers of the core residues of ABLE, including the binding-site residues, overwhelmingly agree with the design model. Superimposing by all heavy atoms of core amino acids, including apixaban, gives an RMSD of 0.98 Å. ABLE buries almost all available apolar surface area (504 Å²) of apixaban, and it also forms most polar interactions included in the design (FIG. 4B and FIG. 4C). Apixaban's conformation is close to that used in the design (0.6 Å heavy atom RMSD), with small deviations that bring it closer to a quantum mechanically optimized geometry (FIG. S17). The rigid-body translation between apixaban's center of mass in the designed versus observed structures is only 0.2 Å, with a rigid body rotation of 6°. The bespoke binding site is specific for apixaban, as shown by fluorescence polarization competition experiments (FIG. 4D), which show that ABLE binds apixaban 20-fold more tightly than a similar factor Xa inhibitor, rivaroxaban.

To assess the extent of preorganization of the protein, we also solved the drug-free structure to 1.3 Å resolution (FIGS. 5A-5I). The structure shows an open, preorganized binding pocket, with an overall Cα RMSD of 0.65 Å to the apixaban-ABLE complex. The unoccupied binding site is solvated by nine ordered water molecules plus an acetate from the buffer (FIG. 5D). Binding of apixaban displaces ordered solvent from this site, suggesting a release of local frustration upon binding. The pocket has a 480 Å² solvent exposed surface area, approximately 30% smaller than that of the liganded protein (680 Å²). The drug-free protein has nearly identical rotamers to that of the drug-bound protein throughout the core and binding site (FIGS. 5G-5I). Unliganded ABLE shows two alternate rotamers for several of the residues that form H-bonds to apixaban (e.g. Tyr46 and His49); binding of apixaban selects one each of these alternate rotamers. Thus, like many natural proteins (29), ABLE has a limited degree of flexibility, which is reduced upon ligand-binding, and the binding event appears to trade configurational entropy for enthalpically favorable interactions.

Insights from the Structure and Function of ABLE

Two of the three keystone interactions identified by COMBS contribute significantly to binding affinity. Substitution of His49 or Gln14 to alanine individually decreases affinity by approximately 1 kcal/mol (˜3-fold, FIG. 6D and FIG. S18). Gln14 was observed in its intended rotamer, while His49 occupied an alternate rotamer that nevertheless maintained the intended position of apixaban's carbonyl relative to the mainchain (FIG. 6A). Indeed, the cluster describing this His/C═O vdM contains multiple His rotamers, each capable of achieving identical placements of C═O relative to mainchain. Thus, we observed vdM convergence, even amidst rotamer divergence.

We also examined the structural consequences of substituting His49 to Ala by solving the crystal structure of the unliganded H49A mutant protein (FIGS. 519A-519C). Although the structures of drug-free ABLE and drug-free H49A are similar (Cα RMSD=1.2 Å), the residues that surround His49 show rotameric differences in the absence of this sidechain; released from the restraints of tight packing, they instead adopt their preferred rotamers. The structure illustrates that global packing of core residues supports the positioning of a key functional group, even when this requires local frustration at individual sites.

Substitution of the third keystone residue, Thr112, to Ala resulted in little change in affinity (FIG. 6D). In the complex, its sidechain did not form the intended H-bond to apixaban, but instead formed an intracelial H-bond to a backbone carbonyl (FIG. 6C). The intended Thr/C═O vdM is favored in the backbone-independent vdM library used in the design of ABLE, but is disfavored in a backbone-dependent vdM library. The lack of engagement with apixaban's carbonyl resulted in some disorder of the terminal oxopiperidine, which has higher b-factors and two alternate conformations (related by a 180° ring flip) in the structure (FIGS. 516A-516B). Thus, backbone-dependent vdM libraries should be used in future applications.

Flexible-backbone sequence design of ABLE recruited two Tyr residues that interact with apixaban (FIG. 6B). One of these interactions was represented in the vdM database (Tyr6/CONH₂, C=0.4) but the other (Tyr46/C═O) was not. The structure of drug-bound ABLE confirmed the H-bond of Tyr6/CONH₂ (FIG. 4C), but an unanticipated water enters the binding site to mediate an H-bond between apixaban and Tyr46 (FIG. 6C). Furthermore, substitution of Tyr6 to Phe or Ala was more destabilizing than the same substitutions of Tyr46, tracking with prevalence in the PDB. Thus, vdMs could be used to filter and rank interactions obtained using a variety of computational methods (30).

Finally, we wondered if ab initio folding predictions (26) might distinguish between successful versus unsuccessful designs. Of the six designs, only two—ABLE and LABLE—were predicted by folding simulations to maintain uncollapsed binding sites (FIGS. S20A-S20C). Moreover, the lowest energy models predicted from ab initio folding simulations of ABLE's sequence largely agree with the crystallographic structure (FIG. 520A). Thus, ab initio folding may be useful as a screen to assure that designs maintain an open, preorganized site. These results emphasize the degree to which the folding and binding problem are intimately coupled.

Previously, the design of de novo proteins that bind in a shape-selective manner to rigid, flat, hydrophobic dyes or lipidic metabolites has been possible, but binding flexible molecules replete with polar atoms has been more challenging (4, 8, 31-33). Natural proteins bind highly functionalized ligands by first accruing the ability to weakly bind fragments within the context of a particular fold (34-36). To mimic this process, we developed the vdM structural unit to directly link the protein fold to statistically preferred binding modes of chemical groups. We sampled vdMs on the backbone of a designable 4-helix bundle to create constellations of chemical groups that, when matched with the shape of apixaban, defined the binding site. This contrasts with previous approaches that search for positional matching of whole ligands, sampled using idealized interaction geometries. Such approaches are highly sensitive to small changes in the interaction geometries, thus requiring an enormous amount of sampling to discover possible binding solutions, many of which may contain interactions not observed in the PDB.

vdMs sample from the experimentally-vetted distribution of observed protein structures. vdMs are surprisingly sparse and discrete (FIG. 1E, FIG. S3, FIG. S4), and they enable facile sampling of sequence space to discover convergent combinations of keystone interactions (Examples and FIGS. S2A-S2D). We consider only the backbone and the orientation of the pendant chemical group, which obviates the need to enumerate a large ensemble of ligand-appended rotamers for each amino acid type at each position of the sequence. We focused here on simple, fully de novo scaffolds rather than redesigning the specificity of natural ligand-binding proteins, because we wished to address the challenge of designing function entirely from scratch. Indeed, ABLE shares no sequence homology to any known proteins (blast E value <0.42 against the nonredundant protein sequence database nr). We used only prevalence to rank vdMs and choose binding sites, but we suspect the true power of vdMs may lay in higher-order correlations of the interactions.

COMBS and vdMs can now be used for a variety of protein engineering applications, and in full partnership with experimental optimization strategies for exploring sequence space. We anticipate that vdMs can also be used to predict chemical-group hotspots of proteins with fixed sequence. vdMs may also enable design of protein—protein interfaces in a self-consistent manner. Finally, because vdMs sample from the distribution of evolved interaction geometries observed in protein structures, it is tempting to view the chemical-group constellations constructed by vdMs as a structural hypothesis of the evolutionary path to acquire binding within the context of a given fold.

### Curation of Van Der Mers

### PDB Database for vdM Generation

We downloaded protein structures from the RCSB with 30% sequence homology, X-ray diffraction resolution ≤2.0 Å, and R_{obs}≤0.3. We used the program Reduce (37) to add hydrogens to the structures and to perform any necessary rotamer-flips of Asn, Gln, and His residues. We then used the program Molprobity (38) to obtain the Molprobity score for each structure. We subsequently constructed biological assemblies of the PDBs with Molprobity score ≤2, using the program Prody (39). The final list of accession codes/chain IDs for van der Mer (vdM) searching can be found in the supporting file, Data S1. The non-redundant structural database contains a total of 8743 PDBs with 9189 unique chains. Note that while we used biological assemblies to search for vdMs, we only searched through the non-redundant chains in the structure, such that contacts could be found across subunits of the assembly, without artificial duplication of vdMs.

### Defining Protein/Chemical Group Contacts for vdM Generation

We approximated chemical groups (CGs) as fragments of amino-acid sidechain or mainchain, in order to increase sampling statistics. For example, our database contains 348,067 residues contacting a carboxamide derived from Asn or Gln sidechains. Of these, 189,849 residues have interactions with carboxamide that are distant in sequence (>7 amino acids away in the linear polypeptide chain), which avoids bias from nonspecific proximity effects. In this work, we further winnowed the number of interacting residues by considering only H-bonded interactions (85,750 residues). To define a vdM, we next categorize the interactions by residue type (e.g., 5,785 Tyr residues H-bond with a carboxamide).

We used the program Probe (40) to determine which amino-acid residues are in van der Waals (vdW) contact with a given chemical group (CG), as well as the nature of the contact (H-bond, close vdW contact, wide vdW contact). For example, to search for vdMs of carboxamide, we iterated through every Asn and Gln residue in each unique protein chain in the database. For each Asn and Gln residue in the chain, we used Probe to detect other residues in the biological assembly that are within vdW contact of the sidechain's carboxamide (e.g., CB, CG, OD1, ND2, HD21, HD22 atoms of Asn). To find vdMs of carbonyl (C═O), we used the backbone carbonyl of Gly and Ala residues. We then used only the subset of vdMs that formed H-bonded interactions. These vdMs were grouped in two ways: by superposition on mainchain for sampling, and by superposition on mainchain and chemical group coordinates for scoring (see text below and FIGS. S2A-S2D).

### vdM Cluster Score

We scored vdMs based on their prevalence in the non-redundant protein structural database. Instead of aligning vdMs exactly by amino-acid backbone atoms, we performed a pair-wise all-against-all superposition of backbone (N, Cα, C) and CG atoms for every vdM of a particular amino-acid type. Using both backbone and CG in the superposition helps to alleviate the lever-arm effect, where small changes in backbone coordinates lead to large changes in the location of a CG. The all-against-all pairwise RMSD matrix was used to cluster vdMs by RMSD <0.5 Å, using a greedy clustering algorithm. Much of the interaction space sampled by proteins in our database is captured in a small number of these clusters. For example, only 31 clusters of Asp/carboxamide vdMs are needed to capture half of the observed interactions (FIG. S3). The corresponding curves for each amino acid are provided in FIG. S4.

A single cluster may use a variety of sidechain rotamers to position the chemical group in the same location relative to the residue's backbone atoms, and the sidechain dihedral angles of vdMs appear to follow the same distribution as canonical rotamers (FIGS. S1A-S1D), which may prove beneficial for generation of synthetic vdMs that employ non-canonical chemical groups. (Many non-canonical chemical groups, such as halogens, can be found in protein-cocrystal structures in the PDB with bound drugs. A limited set of vdMs could be generated based on these structures as well.)

We defined a cluster score (C) of a vdM as a quantitative measure for how representative that cluster's interaction geometry is for that residue type in the PDB. The score is based on placement of a CG relative to the protein backbone, since backbone and CG coordinates are the only coordinates involved in clustering. Sidechain conformation (rotamer) is not explicitly considered in the clustering and therefore not in C. We compare the size of the cluster k to the average cluster size of that vdM type by C^{(k)}=In N^{(k)}/ [symbol] N [symbol] [symbol] where N^{(k)} is the number of members in cluster k and [symbol] N [symbol] | [symbol] N̅ [symbol] is the average cluster size (FIG. 1E). Positive C indicates the location of the CG relative to the backbone, represented by the cluster, is enriched relative to other locations of the CG. We used only interactions with positive C in the design of ABLE.

### vdM Representatives for Sampling

For sampling we used more fine-grained clusters, which would allow sampling over finer elements of conformational space (FIGS. S2A-S2D). To create these sub-clusters, we aligned the vdMs exactly by backbone atoms (N, Cα, C), and tightly clustered them (using a greedy clustering algorithm) by sidechain and CG coordinates (all-heavy-atom RMSD of 0.1 Å). The centroids of each cluster were used directly in sampling of vdMs on protein backbones. We refer to this fine-grained set as “vdM representatives”; by this definition, each vdM can be divided to a smaller number of vdM representatives. In this way, we can sample through representative members of a given vdM cluster without over-sampling very closely related members. In summary, we refer to a vdM as the cluster defined using a 0.5 Å RMSD cutoff, vdM cluster members as the individual members of the set, and vdM representatives as sub-clusters used for sampling.

### Design Protocol

### Generation of Parametric Helical Bundles

We aimed to create a highly stable protein that not only folds to the desired structure but also binds a ligand, which further restrains the sequence space in addition to the requirements for folding. We therefore sought to use a highly designable scaffold that can accommodate many sequences but is still tractable to computationally design from scratch. Consequently, we parametrically generated a small set [32] of antiparallel 4-helix bundles using Crick parameters that are similar to those describing natural heme-binding proteins, such as helical bundles in cytochrome BC1, and to those describing non-natural porphyrin-binding proteins, such as the de novo bundle PS1 (13). Using the CCCP server (10), we sampled parameters on a grid that varied the bundle radius from 7.9 Å to 8.2 Å, and covaried the superhelical phases of two helices by 14°, resulting in bundles that had wide interfaces that varied between 108 and 120° (interhelical Cα distances of ˜8.2-9.8 Å). These parameters were chosen because they result in highly designable backbones that can accommodate a variety of sequences (see structural bioinformatics below), as well as provide a variable-sized binding cavity for the ligand. Bundle parameters can be found in Table S1.

### Structural Bioinformatics of ABLE Parametric Backbone

We used the program Master (41) to query a structural database of approximately 20,000 protein crystal structures filtered at 50% sequence homology and with resolution <2.5 Å (R_{obs}<0.3). A four-helix query of the database (10 residues each helix) returned 319 unique proteins with structural matches with Cα RMSD <2 Å (Table S2). A query of the tightly interfaced helix-helix pair (10 residues each helix) of the parametric backbone returned 1466 unique proteins with structural matches with Cα RMSD <0.7 Å.

The backbone of ABLE was defined by parametric design (28, 42), using a simple algebraic expression with a handful of adjustable parameters to define a highly symmetrical backbone with reasonable bond lengths and angles. The resulting backbone nevertheless served as a scaffold for design of proteins that bind a highly complex and asymmetric ligand. Curious about other proteins that might use this scaffold functionally, we probed the structural similarity of this backbone to natural four-helix bundle proteins in the PDB. We found hundreds of structural matches to a wide variety of proteins both natural and designed, with natural proteins ranging from the meiotic synaptonemal protein complex (43) to a superoxide oxidase (44); and with de novo proteins designed to form internal hydrogen bonds (45) or to bind porphyrins (13) (Table S2). One very recent structure (pdb 5xub) of a domain from a chemotaxis protein (46), deposited subsequently to the design of ABLE, binds citrate in approximately the same location of a four-helix bundle as the location of apixaban in ABLE. This collection of bundles illustrates the emergence of diverse complex functions from relatively minor (<2 Å Cα RMSD) tweaks to an otherwise fully symmetrical scaffold.

### Ligand Conformation

We used the conformation of apixaban from the co-crystal structure with factor Xa (pdb 2p16, FIGS. S6A-S6B). We added hydrogens with the program Avogadro and created a Rosetta params file (see supplementary text) for use in flexible-backbone sequence design. This conformation is similar to its relaxed in vacuo conformation but is slightly higher in energy. The carboxamide of apixaban is internally H-bonded to the pyrazole nitrogen, creating a stable energy well for this conformation, which is observed in all small-molecule crystal structures of apixaban in the Cambridge Structural Database (FIGS. S7A-S7C). Indeed, the conformation of apixaban in complex with ABLE differs slightly from the factor Xa geometry (0.6 Å RMSD, FIG. 4C), but is almost identical to that observed in small-molecule crystal structures, as well as the quantum chemically optimized geometry via DFT (47) using the B3LYP functional and 6-31G* basis set (FIGS. 4A-4D, FIGS. S7A-S7C, and FIG. S17). We also computationally explored three higher-energy alternate conformations of apixaban, related by torsion about the methoxy-phenyl and the terminal 2-oxopiperidine moieties (FIG. S7A). For these conformations, we generated ligand-appended vdMs (see below) and searched for binding sites in the same way as in the design of ABLE and LABLE. These searches did not discover any better-scoring binding sites than those found using the apixaban conformer from factor Xa, so we did not experimentally investigate designs for these alternate-ligand conformations. We ordered apixaban as a solid from Combi-Blocks and made DMSO stock solutions varying from 1 mg/mL to 18 mg/mL.

### COMBS Strategy

The collective process of generating vdMs, loading vdMs on a backbone, sampling ligand poses, and selecting protein—ligand interactions is called COMBS (convergent motifs for binding sites). Below, we describe the process by which COMBS finds binding sites that achieve H-bonded interactions with the ligand apixaban (FIG. S5).

### Interior Vs Exterior Defined by Convex Hull Algorithm

The design process starts with the coordinates of a poly-glycine backbone only. We used a restricted set of residues (H, S, T, Y, W) for sampling buried vdMs of carboxamide and carbonyl in the interior of the protein bundle and used a more expanded set for intermediate and exterior positions (H, S, T, Y, W, Q, N, D, E, R, K). We defined interior, intermediate, and exterior positions with a convex hull algorithm (48). We first make an all-Ala version of the protein, which defines the positions of Cβ atoms. The convex hull algorithm uses Cα and Cβ coordinates of the protein to define two surfaces. If the Cβ atom lies on the surface of the Cβ hull, that residue is exposed. If a Cβ atom lies in the interior of the Cα surface, then that residue is either buried or intermediate. Intermediate residues are those that are also part of the Cβ hull. The algorithm can limit the size of the radius of the sphere (alpha sphere) that is used to define the exterior surface, which limits the surface coarseness. We used an alpha-sphere size of 9 Å.

Sampling of vdMs on a Backbone

We sample vdMs by aligning a set of vdM representatives (see above) to a backbone position. This has the effect of placing a chemical group (CG) in space relative to the backbone (sidechain is also placed). Similar to the program Probe, we use van der Waals radii of the atoms to define clashes of vdM sidechain and CG with the surrounding mainchain atoms, taking into account close approaches due to H-bonding. We do not sample vdMs one at a time in a conventional rotamer-sampling algorithm, but instead load them simultaneously onto a backbone scaffold to concurrently enumerate all possible CG locations (see Nearest neighbors graph of CGs). Multiple vdMs can occupy the same residue position on the backbone.

For sampling, we divided vdMs into 4 interaction types: 1) those making only backbone Cα and/or N—H contacts with the CG (called bbNH vdMs); 2) those making only backbone C═O contacts with the CG (called bbCO vdMs), 3) those making only sidechain contacts with the CG (called SC vdMs); and 4) those making both mainchain and sidechain contacts with the CG (called φψ vdMs). For each parametrically generated helical bundle, we aligned vdMs of each category to the backbone by superposing, respectively, by 1) Cα, N, H atoms, 2) Cα, C, O atoms, 3) N, Cα, C atoms, and 4) N, Cα, C atoms. This allows for a finer sampling of vdMs that have interactions that are dependent on only φ, only ψ, or both φ and ψ. bbNH vdMs are φ-dependent, and bbCO vdMs are ψ-dependent. For sampling, we treated SC vdMs as φ/ψ independent, although φ/ψ dependence of the rotamer is implicitly considered when we remove any vdMs that clash with the mainchain. Because φψ vdMs are inherently φ/ψ dependent, we only sampled them from vdMs with φ/ψ in a bin of ±30° of φ/ψ of the scaffold residue onto which they were aligned.

We sampled vdMs over a 14-residue span of each ˜40 residue helix. We loaded vdMs onto 14×4 residue positions and created an array of CG coordinates for construction of a nearest neighbors graph, which we used to discover vdMs that are consistent with the position of a ligand.

### Nearest Neighbors Graph of CGs

We construct a nearest-neighbors graph from the CG coordinates of the vdMs once they have been superimposed onto the backbone scaffold. For carboxamide, we used an RMSD of 1.0 Å for the CG (Cb, Cg, Od1, Nd2 atoms of Asn, and Cg, Cd, Oe1, Ne2 atoms of Gln). For carbonyl (backbone C and O atoms of Gly and Ala), we used an RMSD of 0.7 Å. We used the nearest-neighbors implementation in the Python package sci-kit learn. This allows for very fast lookups of neighbors given query coordinates, which we take from placed ligands (see below). The neighbors tell us precisely which vdMs place a chemical group within the RMSD threshold of the query coordinates, as well as the RMSD distance of each from the query. The next step in the design process is to determine which of these neighboring vdMs possess sidechains that do not clash with the placed ligand, and then to score the clash-free remainder by C (see above).

### Ligand-Placement Algorithms

Previous computational approaches to sample ligand positions have focused on either geometric overlap of entire ligands (6, 49, 50) or on ligand placement with one user-defined contact (23). For example, after sampling ligand-appended rotamers on protein backbones, candidate binding sites were defined as those that placed the full ligand in the same region of space (6). These approaches suffer from the lever-arm effect, where small deviations in protein— ligand contact geometry amplify to large changes of the ligand position remote from the contacting region. Massive amounts of sampling are required to overcome the lever-arm effect (4, 6, 8, 23), yet only a fraction of the total possible conformational space is available for sampling on a reasonable timeframe, even on large computing clusters. COMBS instead uses a set of ligand-superimposed vdMs to initially place a ligand in the binding site (see below) but then looks for nearest-neighbors vdMs of the ligand's chemical groups, instead of matches to full ligand locations. COMBS currently searches through static conformers only, such that searching through multiple conformers of a ligand requires the generation of a different set of ligand-superimposed vdMs for each conformer. Searches through multiple conformers can then be run in parallel.

### Generation of Ligand Poses

To generate ligand placements relative to the protein backbone, we first curate a set of vdMs with the ligand superimposed by the CG. We remove all vdM/ligand combinations that are clashing after superposition. We then load this set of ligand-superimposed vdMs onto the backbone scaffold in the same way we load vdMs. This has the advantage of placing the ligand with a least one vdM-derived CG contact, that of its superimposed vdM. We remove any ligand-superimposed vdMs with ligand or sidechain that is clashing with the backbone. We further remove any ligand-superimposed vdMs based on ligand burial. For design of ABLE and LABLE, we required at least 60 percent of apixaban's apolar heavy atoms to be buried in the interior of the protein, as defined by the convex hull (see above).

With the coordinates of the other CGs within the ligand now defined relative to the backbone, we use these coordinates as queries to the nearest-neighbors graph of carboxamide and carbonyl. We look for overlap of the ligand's CGs in their respective nearest-neighbors graphs instead of overlap of an entire ligand in order to reduce the lever arm effect, which amplifies small deviations in local geometry to affect large swings in distant parts of a ligand. The use of CG graphs allows us to find binding interactions for a particular ligand location consistent with small local deviations in the interactions that would otherwise be missed by a search for full ligand overlap. By sampling the ligand position with superposed ligands onto vdMs, we experience the lever arm effect only once (during the superposition), instead of multiple times (one time per CG) in the ligand.

### Selection of Ligand Poses for Further Design

We selected poses of apixaban based on ligand burial and satisfaction of H-bonding constraints to its buried CGs. We required that the two carbonyls and the carboxamide of apixaban be engaged in a vdM-derived H-bond if buried in the interior of the protein. We selected individual vdMs (among all the nearest neighbors) for a ligand pose based on maximizing C while avoiding vdW clashes between vdM sidechains. We chose 6 poses based on apixaban burial and ΣC that explored three distinct placements of apixaban (FIG. S8). We further checked for the robustness of the pose by clustering ligand poses by ligand position across the 32 bundles. Poses from large clusters with the same vdM-derived interactions suggested these interactions could be consistent with small-scale structural fluctuations on the order of 1 Å Cα RMSD.

### Flexible Backbone Sequence Design

After vdM-derived ligand placement and H-bonded interactions were found to apixaban, we performed a custom protocol for flexible-backbone sequence design in the program Rosetta (26) (linux version 2018.33.60351). We froze the identities and rotamers of the H-bonded residues, and constrained the H-bond distances using a harmonic potential. We generated a parameter file for apixaban for use in Rosetta, which defines its partial charges (see supplemental text). We did not allow the ligand conformer to be flexible during design.

We automatically generated Rosetta residue files based on burial and secondary structure of each position in the backbone. To do so, we applied the convex hull algorithm described above, as well as the secondary structure assignment program DSSP, to the entire PDB dataset (9,000 proteins) to create burial and secondary structure propensities for each residue type, based on backbone coordinates only. The propensity is defined as p=fₐₐ(burial, ss)/fₐₐ where fₐₐ(burial, ss) is the frequency that amino acid aa occurs in that burial assignment (exposed, intermediate, or buried) with secondary structure ss, and fₐₐ is the frequency of the amino acid aa in the database. We used residues at each position that had a burial and secondary structure propensity p≥0.9. For 3 of the 6 designs, including that of ABLE, we allowed Ala, Ser, Thr, and Val residues at solvent exposed positions during design to lower the surface polarity in order to promote crystallization. Scripts for flexible-backbone sequence design can be found in supplementary text below. The outputted backbones (500 total) varied on average from their starting structure by ˜1 Å Cα RMSD. We selected designs for advancement to the next stage of computation by considering the packing of the core residues (pstat score in Rosetta) and the overall energy (ref2015 weights).

### Loop Construction

Loops connecting helices are selected from a database of natural α-helical protein structures and spliced onto the backbone to minimize Cα distance with the helices (51). The loop sequences were allowed to vary in the flexible backbone design process, with the set of possible residues selected in the automated fashion describe above.

### Negative Design of Surface Residues

We used a simple Monte Carlo protocol to bias the desired folded topology, by searching for charged surface residues that stabilize the desired topology and destabilize the reverse topology (52). The protocol results in a surface pattern of negatively and positively charged residues. We modified the Rosetta residue file to account for this surface patterning by disallowing the opposite charge at positions specified by the surface pattern (The residues were still allowed to be neutral and polar). We find that this protocol results in bundles that exhibit well-defined ab initio folding funnels with single minima (e.g. FIG. S20A). Without this negative design element, folding funnels often show multiple minima representing different folded topologies. Scripts for surface patterning can be found in topology.py within the COMBS software package.

### Model Selection

We selected final, single-chain designs (among 500 total outputted models for each of the 6 designs) by considering the packing of the core residues (pstat score in Rosetta) and the overall energy (ref2015 weights). We used the convex hull algorithm mentioned above and a custom python script based on the program Probe to detect any buried residues with polar atoms not engaged in an H-bond, such as Tyr or Trp residues. We selected designs that did not feature any “unsatisfied” H-bonding residues. Computational models of the designs are freely available at the online repository zenodo (https:/doi.org/10.5281/zenodo.3718920).

### Ab Initio Folding

Rosetta ab initio folding (53) was performed on the final designed sequences. The command line input for folding simulations can be found in the supplementary text. RMSD was calculated to all Cα atoms of the input model. Of the 6 designed sequences, only 2 were predicted to fold to a structure that maintained an open, solvent-accessible binding site, ABLE and LABLE (FIGS. 520A-520C). Three of the other designs showed a collapsed hydrophobic binding site with no space for binding, and one design was predicted not to fold. We expressed and characterized all 6 of them. Interestingly, the two designs with a predicted open binding site tightly bound apixaban. The other designs did not bind apixaban, suggesting that ab initio folding predictions of binding-site collapse are a good indicator for design success. All designs were helical as measured by circular dichroism spectroscopy (FIG. S9).

### Code Availability

The code for COMBS is available at github (https://github.com/npolizzi/combs_pub). The scripts for flexible-backbone sequence design in Rosetta can be found in supplementary text.

### Protein Expression

The genes coding for the 6 protein sequences were ordered from GenScript, and were cloned into the IPTG-inducible pet-11a plasmid (cloning site NdeI-BamHI). The sequence of each design also coded for an N-terminal 6×His-tag followed by a TEV protease cleavage sequence.

### Cloned Gene Sequence of ABLE

ATGCACCACCACCACCACCACGAAAACCTGTACTTCCAGAGCGTGAAGA
GCGAGTATGCGGAAGCTGCGGCGGTTGGTCAAGAAGCGGTGGCGGTTTT
CAACACCATGAAGGCGGCGTTTCAGAACGGCGATAAAGAGGCGGTTGCG
CAATACCTGGCGCGTCTGGCGAGCCTGTATACCCGTCACGAGGAACTGC
TGAACCGTATCCTGGAAAAGGCGCGTCGTGAGGGTAACAAAGAAGCGGT
GACCCTGATGAACGAGTTCACCGCGACCTTTCAGACCGGCAAGAGCATT
TTCAACGCGATGGTTGCGGCGTTTAAAAACGGCGACGATGACAGCTTTG
AGAGCTACCTGCAGGCGCTGGAAAAGGTGACCGCGAAAGGCGAGACCCT
GGCGGACCAAATCGCGAAAGCGCTGTAA

### Expressed Protein Sequence of ABLE

MHHHHHHENLYFQ/SVKSEYAEAAAVGQEAVAVFNTMKAAFQNGDKEAV
AQYLARLASLYTRHEELLNRILEKARREGNKEAVTLMNEFTATFQTGKS
IFNAMVAAFKNGDDDSFESYLQALEKVTAKGETLADQIAKAL

where the “/” defines the cleavage site of TEV protease. TEV-cleaved ABLE is 126 residues. The plasmids were transfected into E. coli BL21(DE3) cells (Invitrogen), which were grown in LB/ampicillin media until OD @ 600 nm=0.6. The cells were then induced with IPTG and allowed to grow for 4 more hours. Cells were then centrifuged and frozen. The frozen cell pellets were thawed and lysed by sonication, purified by Ni NTA affinity column (Invitrogen), and purified protein was confirmed by gel electrophoresis. The buffer was exchanged to a TEV protease buffer (5 mM DTT, 50 mM Tris, 0.5 mM EDTA, pH 8.0), and proteins were incubated with His-tagged TEV protease for 1 day at room temperature. The cleaved protein was collected from the flow-through of a Ni NTA column and concentrated in a stock of 50 mM NaPi, 100 mM NaCl, pH 7.4 buffer. Both TEV-cleaved and His-tagged proteins were used in experiments, as they showed no significant differences in binding. ABLE had an approximate yield of 200 mg/L.

### Expressed Protein Sequence of LABLE

MHHHHHHENLYFQ/SSEEDQLDKLLKEFKAVFNHGKKVFEQMKQAWERM
ASAFKNNQNASELLDELAKYISELNEVTKHGQELAKKIRDAAERANASD
EWRKTFDEAAKVGQAFIKTWEAFVRTWEAFEKAYKNGDDEKNLKAYLEQ
LKKYLEQLESYLRQHDELLQKLEELWKKIKS

### Synthesis of Apx-Peg-FITC

Apixaban-peg-FITC was synthesized from apixaban acid (ApxCOO⁻) by coupling with Boc-(PEG)₂-amine followed by deprotection and reaction with FITC (Scheme S1). To a solution of apixaban acid (200 mg, 0.43 mmol) in DMF (2 mL) were added Boc-(PEG)₂-amine (108 mg, 0.43 mmol), DIPEA (174 uL, 1 mmol) and HCTU (169 mg, 041 mmol). The mixture was stirred at room temperature for 3 h and diluted with ethyl acetate. The organic layer was successively washed with 1 M HCl, sat. NaHCO₃, and brine. After drying over Na₂SO₄, the mixture was concentrated under reduced pressure and a solution of TFA in DCM (50%, 5 mL) was added. The mixture was stirred for 1 h and the volatiles were removed under reduced pressure. To the solution of crude amine in DMF (4 mL) were added DIPEA (134 uL) and FITC (136 mg). After stirring 2 h, the mixture was diluted with ethyl acetate and washed with sat. NaHCO₃. The organic layer was concentrated and purified by RP-HPLC. ¹H NMR (DMSO-d6, 300 MHz) 8.30 (1H, br s), 7.73 (1H, d, J=8.0 Hz), 7.49 (2H, d, J=8.9 Hz, 1H), 7.33 (2H, d, J=8.8 Hz), 7.26 (2H, d, J=8.8 Hz), 7.16 (1H, d, J=8.3 Hz), 6.98 (2H, d, J=9 Hz), 6.67 (2H, s), 6.56 (m, 4H), 4.04 (m, 2H), 3.79 (s, 3H), 3.5-3.53 (m, 12H), 3.42-3.35 (m, 2H), 3.25-3.15 (m, 2H), 2.38 (2H, br s), 1.85-1.83 (4H, m) ESI-MS (MR⁺) 980.5

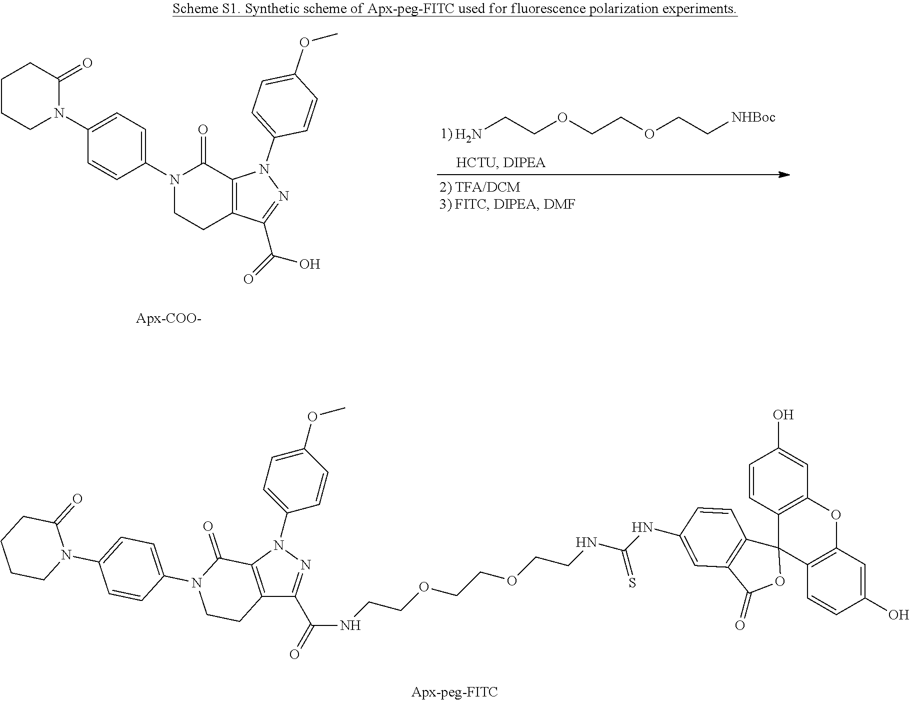 [SMILES: COc1ccc(-n2nc(C(=O)O)c3c2C(=O)N(c2ccc(N4CCCCC4=O)cc2)CC3)cc1 ; CC(C)(C)OC(=O)NCCOCCOCCN ; COc1ccc(-n2nc(C(=O)NCCOCCOCCNC(=S)Nc3ccc4c(c3)C(=O)OC43c4ccc(O)cc4Oc4cc(O)ccc43)c3c2C(=O)N(c2ccc(N4CCCCC4=O)cc2)CC3)cc1]

### Determination of Binding Dissociation Constant

We used spectral titration and fluorescence polarization experiments to determine the binding dissociation constants for ABLE. ABLE was purified via HPLC (C4 reverse phase column), lyophilized, and reconstituted in buffer (50 mM NaPi, 100 mM NaCl, pH 7.4). Aliquots of apixaban from 2 mM, 1 mM, or 0.5 mM stocks in DMSO were serially added to 2 mL solutions of ABLE at 20 μM, 10 μM, and 5 μM concentration, respectively. (Final DMSO concentration was kept below 2%.) Absorbance changes at 305 nm, due to the restricted torsional conformation of apixaban in the bound state, were fit to Equation 1 using a single-site, protein-ligand binding model for the [Apx·ABLE] complex (Equation 2) (FIG. 3G, FIG. 3H and FIGS. 511A-511D). Global parameters of the fit were Δε₃₀₅ [symbol] K_{D}, and N, where Δε₃₀₅ [symbol] s the change in extinction coefficient at 305 nm of the bound complex relative to free apixaban and protein. We used ε₃₀₅ [symbol] the extinction coefficient of apixaban at 305 nm, as a local fitting parameter for each concentration, and the results of these locally fit extinction coefficients were within experimental error of each other (also agreeing with that measured for apixaban alone). Results of the fit are listed in FIGS. 511A-511D (legend). The best-fit value of N was 1.4 (stoichiometry of 0.7 ligand to 1 protein); deviation from unity was either due to experimental errors in concentrations or a small population of the protein that has less affinity toward apixaban. Multiple different starting parameters converged onto those listed in FIGS. 511A-511D. Individual fitting at different concentrations gave good fits, but a limited degree of covariation between N and K_{D}. This was eliminated by global fitting at multiple concentrations. Randomness of the residuals confirmed goodness of fit. While ABLE contains no Trp residues, the presence of several Trp residues in LABLE that spectrally overlap with apixaban precluded the spectral titration experiment with LABLE.

We performed fluorescence anisotropy experiments (54, 55) of ABLE and LABLE using a FITC fluorophore conjugated to apixaban as the fluorescent probe (FIGS. 512A-512D and Scheme 1). We serially diluted a concentrated protein solution containing 25 nM Apx-peg-FITC, holding constant the concentration of Apx-peg-FITC. Parallel and perpendicularly polarized emission at 528 nm (10 nm slit width, 510 nm long pass filter) were integrated for 10 seconds after 485 nm (5 nm slit width) excitation of the FITC fluorophore of Apx-peg-FITC. The data were fit to a single-site binding model (Equation 2, with N=1) (FIG. S12B). We performed ligand competition experiments (54, 55) by adding aliquots of a concentrated ligand stock in DMSO into an approximately 50% bound complex of protein and Apx-peg-FITC (25 nM). The decrease in anisotropy as a function of competing ligand was fit to a competitive binding model (54, 55) (FIG. 4D and FIG. S12C and FIG. S12D). Because effects of DMSO on the anisotropy of bound ABLE/Apx-peg-FITC complex become more pronounced at high concentrations of DMSO (>4%), we fit only the initial data points at low DMSO concentration (% DMSO <2%, FIG. 4D). Comparison of a fit to the full titration with apixaban as competitor showed similar results (FIG. S12C), indicating that DMSO does not significantly affect the K_{D} of apixaban to ABLE. DMSO may have a minor effect on competitive binding with weaker competitors (i.e., apixabanCOO⁻ and rivaroxaban), where high concentrations of competitor are needed to decrease anisotropy. As such, the reported dissociation constants of these weaker competitors can be viewed as a lower limit to the K_{D}. Fluorescence polarization competition with apixaban as competitor gives a K_{D} of 7 (±1) μM for the apixaban/ABLE complex (FIG. 4D), which agrees with that of the more precise spectral titration [K_{D}=5 (±1) μM]. Binding experiments were performed at least twice to confirm reproducibility, and the reported errors correspond to the uncertainty in the fit parameters.

$${OD}_{305} ( {[ A p x ]}_{T} ) = {OD}_{305} ( {[ APX ]}_{T} = 0 ) + {{ε}_{305} [ A p x ]}_{T} + Δ {ε}_{305} [ Apx \cdot ABLE ] ( {[ ABLE ]}_{T} , {[ Apx ]}_{T} , {K}_{D} , N ) \qquad Equation 1$$

$$[ Apx \cdot ABLE ] ( {[ ABLE ]}_{T} , {[ Apx ]}_{T} , {K}_{D} , N ) = \frac{1}{2} [ {K}_{D} + {[ A p x ]}_{T} + \frac{{[ ABLE ]}_{T}}{N} - \sqrt{{( {K}_{D} + {[ A p x ]}_{T} + \frac{{[ A B L E ]}_{T}}{N} )}^{2} - {4 [ A p x ]}_{T} \frac{{[ A B L E ]}_{T}}{N}} ] \qquad Equation 2$$

### Steady-State Electronic Absorption Spectroscopy

Electronic absorption spectra were collected using a HP 8453 spectrophotometer in 1 cm quartz optical cells. The noise level of the instrument was maintained at 0.1 mOD.

### Thermal Stability

CD spectra were collected on a Jasco J-810 CD spectrometer in a 0.1 cm path length quartz cuvette (FIG. S9). Full spectra were collected from 200 nm to 250 nm in continuous scanning mode, with a band width of 2 nm, scanning speed of 50 nm/min, data pitch of 2 nm, response of 8 sec (standard sensitivity), and an average of 3 accumulations. Designs were prepared in 6 or 12 μM concentrations in 50 mM NaPi pH 7.4, 100 mM NaCl buffer. Temperature-dependent data of liganded and unliganded ABLE (FIGS. S15A-S15B) were collected at 222 nm from 20 to 95° C. with an interval of 5° C. and an increase rate of 3° C./minute, and an average of 5 accumulations. ABLE was prepared at 10 μM in 50 mM NaPi pH 7.4, 100 mM NaCl buffer. Apixaban-bound ABLE solution contained 30 μM apixaban (0.27% final concentration of DMSO). To aid in direct comparison to the bound complex, the unliganded protein solution also contained 0.27% DMSO.

### Oligomerization State

We determined oligomerization state by size exclusion chromatography on an Akta Pure FPLC using a Superdex 75 5/150 analytical column. Both drug-free- and drug-bound ABLE eluted at elution volumes equivalent to its molecular weight (FIG. S14).

### X-Ray Crystallography

We screened crystallization conditions for unliganded- and liganded ABLE in 96-well hanging drop trays from Hampton Research. His-tag-cleaved ABLE was concentrated in water at 30 mg/mL. For preparation of drug-bound ABLE, we added 1.1 equivalents of apixaban from a concentrated DMSO stock, resulting in a DMSO concentration of 12%. Both drug-bound- and drug-free ABLE readily crystallized in multiple conditions from Hampton Peg Ion 2 screen and the ammonium sulfate (AmSO₄) screen. We looped the crystals and submerged them in paratone cryoprotectant before freezing them in liquid nitrogen. Diffraction data was collected remotely using an Eiger 16M detector at the 24-IDE (NE-CAT) beamline of the Advanced Photon Source at Fermi Lab. Multiple conditions gave high-quality diffraction with resolution below 2 Å. The well condition that gave the best diffraction for both drug-bound and drug-free ABLE was 2.6 M AmSO₄, 0.1 M Na acetate. Crystals of both proteins diffracted to 1.3 Å resolution in this condition. Reflections were processed and merged using RAPD (https://rapd.nec.aps.anl.gov/). The structures were solved by molecular replacement with Phaser in Phenix, using the design model with apixaban removed. The structures were iteratively refined in Phenix and Coot. Diffraction data and refinement statistics of apixaban-bound- and drug-free ABLE are shown in Table S3. Crystals of the H49A mutant of ABLE were grown in a 24-well hanging drop plate with well solution 0.03 M Citric acid, 0.07 M BIS-TRIS propane/pH 7.6 with 20% w/v Polyethylene glycol 3,350 (Hampton PEG/Ion 2 screen condition 40). Crystals were looped in paratone and frozen in liquid nitrogen, and diffraction data to 1.6 Å resolution was collected on a PILATUS3 6M detector at the 8.3.1 beamline at the Advanced Light Source and Lawrence Berkeley National Labs. Reflections were processed and merged via XDS program and the structure was solved by molecular replacement with Phaser in Phenix, using the drug-free ABLE protein structure as the search model. The structure was iteratively refined in Phenix and Coot. Diffraction data and refinement statistics of unliganded H49A ABLE are shown in Table S4.

### Command Lines and Flags for Flexible Backbone Design Algorithm

~/rosetta_bin_linux_2018.33.60351_bundle/main/source/bin/rosetta_scripts.stat
ic.linuxgccrelease -database
~/rosetta_bin_linux_2018.33.60351_bundle/main/database/ -s input.pdb -nstruct
500 -extra_res_fa APX.params -parser:protocol flexbb_design_protocol.xml -
packing:multi_cool_annealer 10 -packing:linmem_ig 10

### RosettaScript for Flexible Backbone Design (Flexbb_Design_Protocol.Xml)

```
<ROSETTASCRIPTS>
  <SCOREFXNS>
    <ScoreFunction name=″ref15″ weights=″ref2015″>
      <Reweight scoretype=″atom_pair_constraint″ weight=″1″/>
    </ScoreFunction>
    <ScoreFunction name=″ref15_1″ weights=″ref2015″>
      <Reweight scoretype=″aa_composition″ weight=″1″ />
      <Reweight scoretype=″netcharge″ weight=″1.0″ />
      <Reweight scoretype=″atom_pair_constraint″ weight=″1″/>
      <Set aa_composition_setup_file=″no_met_thr_ser_asn.comp″ />
      <Set netcharge_setup_file=″netcharge.charge″ />
    </ScoreFunction>
  </SCOREFXNS>
  <RESIDUE_SELECTORS>
  </RESIDUE_SELECTORS>
  <TASKOPERATIONS>
    <InitializeFromCommandline name=″ifcl″/>
    <ReadResfile name=″resfile″ filename=″resfile.txt″/>
    <ExtraRotamersGeneric name=″extrachi″ ex1=″1″ ex2=″1″
              ex1_sample_level=″1″ ex2_sample_level=″1″
              extrachi_cutoff=″14″/>
    <IncludeCurrent name=″include_curr″ />
  </TASKOPERATIONS>
  <FILTERS>
    <PackStat name=″pstat″ confidence=″0″ threshold=″0″ repeats=″10″/>
    <PackStat name=″pstat_mc″ threshold=″0″ repeats=″10″/>
    <ScoreType name=″total_score_1″ scorefxn=″ref15_1″ score_type=″total_score″
        threshold=″0″/>
    </FILTERS>
  <MOVERS>
    <ConstraintSetMover name=″atomic″ cst_file=″vdM_Hbonds.cst″/>
    <PackRotamersMover name=″pack″ scorefxn=″ref15_1″
            task_operations=″ifcl,resfile,include_curr,extrachi″/>
    <PackRotamersMover name=″pack_fast″ scorefxn=″ref15_1″
            task_operations=″ifcl, resfile,include_curr″/>
    <MinMover name=″min_bb″ scorefxn=″ref15″ tolerance=″0.0000001″ max_iter=″1000″
chi=″false″ bb=″true″>
      <MoveMap name=″map_bb″>
        <Span begin=″1″ end=″125″ bb=″true″ chi=″false″ />
        <Span begin=″126″ end=″999″ bb=″false″ chi=″false″/>
      </MoveMap>
    </MinMover>
    <Idealize name=″idealize″/>
    <MinMover name=″min_sc″ scorefxn=″ref15″ tolerance=″0.0000001″ max_iter=″1000″
chi=″true″ bb=″false″>
      <MoveMap name=″map_sc″>
        <Span begin=″1″ end=″125″ bb=″false″ chi=″true″ />
        <Span begin=″126″ end=″999″ bb=″false″ chi=″false″/>
      </MoveMap>
    </MinMover>
    <MinMover name=″min_sc_bb″ scorefxn=″ref15″ tolerance=″0.0000001″ max_iter=″1000″
chi=″true″ bb=″true″>
      <MoveMap name=″map_sc_bb″>
        <Span begin=″1″ end=″125″ bb=″true″ chi=″true″ />
        <Span begin=″126″ end=″999″ bb=″false″ chi=″false″/>
      </MoveMap>
    </MinMover>
    <ParsedProtocol name=″parsed_pack_fast″ >
      <Add mover_name=″pack_fast″/>
      <Add mover_name=″min_bb″/>
    </ParsedProtocol>
    <ParsedProtocol name=″parsed_pack″ >
      <Add mover_name=″pack″/>
      <Add mover_name=″min_bb″/>
      <Add mover_name=″min_sc″/>
    </ParsedProtocol>
    <GenericMonteCarlo name=″pack_mc″ preapply=″0″ trials=″3″ temperature=″0.03″
              filter_name=″pstat_mc″ sample_type=″high″ mover_name=″parsed_pack″>
        <Filters>
          <AND filter_name=″total_score_1″ temperature=″15″ sample_type=″low″/>
        </Filters>
    </GenericMonteCarlo>
    <GenericMonteCarlo name=″pack_fast_mc″ preapply=″0″ trials=″2″ temperature=″0.03″
              filter_name=″pstat_mc″ sample_type=″high″ mover_name=″parsed_pack_fast″>
        <Filters>
          <AND filter_name=″total_score_1″ temperature=″15″ sample_type=″low″/>
        </Filters>
    </GenericMonteCarlo>
  </MOVERS>
  <APPLY_TO_POSE>
    </APPLY_TO_POSE>
    <PROTOCOLS>
      <Add mover=″atomic″/>
      <Add mover_name=″parsed_pack_fast″/>
      <Add mover_name=″pack_fast_mc″/>
      <Add mover_name=″pack_mc″/>
      <Add mover_name=″min_sc_bb″/>
      <Add filter_name=″pstat″/>
    </PROTOCOLS>
    <OUTPUT scorefxn=″ref15_1″/>
  </ROSETTASCRIPTS>
```

Contents of Constraint File (vdM_Hbonds.cst) for ABLE

### AtomPair NE2 48A O3 1X HARMONIC 2.7 0.3

### AtomPair OG1 111A O2 1X HARMONIC 2.8 0.3

### AtomPair HG1 111A O2 1X HARMONIC 2.0 0.3

### AtomPair HE2 48A O3 1X HARMONIC 1.7 0.3

Contents of Apixaban parameters file (APX.params) for use in Rosetta

NAME APX
IO_STRING APX Z
TYPE LIGAND
AA UNK
ATOM C8 CNH2 X 0.27
ATOM O3 ONH2 X −0.27
ATOM N5 Npro X −0.27
ATOM C7 aroC X 0.04
ATOM C22 aroC X −0.04
ATOM C18 aroC X −0.04
ATOM C16 aroC X 0.04
ATOM N2 Npro X −0.27
ATOM C19 CNH2 X 0.22
ATOM O2 ONH2 X −0.28
ATOM C23 CH2 X 0.02
ATOM C25 CH2 X −0.04
ATOM C21 CH2 X −0.04
ATOM C20 CH2 X 0.02
ATOM H14 Hapo X 0.05
ATOM H15 Hapo X 0.05
ATOM H16 Hapo X 0.03
ATOM H17 Hapo X 0.03
ATOM H23 Hapo X 0.03
ATOM H24 Hapo X 0.03
ATOM H19 Hapo X 0.04
ATOM H20 Hapo X 0.04
ATOM C14 aroC X −0.04
ATOM C44 aroC X −0.04
ATOM H25 Haro X 0.06
ATOM H7 Haro X 0.06
ATOM H13 Haro X 0.06
ATOM H18 Haro X 0.06
ATOM C24 CH2 X 0.02
ATOM C17 CH2 X −0.01
ATOM C12 aroC X 0.02
ATOM C10 aroC X 0.15
ATOM N6 Nhis X −0.16
ATOM N1 Npro X −0.23
ATOM C4 aroC X 0.07
ATOM C3 aroC X −0.03
ATOM C2 aroC X −0.02
ATOM C1 aroC X 0.12
ATOM O4 OH X −0.50
ATOM C15 CH3 X 0.08
ATOM H8 Hapo X 0.07
ATOM H9 Hapo X 0.07
ATOM H10 Hapo X 0.07
ATOM C6 aroC X −0.02
ATOM C5 aroC X −0.03
ATOM H5 Haro X 0.06
ATOM H6 Haro X 0.07
ATOM H1 Haro X 0.07
ATOM H2 Haro X 0.06
ATOM C13 aroC X 0.13
ATOM C11 CNH2 X 0.26
ATOM O1 ONH2 X −0.27
ATOM N3 NH2O X −0.32
ATOM H3 Hpol X 0.15
ATOM H4 Hpol X 0.15
ATOM H11 Hapo X 0.03
ATOM H12 Hapo X 0.03
ATOM H21 Hapo X 0.05
ATOM H22 Hapo X 0.05
BOND_TYPE C1 C2 4
BOND_TYPE C1 O4 1
BOND_TYPE C1 C6 4
BOND_TYPE N1 C4 1
BOND_TYPE N1 N6 4
BOND_TYPE N1 C13 4
BOND_TYPE O1 C11 2
BOND_TYPE C2 C3 4
BOND_TYPE N2 C16 1
BOND_TYPE N2 C19 4
BOND_TYPE N2 C20 1
BOND_TYPE O2 C19 2
BOND_TYPE C3 C4 4
BOND_TYPE N3 C11 4
BOND_TYPE O3 C8 2
BOND_TYPE C4 C5 4
BOND_TYPE O4 C15 1
BOND_TYPE C5 C6 4
BOND_TYPE N5 C7 1
BOND_TYPE N5 C8 4
BOND_TYPE N5 C24 1
BOND_TYPE N6 C10 4
BOND_TYPE C7 C22 4
BOND_TYPE C7 C44 4
BOND_TYPE C8 C13 1
BOND_TYPE C10 C11 1
BOND_TYPE C10 C12 4
BOND_TYPE C12 C13 4
BOND_TYPE C12 C17 1
BOND_TYPE C14 C16 4
BOND_TYPE C14 C44 4
BOND_TYPE C16 C18 4
BOND_TYPE C17 C24 1
BOND_TYPE C18 C22 4
BOND_TYPE C19 C23 1
BOND_TYPE C20 C21 1
BOND_TYPE C21 C25 1
BOND_TYPE C23 C25 1
BOND_TYPE C2 H1 1
BOND_TYPE C3 H2 1
BOND_TYPE N3 H3 1
BOND_TYPE N3 H4 1
BOND_TYPE C5 H5 1
BOND_TYPE C6 H6 1
BOND_TYPE C14 H7 1
BOND_TYPE C15 H8 1
BOND_TYPE C15 H9 1
BOND_TYPE C15 H10 1
BOND_TYPE C17 H11 1
BOND_TYPE C17 H12 1
BOND_TYPE C18 H13 1
BOND_TYPE C20 H14 1
BOND_TYPE C20 H15 1
BOND_TYPE C21 H16 1
BOND_TYPE C21 H17 1
BOND_TYPE C22 H18 1
BOND_TYPE C23 H19 1
BOND_TYPE C23 H20 1
BOND_TYPE C24 H21 1
BOND_TYPE C24 H22 1
BOND_TYPE C25 H23 1
BOND_TYPE C25 H24 1
BOND_TYPE C44 H25 1
CHI 1 C2 C1 O4 C15
CHI 2 N6 N1 C4 C3
CHI 3 C18 C16 N2 C19
CHI 4 C8 N5 C7 C22
CHI 5 C12 C10 C11 O1
NBR_ATOM C8
NBR_RADIUS 10.865233
ICOOR_INTERNAL C8 0.000000 0.000000 0.000000 C8 O3 N5
ICOOR_INTERNAL O3 0.000000 180.000000 1.231236 C8 O3 N5
ICOOR_INTERNAL N5 0.000000 56.912601 1.396232 C8 O3 N5
ICOOR_INTERNAL C7 1.861545 58.604695 1.397270 N5 C8 O3
ICOOR_INTERNAL C22 116.605296 62.137596 1.469823 C7 N5 C8
ICOOR_INTERNAL C18 −175.989024 61.379327 1.445469 C22 C7 N5
ICOOR_INTERNAL C16 0.813034 61.902299 1.464716 C18 C22 C7
ICOOR_INTERNAL N2 179.326815 62.708149 1.369606 C16 C18 C22
ICOOR_INTERNAL C19 84.211713 61.381606 1.362129 N2 C16 C18
ICOOR_INTERNAL O2 −0.750882 57.093251 1.230451 C19 N2 C16
ICOOR_INTERNAL C23 −178.336703 61.046447 1.518330 C19 N2 O2
ICOOR_INTERNAL C25 −22.826129 66.916174 1.522564 C23 C19 N2
ICOOR_INTERNAL C21 54.381415 72.125433 1.513394 C25 C23 C19
ICOOR_INTERNAL C20 −65.929287 72.585866 1.518530 C21 C25 C23
ICOOR_INTERNAL H14 168.321198 71.890144 1.070033 C20 C21 C25
ICOOR_INTERNAL H15 121.939979 73.687057 1.069996 C20 C21 H14
ICOOR_INTERNAL H16 119.682070 70.015091 1.070011 C21 C25 C20
ICOOR_INTERNAL H17 119.410071 69.333109 1.070013 C21 C25 H16
ICOOR_INTERNAL H23 119.750803 70.132011 1.070003 C25 C23 C21
ICOOR_INTERNAL H24 119.538025 69.598481 1.069963 C25 C23 H23
ICOOR_INTERNAL H19 −120.537286 71.438077 1.070005 C23 C19
C25
ICOOR_INTERNAL H20 −121.238076 72.646354 1.069999 C23 C19
H19
ICOOR_INTERNAL C14 179.366426 56.231249 1.466344 C16 C18 N2
ICOOR_INTERNAL C44 0.087443 61.996128 1.445609 C14 C16 C18
ICOOR_INTERNAL H25 −178.446203 59.324002 1.032000 C44 C14
C16
ICOOR_INTERNAL H7 179.999779 59.004934 1.032021 C14 C16 C44
ICOOR_INTERNAL H13 179.998713 59.051198 1.032029 C18 C22 C16
ICOOR_INTERNAL H18 179.997900 59.310494 1.032007 C22 C7 C18
ICOOR_INTERNAL C24 173.707419 60.978627 1.486888 N5 C8 C7
ICOOR_INTERNAL C17 37.416194 65.870980 1.527253 C24 N5 C8
ICOOR_INTERNAL C12 −47.932429 73.211144 1.502706 C17 C24 N5
ICOOR_INTERNAL C10 −148.407600 51.860214 1.395453 C12 C17
C24
ICOOR_INTERNAL N6 −179.979754 74.754024 1.343428 C10 C12 C17
ICOOR_INTERNAL N1 −0.278628 69.048228 1.377285 N6 C10 C12
ICOOR_INTERNAL C4 −175.554672 57.266493 1.413742 N1 N6 C10
ICOOR_INTERNAL C3 −90.725691 59.397750 1.475217 C4 N1 N6
ICOOR_INTERNAL C2 −179.340753 60.676707 1.470124 C3 C4 N1
ICOOR_INTERNAL C1 0.236073 60.608076 1.467605 C2 C3 C4
ICOOR_INTERNAL O4 178.772123 64.434523 1.399458 C1 C2 C3
ICOOR_INTERNAL C15 −176.870679 55.844358 1.427541 O4 C1 C2
ICOOR_INTERNAL H8 179.998785 70.530029 1.070023 C15 O4 C1
ICOOR_INTERNAL H9 −119.996609 70.529409 1.070005 C15 O4 H8
ICOOR_INTERNAL H10 −120.001345 70.528187 1.070054 C15 O4 H9
ICOOR_INTERNAL C6 −179.437977 58.893416 1.473075 C1 C2 O4
ICOOR_INTERNAL C5 0.527365 60.480609 1.459726 C6 C1 C2
ICOOR_INTERNAL H5 −179.954255 59.848491 1.031988 C5 C6 C1
ICOOR_INTERNAL H6 179.997958 59.760967 1.032020 C6 C1 C5
ICOOR_INTERNAL H1 179.999202 59.695644 1.032059 C2 C3 Cl
ICOOR_INTERNAL H2 −179.999632 59.662272 1.032050 C3 C4 C2
ICOOR_INTERNAL C13 176.060856 72.216496 1.373705 N1 N6 C4
ICOOR_INTERNAL C11 −176.364992 51.621280 1.403966 C10 C12 N6
ICOOR_INTERNAL O1 −23.545652 57.511727 1.233689 C11 C10 C12
ICOOR_INTERNAL N3 178.564758 65.559612 1.349119 C11 C10 O1
ICOOR_INTERNAL H3 179.996754 59.997725 0.984485 N3 C11 C10
ICOOR_INTERNAL H4 −179.993947 60.001315 0.984475 N3 C11 H3
ICOOR_INTERNAL H11 119.578039 69.861239 1.069967 C17 C24 C12
ICOOR_INTERNAL H12 119.249436 68.969508 1.070017 C17 C24 H11
ICOOR_INTERNAL H21 −120.690135 71.708371 1.069980 C24 N5 C17
ICOOR_INTERNAL H22 −121.644804 73.257780 1.069992 C24 N5 H21

### Contents of Netcharge.Charge

### DESIRED_CHARGE −5

### PENALTIES_CHARGE_RANGE −10 −1

### PENALTIES 10 0 0 0 0 0 0 0 0 10

### BEFORE_FUNCTION_QUADRATIC

### AFTER_FUNCTION_QUADRATIC

### Contents of No_Met_Thr_Ser_Asn.Comp

### PENALTY_DEFINITION

### TYPE THR

### DELTA_START 0

### DELTA_END 1

### PENALTIES 0 100

### ABSOLUTE 8

### BEFORE_FUNCTION CONSTANT

### AFTER_FUNCTION QUADRATIC

### END_PENALTY_DEFINITION

### PENALTY_DEFINITION

### TYPE SER

### DELTA_START 0

### DELTA_END 1

### PENALTIES 0 100

### ABSOLUTE 8

### BEFORE_FUNCTION CONSTANT

### AFTER_FUNCTION QUADRATIC

### END_PENALTY_DEFINITION

### PENALTY_DEFINITION

### TYPE MET

### DELTA_START 0

### DELTA_END 1

### PENALTIES 0 100

### ABSOLUTE 3

### BEFORE_FUNCTION CONSTANT

### AFTER_FUNCTION QUADRATIC

### END_PENALTY_DEFINITION

### PENALTY_DEFINITION

### TYPE ASN

### DELTA START 0

### DELTA END 1

### PENALTIES 0 100

### ABSOLUTE 12

### BEFORE_FUNCTION CONSTANT

### AFTER_FUNCTION_QUADRATIC

### END_PENALTY_DEFINITION

### Command Line for Ab Initio Folding of Designed Sequences in Rosetta

~/rosetta_bin_linux_2018.33.60351_bundle/main/source/bin/AbinitioRelax.static
.linuxgccrelease -database
~/rosetta_bin_linux_2018.33.60351_bundle/main/database/ -in:file:frag3
aat000_03_05.200_v1_3 -in:file:frag9 aat000_09_05.200_v1_3 -abinitio:relax -
relax:fast -abinitio::increase_cycles 10 -abinitio::rg_reweight 0.5 -
abinitio::rsd_wt_helix 0.5 -abinitio::rsd_wt_loop 0.5 -use_filters true -
psipred_ss2 t000_.psipred_ss2 -kill_hairpins t000_.psipred_ss2 -
out:file:silent silent.out -nstruct 20000 -in:file:native able_design.pdb

### Contents of Residue File for ABLE Flexible-Backbone Sequence Design

start
1 X NATRO
13 A NATRO
48 A NATRO
111 A NATRO
1 A PIKAA ISQYARMDFEKWGPAVNLVH
2 A PIKAA KSQPAVNRHT
3 A PIKAA SQAVPNHT
4 A PIKAA ESQAVPNDHT
5 A PIKAA IGMWSAVYLAVHFT
6 A PIKAA SQAVPNHT
7 A PIKAA ESQAVPNDHT
8 A PIKAA IGMWSAVYLAVHFT
9 A PIKAA KSQPAVNRHT
10 A PIKAA SQAVPNHT
11 A PIKAA ISQYRMDFEKWAVLNVHT
12 A PIKAA IGMWSAVYLAVHFT
14 A PIKAA ESQAVPNDHT
15 A PIKAA IGMWSAVYLAVHFT
16 A PIKAA KSQPAVNRHT
17 A PIKAA SQAVPNHT
18 A PIKAA ISQYRMDFEKWAVLNVHT
19 A PIKAA IGMWSAVYLAVHFT
20 A PIKAA SQAVPNHT
21 A PIKAA ESQAVPNDHT
22 A PIKAA IGMWSAVYLAVHFT
23 A PIKAA KSQPAVNRHT
24 A PIKAA SQAVPNHT
25 A PIKAA SQAVPNHT
26 A PIKAA IGMWSAVYLAVHFT
27 A PIKAA SQAVPNHT
28 A PIKAA ESQAVPNDHT
29 A PIKAA KEGSROPAVNDAT
30 A PIKAA EKGSRQPAVNDHT
31 A PIKAA EKRSQPAVNDHT
32 A PIKAA EKRSQPAVNDHT
33 A PIKAA EKRSQPAVNDHT
34 A PIKAA IGMWSAVYLAVHFT
35 A PIKAA ESQAVPNDHT
36 A PIKAA SQAVPNHT
37 A PIKAA IGWSVAVYLAMHFT
38 A PIKAA IGWSVAVYLAMHFT
39 A PIKAA SQAVPNHT
40 A PIKAA KSQPAVNRHT
41 A PIKAA IGWSVAVYLAMHFT
42 A PIKAA ESQAVPNDHT
43 A PIKAA SQAVPNHT
44 A PIKAA IGWSVAVYLAMHFT
45 A PIKAA IGWSVAVYLAMHFT
46 A PIKAA SQAVPNHT
47 A PIKAA KSQPAVNRHT
49 A PIKAA ESQAVPNDHT
50 A PIKAA ESQAVPNDHT
51 A PIKAA ISQYRMFKWAVLNVHT
52 A PIKAA IGWSVAVYLAMHFT
53 A PIKAA SQAVPNHT
54 A PIKAA KSQPAVNRHT
55 A PIKAA IGWSVAVYLAMHFT
56 A PIKAA IWSVQAVYLNMHFT
57 A PIKAA ESQAVPNDHT
58 A PIKAA EKRSQPAVNDHT
59 A PIKAA IGMWSAVYLAVHFT
60 A PIKAA EKRSQPAVNDHT
61 A PIKAA EKRSQPAVNDHT
62 A PIKAA EKRSQPAVNDHT
63 A PIKAA KEGSROPAVNDAT
64 A PIKAA ISQYARMDFEKWGPAVNLVH
65 A PIKAA KSQPAVNRHT
66 A PIKAA ESQAVPNDHT
67 A PIKAA IGMWSAVYLAVHFT
68 A PIKAA SQAVPNHT
69 A PIKAA ESQAVPNDHT
70 A PIKAA IGMWSAVYLAVHFT
71 A PIKAA IGMWSAVYLAVHFT
72 A PIKAA KSQPAVNRHT
73 A PIKAA ESQAVPNDHT
74 A PIKAA IGMWSAVYLAVHFT
75 A PIKAA KSQPAVNRHT
76 A PIKAA SQAVPNHT
77 A PIKAA IGMWSAVYLAVHFT
78 A PIKAA IGMWSAVYLAVHFT
79 A PIKAA SQAVPNHT
80 A PIKAA ESQAVPNDHT
81 A PIKAA IGMWSAVYLAVHFT
82 A PIKAA KSQPAVNRHT
83 A PIKAA SQAVPNHT
84 A PIKAA ISQYRMDFEKWAVLNVHT
85 A PIKAA IGMWSAVYLAVHFT
86 A PIKAA SQAVPNHT
87 A PIKAA ESQAVPNDHT
88 A PIKAA IGMWSAVYLAVHFT
89 A PIKAA KSQPAVNRHT
90 A PIKAA SQAVPNHT
91 A PIKAA SQAVPNHT
92 A PIKAA IGMWSAVYLAVHFT
93 A PIKAA KSQPAVNRHT
94 A PIKAA ESQAVPNDHT
95 A PIKAA KEGSROPAVNDAT
96 A PIKAA EKGSRQPAVNDHT
97 A PIKAA EKRSQPAVNDHT
98 A PIKAA EKRSQPAVNDHT
99 A PIKAA KSQPAVNRHT
100 A PIKAA IGWSVAVYLAMHFT
101 A PIKAA ESQAVPNDHT
102 A PIKAA SQAVPNHT
103 A PIKAA IGWSVAVYLAMHFT
104 A PIKAA IGWSVAVYLAMHFT
105 A PIKAA SQAVPNHT
106 A PIKAA KSQPAVNRHT
107 A PIKAA IGWSVAVYLAMHFT
108 A PIKAA ESQAVPNDHT
109 A PIKAA KSQPAVNRHT
110 A PIKAA IGWSVAVYLAMHFT
112 A PIKAA SQAVPNHT
113 A PIKAA KSQPAVNRHT
114 A PIKAA IGWSVAVYLAMHFT
115 A PIKAA ESQAVPNDHT
116 A PIKAA KSQPAVNRHT
117 A PIKAA IGWSVAVYLAMHFT
118 A PIKAA IGWSVAVYLAMHFT
119 A PIKAA ESQAVPNDHT
120 A PIKAA SQAVPNHT
121 A PIKAA IGWSVAVYLAMHFT
122 A PIKAA EKRSQPAVNDHT
123 A PIKAA EKRSQPAVNDHT
124 A PIKAA EKRSQPAVNDHT
125 A PIKAA ISQYARMDFEKWGPAVNLVH

### VDM Database/Library

PDB accession codes and chain IDs of the proteins used to compile vdM databases. List of 4-character PDB accession codes followed by a one-letter chain ID. Protein chains searched for curation of van der Mers. For each PDB, the biological assembly was constructed and protein contacts with the labeled chain were assessed.

1a1xA; 1a2 pA; 1a92A; 1abaA; 1ae9A; 1at0A; 1atzA; 1b0bA; 1b5eA; 1b6aA; 1b93A; 1bgfA; 1bm8A; 1bquA; 1bu8A; 1bx7A; 1byiA; 1bz4A; 1c1dA; 1c4oA; 1c4qA; 1c5eA; 1c75A; 1c7cA; 1c7kA; 1cc8A; 1ceoA; 1cfbA; 1chdA; 1cmcA; 1cqmA; 1cruA; 1cs6A; 1cv8A; 1cxqA; 1czaN; 1d0dA; 1d2 nA; 1d2sA; 1d2tA; 1d2zA; 1d2zB; 1d4oA; 1d5tA; 1dcsA; 1dj0A; 1dk8A; 1dkiA; 1d15A; 1d1yA; 1dmgA; 1doiA; 1dowA; 1dowB; 1dqgA; 1dusA; 1dwkA; 1dypA; 1dzfA; 1e29A; 1e2wA; 1e58A; 1e71A; 1eajA; 1eaqA; 1earA; 1egpA; 1egpB; 1e16A; 1e1kA; 1e1uA; 1e1wA; 1es5A; 1euvA; 1euwA; 1ev1A; 1ez3A; 1ezgA; fNiA; 1fDIA; 1f1eA; 1f1 mA; 1f1uA; 1f2tA; 1f2tB; 1f39A; 1f46A; 1f5 nA; 1f7A; 1f9zA; 1fhgA; 1fm0E; 1fm0D; 1fo8A; 1fobA; 1fp2A; 1fpoA; 1fxdA; 1fxoA; 1fyeA; 1g1jA; 1g1tA; 1g2bA; 1g3kA; 1g3 pA; 1g5aA; 1g60A; 1g61A; 1g66A; 1g6gA; 1g6uA; 1g8aA; 1ga6A; 1ga8A; 1gciA; 1gcqC; 1g12A; 1g12C; 1g14A; 1g14B; 1gn1A; 1gnyA; 1go3E; 1go3F; 1goiA; 1gp0A; 1gp6A; 1gppA; 1gpqA; 1gr3A; 1gsaA; 1gttA; 1guiA; 1gutA; 1gv2A; 1gv3A; 1gv9A; 1gvdA; 1gvfA; 1gvjA; 1gvnB; 1gvnA; 1gweA; 1gxmA; 1gxrA; 1gxyA; 1h0bA; 1h0hB; 1h12A; 1h16A; 1h2eA; 1h2sB; 1h2vZ; 1h4lA; 1h4aX; 1h6hA; 1h6kA; 1h6kX; 1h72C; 1h7eA; 1h80A; 1h8 pA; 1h97A; 1h98A; 1h99A; 1hdhA; 1hdoA; 1hh8A; 1hm1A; 1hq0A; 1hx6A; 1hxiA; 1hyoA; 1hztA; 1i0rA; 1i12A; 1i24A; 1i27A; 1i2hA; 1i2tA; 1i4uA; 1i71A; 1i7wB; 1i8aA; 1i9sA; 1iapA; 1igqA; 1ihgA; 1ihjA; 1iibA; 1ijbA; 1ikoP; 1ikpA; 1in4A; 1in1A; 1iomA; 1iq6A; 1iqzA; 1isuA; 1ituA; 1itxA; 1iu1A; 1iuqA; 1ix9A; 1ixhA; 1izcA; 1j0pA; 1j2jB; 1j2rA; 1j30A; 1j34C; 1j3aA; 1j3wA; 1j5uA; 1j5wA; 1j77A; 1j8uA; 1j97A; 1j98A; 1j9bA; 1jayA; 1jc1A; 1jd5A; 1jg1A; 1jhgA; 1jiwI; 1jjfA; 1jkeA; 1j10A; 1jm1A; 1jniA; 1jnrA; 1jnrB; 1jp4A; 1jpeA; 1jqeA; 1juhA; 1juvA; 1jvwA; 1jw9B; 1jx6A; 1jyhA; 1jztA; 1k2xB; 1k2xA; 1k3iA; 1k4 nA; 1k5cA; 1k5nB; 1k5 nA; 1k6dA; 1k77A; 1k7cA; 1ka1A; 1kb0A; 1kcqA; 1kdgA; 1keaA; 1kgdA; 1khiA; 1k19A; 1ki1A; 1k1xA; 1kmtA; 1knmA; 1koeA; 1kp6A; 1kq3A; 1kqfA; 1kqfB; 1kqfC; 1kqpA; 1kr4A; 1kwfA; 1kwgA; 1kwmA; 1kzfA; 1kzqA; 1l2 pA; 1l3kA; 1l3 pA; 1l6rA; 1l91A; 1l9xA; 1lamA; 1lf7A; 1lj8A; 1lj9A; 1ljoA; 1l1fA; 1lm1A; 1lniA; 1lo7A; 1lqtA; 1lqvA; 1lqvC; 1lr0A; 1lr5A; 1lr7A; 1ls1A; 1luaA; 1lwbA; 1m0dA; 1m0uA; 1m0wA; 1m15A; 1mifA; 1m1qA; 1m22A; 1m2dA; 1m3uA; 1m65A; 1m6sA; 1m70A; 1m9zA; 1maiA; 1mbmA; 1mc2A; 1mgqA; 1mgtA; 1mhnA; 1mixA; 1mj4A; 1mj5A; 1mkkA; 1mkzA; 1mo9A; 1mofA; 1mpgA; 1mpxA; 1mrzA; 1mugA; 1munA; 1muwA; 1mv1A; 1mw9X; 1mwqA; 1mxrA; 1n08A; 1n0qA; 1n12A; 1n13B; 1n13A; 1n2 mA; 1n62A; 1n62B; 1n62C; 1n71A; 1n7hA; 1n7sD; 1n7sB; 1n7sC; 1n8vA; 1n93X; 1n9 pA; 1na3A; 1nb9A; 1nbaA; 1nc7A; 1ne2A; 1ne7A; 1nepA; 1nfpA; 1ng2A; 1ng6A; 1njhA; 1nkiA; 1nifA; 1nnfA; 1nnhA; 1nniA; 1nnwA; 1noxA; 1nr0A; 1nrjB; 1nrjA; 1ns5A; 1ntvA; 1ntyA; 1nwaA; 1nwwA; 1nwzA; 1nxcA; 1nxmA; 1nycA; 1nykA; 1nz0A; 1nziA; 1nzjA; 1o13A; 1o1yA; 1o22A; 1o2dA; 1o3uA; 1o4wA; 1o4yA; 1o50A; 1o54A; 1o5uA; 1o6aA; 1o75A; 1o7iA; 1o8xA; 1o91A; 1o94A; 1o9gA; 1o9iA; 1oaaA; 1obbA; 1oc7A; 1odmA; 1odvA; 1of8A; 1ofcX; 1of1A; 1ofwA; 1oh0A; 1oh4A; 1oi0A; 1oi2A; 1oi6A; 1oi7A; 1ojhA; 1ok0A; 1okiA; 1oksA; 1onwA; 1ooeA; 1opkA; 1oq1A; 1oqjA; 1oqvA; 1oqwA; 1or0B; 1or0A; 1oruA; 1otkA; 1ou8A; 1ov3A; 1ow1A; 1ow3A; 1ox3A; 1oygA; 1oywA; 1oz2A; 1oz9A; 1oznA; 1p0hA; 1p0zA; 1p1jA; 1p1mA; 1p3cA; 1p3dA; 1p57A; 1p5vB; 1p5zB; 1p6oA; 1p99A; 1p9aG; 1p9gA; 1pb7A; 1pbjA; 1pdoA; 1pe9A; 1pg6A; 1pgxA; 1pj5A; 1pmhX; 1pn0A; 1pn2A; 1pprM; 1ppyA; 1pqhA; 1pswA; 1ptqA; 1pu6A; 1pucA; 1puoA; 1pvgA; 1pwaA; 1pwbA; 1pz4A; 1pzsA; 1q08A; 1q0pA; 1q0rA; 1q35A; 1q40B; 1q42A; 1q4uA; 1q5yA; 1q5zA; 1q6hA; 1q6oA; 1q74A; 1q71A; 1q71B; 1q8bA; 1q8dA; 1q9uA; 1qb0A; 1qbaA; 1qbzA; 1qcsA; 1qcxA; 1qgeD; 1qgeE; 1qkrA; 1qksA; 1qnxA; 1qq5A; 1qreA; 1qs1A; 1qsaA; 1qupA; 1qusA; 1qwdA; 1qwgA; 1qwoA; 1qwrA; 1qwyA; 1qz9A; 1r0dA; 1r0r1; 1r0uA; 1r1tA; 1r29A; 1r45A; 1r4vA; 1r4xA; 1r5 mA; 1r62A; 1r6wA; 1r6xA; 1r77A; 1r7aA; 1r7jA; 1r89A; 1r9hA; 1ra0A; 1rc9A; 1rfeA; 1rfsA; 1rfyA; 1rg8A; 1rhfA; 1ri6A; 1rjdA; 1rk8A; 1rk8C; 1rkiA; 1rkuA; 1r1hA; 1r1iA; 1rm6B; 1rmgA; 1rp0A; 1rqbA; 1rtqA; 1rttA; 1ru4A; 1rutX; 1rw1A; 1rwhA; 1rwjA; 1rwrA; 1rxqA; 1ry9A; 1ryiA; 1ryqA; 1rz3A; 1sidA; 1s29A; 1s4kA; 1s5aA; 1s5dA; 1s7qA; 1s8 nA; 1s99A; 1s9rA; 1s9uA; 1sauA; 1sbxA; 1sdiA; 1sfxA; 1sg4A; 1sgvA; 1sgwA; 1sh8A; 1shuX; 1sjyA; 1skzA; 1smxA; 1sngA; 1so7A; 1sr4A; 1sr4C; 1sraA; 1ss4A; 1stmA; 1sumB; 1suuA; 1syyA; 1szhA; 1sznA; 1szwA; 1t7A; 1t0bA; 1t0fA; 1t0fC; 1t0hB; 1t0hA; 1t0tV; 1tijA; 1tivA; 1t3yA; 1t44G; 1t4aA; 1t61A; 1t6cA; 1t6oA; 1t6oB; 1t6t1; 1t8kA; 1t92A; 1tafB; 1tafA; 1tazA; 1tbfA; 1tc5A; 1te2A; 1tfeA; 1tgrA; 1th7A; 1tigA; 1tjvA; 1tkeA; 1tovA; 1tp6A; 1tr0A; 1ts9A; 1tt8A; 1tu1A; 1tu9A; 1tuaA; 1tuhA; 1tvfA; 1twdA; 1txgA; 1tzpA; 1u07A; 1u0kA; 1u53A; 1u5dA; 1u5fA; 1u5uA; 1u60A; 1u69A; 1u6zA; 1u7iA; 1u7kA; 1u71A; 1u7 pA; 1u84A; 1u8vA; 1u9cA; 1ua4A; 1uaiA; 1ucrA; 1ucsA; 1uebA; 1uekA; 1ufyA; 1ui0A; 1uiiA; 1uixA; 1uj2A; 1ujcA; 1ukfA; 1ukuA; 1u1kA; 1uirA; 1uoyA; 1upsA; 1urqA; 1us0A; 1us3A; 1us5A; 1uscA; 1useA; 1ut7A; 1uujA; 1uuqA; 1uuyA; 1uwkA; 1ux6A; 1uxyA; 1v05A; 1v0aA; 1v2zA; 1v30A; 1v37A; 1v4 pA; 1v5iB; 1v5vA; 1v6tA; 1v70A; 1v76A; 1v7wA; 1v7zA; 1v8cA; 1v8hA; 1v9fA; 1v9yA; 1vbkA; 1vbwA; 1vcdA; 1vc1A; 1vctA; 1vd6A; 1ve4A; 1vj0A; 1vj1A; 1vjnA; 1vjvA; 1vk1A; 1vk4A; 1vkcA; 1vkeA; 1vkfA; 1vkhA; 1vkiA; 1vkkA; 1vkuA; 1vkwA; 1vkyA; 1v11A; 1v14A; 1v15A; 1v17A; 1v1aA; 1v1rA; 1v1yA; 1vmbA; 1vmgA; 1vmhA; 1vp8A; 1vpbA; 1vpkA; 1vpmA; 1vprA; 1vq3A; 1vqsA; 1vr6A; 1vr8A; 1vr9A; 1vraA; 1vyiA; 1vykA; 1vyrA; 1w07A; 1w0hA; 1w0pA; 1w1hA; 1w23A; 1w2wA; 1w2wB; 1w2yA; 1w4sA; 1w53A; 1w5fA; 1w5rA; 1w66A; 1w6sA; 1w6sB; 1w7cA; 1w851; 1w8oA; 1w8sA; 1w99A; 1w9aA; 1w9eA; 1wb0A; 1wb4A; 1wckA; 1wd3A; 1wd5A; 1wddA; 1wddS; 1wdjA; 1wehA; 1whiA; 1whzA; 1wiwA; 1wjxA; 1wkaA; 1wkcA; 1wkoA; 1wkyA; 1w18A; 1w1uA; 1w1zA; 1wmdA; 1wmhA; 1wmhB; 1wmwA; 1wn2A; 1wnaA; 1woqA; 1wouA; 1wovA; 1wpaA; 1wpbA; 1wpnA; 1wqjI; 1wqjB; 1wr8A; 1wrdA; 1wtjA; 1wu9A; 1wvqA; 1wvvA; 1wwiA; 1wwzA; 1wyxA; 1wz3A; 1wzdA; 1wmA; 1wzoA; 1x2iA; 1x54A; 1x6oA; 1x6vA; 1x7dA; 1x7vA; 1x8qA; 1x91A; 1x9dA; 1x9iA; 1x9uA; 1xawA; 1xd3A; 1xdnA; 1xdzA; 1xe1A; 1xe7A; 1xerA; 1xffA; 1xfkA; 1xfsA; 1xg0C; 1xg0B; 1xg0A; 1xg7A; 1xgkA; 1xgsA; 1xiwA; 1xiwB; 1xjuA; 1xkiA; 1xkpA; 1xkpC; 1xkpB; 1xmkA; 1xmtA; 1xnfA; 1xo5A; 1xocA; 1xovA; 1xqaA; 1xqoA; 1xrkA; 1xruA; 1xsvA; 1xt5A; 1xt8A; 1xtpA; 1xu1A; 1xu1R; 1xubA; 1xv2A; 1xw3A; 1y02A; 1y07A; 1y0bA; 1y0hA; 1y0kA; 1y0pA; 1y0uA; 1y12A; 1y2 mA; 1y43B; 1y43A; 1y63A; 1y71A; 1y7wA; 1y81A; 1y88A; 1y89A; 1y8aA; 1y91A; 1y9qA; 1y9zA; 1yacA; 1yar0; 1yb0A; 1yb3A; 1ybkA; 1ybxA; 1ybzA; 1yc9A; 1ycdA; 1yd0A; 1ydyA; 1ye8A; 1yf9A; 1yfqA; 1ygtA; 1yhfA; 1yhtA; 1yj7A; 1ykdA; 1ykiA; 1y1eA; 1y1mA; 1y1xA; 1ym3A; 1ymtA; 1yn3A; 1ynbA; 1yoaA; 1yocA; 1yphE; 1yphC; 1ypyA; 1yq5A; 1yqhA; 1yqsA; 1yrkA; 1ysiX; 1ysrA; 1yt3A; 1yt8A; 1yu0A; 1yu5X; 1yu6C; 1yuzA; 1ywfA; 1ywmA; 1yxiA; 1yyhA; 1yzfA; 1yzmA; 1z05A; 1z0jB; 1z0kB; 1z0nA; 1z0pA; 1z0sA; 1z21A; 1z2nX; 1z3eA; 1z3eB; 1z4eA; 1z4rA; 1z67A; 1z6 mA; 1z6 nA; 1z6oA; 1z6oM; 1z70X; 1z72A; 1z7aA; 1z96A; 1z91A; 1z9tA; 1za0A; 1za4A; 1zarA; 1zceA; 1zczA; 1ze1A; 1zgkA; 1zgxA; 1zgxB; 1zhvA; 1zhxA; 1zi8A; 1zjcA; 1zk5A; 1zkeA; 1zkiA; 1zkpA; 1zl0A; 1zidA; 1z1hB; 1zm8A; 1zmaA; 1zn6A; 1zpsA; 1zpvA; 1zq9A; 1zr6A; 1zruA; 1zs9A; 1zswA; 1zsyA; 1zt3A; 1ztdA; 1zuoA; 1zv1A; 1zvaA; 1zvtA; 1zxxA; 1zy7A; 1zzgA; 2a14A; 2a15A; 2a26A; 2a35A; 2a3 mA; 2a3 nA; 2a40B; 2a40C; 2a4aA; 2a50B; 2a50A; 2a5 dB; 2a61A; 2a6sA; 2a6yA; 2a6zA; 2a71A; 2a9dA; 2a9iA; 2a9sA; 2aanA; 2abkA; 2absA; 2ae0X; 2aeeA; 2aexA; 2ah2A; 2ah5A; 2ahfA; 2ahuA; 2aibA; 2akzA; 2amhA; 2an1A; 2anxA; 2ao9A; 2ap3A; 2apjA; 2apoB; 2apoA; 2ar1A; 2asfA; 2askA; 2au3A; 2axcA; 2axqA; 2axwA; 2aydA; 2ayhA; 2azwA; 2b06A; 2b0aA; 2b0cA; 2b0tA; 2b0vA; 2b18A; 2b1eA; 2b1kA; 2b1yA; 2b3fA; 2b4hA; 2b4vA; 2b5aA; 2b69A; 2b7kA; 2b82A; 2b8iA; 2b8 mA; 2b9eA; 2b9wA; 2bayA; 2bbaA; 2bbeA; 2bbrA; 2bcmA; 2bdrA; 2bezC; 2bezF; 2bf6A; 2bfdB; 2bfdA; 2bfwA; 2bh8A; 2bhuA; 2bi0A; 2biiA; 2bjdA; 2bjiA; 2bjqA; 2bk9A; 2bkaA; 2bkfA; 2bkrA; 2bkxA; 2b10A; 2b18A; 2b19A; 2b1fB; 2b1nA; 2bm5A; 2bmoA; 2bmoB; 2bn1A; 2bnmA; 2bo4A; 2bo9B; 2bogX; 2brfA; 2bryA; 2bsjA; 2bt6A; 2btiA; 2bu3A; 2bueA; 2bv2A; 2bvfA; 2bwrA; 2bz1A; 2bzvA; 2c0aA; 2c0gA; 2c0nA; 2c0zA; 2c1iA; 2c21A; 2c2iA; 2c2 pA; 2c2uA; 2c3fA; 2c3 nA; 2c42A; 2c43A; 2c46A; 2c4bA; 2c4eA; 2c4xA; 2c5aA; 2c5qA; 2c60A; 2c78A; 2c81A; 2c92A; 2c9wA; 2ca1A; 2cayA; 2cb8A; 2cb9A; 2cc0A; 2cc6A; 2cchB; 2cdcA; 2cf7A; 2cgqA; 2ch5A; 2chcA; 2cisA; 2ciuA; 2ciwA; 2cj4A; 2cjjA; 2cjtA; 2ckkA; 2c13A; 2cm5A; 2co3A; 2covD; 2cs7A; 2cu1A; 2cvbA; 2cveA; 2cw9A; 2cwyA; 2cwzA; 2cx1A; 2cx7A; 2cxhA; 2cxiA; 2cxkA; 2cxxA; 2cxyA; 2cy5A; 2czqA; 2d0A; 2d0B; 2d1cA; 2d1sA; 2d1zA; 2d2eA; 2d3dA; 2d4 pA; 2d58A; 2d59A; 2d5 mA; 2d68A; 2d7cC; 2d81A; 2d8dA; 2db7A; 2dbnA; 2dbyA; 2dc3A; 2dc4A; 2ddfA; 2ddrA; 2ddxA; 2de3A; 2de6A; 2dejA; 2dg1A; 2dgaA; 2djfB; 2djfA; 2dkaA; 2dkhA; 2dkjA; 2dkoB; 2dkoA; 2dkvA; 2dm9A; 2dokA; 2dp9A; 2dpfA; 2dp1A; 2dq1A; 2ds5A; 2dskA; 2dsyA; 2dt4A; 2dtcA; 2dtjA; 2du1A; 2duyA; 2dvkA; 2dvmA; 2dwkA; 2dxaA; 2dxqA; 2dxuA; 2dy0A; 2dy1A; 2dyiA; 2dyjA; 2e11A; 2e1nA; 2e1vA; 2e26A; 2e2oA; 2e3hA; 2e3 nA; 2e4tA; 2e5fA; 2e6fA; 2e7zA; 2e8eA; 2eabA; 2eb1A; 2ebbA; 2efvA; 2eg4A; 2egdA; 2egjA; 2eh3A; 2ehpA; 2ehzA; 2ei5A; 2eiyA; 2ej8A; 2ejnA; 2e1cA; 2endA; 2eo4A; 2epiA; 2ep1X; 2eq7C; 2eq8C; 2erfA; 2er1A; 2ervA; 2erwA; 2essA; 2et1A; 2etbA; 2etvA; 2ev1A; 2ew0A; 2ewhA; 2ewtA; 2ez1A; 2fD1A; 2fDcA; 2f1kA; 2f2bA; 2f46A; 2f4 pA; 2f60K; 2f62A; 2f68X; 2f6eA; 2f6rA; 2f7bA; 2f7vA; 2f9iA; 2f9iB; 2faoA; 2fb5A; 2fb6A; 2fbaA; 2fbnA; 2fboJ; 2fbqA; 2fcjA; 2fc1A; 2fcoA; 2fctA; 2fcwB; 2fcwA; 2fd5A; 2fdrA; 2fdvA; 2feaA; 2fefA; 2fexA; 2ff3A; 2ff4A; 2ffuA; 2ffyA; 2fh1A; 2fh7A; 2fhfA; 2fhpA; 2fhzB; 2fhzA; 2fi1A; 2fipA; 2fiuA; 2fj8A; 2fk5A; 2fk8A; 2fk9A; 2fkkA; 2f14A; 2fm9A; 2fmaA; 2fmmE; 2fnaA; 2fnoA; 2fnuA; 2fo3A; 2fomB; 2fomA; 2fozA; 2fp1A; 2fp7A; 2fp7B; 2fprA; 2fq3A; 2fq4A; 2fqpA; 2fqxA; 2fr2A; 2fr5A; 2freA; 2frgP; 2fsqA; 2fsrA; 2fsxA; 2fM0A; 2ftrA; 2fu4A; 2fueA; 2fukA; 2fupA; 2furA; 2fvhA; 2fvvA; 2fvyA; 2fwvA; 2fy6A; 2fy7A; 2fyfA; 2fygA; 2fzpA; 2fzvA; 2g0cA; 2g0iA; 2g0wA; 2g1uA; 2g30A; 2g3aA; 2g3bA; 2g40A; 2g45A; 2g5gX; 2g5xA; 2g62A; 2g7cA; 2g7oA; 2g81I; 2g84A; 2g8sA; 2g9wA; 2ga1A; 2gaiA; 2gakA; 2gauA; 2gaxA; 2gb4A; 2ge7A; 2gecA; 2genA; 2geyA; 2gf6A; 2gffA; 2gfhA; 2gfnA; 2gfqA; 2ggcA; 2ghsA; 2gibA; 2gj4A; 2gkeA; 2gkgA; 2gkpA; 2g19A; 2g1zA; 2gmwA; 2gmyA; 2gnoA; 2gomA; 2gpiA; 2gq0A; 2gqtA; 2gqwA; 2gr8A; 2gs5A; 2gsoA; 2gu3A; 2gu9A; 2guiA; 2gviA; 2gwgA; 2gwmA; 2gwnA; 2gxgA; 2gyqA; 2gz4A; 2gz6A; 2h0uA; 2h1cA; 2h1tA; 2h1vA; 2h2tB; 2h7oA; 2h88A; 2h88B; 2h88C; 2h88D; 2h8eA; 2h8gA; 2h8oA; 2h98A; 2ha8A; 2hbaA; 2hboA; 2hbwA; 2hc8A; 2hc9A; 2hcfA; 2hcmA; 2hd9A; 2hdoA; 2hdwA; 2hdzA; 2hekA; 2heuA; 2hewF; 2heyR; 2hf2A; 2hfsA; 2hhcA; 2hhgA; 2hhzA; 2hi0A; 2hinA; 2hiqA; 2hiyA; 2hjeA; 2hjnA; 2hkjA; 2hkvA; 2h17A; 2h1jA; 2h1rA; 2h1yA; 2hngA; 2hoxA; 2hp0A; 2hpjA; 2hpsA; 2hq7A; 2hq9A; 2hqqA; 2hqsA; 2hqsC; 2hqxA; 2hqyA; 2hrzA; 2hs1A; 2hsbA; 2hsiA; 2hsjA; 2ht9A; 2htaA; 2htdA; 2hu9A; 2huhA; 2hujA; 2hv8D; 2hvwA; 2hw4A; 2hx0A; 2hxiA; 2hxvA; 2hy1A; 2hy5A; 2hy5C; 2hy5B; 2hy7A; 2hytA; 2hzqA; 2i0kA; 2i0zA; 2i2oA; 2i3dA; 2i49A; 2i4lA; 2i51A; 2i5hA; 2i5iA; 2i5uA; 2i5v0; 2i6hA; 2i71A; 2i7aA; 2i7fA; 2i8bA; 2i8dA; 2i8tA; 2i9aA; 2i9cA; 2i9iA; 2i9wA; 2i9xA; 2ia1A; 2ia7A; 2iabA; 2iaiA; 2iayA; 2ib0A; 2ibdA; 2ib1A; 2ibnA; 2ic6A; 2icgA; 2ichA; 2icuA; 2id3A; 2id6A; 2id1A; 2ie1A; 2ieqA; 2iewA; 2if6A; 2ifxA; 2ig8A; 2igiA; 2igpA; 2ih3C; 2ihtA; 2ii1A; 2ii2A; 2iidA; 2ij2A; 2ijqA; 2ikbA; 2iksA; 2i1rA; 2im9A; 2imfA; 2imhA; 2imjA; 2imqX; 2imrA; 2imzA; 2in3A; 2inwA; 2ionA; 2ip1A; 2ip6A; 2iqjA; 2iruA; 2is9A; 2isbA; 2it2A; 2it9A; 2iu1A; 2iu5A; 2iumA; 2iuwA; 2ivfA; 2ivfB; 2ivfC; 2ivyA; 2iw0A; 2iw1A; 2iwaA; 2ixdA; 2ixsA; 2iy2A; 2iy9A; 2iyjA; 2iyvA; 2izxA; 2j0aA; 2j1aA; 2j1vA; 2j2jA; 2j5gA; 2j5yA; 2j6aA; 2j6bA; 2j6yA; 2j73A; 2j7qA; 2j89A; 2j8bA; 2j8hA; 2j8kA; 2j91A; 2j97A; 2j9oA; 2j9wA; 2jaeA; 2jayA; 2jc9A; 2jdaA; 2jdcA; 2jdjA; 2je6B; 2je6A; 2je6I; 2je8A; 2jekA; 2jenA; 2jfrA; 2jg0A; 2jgsA; 2jh1A; 2jhnA; 2jjqA; 2jjsC; 2jkbA; 2jkuA; 2j1iA; 2j1jA; 2mcmA; 2mhrA; 2msbA; 2n1vA; 2nm1A; 2nnuA; 2nnuB; 2no4A; 2np5A; 2nptA; 2nptB; 2nq3A; 2ngtA; 2nqwA; 2nr5A; 2nr7A; 2nrkA; 2nrrA; 2nrtA; 2nszA; 2nt0A; 2ntpA; 2nujA; 2nvaA; 2nw8A; 2nwfA; 2nx2A; 2nx4A; 2nxcA; 2nxfA; 2nxvA; 2nxwA; 2nyiA; 2nz7A; 2nzcA; 2nz1A; 2o0 mA; 2o0yA; 2o16A; 2o1aA; 2o1kA; 2o1qA; 2o28A; 2o2gA; 2o2kA; 2o2vB; 2o2xA; 2o30A; 2o3fA; 2o4dA; 2o4jA; 2o4tA; 2o4xB; 2o57A; 2o5hA; 2o5uA; 2o5vA; 2o62A; 2o66A; 2o6fA; 2o61A; 2o6 pA; 2o6xA; 2o70A; 2o7rA; 2o8 nA; 2o8 pA; 2o8qA; 2o90A; 2o9sA; 2oa2A; 2oafA; 2ob0A; 2ob3A; 2ob5A; 2obpA; 2oczA; 2od0A; 2od4A; 2od5A; 2od6A; 2odfA; 2odkA; 2od1A; 2oebA; 2ofkA; 2ofzA; 2og2A; 2og4A; 2ogfA; 2oh1A; 2oh3A; 2ohwA; 2oikA; 2oiwA; 2oixA; 2oizD; 2oizA; 2oj6A; 2ojhA; 2okfA; 2okgA; 2okqA; 2oktA; 2okuA; 2o1nA; 2o1rA; 2o1tA; 2o1wA; 2omdA; 2omkA; 2om1A; 2onfA; 2ooaA; 2oocA; 2oojA; 2ookA; 2opjA; 2op1A; 2opoA; 2opwA; 2oqbA; 2oqkA; 2oqmA; 2oqzA; 2orwA; 2os0A; 2osoA; 2osxA; 2otmA; 2ou3A; 2ou5A; 2ou6A; 2ouwA; 2ov0A; 2ov9A; 2ovgA; 2ovjA; 2owaA; 2ownA; 2owpA; 2ox6A; 2oxgB; 2oxgA; 2ox1A; 2oy7A; 2oy9A; 2oyaA; 2oyoA; 2oyzA; 2ozgA; 2ozhA; 2oznA; 2oznB; 2oztA; 2ozvA; 2p09A; 2p0aA; 2p0kA; 2p0nA; 2p0sA; 2p0wA; 2p14A; 2p17A; 2p18A; 2p1mA; 2p1mB; 2p25A; 2p2sA; 2p2vA; 2p35A; 2p3hA; 2p3 pA; 2p3wA; 2p3yA; 2p4eA; 2p4oA; 2p4 pA; 2p51A; 2p57A; 2p58A; 2p58C; 2p58B; 2p5kA; 2p5 mA; 2p65A; 2p6vA; 2p6wA; 2p7iA; 2p8gA; 2p8iA; 2p8jA; 2p8tA; 2p97A; 2p9wA; 2pagA; 2pb7A; 2pbcA; 2pbdV; 2pbiA; 2pb1A; 2pc1A; 2pcnA; 2pd1A; 2pdrA; 2pebA; 2petA; 2pfiA; 2pfzA; 2pgeA; 2pgfA; 2pgoA; 2ph0A; 2phnA; 2pieA; 2pjsA; 2pjzA; 2pk8A; 2pkeA; 2pkfA; 2pkhA; 2p1iA; 2pn0A; 2pn1A; 2pn2A; 2pn6A; 2pndA; 2pneA; 2pnwA; 2pokA; 2posA; 2ppqA; 2ppvA; 2ppxA; 2pq7A; 2pqvA; 2pr5A; 2pr7A; 2prvA; 2pstX 2pt6A; 2pttB; 2pttA; 2pu3A; 2puzA; 2pv4A; 2pv7A; 2pwwA; 2pyqA; 2pytA; 2pywA; 2pyxA; 2pzeA; 2q03A; 2q0sA; 2q0tA; 2q0yA; 2q12A; 2q1sA; 2q24A; 2q2fA; 2q2hA; 2q30A; 2q35A; 2q3xA; 2q5cA; 2q5wD; 2q6kA; 2q73A; 2q79A; 2q7bA; 2q7sA; 2q86A; 2q88A; 2q8kA; 2q8 pA; 2q8rE; 2q9kA; 2q9rA; 2q9vA; 2qb7A; 2qbwA; 2qckA; 2qe6A; 2qe8A; 2qe9A; 2qebA; 2qecA; 2qedA; 2qeeA; 2qeuA; 2qf4A; 2qf7A; 2qfaA; 2qfaB; 2qfaC; 2qfeA; 2qg3A; 2qg8A; 2qgmA; 2qguA; 2qgyA; 2qhfA; 2qhkA; 2qhoB; 2qhpA; 2qhqA; 2qhsA; 2qhtA; 2qibA; 2qihA; 2qikA; 2qipA; 2qiwA; 2qiyA; 2qiyC; 2qj1A; 2qjvA; 2qjwA; 2qjzA; 2qkdA; 2qkhB; 2qkhA; 2qkpA; 2q18A; 2q1tA; 2q1wA; 2q1xA; 2qm8A; 2qm1A; 2qmqA; 2qngA; 2qniA; 2qnkA; 2qn1A; 2qntA; 2qo1A; 2qp2A; 2qpwA; 2qpxA; 2qq5A; 2qqiA; 2qqmA; 2qqyA; 2qr6A; 2qruA; 2qs9A; 2qsbA; 2qsiA; 2qskA; 2qsxA; 2qt1A; 2qtdA; 2qtqA; 2qtwB; 2qtzA; 2qudA; 2qupA; 2qv5A; 2qvgA; 2qvkA; 2qw5A; 2qwoB; 2qwuA; 2qy6A; 2qycA; 2qywA; 2qzcA; 2qzqA; 2qztA; 2r01A; 2r09A; 2r0hA; 2r0xA; 2r0yA; 2r16A; 2r1iA; 2r2cA; 2r2oA; 2r2zA; 2r37A; 2r3aA; 2r3bA; 2r44A; 2r4fA; 2r4gA; 2r4iA; 2r4qA; 2r58A; 2r5oA; 2r5uA; 2r6qA; 2r6vA; 2r6zA; 2r78A; 2r7hA; 2r85A; 2r8eA; 2r8uA; 2r9fA; 2ra9A; 2rafA; 2rasA; 2rauA; 2rb7A; 2rbbA; 2rbcA; 2rbdA; 2rbgA; 2rbkA; 2rc3A; 2rccA; 2rciA; 2rczA; 2rdcA; 2rdgA; 2rdqA; 2re2A; 2reeA; 2rfaA; 2rffA; 2rfmA; 2rfqA; 2rfrA; 2rg8A; 2rgqA; 2rh0A; 2rh2A; 2rh3A; 2rhkA; 2rhkC; 2rhmA; 2rhwA; 2rijA; 2ri1A; 2riqA; 2rivB; 2rj2A; 2rjiA; 2rkhA; 2rk1A; 2rkqA; 2rkvA; 2r1dA; 2sakA; 2tpsA; 2uu8A; 2uurA; 2uuyB; 2uv4A; 2uvkA; 2uvoA; 2uw1A; 2uxwA; 2uyoA; 2uytA; 2uz1A; 2v03A; 2v05A; 2v0cA; 2v1mA; 2v1oA; 2v27A; 2v2fF; 2v2fA; 2v2kA; 2v33A; 2v3gA; 2v3iA; 2v4xA; 2v57A; 2v6vA; 2v6xB; 2v75A; 2v76A; 2v7fA; 2v7kA; 2v7sA; 2v89A; 2v8fA; 2v8fC; 2v8iA; 2v8tA; 2v94A; 2v9vA; 2vacA; 2vb1A; 2vbkA; 2vchA; 2vc1A; 2vcnA; 2vdjA; 2ve8A; 2vfoA; 2vfrA; 2vfxA; 2vgxA; 2vh3A; 2vhjA; 2vhkA; 2vifA; 2vjwA; 2vk8A; 2vkjA; 2vk1A; 2v1gA; 2v1iA; 2v1qA; 2vm5A; 2vn4A; 2vngA; 2vn1A; 2vo8A; 2vo9A; 2vovA; 2vpaA; 2vpbB; 2vpbA; 2vpnA; 2vptA; 2vq2A; 2vqgA; 2vqpA; 2vqxA; 2vrsA; 2vrwB; 2vsmA; 2vt1A; 2vt1B; 2vtwA; 2vunA; 2vveA; 2vvmA; 2vvwA; 2vw8A; 2vwsA; 2vxgA; 2vxnA; 2vxtI; 2vxzA; 2vy8A; 2vyoA; 2vzcA; 2vzpA; 2vzsA; 2vzyA; 2w0bA; 2w0iA; 2w15A; 2w1rA; 2w1sA; 2w1vA; 2w2aA; 2w2kA; 2w2rA; 2w31A; 2w39A; 2w3gA; 2w3 pA; 2w3qA; 2w3yA; 2w3zA; 2w40A; 2w4eA; 2w50A; 2w56A; 2w5eA; 2w5fA; 2w5qA; 2w61A; 2w6aA; 2w7aA; 2w87A; 2w8tA; 2w8xA; 2w91A; 2w9yA; 2waaA; 2wanA; 2waoA; 2wawA; 2wb3A; 2wb6A; 2wbfX; 2wbmA; 2wbnA; 2wbqA; 2wbxA; 2wciA; 2wcrA; 2wdcA; 2wdsA; 2we5A; 2wf7A; 2wfiA; 2wfoA; 2wfpA; 2wg7A; 2wgpA; 2wh7A; 2wi8A; 2wiyA; 2wj5A; 2wj9A; 2wjeA; 2wjnM; 2wjnL; 2wjnH; 2wjnC; 2wjrA; 2wk1A; 2wkkA; 2wkqA; 2w1cA; 2w1rA; 2w1uA; 2wm3A; 2wm8A; 2wn9A; 2wnfA; 2wnpF; 2wnsA; 2wnwA; 2wnxA; 2wnyA; 2wo1A; 2woyA; 2wp7A; 2wpvA; 2wpvB; 2wq4A; 2wqfA; 2wqkA; 2wsdA; 2wshA; 2wt0A; 2wtaA; 2wteA; 2wtgA; 2wtmA; 2wuqA; 2wv3A; 2wvfA; 2wvxA; 2wweA; 2wwxB; 2wxuA; 2wy8Q; 2wyhA; 2wz8A; 2wz9A; 2wzbA; 2wzoA; 2wzvA; 2x0kA; 2x0qA; 2x1dA; 2x26A; 2x2sA; 2x2uA; 2x32A; 2x3cA; 2x3gA; 2x3jA; 2x31A; 2x3 mA; 2x3 nA; 2x46A; 2x49A; 2x4dA; 2x4jA; 2x4kA; 2x4lA; 2x55A; 2x5cA; 2x5fA; 2x5hA; 2x5 nA; 2x5oA; 2x5 pA; 2x5rA; 2x5xA; 2x5yA; 2x61A; 2x7 mA; 2x7qA; 2x8hA; 2x8sA; 2x98A; 2x9oA; 2x9xA; 2x9zA; 2xbgA; 2xc1A; 2xcjA; 2xdgA; 2xdjA; 2xdpA; 2xdwA; 2xe4A; 2xedA; 2xepA; 2xetA; 2xf7A; 2xfdA; 2xfgB; 2xfnA; 2xfrA; 2xfvA; 2xg5B; 2xgrA; 2xhfA; 2xhgA; 2xhnA; 2xi8A; 2xi9A; 2xigA; 2xijA; 2xioA; 2xkiA; 2x1gA; 2xm5A; 2xmxA; 2xmzA; 2xn6A; 2xn6B; 2xnqA; 2xocA; 2xodA; 2xo1A; 2xovA; 2xppA; 2xppB; 2xpwA; 2xqoA; 2xrhA; 2xryA; 2xsaA; 2xseA; 2xsgA; 2xskA; 2xsqA; 2xswA; 2xt2A; 2xtpA; 2xtyA; 2xu3A; 2xu8A; 2xuvA; 2xvsA; 2xvyA; 2xw6A; 2xwsA; 2xwtC; 2xwvA; 2xx1A; 2xxmA; 2xxpA; 2xxzA; 2xy2A; 2xyiA; 2xyiB; 2xyqA; 2xz2A; 2xz8A; 2xz9A; 2xzeA; 2xzeQ; 2xziA; 2y0oA; 2y27A; 2y2 mA; 2y2zA; 2y32A; 2y3cA; 2y43A; 2y44A; 2y53A; 2y5fL; 2y6uA; 2y6xA; 2y71A; 2y78A; 2y7bA; 2y7 pA; 2y8gA; 2y8kA; 2y8 nA; 2y8nB; 2y8uA; 2y9uA; 2ya0A; 2yanA; 2yavA; 2yb1A; 2ybqA; 2ybyA; 2yc3A; 2ycdA; 2yd6A; 2yeqA; 2yfaA; 2yfoA; 2yfrA; 2yfuA; 2yg2A; 2yg9A; 2ygnA; 2ygoA; 2yh9A; 2yhaA; 2yhgA; 2yhsA; 2yhwA; 2yi1A; 2yimA; 2yjgA; 2yk4A; 2y1eA; 2y1eB; 2y1nA; 2ymmA; 2ymuA; 2ymvA; 2ymyA; 2yn0A; 2yn5A; 2ynaA; 2ynyA; 2ynzA; 2yp6A; 2ypoA; 2yqyA; 2yqzA; 2yskA; 2yv4A; 2yv9A; 2yviA; 2yvtA; 2yw3A; 2ywiA; 2yw1A; 2yxnA; 2yxoA; 2yxzA; 2yyhA; 2yyyA; 2yzjA; 2yzkA; 2yzqA; 2yzsA; 2yztA; 2yzyA; 2z0bA; 2z0dA; 2z0jA; 2z0qA; 2z0tA; 2z14A; 2zlcA; 2z26A; 2z30B; 2z3hA; 2z3zA; 2z5eA; 2z62A; 2z6dA; 2z6iA; 2z6oA; 2z6rA; 2z72A; 2z7fI; 2z84A; 2z8fA; 2z8xA; 2z98A; 2z9wA; 2za4B; 2zb1A; 2zcaA; 2zcwA; 2zdpA; 2zexA; 2zf9A; 2zfdA; 2zfdB; 2zfuA; 2zfyA; 2zfzA; 2zhjA; 2zkmX; 2znrA; 2zonG; 2zouA; 2zp1A; 2zpmA; 2zptX; 2zq5A; 2zqeA; 2zqmA; 2zqoA; 2zs0A; 2zsjA; 2zuvA; 2zuxA; 2zvcA; 2zw2A; 2zwaA; 2zx2A; 2zxqA; 2zxyA; 2zycA; 2zzdB; 2zzdA; 2zzjA; 3a02A; 3a07A; 3a0sA; 3a0yA; 3a1fA; 3a1sA; 3a21A; 3a2qA; 3a2vA; 3a3dA; 3a4uB; 3a54A; 3a57A; 3a5fA; 3a5 pA; 3a6rA; 3a72A; 3a8gA; 3a8gB; 3a9fA; 3a9iA; 3a91A; 3a9sA; 3aa0B; 3aa0A; 3aa0C; 3ab8A; 3abdA; 3abdX; 3achA; 3acxA; 3adgA; 3adoA; 3aehA; 3afoA; 3ag7A; 3agnA; 3agyA; 3ahcA; 3ahnA; 3ai4A; 3ai5A; 3aiaA; 3aiiA; 3aj3A; 3aj4A; 3aj7A; 3ajdA; 3ajfA; 3ak8A; 3akbA; 3akeA; 3akhA; 3a12A; 3amnA; 3anpA; 3aofA; 3apqA; 3aq2A; 3aqiA; 3aqjA; 3arxA; 3as5A; 3as1A; 3atsA; 3au8A; 3awuA; 3awuB; 3ax2A; 3axbA; 3axgA; 3ay2A; 3ayjA; 3ayvA; 3azoA; 3b02A; 3b08B; 3b0fA; 3b0gA; 3b0pA; 3b0xA; 3b1bA; 3b2yA; 3b40A; 3b42A; 3b4 nA; 3b4qA; 3b4uA; 3b5eA; 3b5 mA; 3b5oA; 3b64A; 3b6eA; 3b6hA; 3b79A; 3b7cA; 3b7eA; 3b8bA; 3b8fA; 3b81A; 3b9tA; 3b9wA; 3ba3A; 3bb0A; 3bb7A; 3bb9A; 3bc1B; 3bc8A; 3bc9A; 3bcwA; 3bcyA; 3bd1A; 3bdeA; 3bdiA; 3bduA; 3bdvA; 3be6A; 3bemA; 3bexA; 3bf5A; 3bf7A; 3bfmA; 3bfoA; 3bg2A; 3bgeA; 3bguA; 3bgyA; 3bh4A; 3bh7B; 3bhdA; 3bhgA; 3bhnA; 3bhqA; 3biqA; 3biyA; 3bj4A; 3bjdA; 3bjeA; 3bjkA; 3bjnA; 3bk5A; 3bkbA; 3bkpA; 3bkrA; 3bkwA; 3bkxA; 3b19A; 3b1nA; 3b1zA; 3bm7A; 3bmvA; 3bmxA; 3bmzA; 3bn0A; 3bn7A; 3bnjA; 3bo6A; 3bodA; 3boeA; 3bofA; 3bonA; 3bosA; 3bp3A; 3bpjA; 3bpkA; 3bptA; 3bpvA; 3bpzA; 3bq3A; 3bqkA; 3bqxA; 3brcA; 3bs4A; 3bs5A; 3bs5B; 3bs6A; 3bt5A; 3butA; 3buuA; 3bv6A; 3bv8A; 3bvuA; 3bwhA; 3bw1A; 3bwsA; 3bwvA; 3bwxA; 3bwzA; 3bx4B; 3by4A; 3byqA; 3bzhA; 3bztA; 3bzwA; 3c05A; 3c0cA; 3c0fB; 3c18A; 3c1aA; 3c1dA; 3c1qA; 3c1vA; 3c24A; 3c26A; 3c2qA; 3c2uA; 3c37A; 3c3 pA; 3c4bA; 3c4 mA; 3c4mC; 3c4sA; 3c57A; 3c5eA; 3c5hA; 3c5 nA; 3c5rA; 3c5vA; 3c6aA; 3c6kA; 3c6vA; 3c6wA; 3c7fA; 3c7xA; 3c85A; 3c8cA; 3c8eA; 3c8iA; 3c81A; 3c8uA; 3c8wA; 3c8zA; 3c9aA; 3c9fA; 3c9hA; 3c9 pA; 3c9uA; 3c9zA; 3ca7A; 3caiA; 3canA; 3cawA; 3cbnA; 3cbwA; 3cc1A; 3cc8A; 3ccdA; 3ccfA; 3ccgA; 3ce7A; 3cecA; 3cetA; 3cexA; 3cg6A; 3cggA; 3cgxA; 3ch0A; 3chjA; 3chmA; 3ci3A; 3ci6A; 3cimA; 3cinA; 3citA; 3cj1A; 3cjdA; 3cjeA; 3cjmA; 3cjnA; 3cjsB; 3cjsA; 3cjyA; 3ck1A; 3ck6A; 3ckcA; 3ckjA; 3ckkA; 3ckmA; 3c15A; 3c1mA; 3cmbA; 3cmgA; 3cnhA; 3cnqP; 3cnuA; 3cnvA; 3cnyA; 3covA; 3cp0A; 3cp5A; 3cp7A; 3cq1A; 3cqvA; 3cryA; 3cs1A; 3csgA; 3ct5A; 3ct6A; 3ctpA; 3ctzA; 3cu2A; 3cu3A; 3cu9A; 3cuzA; 3cv0A; 3cveA; 3cvjA; 3cvoA; 3cwiA; 3cwnA; 3cwrA; 3cwvA; 3cwwA; 3cx2A; 3cxkA; 3cxnA; 3cypB; 3cz1A; 3cz7A; 3czpA; 3czxA; 3czzA; 3d00A; 3d0A; 3d02A; 3d06A; 3d0fA; 3d0jA; 3d0wA; 3d1pA; 3d1rA; 3d22A; 3d21A; 3d2qA; 3d30A; 3d32A; 3d33A; 3d3bJ; 3d3bA; 3d3yA; 3d40A; 3d4eA; 3d59A; 3d5 pA; 3d6jA; 3d6 mA; 3d6mB; 3d79A; 3d7aA; 3d7iA; 3d7jA; 3d8tA; 3d9 nA; 3d9xA; 3da0A; 3da8A; 3dacA; 3dacB; 3da1A; 3daoA; 3db0A; 3db7A; 3dcdA; 3dcxA; 3dcyA; 3dczA; 3dd7A; 3dd7B; 3ddcB; 3ddhA; 3ddjA; 3ddtA; 3defA; 3de1B; 3deoA; 3dewA; 3df7A; 3df8A; 3dffA; 3dgpB; 3dgpA; 3dhaA; 3dhoA; 3dhuA; 3di4A; 3dj1A; 3dk9A; 3dkmA; 3d1cA; 3d1qI; 3d1qR; 3d1uA; 3dm8A; 3dmcA; 3dmeA; 3dmgA; 3dn7A; 3dnjA; 3dnpA; 3dnuA; 3dnxA; 3do6A; 3do8A; 3douA; 3dqgA; 3dqpA; 3dqyA; 3draB; 3drfA; 3drzA; 3ds4A; 3ds8A; 3dsbA; 3dskA; 3dsmA; 3dsoA; 3dssB; 3dssA; 3dttA; 3dtzA; 3dupA; 3dv9A; 3dwgC; 3dxeA; 3dxeB; 3dx1A; 3dxyA; 3dyjA; 3dz1A; 3dzaA; 3e03A; 3e05A; 3e0iA; 3e0xA; 3e0zA; 3e10A; 3e11A; 3e15A; 3e18A; 3e19A; 3e23A; 3e2dA; 3e2oA; 3e2qA; 3e2vA; 3e3uA; 3e46A; 3e48A; 3e4gA; 3e4vA; 3e57A; 3e58A; 3e5uA; 3e6qA; 3e7hA; 3e7rL; 3e8 mA; 3e8oA; 3e8tA; 3e96A; 3e99A; 3e9fA; 3e9kA; 3e9vA; 3ea6A; 3eafA; 3ebbA; 3ebtA; 3ebyA; 3ec4A; 3ec6A; 3ecfA; 3echA; 3echC; 3ed4A; 3edfA; 3edhA; 3edoA; 3edvA; 3ee4A; 3eeaA; 3eehA; 3eerA; 3eesA; 3eetA; 3ef8A; 3efgA; 3efyA; 3eg4A; 3egaA; 3eggC; 3egoA; 3egvB; 3ehgA; 3ehrA; 3eifA; 3einA; 3ej9A; 3ej9B; 3ejgA; 3ejkA; 3ejvA; 3ek3A; 3ekgA; 3e1bA; 3e1fA; 3e1kA; 3en0A; 3en8A; 3eo6A; 3eo7A; 3eofA; 3eoiA; 3eojA; 3ep6A; 3ep6B; 3eqxA; 3er6A; 3er7A; 3erbA; 3es1A; 3es4A; 3es1A; 3esmA; 3essA; 3etnA; 3etoA; 3etvA; 3etzA; 3euaA; 3euoA; 3eurA; 3evpA; 3evyA; 3ewnA; 3ewyA; 3exeA; 3exmA; 3exnA; 3exqA; 3ey8A; 3eyeA; 3eytA; 3ezuA; 3fDdA; 3f0hA; 3f0iA; 3f14A; 3f2eA; 3f2zA; 3f3kA; 3f40A; 3f42A; 3f43A; 3f44A; 3f47A; 3f4aA; 3f4 mA; 3f4sA; 3f52A; 3f59A; 3f5bA; 3f5hA; 3f5oA; 3f5rA; 3f62A; 3f6cA; 3f6oA; 3f6vA; 3f6wA; 3f6yA; 3f75P; 3f7cA; 3f7eA; 3f71A; 3f7qA; 3f7wA; 3f7xA; 3f81A; 3f8bA; 3f8kA; 3f8 mA; 3f8tA; 3f8xA; 3f95A; 3f9sA; 3f9xA; 3fajA; 3fanA; 3fb9A; 3fb1A; 3fbuA; 3fcnA; 3fd3A; 3fdbA; 3fdhA; 3fdjA; 3fdrA; 3fedA; 3fegA; 3fDA; 3ff1A; 3f2A; 3ffrA; 3fg1A; 3fg8A; 3fg9A; 3fgeA; 3fgrB; 3fgrA; 3fgvA; 3fgyA; 3fh1A; 3fhdA; 3fh1A; 3fiaA; 3fidA; 3fiA; 3fj1A; 3fj2A; 3fjsA; 3fjuB; 3fjvA; 3fkaA; 3f12A; 3f1aA; 3f1jA; 3fn0A; 3fm2A; 3fmcA; 3fmyA; 3fn2A; 3fn5A; 3fncA; 3fndA; 3fo3A; 3fo5A; 3fo8D; 3fojA; 3fotA; 3fp3A; 3fp5A; 3fp7J; 3fpcA; 3fpfA; 3fprA; 3fpwA; 3fpzA; 3fqgA; 3fqmA; 3fr7A; 3frhA; 3frqA; 3fryA; 3fsaA; 3fsdA; 3fseA; 3fsgA; 3fsoA; 3fssA; 3fstA; 3ft1A; 3ftdA; 3fuyA; 3fvbA; 3fwkA; 3fwuA; 3fwyA; 3fwzA; 3fx7A; 3fxaA; 3fxgA; 3fxhA; 3fxqA; 3fybA; 3fymA; 3fynA; 3fyqA; 3fzeA; 3fzgA; 3fzyA; 3g0kA; 3g0mA; 3g0tA; 3g14A; 3g16A; 3g1jA; 3g1pA; 3g21A; 3g23A; 3g2eA; 3g2 mA; 3g36A; 3g3sA; 3g3tA; 3g40A; 3g46A; 3g48A; 3g5bA; 3g5jA; 3g5oA; 3g5oB; 3g5sA; 3g5tA; 3g68A; 3g7 nA; 3g7 pA; 3g7qA; 3g7rA; 3g7uA; 3g85A; 3g89A; 3g8yA; 3g8zA; 3g98A; 3ga3A; 3ga4A; 3ga8A; 3gaeA; 3gagA; 3gb5A; 3gbwA; 3gd0A; 3gd6A; 3gdbA; 3gdcA; 3gdhA; 3gdwA; 3ge3A; 3ge3B; 3ge3C; 3ge3E; 3gewB; 3gf3A; 3gf6A; 3gfaA; 3gg7A; 3gg9A; 3ghdA; 3ghjA; 3gi7A; 3giuA; 3giwA; 3gj8B; 3gjyA; 3gk6A; 3gkeA; 3gkjA; 3gkrA; 3gmfA; 3gmgA; 3gmiA; 3gmsA; 3gmxA; 3gn6A; 3gneA; 3gn1A; 3gnzP; 3go2A; 3go5A; 3go9A; 3gocA; 3gohA; 3gonA; 3gp4A; 3gpiA; 3gpkA; 3gqhA; 3gqqA; 3gqvA; 3gr3A; 3grdA; 3grhA; 3gmA; 3gruA; 3grzA; 3gs9A; 3guzA; 3gv1A; 3gveA; 3gwbA; 3gwcA; 3gwiA; 3gwkC; 3gwqA; 3gwyA; 3gx8A; 3gxhA; 3gy9A; 3gybA; 3gycA; 3gydA; 3gykA; 3gzaA; 3gzbA; 3gzkA; 3gzrA; 3h01A; 3h05A; 3h09A; 3h0hA; 3h0nA; 3h0oA; 3h0uA; 3h1dA; 3h1nA; 3h20A; 3h2bA; 3h2gA; 3h2sA; 3h2yA; 3h31A; 3h36A; 3h3hA; 3h31A; 3h4lA; 3h4oA; 3h4tA; 3h4xA; 3h51A; 3h51A; 3h5 nA; 3h6jA; 3h6 nA; 3h6pC; 3h6 pA; 3h74A; 3h7aA; 3h7cX; 3h7iA; 3h87A; 3h87C; 3h8gA; 3h8tA; 3h8uA; 3h8zA; 3h95A; 3h9cA; 3h9 mA; 3ha2A; 3ha9A; 3hbnA; 3hc1A; 3hcgA; 3hcjA; 3hdjA; 3hdxA; 3he5B; 3he5A; 3hfoA; 3hftA; 3hfwA; 3hh1A; 3hhiA; 3hhyA; 3hi2B; 3hi7A; 3hj4A; 3hj9A; 3hjeA; 3hkwA; 3h11A; 3h18A; 3h1xA; 3h1zA; 3hm4A; 3hmzA; 3hn0A; 3hn3A; 3hn5A; 3hn7A; 3ho6A; 3hoiA; 3ho1A; 3hp7A; 3hpcX; 3hq1A; 3hqxA; 3hr0A; 3hr6A; 3hrgA; 3hr1A; 3hroA; 3hrpA; 3hs3A; 3hssA; 3hsyA; 3ht1A; 3htnA; 3htvA; 3htyA; 3hu5A; 3huhA; 3huuA; 3hvwA; 3hvyA; 3hwpA; 3hwuA; 3hx3A; 3hx8A; 3hx9A; 3hx1A; 3hxsA; 3hynA; 3hz6A; 3hzpA; 3i09A; 3i0yA; 3i0zA; 3i10A; 3i18A; 3i1aA; 3i2kA; 3i2 nA; 3i2vA; 3i36A; 3i3fA; 3i3gA; 3i45A; 3i4gA; 3i4oA; 3i4zA; 3i57A; 3i5cA; 3i7 mA; 3i83A; 3i84A; 3i8bA; 3i94A; 3iarA; 3ib5A; 3ib7A; 3ibmA; 3ibzA; 3ic3A; 3ic4A; 3icvA; 3idfA; 3iduA; 3idwA; 3ie4A; 3ieeA; 3ieiA; 3iezA; 3ifnP; 3ig9A; 3ighX; 3igrA; 3ihsA; 3ihtA; 3ihuA; 3ihvA; 3ii2A; 3ii7A; 3iibA; 3iiiA; 3iijA; 3iisM; 3ij3A; 3ij6A; 3ijdA; 3ijmA; 3ijwA; 3ikwA; 3i1wA; 3i1xA; 3im1A; 3im3A; 3im6A; 3imhA; 3imkA; 3imoA; 3iosA; 3ioxA; 3ip0A; 3ipcA; 3iq2A; 3iqtA; 3iquA; 3ir4A; 3irbA; 3irpX; 3irsA; 3is6A; 3isaA; 3isqA; 3isrA; 3isxA; 3it3A; 3it4B; 3it4A; 3iteA; 3itfA; 3itqA; 3iu0A; 3iu5A; 3iu6A; 3iufA; 3iugA; 3iukA; 3iuoA; 3iupA; 3iusA; 3iuwA; 3iuzA; 3iv0A; 3iveA; 3ivfA; 3ivvA; 3iwfA; 3iwtA; 3ix3A; 3ixcA; 3ix1A; 3ixsA; 3ixsB; 3jq0A; 3jq1A; 3jqwA; 3jqyA; 3jr7A; 3jrvA; 3jrvC; 3jsyA; 3jszA; 3jtmA; 3jtwA; 3jtxA; 3jtzA; 3ju3A; 3ju4A; 3judA; 3juiA; 3jumA; 3juuA; 3jx9A; 3jxoA; 3jygA; 3jyoA; 3jysA; 3jyzA; 3jz0A; 3jz9A; 3jzyA; 3k01A; 3k05A; 3k0bA; 3k0zA; 3k1 1A; 3k12A; 3k13A; 3k1hA; 3k1tA; 3k1zA; 3k29A; 3k2oA; 3k2vA; 3k2zA; 3k3cA; 3k4iA; 3k50A; 3k5jA; 3k67A; 3k69A; 3k6 mA; 3k6oA; 3k6qA; 3k6yA; 3k7cA; 3k7iB; 3k7 pA; 3k7xA; 3k8uA; 3k8wA; 3k9oA; 3ka5A; 3ka7A; 3kb9A; 3kbgA; 3kbqA; 3kbyA; 3kc2A; 3kciA; 3kcpA; 3kcwA; 3kd3A; 3kd4A; 3kdwA; 3ke7A; 3kebA; 3keoA; 3kepA; 3kevA; 3kewA; 3kffA; 3kfoA; 3kg0A; 3kg4A; 3kgkA; 3kgrA; 3kgwA; 3kgyA; 3kgzA; 3kh1A; 3kh7A; 3kh8A; 3khiA; 3kizA; 3kk4A; 3kkfA; 3kkgA; 3kkiA; 3kkwA; 3kkzA; 3k1kA; 3k1qA; 3km5A; 3kmaA; 3kmhA; 3kmuA; 3kmvA; 3knvA; 3kogA; 3ko1A; 3kopA; 3korA; 3kosA; 3kp8A; 3kpbA; 3kpeA; 3kpeB; 3kq0A; 3kq5A; 3kqiA; 3ks6A; 3ksmA; 3ksxA; 3kt7A; 3kt9A; 3ktaA; 3ktaB; 3ktcA; 3ktoA; 3ku3A; 3ku3B; 3kvhA; 3kvtA; 3kweA; 3kw1A; 3kwrA; 3kwsA; 3kwuA; 3kxwA; 3kyjA; 3kyzA; 3kzdA; 3kzpA; 3kzxA; 3l00A; 3l0qA; 3l15A; 3l1eA; 3l1 nA; 3l1wA; 3l2hA; 3l34A; 3l39A; 3l3fX; 3l3uA; 3l4lA; 3l46A; 3l4aA; 3l4eA; 3l4hA; 3l4 nA; 3l4rA; 3l51B; 3l51A; 3l5iA; 3l60A; 3l6aA; 3l6bA; 3l6gA; 3l7xA; 3l81A; 3l8dA; 3l8eA; 3l8hA; 3l8qA; 3l8uA; 3l9aX; 3l9fA; 3l9uA; 3l9wA; 3laaA; 3lagA; 3lasA; 3lbeA; 3lccA; 3lcrA; 3ld7A; 3ldcA; 3ldvA; 3le4A; 3ledA; 3leqA; 3letA; 3lewA; 3lf9A; 3lfjA; 3lfrA; 3ftA; 3lg3A; 3lgbA; 3lgdA; 3lgiA; 3lheA; 3lhiA; 3lhnA; 3lhoA; 3lhrA; 3lhxA; 3li9A; 3lidA; 3ljkA; 3ljuX; 3lk5A; 3lk7A; 3lkeA; 3lkkA; 3lkmA; 3l17A; 3l1bA; 3l1cA; 3l1oA; 3l1 pA; 3l1uA; 3l1vA; 3lm2A; 3lm3A; 3lm4A; 3lmaA; 3lmoA; 3lmzA; 3ln1A; 3lo8A; 3logA; 3lopA; 3louA; 3lpeB; 3lpeA; 3lpwA; 3lpzA; 3lq0A; 3lqbA; 3lqhA; 3lqyA; 3lr0A; 3lrkA; 3lrtA; 3ls9A; 3lsnA; 3lssA; 3ltiA; 3lufA; 3lu1A; 3lumA; 3lurA; 3luuA; 3lvuA; 3lwaA; 3lwxA; 3lx3A; 3lx4A; 3lxqA; 3lxrF; 3ly0A; 3ly1A; 3ly7A; 3lydA; 3lygA; 3lyhA; 3lyyA; 3lzaA; 3lz1A; 3m07A; 3m0mA; 3m0zA; 3m1dA; 3m1eA; 3m1tA; 3m1uA; 3m1xA; 3m3hA; 3m3 pA; 3m4iA; 3m4rA; 3m5rA; 3m66A; 3m6jA; 3m6wA; 3m6zA; 3m70A; 3m73A; 3m7aA; 3m7fB; 3m7oA; 3m86A; 3m8eA; 3m8jA; 3m97X; 3m91A; 3m9qA; 3mabA; 3naoA; 3mazA; 3mbkA; 3mc3A; 3mcfA; 3mcwA; 3mcxA; 3mczA; 3md7A; 3md9A; 3mdmA; 3mdpA; 3mdqA; 3mduA; 3me5A; 3me7A; 3meaA; 3mewA; 3mf7A; 3mgdA; 3mggA; 3mi1A; 3mjfA; 3mjoA; 3mk1A; 3m11A; 3m11B; 3mm5A; 3mm5B; 3mmgA; 3mmhA; 3mmyA; 3mmyB; 3mn7S; 3mozA; 3mp6A; 3mprA; 3mq0A; 3mq2A; 3mqdA; 3mqhA; 3mqqA; 3mqzA; 3mstA; 3mswA; 3msxB; 3mt0A; 3mtqA; 3mtrA; 3mtwA; 3mu7A; 3mtujA; 3mvcA; 3mvnA; 3mvpA; 3mvsA; 3mvuA; 3mw8A; 3mwbA; 3mwpA; 3mwqA; 3mwxA; 3mwzA; 3mx7A; 3mxnA; 3mxnB; 3mxoA; 3mxzA; 3myfA; 3myuA; 3myxA; 3mz2A; 3mzfA; 3mzoA; 3n01A; 3n08A; 3n0rA; 3n0uA; 3n10A; 3n1eA; 3n27A; 3n29A; 3n2wA; 3n3 mA; 3n4jA; 3n6tA; 3n6yA; 3n6zA; 3n72A; 3n75A; 3n77A; 3n79A; 3n8bA; 3n90A; 3n9bA; 3n9uC; 3na6A; 3nbcA; 3nbmA; 3nd1A; 3ndcA; 3ndqA; 3ne0A; 3ne8A; 3nehA; 3neuA; 3nf5A; 3nfiA; 3nfkA; 3nfqA; 3nftA; 3nfwA; 3ng2A; 3ng7X; 3nggA; 3nheA; 3ni0A; 3nirA; 3nj2A; 3njdA; 3njeA; 3njnA; 3nk4A; 3nk4C; 3nkdA; 3nkeA; 3nkgA; 3nk1A; 3nksA; 3n19A; 3nnbA; 3no0A; 3no2A; 3no3A; 3no4A; 3no6A; 3no7A; 3nohA; 3nojA; 3nokA; 3noqA; 3npdA; 3npfA; 3nqiA; 3ngnA; 3nr5A; 3nreA; 3nrfA; 3nrhA; 3nrrA; 3nrvA; 3nrwA; 3nrxA; 3ns6A; 3nswA; 3nsxA; 3nt1A; 3ntuA; 3ntvA; 3nuaA; 3nufA; 3nuqA; 3nvsA; 3nvtA; 3nvwC; 3nvwB; 3nvxA; 3nwcA; 3nwoA; 3ny7A; 3nycA; 3nymA; 3nytA; 3nyvA; 3nyyA; 3nzeA; 3nz1A; 3nzmA; 3nmA; 3nztA; 3nzzA; 3o0fA; 3o0qA; 3o0yA; 3o10A; 3o12A; 3o14A; 3o1cA; 3o22A; 3o2eA; 3o2hA; 3o2rA; 3o2tA; 3o2uA; 3o3 mA; 3o3mB; 3o3uN; 3o3xA; 3o48A; 3o53A; 3o5qA; 3o6cA; 3o6 nA; 3o6 pA; 3o70A; 3o7bA; 3o7iA; 3o8bA; 3o8 mA; 3o94A; 3oa5A; 3oa8A; 3oa8B; 3oajA; 3obeA; 3obqA; 3oc9A; 3ocjA; 3ocmA; 3ocrA; 3ocuA; 3od1A; 3odtA; 3oe3A; 3oepA; 3of4A; 3of5A; 3of7A; 3ofgA; 3og2A; 3og9A; 3ogaA; 3oghA; 3ognA; 3oheA; 3ohgA; 3ohrA; 3oi8A; 3oioA; 3oisA; 3oizA; 3oj0A; 3oj7A; 3okpA; 3okxA; 3o10A; 3o13A; 3om0A; 3omdA; 3omtA; 3omyA; 3on2A; 3on4A; 3on9A; 3ondA; 3onjA; 3oo8A; 3oopA; 3oosA; 3oouA; 3ooxA; 3op6A; 3op7A; 3op8A; 3op9A; 3oqiA; 3oqpA; 3oqvA; 3oruA; 3oseA; 3osqA; 3osrA; 3ostA; 3ot1A; 3ot2A; 3otiA; 3otmA; 3otnA; 3ou2A; 3ougA; 3ouiA; 3ouvA; 3ov8A; 3ov9A; 3ovkA; 3owaA; 3owcA; 3owrA; 3owtC; 3owvA; 3oxhA; 3oxpA; 3oyvA; 3oz2A; 3p02A; 3p0bA; 3p0fA; 3p0yA; 3p1vA; 3p1xA; 3p24A; 3p2hA; 3p2tA; 3p3cA; 3p3vA; 3p42A; 3p4gA; 3p4hA; 3p4lA; 3p5 pA; 3p6bA; 3p6iA; 3p61A; 3p8aA; 3p8kA; 3p9aA; 3p9 nA; 3p9vA; 3pa6A; 3pajA; 3pasA; 3pb6X; 3pc3A; 3pc6A; 3pc7A; 3pd7A; 3pddA; 3pdfA; 3pdtA; 3pe6A; 3pe7A; 3pe9A; 3pesA; 3pf2A; 3pf6A; 3pf7A; 3pf9A; 3pfeA; 3pfgA; 3pfoA; 3pfsA; 3pfyA; 3pg0A; 3pg6A; 3pguA; 3ph9A; 3phsA; 3pi7A; 3picA; 3pidA; 3pijA; 3pimA; 3piwA; 3pj0A; 3pj1A; 3pjpA; 3pjvD; 3pjyA; 3pkvA; 3pkzA; 3p10A; 3p18A; 3p1wA; 3pm2A; 3pmcA; 3pmmA; 3pmoA; 3pn3A; 3pnaA; 3pnnA; 3pnxA; 3pnzA; 3po8A; 3pohA; 3pojA; 3powA; 3pp2A; 3pp5A; 3pp9A; 3pp1A; 3ppmA; 3pqcA; 3pr6A; 3pr9A; 3proC; 3ps0A; 3pshA; 3psmA; 3pstA; 3pt1A; 3pt3A; 3pt5A; 3ptyA; 3pu9A; 3puaA; 3pveA; 3pwfA; 3pywA; 3pzfA; 3pzjA; 3q13A; 3q18A; 3q1cA; 3q1nA; 3q1xA; 3q27A; 3q2eA; 3q2uA; 3q3qA; 3q49B; 3q60A; 3q64A; 3q6bA; 3q6sA; 3q7cA; 3q7 mA; 3q7rA; 3q8gA; 3q98A; 3qaoA; 3qayA; 3qb8A; 3qbmA; 3qbtB; 3qbyA; 3qc0A; 3qc5X; 3qc7A; 3qdhA; 3qd1A; 3qe2A; 3qekA; 3qf2A; 3qf7A; 3qf7C; 3qf1A; 3qfmA; 3qftA; 3qguA; 3qh4A; 3qh6A; 3qhbA; 3qhoA; 3qhpA; 3qhqA; 3qi7A; 3qioA; 3qitA; 3qktA; 3q16A; 3q19A; 3q1eA; 3q1jA; 3qnmA; 3qnsA; 3qooA; 3qorA; 3qp3A; 3qp4A; 3qp6A; 3qpaA; 3qraA; 3qr1A; 3qs2A; 3qsgA; 3qsjA; 3qs1A; 3qtaA; 3qu3A; 3qu5A; 3qufA; 3qv1A; 3qvpA; 3qvsA; 3qw3A; 3qw9A; 3qwgA; 3qw1A; 3qxbA; 3qxhA; 3qy1A; 3qy3A; 3qy7A; 3qyfA; 3qzbA; 3r0nA; 3r0vA; 3r1pA; 3r2vA; 3r3qA; 3r3rA; 3r4zA; 3r5dA; 3r5tA; 3r5zA; 3r62A; 3r6aA; 3r6dA; 3r6fA; 3r72A; 3r7aA; 3r87A; 3r89A; 3r8eA; 3r8jA; 3r8yA; 3r90A; 3r9fA; 3r9 mA; 3ragA; 3rayA; 3razA; 3rbsA; 3rc1A; 3rcoA; 3rd5A; 3rd7A; 3rdeA; 3rdoA; 3rdvA; 3rdyA; 3regA; 3renA; 3retA; 3rfeA; 3rfiA; 3rftA; 3rg9A; 3rgaA; 3rh0A; 3rhbA; 3rhgA; 3rhtA; 3rioA; 3rjtA; 3rjuA; 3rjvA; 3rk6A; 3rkgA; 3rk1A; 3r15A; 3r1gA; 3r1kA; 3r1oA; 3r1sA; 3rm3A; 3rmhA; 3rmjA; 3rmqA; 3rmuA; 3m1A; 3mqA; 3mqB; 3mrA; 3robA; 3rofA; 3rotA; 3rp8A; 3rpcA; 3rpdA; 3rpfA; 3rpfC; 3rpjA; 3rppA; 3rpwA; 3rpzA; 3rq4A; 3rq9A; 3rqiA; 3rgtA; 3rqzA; 3rr6A; 3rriA; 3rrxA; 3rtaA; 3rt1A; 3ru6A; 3ruiA; 3rv1A; 3rvcA; 3rwaA; 3rwnA; 3rx9A; 3rxyA; 3ry4A; 3rykA; 3rznA; 3rzvA; 3s06A; 3sOaA; 3s1xA; 3s25A; 3s2jA; 3s2rA; 3s3tA; 3s3zA; 3s44A; 3s4eA; 3s4yA; 3s5bA; 3s5 mA; 3s5qA; 3s5wA; 3s6bA; 3s6eA; 3s83A; 3s8gA; 3s8gB; 3s8gC; 3s8iA; 3s8 mA; 3s8sA; 3s90C; 3s9fA; 3s9jA; 3s9xA; 3sb1A; 3sb4A; 3sbmA; 3sbtA; 3sbtB; 3sc7X; 3scyA; 3sd7A; 3sdbA; 3sdeA; 3se5A; 3seeA; 3sfvB; 3sfxB; 3sg0A; 3sggA; 3sh4A; 3shgB; 3shgA; 3shoA; 3shqA; 3sibA; 3si1A; 3sjhB; 3sj1D; 3sj1A; 3sjqC; 3sk2A; 3sk7A; 3sk9A; 3skxA; 3s11A; 3s12A; 3s1rA; 3s1zA; 3smaA; 3smdA; 3smvA; 3smzA; 3snoA; 3so5A; 3so6A; 3soeA; 3sojA; 3sonA; 3soyA; 3sp7A; 3sp8A; 3sq7A; 3sqfA; 3sqzA; 3sreA; 3sriA; 3sriB; 3ss7X; 3ssbC; 3ssbI; 3ssoA; 3su6A; 3sukA; 3sumA; 3suvA; 3sw0X; 3swoA; 3swyA; 3sxmA; 3sxuA; 3sxuB; 3sxyA; 3sy1A; 3sy6A; 3sz3A; 3sz7A; 3szaA; 3szvA; 3szyA; 3t0hA; 3t0A; 3t31A; 3t3wA; 3t4lA; 3t43A; 3t47A; 3t49A; 3t4lA; 3t64A; 3t6 pA; 3t6sA; 3t7dA; 3t7hA; 3t71A; 3t8kA; 3t8wA; 3t92A; 3t94A; 3t9yA; 3tbjA; 3tbmA; 3tbnA; 3tboA; 3tc3A; 3tc5A; 3tc8A; 3tcqA; 3tcvA; 3tdsA; 3te8A; 3teeA; 3tejA; 3tekA; 3tewA; 3tg0A; 3tg2A; 3tghA; 3thgA; 3thkA; 3ti2A; 3tiaA; 3tjeF; 3tjmA; 3tjyA; 3tk8A; 3tm4A; 3tm8A; 3tosA; 3towA; 3tp4A; 3tr9A; 3trbA; 3trdA; 3trgA; 3trqA; 3ts3A; 3ts9A; 3tt9A; 3ttcA; 3ttgA; 3tu3B; 3tu3A; 3tu8A; 3tuoA; 3tvjA; 3tvqA; 3txsA; 3ty1A; 3typA; 3tysA; 3u0bA; 3u1dA; 3u1jA; 3u11A; 3u1uA; 3u1wA; 3u2aA; 3u2sC; 3u2uA; 3u31C; 3u3zA; 3u4gA; 3u4vA; 3u5vA; 3u65A; 3u6gA; 3u7qA; 3u7qB; 3u7zA; 3u8vA; 3u97A; 3u99A; 3u9gA; 3u9rB; 3u9wA; 3uanA; 3ub1A; 3ub6A; 3uc9A; 3ucjA; 3ucpA; 3ucsC; 3ucsA; 3ud1A; 3udfA; 3ue2A; 3uejA; 3uekA; 3uenA; 3uf6A; 3uf8A; 3ufbA; 3ufeA; 3ugfA; 3uguA; 3uidA; 3ujcA; 3u1bA; 3u1jA; 3u1tA; 3umhA; 3umoA; 3unnA; 3uoaB; 3up3A; 3up1A; 3upsA; 3upvA; 3uq8A; 3ur8A; 3urgA; 3urrA; 3ushA; 3utkA; 3utmA; 3utmC; 3uueA; 3uu1A; 3uuwA; 3uv0A; 3uv4A; 3uv9A; 3ux2A; 3uxfA; 3uxjA; 3v0dA; 3v0rA; 3v1aA; 3v1vA; 3v34A; 3v31A; 3v46A; 3v4cA; 3v4gA; 3v4kA; 3v5cA; 3v68A; 3v6oA; 3v75A; 3v7bA; 3v7 nA; 3v7qA; 3v9oA; 3vaaA; 3vbcA; 3vc1A; 3vcaA; 3vcxA; 3vdjA; 3vejA; 3venA; 3vfzA; 3vgiA; 3vgpA; 3vgzA; 3vh8G; 3vhjA; 3vi6A; 3viiA; 3vj9A; 3vk0A; 3vk5A; 3vk6A; 3v1aA; 3vmnA; 3vmvA; 3vn0A; 3vn3A; 3vn5A; 3vocA; 3vorA; 3votA; 3vp5A; 3vpbA; 3vpbE; 3vpyA; 3vpzA; 3vgjA; 3vqtA; 3vrdB; 3vrdA; 3vsnA; 3vthA; 3vtoA; 3vtxA; 3vubA; 3vupA; 3vv1A; 3vvvA; 3vvyA; 3vwnX; 3vxcA; 3vypA; 3vz9B; 3vz9D; 3w06A; 3w08A; 3w0fA; 3w0kA; 3w0oA; 3w0tA; 3w15A; 3w15B; 3w1oA; 3w20A; 3w36A; 3w42A; 3w4rA; 3w4sA; 3w57A; 3w5fA; 3w5jA; 3w5 nA; 3w5sA; 3w5xA; 3w63A; 3w6bA; 3w6sA; 3w6wA; 3w7tA; 3w9kA; 3wa1A; 3wa2X; 3wa5B; 3waiA; 3warA; 3wasA; 3wdhA; 3wdqA; 3we5A; 3we9A; 3weoA; 3weuA; 3wfvA; 3wg3A; 3wgxA; 3wgxC; 3wh1A; 3wh2A; 3whjA; 3wiaA; 3wihA; 3wisA; 3witA; 3wiwA; 3wjpA; 3wkgA; 3wkyA; 3w1iA; 3wmiA; 3wmiB; 3wmqA; 3wmtA; 3wmvA; 3wmyA; 3wndA; 3wnoA; 3wnzA; 3wpaA; 3wppA; 3wpuA; 3wqbA; 3wqbB; 3wqcA; 3wsgA; 3wt0A; 3wu4A; 3wurA; 3wuzA; 3wv7A; 3wvaA; 3wvqA; 3wvtA; 3wvzA; 3ww8A; 3ww9A; 3wwaA; 3wwcA; 3wwhA; 3wwIA; 3wwnA; 3wwqC; 3wx4A; 3wx7A; 3wxyA; 3wydA; 3wz3A; 3wzsA; 3x0iA; 3x0tA; 3x0uA; 3x0vA; 3x2 mA; 3x30A; 3x34A; 3x38A; 3zbdA; 3zbqA; 3zdmA; 3zdsA; 3zeuA; 3zeuB; 3zg1A; 3zg9B; 3zg9A; 3zh0A; 3zhiA; 3zhnA; 3zi1A; 3zibA; 3zidA; 3zitA; 3zj0A; 3zjaA; 3zjbA; 3zl8A; 3zmdA; 3zmrA; 3zn4A; 3zn6A; 3znvA; 3zojA; 3zoqB; 3zpxA; 3zpyA; 3zqkA; 3zqoA; 3zrdA; 3zrgA; 3zriA; 3zrqA; 3zrxA; 3zscA; 3zsjA; 3zsuA; 3zt9A; 3ztvA; 3zucA; 3zuiA; 3zuzA; 3zv1A; 3zvqA; 3zvqB; 3zvsA; 3zw5A; 3zx1A; 3zx4A; 3zxcA; 3zxkA; 3zxnA; 3zxoA; 3zxyA; 3zy7A; 3zy1A; 3zypA; 3zyqA; 3zywA; 3zzoA; 3zzpA; 3zzsA; 4a02A; 4a0dA; 4a0pA; 4a0tA; 4a0zA; 4a1iA; 4a29A; 4a2vA; 4a35A; 4a37A; 4a3 pA; 4a3xA; 4a3zA; 4a42A; 4a4jA; 4a56A; 4a57A; 4a5 nA; 4a5sA; 4a5uB; 4a5xA; 4a6hA; 4a6qA; 4a7kA; 4a7wA; 4a8uA; 4a8xA; 4a8xB; 4a94C; 4aajA; 4aanA; 4ab1A; 4ac1X; 4ac7C; 4ac7A; 4ac7B; 4aciA; 4acjA; 4adiA; 4admA; 4adnA; 4adzA; 4ae2A; 4ae5A; 4ae7A; 4aeqA; 4afA 4affA; 4afkA; 4afmA; 4aghA; 4agkA; 4aivA; 4ajyC; 4ajyV; 4ajyB; 4ak1A; 4ak2A; 4ak5A; 4a10A; 4a1zA; 4ammA; 4ap9A; 4apoA; 4apxB; 4aq1A; 4aqoA; 4aqrD; 4ar9A; 4armA; 4ascA; 4asmB; 4at0A; 4at7B; 4at7A; 4ateA; 4atgA; 4athA; 4atmA; 4aumA; 4avaA; 4avrA; 4avsA; 4aw2A; 4aw7A; 4ax2A; 4axiA; 4ay0A; 4ay7A; 4aycA; 4ay1A; 4az6A; 4b0aA; 4b0mA; 4b1mA; 4b29A; 4b2fA; 4b2hA; 4b46A; 4b4cA; 4b4sA; 4b4uA; 4b60A; 4b62A; 4b6gA; 4b6iA; 4b6 mA; 4b89A; 4b8eA; 4b8vA; 4b93B; 4b93A; 4b9dA; 4b9gA; 4b9iA; 4b9kA; 4ba0A; 4bb9A; 4bc3A; 4bdxA; 4be3A; 4begA; 4beuA; 4bfaA; 4bfcA; 4bfoA; 4bg7A; 4bgbA; 4bgcA; 4bgoA; 4bgpA; 4bh5A; 4bhuA; 4bi3A; 4bi8A; 4bixA; 4bj0A; 4bjaA; 4bjiA; 4bjsA; 4bjtA; 4bjtD; 4bjzA; 4bk0A; 4bk8A; 4b10B; 4b1pA; 4bmhA; 4bnrI; 4bo1A; 4boqA; 4bouA; 4bpfA; 4bpsA; 4bpyA; 4bq2A; 4bqhA; 4bqnA; 4bt7A; 4bt9A; 4bu0A; 4bv1A; 4bvaA; 4bvwA; 4bvxB; 4bvxA; 4bwcA; 4bwrA; 4byzA; 4bz4A; 4bz7A; 4bzaA; 4bzpA; 4c08A; 4c0nA; 4c12A; 4c18A; 4c1oA; 4c1uA; 4c1wA; 4c24A; 4c2eA; 4c3sA; 4c4aA; 4c4pB; 4c5cA; 4c5eA; 4c5eE; 4c5wA; 4c6aA; 4c6eA; 4c6sA; 4c76A; 4c97A; 4c9bB; 4c9sA; 4cahB; 4cayA; 4cayB; 4cayC; 4cbeA; 4cbuA; 4cbuG; 4ccdA; 4ccsA; 4ccvA; 4ccwA; 4cd8A; 4cdjA; 4cdpA; 4ce7A; 4cfiA; 4cfqQ; 4cfsA; 4cgoA; 4cgqA; 4cgsA; 4ch7A; 4cheA; 4ci7A; 4ci9A; 4cicA; 4ci1A; 4citA; 4cj0B; 4cj0A; 4cj2A; 4ck4A; 4c11A; 4cmrA; 4cn0A; 4cn9A; 4cndA; 4cngA; 4cn1A; 4co8A; 4cogA; 4cosA; 4cpyA; 4cq4A; 4cqbA; 4cqiA; 4crhA; 4cruB; 4cruA; 4crwA; 4cs4A; 4csrA; 4csrB; 4ct3A; 4cu9A; 4cuaA; 4cv4A; 4cv7A; 4cvbA; 4cvdA; 4cvoA; 4cvrA; 4cvuA; 4cw4A; 4cxfA; 4cxfB; 4cybA; 4czgA; 4czuA; 4czxA; 4czxB; 4d02A; 4d05A; 4d0pA; 4d0qA; 4d11A; 4d3xA; 4d40A; 4d4zA; 4d53A; 4d5aA; 4d5bA; 4d5rA; 4d6gA; 4d73A; 4d77A; 4d7jA; 4d7 pA; 4d86A; 4d8bA; 4d9bA; 4d9iA; 4da2A; 4damA; 4db5A; 4dbbA; 4dd5A; 4ddpA; 4de9A; 4devA; 4deyB; 4df0A; 4dgfA; 4dh2B; 4dhiB; 4di9A; 4dipA; 4dixA; 4djaA; 4djgA; 4dk2A; 4dkaC; 4dkcA; 4d1hA; 4d1qA; 4dm1A; 4dm5A; 4dm7A; 4dmgA; 4dmiA; 4dmvA; 4dn7A; 4dnyA; 4do4A; 4do7A; 4dpbX; 4dpzX; 4dq6A; 4dq9A; 4dqaA; 4dqjA; 4driB; 4dt4A; 4dt5A; 4dthA; 4dv8A; 4dvcA; 4dwsA; 4dyqA; 4dz1A; 4dz4A; 4dziA; 4dzoA; 4dzzA; 4e01A; 4e0aA; 4e15A; 4e19A; 4e1bA; 4e1oA; 4e1pA; 4e29A; 4e2aA; 4e2gA; 4e2uA; 4e2zA; 4e3eA; 4e3yA; 4e40A; 4e45A; 4e45B; 4e45E; 4e4rA; 4e57A; 4e5rA; 4e5vA; 4e6fA; 4e6kG; 4e6uA; 4e74A; 4e8dA; 4e97A; 4e91A; 4e9sA; 4e9xA; 4ea9A; 4eadA; 4eaeA; 4ebbA; 4ebgA; 4ebjA; 4ebyA; 4ecfA; 4ed9A; 4edhA; 4edpA; 4edqA; 4ee6A; 4eetB; 4efoA; 4efpA; 4egcB; 4egcA; 4egdA; 4eguA; 4ehcA; 4ehsA; 4ehuA; 4ehxA; 4ei0A; 4eibA; 4eicA; 4eiuA; 4eivA; 4ekfA; 4ekxA; 4e16A; 4e11A; 4emdA; 4emtA; 4eo0A; 4eo1A; 4eo3A; 4ep4A; 4ep8B; 4epiA; 4epsA; 4epzA; 4eq8A; 4eqaC; 4eqaA; 4eqbA; 4eqpA; 4eqsA; 4ercA; 4errA; 4eryA; 4es1A; 4es8A; 4esmA; 4esqA; 4esrA; 4esuA; 4eswA; 4etyA; 4eu9A; 4eukA; 4eunA; 4euoA; 4euuA; 4ev8A; 4evfA; 4evmA; 4evqA; 4evuA; 4evwA; 4evxA; 4evyA; 4ew5A; 4ew7A; 4eweA; 4ewfA; 4ex6A; 4exkA; 4exoA; 4exrA; 4eyzA; 4ezgA; 4eziA; 4fD1A; 4fD3A; 4fD6A; 4fDjA; 4fDwA; 4fDzC; 4f1jA; 4f1vA; 4f27A; 4f2eA; 4f21A; 4f2 nA; 4f3 mA; 4f3 nA; 4f3vA; 4f4hA; 4f54A; 4f55A; 4f67A; 4f6 pA; 4f6tA; 4f7uB; 4f7uG; 4f7uA; 4f7uE; 4f7uF; 4f7uP; 4f80A; 4f87A; 4f8cA; 4f8kA; 4f98A; 4f9dA; 4faiA; 4fayA; 4fb2A; 4fb7A; 4fbcA; 4fbjA; 4fbrA; 4fbsA; 4fc9A; 4fcgA; 4fchA; 4fcjA; 4fd4A; 4fd7A; 4fdbA; 4fe3A; 4fekA; 4fetA; 4fevA; 4ff5A; 4ff1A; 4ffuA; 4fg1A; 4fgqA; 4fh0A; 4fhgA; 4fhrA; 4fhrB; 4fibA; 4fk9A; 4fkbA; 4fkeA; 4f13A; 4f1bA; 4fm1A; 4fmzA; 4fn7A; 4fnvA; 4fojA; 4fp5D; 4fqgA; 4fqnA; 4fr9A; 4fs7A; 4fs8A; 4ftdA; 4ftfA; 4fvdA; 4fvgA; 4fw1A; 4fx5A; 4fxiA; 4fxqA; 4fypA; 4fzoA; 4fzvA; 4fzvB; 4g0xA; 4g1iA; 4g1qA; 4g1qB; 4g22A; 4g26A; 4g29A; 4g2uA; 4g38A; 4g3aA; 4g3fA; 4g3 nA; 4g3oA; 4g3vA; 4g4gA; 4g4kA; 4g5aA; 4g68A; 4g6tA; 4g6tB; 4g6xA; 4g75A; 4g78A; 4g79A; 4g71A; 4g7xB; 4g7xA; 4g94B; 4g94A; 4g9eA; 4g9 mA; 4g9qA; 4g9sA; 4g9sB; 4ga2A; 4gakA; 4gb5A; 4gb7A; 4gbfA; 4gbmA; 4gc3A; 4gc5A; 4gcoA; 4gd5A; 4gdaA; 4gdoA; 4gdzA; 4gehA; 4gehB; 4gekA; 4ge1A; 4gf3A; 4gf3B; 4gftA; 4ggcA; 4ggfC; 4gggA; 4ggjA; 4gh9A; 4ghnA; 4gi3C; 4gi5A; 4gimA; 4gj4A; 4gjzA; 4gk6A; 4gkgA; 4g1qA; 4gm6A; 4gmqA; 4gmuA; 4gn0A; 4gn4B; 4gneA; 4gnrA; 4gofA; 4goqA; 4gosA; 4gpvA; 4gq4A; 4gqmA; 4gqnA; 4gqzA; 4gr2A; 4gr6A; 4grdA; 4gt6A; 4gt8A; 4gt9A; 4gucA; 4gudA; 4gvbB; 4gvfA; 4gvqA; 4gwbA; 4gwgA; 4gwmA; 4gx8A; 4gxbA; 4gxbB; 4gxtA; 4gxwA; 4gy7A; 4gymA; 4gywA; 4gzcA; 4gzkA; 4h08A; 4h0aA; 4h0cA; 4h0sA; 4h14A; 4h18A; 4h1xA; 4h27A; 4h2gA; 4h2wA; 4h3uA; 4h3vA; 4h3wA; 4h4lA; 4h4dA; 4h4 nA; 4h59A; 4h5bA; 4h5iA; 4h5sA; 4h6cA; 4h6qA; 4h6xA; 4h79A; 4h7uA; 4h7wA; 4h7yA; 4h87A; 4h89A; 4h8eA; 4h9nC; 4hadA; 4haeA; 4hamA; 4hatC; 4hatB; 4hb9A; 4hbqA; 4hbzA; 4hc5A; 4hchA; 4hcjA; 4hcsA; 4hczA; 4hddA; 4hdeA; 4hdoA; 4he6A; 4heiA; 4hemA; 4hesA; 4hf0A; 4hfqA; 4hfsA; 4hfvA; 4hg2A; 4hguA; 4hh3A; 4hh3C; 4hh5A; 4hhjA; 4hhrA; 4hhvA; 4hi8A; 4hi8B; 4hiaA; 4hjfA; 4hjgE; 4hjiA; 4hjzA; 4hkgA; 4hkhA; 4h12A; 4h1bA; 4hmsA; 4hn9A; 4hnoA; 4hpmA; 4hpmB; 4hq1A; 4hqzA; 4hroA; 4hs1A; 4hs2A; 4hsqA; 4hstB; 4hstA; 4htfA; 4ht1A; 4htrA; 4hu2A; 4hu8A; 4hujA; 4hvkA; 4hvtA; 4hvyA; 4hw0A; 4hw6A; 4hwhA; 4hwmA; 4hwvA; 4hxfB; 4hxyA; 4hy4A; 4hyeA; 4hziA; 4hzoA; 4i05A; 4i0oA; 4i0wB; 4i0wA; 4i0xB; 4i0xA; 4i17A; 4i1fA; 4i3gA; 4i4cA; 4i4kA; 4i4oA; 4i66A; 4i68A; 4i6 nA; 4i6rA; 4i6xA; 4i6yA; 4i71A; 4i79A; 4i84A; 4i8iA; 4i90A; 4i93A; 4i9oA; 4ia6A; 4iabA; 4iajA; 4iauA; 4ibgA; 4ibnA; 4ic3A; 4ic9A; 4ichA; 4iciA; 4icvA; 4id9A; 4idhA; 4idiA; 4iejA; 4ienA; 4ifaA; 4igiA; 4igkA; 4igvA; 4iibA; 4iikA; 4ii1A; 4iiyA; 4ij5A; 4ijnA; 4ijrA; 4ik8A; 4ikbA; 4ikdA; 4iknA; 4ikvA; 4i17A; 4i1fA; 4indA; 4ineA; 4inkA; 4inwA; 4inzA; 4ipcA; 4ipiA; 4ipuA; 4iq0A; 4ignA; 4iqyA; 4irgA; 4it6A; 4iu2A; 4iu6A; 4iujA; 4iumA; 4iupA; 4iusA; 4iuwA; 4ivgA; 4ivkA; 4ivnA; 4iwbA; 4ix3A; 4ixjA; 4iyaA; 4iyjA; 4iykA; 4iysA; 4iz7B; 4izxA; 4j0dA; 4j0wA; 4j25A; 4j27A; 4j32A; 4j32B; 4j33A; 4j37A; 4j3hA; 4j3vA; 4j42A; 4j44A; 4j4hA; 4j4zA; 4j6oA; 4j78A; 4j7hA; 4j7 nA; 4j7qA; 4j7zA; 4j87A; 4j8cA; 4j81A; 4j8sA; 4j9tA; 4j9yB; 4jaqA; 4jb3A; 4jb7A; 4jb8A; 4jbbA; 4jbdA; 4jbeA; 4jbuA; 4jccA; 4jdeA; 4jdnA; 4jduA; 4je5A; 4jeaA; 4jejA; 4jemA; 4jf3A; 4jf8A; 4jg2A; 4jgiA; 4jg1A; 4jhnA; 4jhyA; 4jifA; 4jifB; 4jiuA; 4jivD; 4jj7A; 4jjaA; 4jjoA; 4jk8A; 4jm1A; 4jmdA; 4jmgA; 4jmpA; 4jmqA; 4jn7A; 4jndA; 4jnuA; 4jo5A; 4jo7A; 4joqA; 4jp0A; 4jp6A; 4jqfA; 4jqpA; 4jquB; 4jr6A; 4jrfA; 4jtmA; 4jvoA; 4jvuA; 4jwjA; 4jwoA; 4jxeA; 4jxhA; 4jxrA; 4jykA; 4jysA; 4jz5A; 4jzzA; 4jzzR; 4k00A; 4k02A; 4k05A; 4k0nA; 4k12B; 4k12A; 4k22A; 4k2 mA; 4k2 pA; 4k35A; 4k3zA; 4k4kA; 4k6 nA; 4k70A; 4k73A; 4k7bA; 4k7cA; 4k7jA; 4k82A; 4k84A; 4k8wA; 4k90A; 4k90B; 4k9zA; 4ka1A; 4kcaA; 4kdrA; 4kdwA; 4ke2A; 4kefA; 4kemA; 4kfuA; 4kg3A; 4kg7A; 4kgdA; 4kh8A; 4kh9A; 4kiaA; 4kjmA; 4kk7A; 4k10A; 4k1kA; 4k1xA; 4km6A; 4kmdA; 4km1A; 4kmnA; 4kmrA; 4kn8A; 4knkA; 4ko8A; 4kopA; 4kq7A; 4kq9A; 4kqdA; 4kqiA; 4kqpA; 4krdB; 4krgA; 4kruA; 4ksnA; 4ksyA; 4kt3A; 4kt3B; 4kt6A; 4kt6B; 4ktwA; 4ku0A; 4ku0D; 4kuiA; 4kukA; 4kunA; 4kv2A; 4kv7A; 4kv9A; 4kvgB; 4kwaA; 4kxvA; 4kyqA; 4kyxA; 4kzkA; 4kzpA; 4l0cA; 4l0jA; 4l0 nA; 4l1jA; 4l2hA; 4l2iB; 4l2iA; 4l3uA; 4l51A; 4l57A; 4l58A; 4l5eA; 4l63A; 4l68A; 4l6hA; 4l7gA; 4l7 mA; 4l7 nA; 4l8aA; 4l8 pA; 4l9aA; 4l9bA; 4l9eA; 4l9hA; 4l9 nA; 4l9oA; 4l9 pA; 4l9pB; 4l9uA; 4la2A; 4lanA; 4layA; 4lbaA; 4lbhA; 4lc1A; 4ld1A; 4ld6A; 4ld8A; 4ldfA; 4ldmA; 41dvA; 4e1A; 4lebA; 4lerA; 4lDA; 4lftA; 4lg1A; 4lg8A; 4lgjA; 4lgoA; 4lgxA; 4lgyA; 4lhdA; 4lhsA; 4lijA; 4limA; 4lixA; 4lizA; 4lj1A; 4lj9A; 4ljiA; 4ljoA; 41ldA; 41ldB; 41loB; 41loA; 41lqB; 41lyA; 4lm6A; 4lm8A; 4lmiA; 4lmsA; 4lmyA; 4ln7A; 4losA; 4lowA; 4lpqA; 4lpsA; 4lq6A; 4lqbA; 4lqkA; 4lqzA; 4lrdA; 4lrjA; 4lrtA; 4lrtB; 4ls3A; 4lsbA; 4ltnA; 4lttA; 4ltyA; 4luaA; 4lukA; 4lunU; 4lv5B; 4lvfA; 4lw1A; 4lx2A; 4lx2B; 4lx3A; 4lx3B; 4lxoA; 4lxqA; 4ly7A; 4lypA; 4lzhA; 4lzkA; 4lz1A; 4lzxB; 4m02A; 4m0nA; 4m0wA; 4m1bA; 4m1gA; 4m1uA; 4m1xA; 4m20A; 4m2 mA; 4m37A; 4m3 pA; 4m51A; 4m5bA; 4m5dA; 4m5 dB; 4m5eA; 4m5rA; 4m6bA; 4m6bC; 4m7tA; 4m82A; 4m85A; 4m88A; 4m8aA; 4m8dA; 4m8iA; 4m8kA; 4m91A; 4m9 pA; 4maaA; 4maiA; 4makA; 4mamA; 4maqA; 4maxA; 4mb4A; 4mboA; 4mbyA; 4mc3A; 4mcoA; 4mdaA; 4mdwA; 4mdyA; 4me2A; 4me3A; 4mesA; 4mewA; 4mfiA; 4mfkA; 4mg4A; 4mgeA; 4mgqA; 4mhxA; 4mi4A; 4mi5A; 4mi7A; 4mixA; 4miyA; 4mjdA; 4mjfA; 4mkmA; 4mkoA; 4m11A; 4m17B; 4m19A; 4m1mA; 4m1oA; 4m1sA; 4m1vA; 4m1zA; 4mn5A; 4mn7A; 4mncA; 4mnoA; 4mnrA; 4mo4A; 4mo7A; 4mo9A; 4mpoA; 4mq3A; 4mrtC; 4ms4A; 4ms8A; 4mspA; 4msxA; 4mt8A; 4mt1A; 4mtuA; 4mu9A; 4muoA; 4mupA; 4muqA; 4murA; 4muvA; 4mv4A; 4mvfA; 4mwaA; 4mwiA; 4mxnA; 4mxpA; 4mxtA; 4mydA; 4my1A; 4mymA; 4myyA; 4myzA; 4mz7A; 4mzaA; 4mzcA; 4mzdA; 4mzgB; 4mzvA; 4mzyA; 4n01A; 4n02A; 4n03A; 4n0dA; 4n0hB; 4n0hF; 4n0kA; 4n0nA; 4n0pA; 4n0rA; 4n13A; 4n1iA; 4n11A; 4n21A; 4n2kA; 4n2 pA; 4n2xA; 4n30A; 4n3sA; 4n3tA; 4n49A; 4n4jA; 4n58A; 4n5hX; 4n5uA; 4n67A; 4n6aA; 4n6hA; 4n6jA; 4n6oB; 4n6qA; 4n6tA; 4n6wA; 4n77A; 4n7cA; 4n7iA; 4n7wA; 4n82A; 4n8 mA; 4n8 nA; 4n9oA; 4n9wA; 4naoA; 4nb5A; 4nbmA; 4nbpA; 4nbxA; 4nc6A; 4nc7A; 4nczA; 4ndjA; 4ndoB; 4ndsA; 4ne2A; 4ne3B; 4ne3A; 4necA; 4nexA; 4nf1A; 4ng0A; 4nhbA; 4ni2A; 4ni6A; 4nirA; 4njhA; 4njyA; 4nkpA; 4n19A; 4n19C; 4n1mA; 4nm9A; 4nmuA; 4nmwA; 4nmyA; 4nn2A; 4nn5B; 4nn5C; 4nn5A; 4nnbA; 4nnoA; 4nnrA; 4noaA; 4nobA; 4nohA; 4npdA; 4npfX; 4np1A; 4npnA; 4npsA; 4npxA; 4nrpA; 4ns5A; 4nsvA; 4nt1A; 4ntcA; 4ntdA; 4ntkA; 4nu3A; 4nuaA; 4nurA; 4nutB; 4nuuA; 4nuuC; 4nuzA; 4nv0A; 4nv4A; 4nwbA; 4nwkA; 4nwyA; 4nx8A; 4nxyA; 4nyhA; 4nyqA; 4nzjA; 4nzkA; 4nz1B; 4nznA; 4nzrM; 4nzvA; 4o06A; 4o0cA; 4o1eA; 4o1rA; 4o1wA; 4o36B; 4o3vA; 4o4fA; 4o4oA; 4o590; 4o5fA; 4o5qA; 4o65A; 4o66A; 4o6 mA; 4o6uA; 4o6yA; 4o7kA; 4o7qA; 4o87A; 4o8aA; 4o8oA; 4o8vA; 4o8yA; 4o9dA; 4o91A; 4oa3A; 4oahA; 4ob0A; 4ob1A; 4ocvA; 4od6A; 4od8A; 4od8C; 4od9B; 4od9A; 4odkA; 4odpA; 4oe9A; 4oebA; 4oe1B; 4oe1A; 4oevA; 4of8A; 4offA; 4ofkA; 4oh7A; 4ohjA; 4ohxA; 4oi3A; 4oieA; 4ojuA; 4ojxA; 4ok4A; 4okeA; 4okiA; 4okvE; 4okzA; 4o19A; 4o1tA; 4om8A; 4ombA; 4omfA; 4omfB; 4omfG; 4omgA; 4omjA; 4oncA; 4onmA; 4onrA; 4onwA; 4ooaA; 4oopA; 4ooxA; 4opcA; 4opmA; 4opwA; 4oq1A; 4oqjA; 4oqpA; 4oqvA; 4ou0A; 4ou9A; 4ouhA; 4oujA; 4ounA; 4ousA; 4ov4A; 4ovjA; 4ovkA; 4ovyA; 4ow1A; 4owfA; 4owfG; 4owkA; 4owtA; 4owtB; 4owtC; 4ox3A; 4ox5A; 4ox6A; 4oxwA; 4oyuA; 4ozjA; 4ozuA; 4ozwA; 4ozxA; 4p09A; 4p0dA; 4p0gA; 4p0tA; 4p0zA; 4p17A; 4p1mA; 4p29A; 4p2iA; 4p3aA; 4p3fA; 4p3hA; 4p3vA; 4p3wA; 4p3wG; 4p40A; 4p5eA; 4p5 nA; 4p6bA; 4p6qA; 4p7cA; 4p7oA; 4p7tA; 4p7xA; 4p82A; 4p8 nA; 4p98A; 4p99A; 4p9iA; 4pabA; 4pagA; 4pasA; 4pasB; 4pauA; 4pbdA; 4pc3C; 4pdcE; 4pdnA; 4pdyA; 4pe6A; 4peoA; 4petA; 4peuA; 4pf3A; 4pfoA; 4pfyA; 4pgrA; 4ph2A; 4ph8A; 4phjA; 4phqA; 4phrA; 4pi8A; 4pibA; 4pioA; 4pj2C; 4pj2A; 4pjrA; 4pk9A; 4pkfA; 4pkfC; 4pkfB; 4pkgG; 4pk1A; 4pkmA; 4p16A; 4p18H; 4p19A; 4p1zA; 4pmoA; 4pmqA; 4pmxA; 4pneA; 4pnoA; 4po6B; 4ponA; 4pp4A; 4pp8C; 4pprA; 4pq8A; 4pqdA; 4pqgA; 4pqhA; 4pqjA; 4pqqA; 4ps2A; 4ps6A; 4psfA; 4psrA; 4psyA; 4ptzA; 4pu5A; 4pu7A; 4puhA; 4puiA; 4puxA; 4pv2B; 4pv2A; 4pvkA; 4pw0A; 4pw2A; 4pwoA; 4pwwA; 4pwyA; 4pxeA; 4pxwA; 4pxyA; 4pyaA; 4pyhA; 4pyrA; 4pysA; 4pz0A; 4pz1A; 4pzaA; 4pzjA; 4q0yA; 4q2qA; 4q2sA; 4q2uA; 4q2wA; 4q3jA; 4q3kA; 4q3oA; 4q4gX; 4q51A; 4q53A; 4q5eA; 4q5wA; 4q62A; 4q63A; 4q68A; 4q6kA; 4q6uA; 4q6vA; 4q7fA; 4q7oA; 4q7qA; 4q82A; 4q8kA; 4q8rA; 4q8wA; 4q98A; 4q9bA; 4qa8A; 4qasA; 4qb0A; 4qbbA; 4qb1A; 4qbnA; 4qboA; 4qbsA; 4qbuA; 4qc6A; 4qcjA; 4qdcA; 4qdjA; 4qdnA; 4qe0A; 4qekA; 4qfuA; 4qgoA; 4qgpA; 4qgsA; 4qhjA; 4qhqA; 4qhwA; 4qi3A; 4qiuA; 4qjkA; 4qjvA; 4qjvB; 4qkdA; 4qkwA; 4q1pA; 4q1pB; 4qmaA; 4qmdA; 4qmhA; 4qn8A; 4qndA; 4qo5A; 4qp5A; 4qpkA; 4qp1A; 4qpnA; 4qptA; 4qpvA; 4qpwA; 4qq0A; 4qq4A; 4qrhA; 4qrkA; 4qr1A; 4qmA; 4qrvA; 4qt6A; 4qtcA; 4qtpA; 4qtqA; 4qttB; 4qucA; 4qusA; 4qvhA; 4qwoA; 4qx5A; 4qxbA; 4qxbB; 4qx1A; 4qy7A; 4r03A; 4r0jA; 4r12A; 4r1dB; 4r1dA; 4r1hA; 4r1jA; 4r2fA; 4r2xA; 4r33A; 4r38A; 4r31A; 4r3 nA; 4r3qA; 4r42A; 4r4kA; 4r4 mA; 4r4xA; 4r52A; 4r5rA; 4r6fA; 4r6hA; 4r6kA; 4r6yA; 4r75A; 4r78A; 4r7kA; 4r7qA; 4r81A; 4r82A; 4r84A; 4r8xA; 4r9fA; 4r9iA; 4r9kA; 4r9 pA; 4rajA; 4raxA; 4rayA; 4rbrA; 4rcoA; 4rd4A; 4rd8A; 4rdbA; 4re5A; 4rekA; 4re1A; 4reoA; 4repA; 4rexA; 4rf6A; 4rfuA; 4rg1A; 4rgdA; 4rgiA; 4rguA; 4rgyA; 4rhaA; 4rhsA; 4ri5A; 4jwA; 4rjzA; 4rk2A; 4rk4A; 4rk6A; 4rkqA; 4rksA; 4r11A; 4r13A; 4r1cA; 4r1jB; 4r1jA; 4rm6A; 4rm8A; 4rmkA; 4rmxA; 4mcA; 4mwA; 4rnxA; 4rnzA; 4ro3A; 4rojA; 4rp3A; 4rp9A; 4rpmA; 4rptA; 4rriA; 4rs7A; 4rscA; 4rswA; 4rt1A; 4rthA; 4ru1A; 4ru3A; 4ru5A; 4rugA; 4ruqA; 4ruwA; 4rv0B; 4rv5A; 4rvcA; 4rvqA; 4rw0A; 4rwfA; 4rwfB; 4rwhA; 4rwuA; 4rxiA; 4rxvA; 4ry1A; 4ry8A; 4ryaA; 4ryeA; 4ryoA; 4rz3A; 4rz9A; 4rzaA; 4rzyA; 4s12A; 4s1aA; 4s1hA; 4s1pA; 4s28A; 4s36A; 4s39A; 4s3iA; 4s3jA; 4s3oB; 4tjvA; 4tkbA; 4tkcA; 4tkxL; 4t16A; 4t1vA; 4tmdA; 4tmeA; 4tmxA; 4tndA; 4tnsA; 4tpsA; 4tpsB; 4tpvA; 4tq1A; 4tq1B; 4tqxA; 4tr3A; 4tr6A; 4trkA; 4trtA; 4tsdA; 4tshB; 4tshA; 4ttwA; 4tv5A; 4tvcA; 4tveA; 4tvvA; 4tx5A; 4txdA; 4txgA; 4txrA; 4txrB; 4txrC; 4txvB; 4txwA; 4tyzA; 4tzhA; 4u09A; 4u0pB; 4u12A; 4u1eI; 4u1eB; 4u1eG; 4u31A; 4u3eA; 4u3sB; 4u3vA; 4u4pB; 4u4 pA; 4u5hA; 4u5qA; 4u5rA; 4u5wA; 4u5wB; 4u5yD; 4u63A; 4u68A; 4u72A; 4u7aA; 4u7iA; 4u89A; 4u8fA; 4u98A; 4u9cA; 4u9hL; 4u9hS; 4u9oA; 4u9 pA; 4u9uA; 4u9vB; 4ua3A; 4uabA; 4uafB; 4uafE; 4uapA; 4uasA; 4uavA; 4uc1A; 4uc8A; 4ud4A; 4udgA; 4udqA; 4udsA; 4udxX; 4ue0A; 4ue8A; 4ue8B; 4uf0A; 4uf7C; 4uf7A; 4ufqA; 4ug1A; 4uhcA; 4uhoA; 4uhqA; 4uhtA; 4uiqA; 4uj7A; 4uj8A; 4um7A; 4umgA; 4umiA; 4um1A; 4un2B; 4unuA; 4uobA; 4up0A; 4up3A; 4upiA; 4uqxA; 4uqzB; 4usaA; 4usiA; 4uskA; 4usoA; 4ut1A; 4utuA; 4uu3A; 4uuuA; 4uuxA; 4uvqA; 4uwxA; 4uwxC; 4uxeA; 4uybA; 4uydA; 4uyiA; 4uyrA; 4uytA; 4uz1A; 4uz3A; 4v00A; 4v0hA; 4v0wB; 4v0xB; 4v12A; 4v17A; 4v1gA; 4v1kA; 4v1sA; 4v24A; 4v2xA; 4v33A; 4v3iA; 4v3 1C; 4w1tA; 4w4 kB; 4w4kA; 4w4oC; 4w4tA; 4w5xA; 4w5zA; 4w64A; 4w6yA; 4w78A; 4w78B; 4w79A; 4w71A; 4w7wA; 4w82A; 4w8bA; 4w8hA; 4w8 pA; 4w8pB; 4w8qA; 4w9wA; 4wa0A; 4wb7A; 4wbdA; 4wbjA; 4wbsA; 4wbtA; 4wbyA; 4wcjA; 4wckA; 4wctA; 4wcxA; 4wd1A; 4wdcA; 4we2A; 4weeA; 4wesB; 4wesA; 4wfoA; 4wftA; 4wh5A; 4wh9A; 4whiA; 4whsB; 4whsA; 4wi1A; 4wi1A; 4wiqA; 4wjiA; 4wjsA; 4wjtA; 4wk0B; 4wk0A; 4wkaA; 4wksC; 4wksA; 4wkyA; 4wkzB; 4wkzA; 4w1hA; 4w1iA; 4w1rA; 4wIrB; 4wmaA; 4wmaD; 4wmuA; 4wmyA; 4wn5A; 4wndA; 4wndB; 4wp2A; 4wp3A; 4wp4A; 4wp6A; 4wp9A; 4wpkA; 4wpyA; 4wqdA; 4wqmA; 4wriA; 4wrpA; 4wsfA; 4wt3A; 4wtpA; 4wtvA; 4wtxA; 4wu0A; 4wubA; 4wuiA; 4wv4A; 4wv4B; 4wvaA; 4wveA; 4wviA; 4wvrA; 4ww7B; 4ww7A; 4wwrA; 4wwrB; 4wx0A; 4wxwA; 4wy4A; 4wy4C; 4wy4D; 4wy4B; 4wy9A; 4wz0A; 4wzaE; 4wzxE; 4wzxA; 4x00A; 4x0jA; 4x1fA; 4x1oA; 4x1zA; 4x28C; 4x2cA; 4x2hB; 4x2hA; 4x2hC; 4x2rA; 4x33A; 4x33B; 4x31A; 4x3 nA; 4x4wA; 4x5 mA; 4x5 pA; 4x5wA; 4x6gA; 4x7gA; 4x7kA; 4x84A; 4x86B; 4x86A; 4x8eA; 4x8qA; 4x8yA; 4x90A; 4x9cA; 4x9kA; 4x9rA; 4x9tA; 4x9xA; 4x9zA; 4xa7A; 4xa9A; 4xabA; 4xb4A; 4xb6B; 4xb6C; 4xb6D; 4xb6A; 4xbaA; 4xcbA; 4xd1A; 4xdiA; 4xduA; 4xdxA; 4xe7A; 4xeaA; 4xedA; 4xekA; 4xemA; 4xezA; 4xfjA; 4xfkA; 4xfmA; 4xg1A; 4xgoA; 4xgwA; 4xh7A; 4xhfA; 4xhmA; 4xhtA; 4xinA; 4xizM; 4xj5A; 4xjyA; 4xkbA; 4xkzA; 4x1gA; 4x1gB; 4x1oA; 4x1zA; 4xmrA; 4xo9A; 4xomA; 4xosA; 4xotA; 4xp7A; 4xp1A; 4xpqA; 4xpxA; 4xpzA; 4xq7A; 4xqaA; 4xqcA; 4xqmA; 4xrbA; 4xrwA; 4xsjA; 4xs1A; 4xsqA; 4xtbA; 4xtvA; 4xu4A; 4xuoA; 4xurA; 4xuwA; 4xvvA; 4xw3A; 4xwxA; 4xxfA; 4xx1A; 4xxtA; 4xxuA; 4xxxA; 4xy5A; 4xybA; 4xzaA; 4xzdA; 4y04A; 4y0cA; 4y0gA; 4y0hA; 4y0xA; 4y1bA; 4y1rA; 4y1sA; 4yiwA; 4y2fA; 4y63A; 4y6wA; 4y7dA; 4y71A; 4y7mC; 4y7sA; 4y88A; 4y93A; 4y99B; 4y99C; 4y9iA; 4y9jA; 4y9tA; 4y9vA; 4y9wA; 4yaaA; 4yagA; 4yahX; 4yamA; 4yapA; 4yb8A; 4yb8B; 4ybaA; 4ybgA; 4ybnA; 4yc5A; 4ycbA; 4ycsA; 4yd8A; 4ydrA; 4ydxA; 4ye7A; 4yepA; 4yf1A; 4yf4A; 4yg0A; 4ygbB; 4ygsA; 4yh8A; 4yh8B; 4yhbA; 4yheA; 4yhsA; 4yhvA; 4yi8A; 4yifA; 4yiiA; 4yivA; 4yj6A; 4yjmA; 4yjwA; 4ykdA; 4y14A; 4y18A; 4y18B; 4y1aA; 4y1eA; 4y1qL; 4y1qT; 4ymhA; 4ymiA; 4ymyA; 4yn1A; 4yn3A; 4yn3B; 4yn5A; 4ynhA; 4ynxA; 4yodA; 4yonA; 4yorA; 4yp6A; 4ypmA; 4ypoA; 4yqdA; 4ys0A; 4ysiA; 4ys1A; 4yt2A; 4ytbA; 4ytdA; 4ytkA; 4yt1A; 4ytwB; 4ytwA; 4yu8A; 4yucA; 4yv4A; 4yvdA; 4yvoA; 4ywaA; 4ywfA; 4ywkA; 4ywzA; 4yx1A; 4yx6A; 4yxpA; 4yy2A; 4yy8A; 4yycA; 4yyfA; 4yz0A; 4yz6B; 4yz6A; 4yzgA; 4yzoA; 4yztA; 4yzzA; 4z04A; 4z0gA; 4z0oA; 4z0vA; 4z0yA; 4zl3A; 4z1pA; 4z24A; 4z2oA; 4z2zA; 4z39A; 4z3gA; 4z3tA; 4z3xA; 4z3xE; 4z48A; 4z4aA; 4z54A; 4z5sA; 4z67A; 4z6 mA; 4z79A; 4z7aA; 4z7eA; 4z7xA; 4z80A; 4z80C; 4z8tA; 4z8tB; 4z8wA; 4z9dA; 4z9hA; 4z9 nA; 4z9 pA; 4za6A; 4za9A; 4zaiA; 4zavA; 4zbgA; 4zbhA; 4zboA; 4zbyA; 4zc3A; 4zcdA; 4zceA; 4zcnA; 4zcrA; 4zdfA; 4zdjA; 4zdsA; 4zdtA; 4zdtB; 4ze8A; 4zevA; 4zeyA; 4zf5A; 4zf7A; 4zf1A; 4zfoF; 4zfvA; 4zgfA; 4zgmB; 4zgmA; 4zgpA; 4zh0A; 4zh5A; 4zhbA; 4zhwA; 4zhyA; 4zi3C; 4zi5A; 4zi8A; 4zieA; 4zi1A; 4ziyA; 4zjhA; 4zjnA; 4zkqA; 4zlaA; 4zlfA; 4zlhA; 4zmhA; 4zmkA; 4zmyA; 4znkA; 4znmA; 4zo2A; 4zotA; 4zoxA; 4zoxB; 4zoyA; 4zp0A; 4zp6A; 4zq8A; 4zqaA; 4zqxA; 4zr8A; 4zrsA; 4zrxA; 4zs9A; 4zsiA; 4zu4A; 4zurA; 4zv0B; 4zv0A; 4zv5A; 4zv9A; 4zvaA; 4zvcA; 4zvfA; 4zw9A; 4zx2A; 4zy7A; 4zy9A; 4zyaA; 4zz1A; 5a0dA; 5a0A; 5a0nA; 5a0yA; 5a0yB; 5a0yC; 5a10A; 5a12A; 5a1iA; 5a1mA; 5a1qA; 5a2bA; 5a2fA; 5a35A; 5a3aA; 5a3yA; 5a4aA; 5a4oA; 5a51A; 5a57A; 5a61A; 5a62A; 5a67A; 5a6 mA; 5a6wA; 5a6wC; 5a71A; 5a7 mA; 5a7vA; 5a89A; 5a8cA; 5a8iA; 5a8jA; 5a96A; 5a98A; 5a99A; 5a9tA; 5aarA; 5absA; 5abxB; 5aduS; 5ae0A; 5aeaA; 5aecA; 5aegA; 5aeiA; 5aeoA; 5af3A; 5afdA; 5afwA; 5ag8A; 5agdA; 5agrA; 5agvA; 5ahiA; 5ahnA; 5ai1A; 5aimA; 5aizA; 5ajgA; 5ajjA; 5ajjB; 5ajoA; 5akrA; 5a16A; 5am2A; 5ambA; 5amhA; 5amtA; 5an4A; 5anpA; 5anzA; 5ao9A; 5aogA; 5aohA; 5aonA; 5aotA; 5aozA; 5apgA; 5apuA; 5aq0A; 5aqbA; 5aqcA; 5aqmB; 5aunB; 5aunA; 5awoA; 5ax6A; 5axgA; 5ay6A; 5ayvA; 5azbA; 5azpA; 5azwA; 5azxA; 5b08A; 5b0hA; 5b0rA; 5b0uA; 5b1nA; 5b1qA; 5b1rA; 5b3gB; 5b3 pA; 5b42A; 5b4bA; 5b4sA; 5b4zA; 5b5iA; 5b51A; 5b5qA; 5b5zA; 5b68A; 5b6cA; 5b6dA; 5b78B; 5b78A; 5b7gA; 5b7hA; 5b7yA; 5b82A; 5b89A; 5b8dA; 5bjxA; 5bmnA; 5bmoA; 5bmtA; 5bn3A; 5bn3B; 5bn8A; 5bnzA; 5bo7A; 5bobA; 5boiA; 5bopB; 5bovA; 5bowA; 5bp3A; 5bp8A; 5bp9A; 5bpkC; 5bpkA; 5bpxA; 5bq8A; 5bqpA; 5br4A; 5brhA; 5br1A; 5bs1A; 5bseA; 5btoA; 5btwA; 5btyA; 5bu3A; 5bu6A; 5bukA; 5buwA; 5bv8A; 5bvaA; 5bvrA; 5bw0A; 5bw0B; 5bxaA; 5bxdA; 5bxgA; 5bxrA; 5by1A; 5by5A; 5by7A; 5by8A; 5by8B; 5bykA; 5bzaA; 5c05A; 5c0pA; 5c12A; 5c17A; 5c1zA; 5c2iA; 5c2kA; 5c2 mA; 5c2 nA; 5c2uA; 5c30A; 5c33A; 5c3uA; 5c40A; 5c4yA; 5c50A; 5c50B; 5c54A; 5c5aA; 5c5cA; 5c5gA; 5c5rA; 5c5rC; 5c5tA; 5c5zA; 5c67C; 5c68A; 5c6kA; 5c6sA; 5c79A; 5c7rA; 5c86A; 5c8gA; 5c8qB; 5c8wA; 5c8zA; 5c90A; 5c98A; 5c9iA; 5c9oA; 5cajA; 5cc1A; 5cd2A; 5cdkA; 5cdvA; 5cecA; 5cecB; 5cegB; 5cegA; 5cfjA; 5cftA; 5cgqA; 5cgqB; 5chhA; 5chiA; 5chsA; 5cj3A; 5cjzA; 5ck4A; 5ck1A; 5cm7A; 5cm1A; 5cnwA; 5cofA; 5cotA; 5cowA; 5coyA; 5cozA; 5cpgA; 5cphA; 5cr4A; 5cr9A; 5crbA; 5crwA; 5csdA; 5csmA; 5csrA; 5ctaA; 5ctdA; 5ctmA; 5cttB; 5ctvA; 5cu7A; 5cuoA; 5cv0A; 5cvdA; 5cvwA; 5cwgA; 5cx7A; 5cxmA; 5cxwA; 5cxxA; 5cyaA; 5cyvA; 5cywB; 5cyzA; 5cyzC; 5cz1A; 5czcA; 5czwA; 5d08A; 5d0iA; 5d16A; 5d1iA; 5d1mB; 5d22A; 5d2eA; 5d2kA; 5d3kA; 5d3qA; 5d3xA; 5d4 nA; 5d4vA; 5d5 kB; 5d5yA; 5d66A; 5d6eA; 5d74A; 5d78A; 5d7uA; 5d7wA; 5d7zA; 5d88A; 5d8 mA; 5dagA; 5dazA; 5db1A; 5dc1A; 5dcqD; 5dcuA; 5decA; 5deqA; 5df6A; 5dfyA; 5dggA; 5dgjA; 5dgqA; 5dhmA; 5di0A; 5dicA; 5diiA; 5djeA; 5djhA; 5djoA; 5djtA; 5dkaA; 5dkxA; 5d1dA; 5d1eA; 5d1kA; 5d1tA; 5dm2A; 5dmaA; 5dmdA; 5dmmA; 5dmpA; 5dn8A; 5do6A; 5docA; 5dofA; 5domA; 5dp2A; 5dpoA; 5dqvA; 5dtcA; 5dthA; 5du9A; 5dutA; 5dv4A; 5dviA; 5dvwA; 5dwdA; 5dx6A; 5dx1A; 5dymA; 5dyqA; 5dzeA; 5dzoA; 5e0uD; 5e0yA; 5e0zA; 5e10A; 5e13A; 5e16A; 5e1qA; 5e1wA; 5e2cA; 5e37A; 5e3bA; 5e3eB; 5e3eA; 5e3qA; 5e4bA; 5e4gA; 5e50A; 5e56A; 5e57A; 5e5uB; 5e5yA; 5e68A; 5e6vA; 5e6xA; 5e6zA; 5e72A; 5e75A; 5e7hA; 5e71A; 5e8sA; 5e9 nA; 5e9 pA; 5ec6A; 5ecuA; 5edfA; 5ed1A; 5ee2A; 5eehA; 5efrA; 5efzA; 5eh1A; 5ehaA; 5ehrA; 5eipA; 5eiuA; 5ej3A; 5ej8A; 5ejrA; 5ejyA; 5ekiA; 5ekzA; 5e13A; 5e19A; 5e1bA; 5e1nA; 5embA; 5emiA; 5emxA; 5enfA; 5enqA; 5enuA; 5eovA; 5ep0A; 5ep2A; 5epeA; 5epwA; 5eq0A; 5eq7A; 5eqzA; 5er9A; 5ereA; 5erqA; 5erxA; 5escA; 5eu0A; 5eu0B; 5eurA; 5evcA; 5evfA; 5evhA; 5ewoA; 5ewpA; 5ewuA; 5ewyA; 5ex2A; 5exeA; 5exeB; 5exeC; 5exjA; 5expA; 5ey0A; 5eynA; 5ezqA; 5ezuA; 5fUeB; 5f18A; 5f1sA; 5f2kA; 5f30A; 5f3kA; 5f3 pA; 5f47A; 5f4cA; 5f4wA; 5f5 nA; 5f67A; 5f61A; 5f61B; 5f61J; 5f6rA; 5f7rA; 5f7uA; 5f7vA; 5f86A; 5f8cA; 5fa8A; 5faaA; 5faiA; 5favA; 5fbfA; 5fc2B; 5fc9A; 5fceA; 5fcfA; 5fcnA; 5fcuG; 5fd5A; 5fd9A; 5fewA; 5ffaA; 5ffdA; 5ffiA; 5ffqA; 5ffxA; 5fg3A; 5fg6A; 5fgpA; 5fgsA; 5fguA; 5fgwA; 5fhkA; 5fiaA; 5fidA; 5fieA; 5figA; 5fiiA; 5fisA; 5fjdA; 5fj1A; 5fjnA; 5fktA; 5f1jA; 5f1wA; 5f1yA; 5fmdA; 5fmrA; 5fmuA; 5fnpA; 5focA; 5fp1A; 5fpzA; 5fq1A; 5fq4A; 5fqeA; 5fr7A; 5frdA; 5fs8A; 5fsvA; 5ftbA; 5fu5A; 5fuiA; 5fukA; 5fusA; 5fvdA; 5fvdB; 5fvjA; 5fvkA; 5fvkC; 5fvnA; 5fwaA; 5fwsA; 5fydA; 5fypA; 5fyzA; 5fzoA; 5fzpA; 5fzsA; 5g0aA; 5g0hA; 5g0xA; 5g1aA; 5g11A; 5g1xB; 5g2uA; 5g2vA; 5g38A; 5g3 pA; 5g3qA; 5g3tA; 5g3xA; 5g3yA; 5g4zA; 5g51A; 5g5cA; 5g5gC; 5g5gA; 5g5oA; 5ggbA; 5ggnA; 5gheA; 5gi7A; 5gj7A; 5gjiA; 5gjoA; 5gk1A; 5gkvA; 5g15A; 5g1gA; 5gm9A; 5gmbA; 5gmdA; 5gmtA; 5gmzA; 5gn1A; 5gn2A; 5gnfA; 5gngA; 5gofA; 5gpiA; 5gpoA; 5gqfA; 5gqiA; 5gqwA; 5grmA; 5groA; 5grqA; 5grqC; 5gs7A; 5gsmA; 5gt1A; 5gt5A; 5gtfA; 5gtqA; 5gtuA; 5gtuB; 5gu6A; 5guaA; 5gudA; 5guqA; 5gv0A; 5gvaA; 5gvdA; 5gviA; 5gvvA; 5gwnA; 5gwtA; 5gxeA; 5gxxA; 5gycA; 5gz2A; 5gz3A; 5gzaA; 5gzfA; 5gzkA; 5h02A; 5h06A; 5h0jA; 5h0mA; 5h0qA; 5h18A; 5hinA; 5h28A; 5h2dA; 5h3jA; 5h3jB; 5h3kA; 5h4lA; 5h4eA; 5h4gA; 5h4sA; 5h5fA; 5h62A; 5h66A; 5h66B; 5h66C; 5h68A; 5h6kA; 5h6 nA; 5h6tA; 5h6xA; 5h6zA; 5h78A; 5h7eA; 5h7rD; 5h8iA; 5h9cA; 5h9iA; 5h9 nA; 5h9yA; 5hb6A; 5hb7A; 5hbpA; 5hctA; 5hd9A; 5hdkA; 5hdmA; 5he9A; 5he9E; 5heaA; 5heeA; 5heyA; 5hfgA; 5hfsA; 5hgzA; 5hh0A; 5hh7A; 5hhaA; 5hheA; 5hhjA; 5hi4A; 5hi8A; 5hifA; 5hj1A; 5hj9A; 5hjfA; 5hjmA; 5hkjA; 5hk1A; 5hkqA; 5hkqI; 5hkxA; 5h13A; 5h18A; 5hm7A; 5hm1A; 5hnoA; 5hnvA; 5hobA; 5hokA; 5hopA; 5hqhA; 5hqtA; 5hr5A; 5hs7A; 5hsiA; 5hsmA; 5hspA; 5hsqA; 5hsxA; 5ht2A; 5ht7A; 5ht1A; 5htxA; 5hu3B; 5hubA; 5husA; 5hwaA; 5hweA; 5hwhA; 5hwkA; 5hwnA; 5hwtA; 5hx0A; 5hxiA; 5hxkA; 5hx1A; 5hyaA; 5hy1A; 5hyvA; 5hyzA; 5hz7A; 5hzdA; 5i0fB; 5i0zA; 5i14A; 5i21A; 5i29A; 5i2cA; 5i2hA; 5i21A; 5i34A; 5i39A; 5i3eA; 5i41B; 5i45A; 5i4cA; 5i4dA; 5i5 mA; 5i5 nA; 5i62A; 5i8gA; 5i8jA; 5i8tA; 5i90A; 5i95A; 5i9jA; 5ia8A; 5iaaC; 5iaaA; 5iaiA; 5ib0A; 5iboA; 5ibzA; 5ic0A; 5icqA; 5icuA; 5icvA; 5idhA; 5idkA; 5idmA; 5idvA; 5ig0A; 5ig6A; 5igiA; 5ihfA; 5ihsA; 5ihwA; 5ii5A; 5ii6A; 5ii8A; 5ijaA; 5ijiA; 5ijjA; 5ijmA; 5ik4A; 5ikuA; 5i16A; 5i1bA; 5i1uA; 5imkA; 5imuA; 5in1A; 5in3A; 5in4A; 5inbB; 5inrA; 5io9A; 5ipyA; 5iqjA; 5ir4A; 5irbA; 5ircA; 5irsA; 5is2A; 5isvA; 5iswA; 5it3A; 5itjA; 5itmA; 5iu0I; 5iu1A; 5iu4A; 5iucA; 5iufA; 5ivbA; 5ivgA; 5iwbA; 5iwbB; 5iwhA; 5ix8A; 5ixbA; 5ixgA; 5ixhA; 5ixoA; 5ixpA; 5iyzA; 5iyzE; 5iyzF; 5izaA; 5izeA; 5iztA; 5j03A; 5j07A; 5j08A; 5j09A; 5j0cA; 5j0fA; 5j0kA; 5j1gA; 5j1jA; 5j1kA; 5j1nA; 5j1sB; 5j1sA; 5j39A; 5j3tA; 5j3tB; 5j3tC; 5j3uA; 5j4lA; 5j47A; 5j49A; 5j4aA; 5j4aB; 5j4lA; 5j4oA; 5j4uA; 5j53A; 5j51A; 5j6yA; 5j71A; 5j72A; 5j81A; 5j8eA; 5j8yC; 5j8yA; 5j90A; 5j93A; 5j9iA; 5ja5A; 5ja9C; 5jawA; 5jazA; 5jbdA; 5jb1A; 5jbnA; 5jbrA; 5jbsA; 5jbxA; 5jcaL; 5jcaS; 5jciA; 5jd5A; 5jdaA; 5jddA; 5jdkA; 5jdtA; 5je2A; 5je1A; 5je1B; 5jffA; 5jffB; 5jg7A; 5jgfA; 5jgkA; 5jh8A; 5jhxA; 5ji7A; 5jiaA; 5jicA; 5jioA; 5jipA; 5jirA; 5jiwA; 5jixA; 5jj2A; 5jjeB; 5jjoA; 5jjsA; 5jjxA; 5j1bA; 5j1vC; 5jmuA; 5jn5A; 5jnmA; 5jo8A; 5joqA; 5jovA; 5jp6A; 5jphA; 5jpoA; 5jpoE; 5jqmA; 5jqnA; 5jqyA; 5jrjA; 5jrtA; 5jryA; 5js4A; 5jsiA; 5jskA; 5jufA; 5jugA; 5juhA; 5jv4A; 5jviE; 5jvmA; 5jvoA; 5jw9B; 5jw9A; 5jwoB; 5jxgA; 5jxmA; 5jxzA; 5jysA; 5k08A; 5k0aA; 5k26A; 5k21A; 5k2xA; 5k34A; 5k3qA; 5k3xA; 5k3yC; 5k4bA; 5k62A; 5k68A; 5k69A; 5k6dA; 5k6kA; 5k61A; 5k7fA; 5k7wA; 5k7wB; 5k87A; 5k8cA; 5k8gA; 5k8jB; 5k8jA; 5k8sA; 5k9gA; 5ka5A; 5kakA; 5karA; 5kaxA; 5kayA; 5kbzA; 5kc8A; 5kciA; 5kcnA; 5kd5A; 5kdgA; 5kdiA; 5kdoB; 5kdoG; 5kdsA; 5kdwA; 5ke1A; 5kecA; 5kf6A; 5kf9A; 5kiqA; 5kivA; 5kkoA; 5k1eA; 5k1hA; 5k1pA; 5km9A; 5knhI; 5ko4A; 5ko5A; 5ko9A; 5koeA; 5koxA; 5kpgA; 5kprA; 5kqrA; 5ktcA; 5ktkA; 5ktnA; 5kueA; 5kukA; 5kutA; 5kuxA; 5kvaA; 5kvbA; 5kvgE; 5kvrA; 5kvsA; 5kwnA; 5kxhA; 5kycB; 5kz6A; 5kzaA; 5kzzA; 5l01A; 5l09A; 5l01A; 5l0 nA; 5l0vB; 5l0vA; 5l20A; 5l33A; 5l37A; 5l44A; 5l41A; 5l74A; 5l77A; 5l7eA; 5l87A; 5l8hA; 5l8xA; 5l9zA; 5l9zB; 5la4A; 5lacA; 5la1A; 5lb3B; 5lb6A; 5lb7B; 5lb7A; 5lbdA; 5lbkA; 5lc2A; 5lc9A; 5ld9A; 5ldaB; 5ldqA; 5le5A; 5le5K; 5le5H; 5le5L; 5le5I; 5le5J; 5le5M; 5leoA; 5lf2A; 5lf9A; 5lfzA; 5lhxA; 5lirA; 5lj8A; 5ljmA; 5ljpA; 5ljwA; 5ljxA; 5lkbA; 5lkvA; 5llbA; 5lljA; 5lmgA; 5lnnA; 5lnrA; 5lp0A; 5lp9A; 5lpaA; 5lpgA; 5lpiA; 5lq5A; 5lq6A; 5lq1A; 5lrtA; 5lrwA; 5ls4A; 5ls7D; 5ls7A; 5ls7B; 5lsiD; 5lsiE; 5ls1E; 5lsvA; 5lt5A; 5lteA; 5ltgA; 5ltjA; 5ltnA; 5lu5A; 5lunA; 5lusA; 5lw0A; 5lw3A; 5lwaA; 5lx8A; 5lxeA; 5lxfA; 5lxxA; 5lxzB; 5ly0A; 5ly3A; 5ly5A; 5ly8A; 5ly9A; 5lyeA; 5lz1A; 5lzkA; 5lznA; 5m02B; 5m04A; 5m0nA; 5m0wA; 5m0yB; 5m10A; 5m17A; 5m1iA; 5m1mA; 5m1pA; 5m1xA; 5m23A; 5m26B; 5m26A; 5m29A; 5m2oA; 5m2 pA; 5m2yA; 5m31A; 5m33A; 5m3 nA; 5m43A; 5m45B; 5m45A; 5m45C; 5m5tA; 5m5zA; 5m6qA; 5m72A; 5m72B; 5m77A; 5m7dA; 5m7yA; 5m90A; 5m97A; 5m99A; 5m9fA; 5m9 nA; 5ma4A; 5ma1A; 5maoA; 5mawD; 5mawE; 5mbxA; 5mc1A; 5mc7A; 5mdrA; 5me4A; 5me5A; 5me5B; 5mebA; 5medA; 5mekA; 5mfaA; 5mfiA; 5mfoA; 5mfpA; 5mfrA; 5mgwA; 5mgzA; 5mi4A; 5mixA; 5mj7A; 5mjhA; 5mjrA; 5mk2A; 5mk2C; 5mk9A; 5mkwA; 5m13B; 5m1dA; 5m1kA; 5m1tA; 5m1zA; 5mobA; 5mo1A; 5mp0D; 5mptA; 5mpwA; 5mqiA; 5mqnA; 5mqpA; 5mr1A; 5mr5C; 5mriA; 5mrvA; 5msnA; 5msoA; 5mt2A; 5mteA; 5mu9A; 5mu1A; 5munA; 5muzA; 5mv0A; 5mvwA; 5mvwC; 5mx9A; 5mxcA; 5my5A; 5my7A; 5myfA; 5mypA; 5mzwA; 5mzwB; 5n07A; 5n22A; 5n2bA; 5n2cA; 5n2iA; 5n3uA; 5n3uB; 5n40A; 5n4lA; 5n6xA; 5n6yC; 5n7eB; 5n81A; 5n88D; 5n8aX; 5n8bA; 5na2A; 5na6A; 5naaA; 5nakA; 5nbfA; 5nboA; 5ncjA; 5ncwB; 5ng9A; 5nggA; 5ng1A; 5nh5A; 5nioA; 5nj9B; 5nj9A; 5nj1A; 5n19A; 5nmoA; 5nn4A; 5nnyA; 5no8A; 5noaA; 5nodA; 5nohA; 5nonA; 5nopA; 5nqoA; 5nqvA; 5nrkB; 5nrkA; 5nryA; 5nsaA; 5nt7B; 5nt7A; 5nuvA; 5nvmB; 5nw3A; 5nx7A; 5nxfA; 5nypA; 5nzpA; 5nzxA; 5o0sA; 5o15A; 5o11A; 5o1xA; 5o2dA; 5o2xA; 5o33B; 5o37A; 5o5sA; 5o75A; 5o8wB; 5o95A; 5o99A; 5o9eA; 5o9eB; 5o9 mA; 5oaqL; 5oc7A; 5od4A; 5odjA; 5odkA; 5oduA; 5oe3A; 5oemA; 5oh5A; 5ohjA; 5ohoA; 5ohqA; 5oj7A; 5ok8A; 5okpA; 5o17A; 5o18A; 5o19A; 5o1pA; 5o1rA; 5o1uA; 5ombC; 5omkA; 5ompA; 5omtA; 5oniA; 5oo9A; 5op0A; 5opqA; 5opzA; 5oq3A; 5oswA; 5ovoA; 5owuA; 5owuB; 5p9vA; 5paxA; 5phjA; 5px1A; 5suiA; 5suyA; 5sv2A; 5sv5A; 5sv6A; 5svyA; 5swcA; 5swkA; 5swkC; 5sy4A; 5sy80; 5syrA; 5sz8A; 5szbA; 5szdA; 5t05A; 5t07A; 5t1iA; 5t1pA; 5t39A; 5t3bA; 5t40A; 5t46A; 5t46B; 5t4xA; 5t5iA; 5t5iB; 5t5iF; 5t5iC; 5t5iD; 5t5iG; 5t6jB; 5t6jA; 5t77A; 5t7aA; 5t7dA; 5t7oA; 5t86I; 5t86A; 5t88A; 5t8cA; 5t9 pA; 5t9yA; 5ta0A; 5tabA; 5tcbA; 5tdaA; 5tdeA; 5tdeB; 5tedA; 5teeA; 5teyA; 5teyB; 5tf3A; 5tfpA; 5tg0A; 5tgfA; 5tgnA; 5thxA; 5tipA; 5tj3A; 5tjzA; 5tk2A; 5tk8A; 5tkwA; 5td4A; 5td5A; 5t1eA; 5tnvA; 5toqA; 5tpiA; 5tprA; 5tqiA; 5trbA; 5troA; 5trqA; 5ts9A; 5tt5A; 5ttaA; 5ttdA; 5ttyA; 5tuiA; 5tuxA; 5tv2A; 5tvdA; 5tvoA; 5tvoB; 5tvyA; 5tw9A; 5twaA; 5twaC; 5txuA; 5tz5A; 5tjA; 5tzpA; 5tzpB; 5u19A; 5u1hA; 5u22A; 5u21A; 5u2 pA; 5u35A; 5u3aA; 5u47A; 5u4hA; 5u4uA; 5u69A; 5u75A; 5u7fA; 5u9zA; 5uamA; 5uavA; 5uazA; 5ub3A; 5ubdA; 5ub1A; 5uc0A; 5ucbB; 5ucvA; 5udnA; 5ue0A; 5ue3A; 5uebA; 5uejA; 5ufhA; 5ufnA; 5ufyA; 5ugrA; 5uh0A; 5ui9A; 5uizA; 5uj6A; 5tujcA; 5ukhA; 5ukvA; 5u13A; 5um2A; 5umfA; 5umhA; 5umrA; 5umsA; 5umuA; 5umvA; 5uncA; 5uouA; 5upbA; 5upiA; 5uq6A; 5uqdA; 5uqjA; 5uroA; 5uswA; 5uttA; 5uuiA; 5uvdA; 5uvgA; 5uwaA; 5uy7A; 5uytA; 5uzgA; 5uznA; 5v01A; 5v0zA; 5v13A; 5v1yA; 5v2cC; 5v2cA; 5v2cD; 5v2cE; 5v2cB; 5v2cI; 5v2cL; 5v2cT; 5v2cZ; 5v2cJ; 5v2c0; 5v2cM; 5v2cH; 5v2cF; 5v2cX; 5v2cK; 5v2cU; 5v2cY; 5v2iA; 5v2oA; 5v2qA; 5v3 nA; 5v3nB; 5v3sA; 5v3wA; 5v44A; 5v5hA; 5v5yA; 5v6bA; 5v6fA; 5v77A; 5v86A; 5v87A; 5v89C; 5v8dA; 5v8sA; 5vacA; 5vapA; 5vbbA; 5vbdA; 5vccA; 5ve3A; 5vegA; 5veiA; 5vf5A; 5vfbA; 5vg3A; 5vgbA; 5vgbB; 5vg1A; 5vgtA; 5vhgA; 5vhtA; 5vi6A; 5viaA; 5vipB; 5vipA; 5vixA; 5vjiA; 5vjiC; 5v1iC; 5vmrC; 5vn4A; 5vnyA; 5vo5A; 5vogA; 5vo1A; 5vpuA; 5vqeA; 5vr2A; 5vscA; 5vsmA; 5vtgA; 5vugA; 5vwmA; 5vx1A; 5vxcA; 5vxvA; 5vyqA; 5vyrA; 5vz3A; 5vzvA; 5w2fA; 5w2iA; 5w21A; 5w3rA; 5w3xA; 5w3xB; 5w53A; 5w5bA; 5w5cF; 5w5cE; 5w5cA; 5w6yA; 5w7bC; 5w7bA; 5w7dA; 5w83A; 5w83B; 5w8eA; 5w8oA; 5w8qA; 5w93D; 5w93A; 5w95A; 5w98A; 5wanA; 5wcjA; 5wd9A; 5wf2A; 5wfbA; 5wgiA; 5wgxA; 5whmA; 5whtA; 5whxA; 5wi4A; 5wo2A; 5wofA; 5woqA; 5wp4A; 5wq3A; 5wqcA; 5wqjA; 5wqwA; 5wriA; 5wrvA; 5ws7A; 5wsfA; 5wsyA; 5wtqA; 5wucA; 5wvoC; 5wvoD; 5wwdA; 5wx1A; 5wx1B; 5wy0A; 5wzfA; 5wzqA; 5x13A; 5x1eB; 5x1eA; 5x1eC; 5x1uA; 5x2eA; 5x3dA; 5x40A; 5x42A; 5x42B; 5x4bA; 5x4rA; 5x4tA; 5x57A; 5x5 mA; 5x5vA; 5x6sA; 5x7 nA; 5x7qA; 5x89A; 5x9kA; 5x9oA; 5xa5A; 5xa5B; 5xauA; 5xauC; 5xauB; 5xavA; 5xb7A; 5xbcA; 5xbfA; 5xbfB; 5xctA; 5xdcA; 5xdtA; 5xdzB; 5xevA; 5xfoA; 5xg2A; 5xg5A; 5xgsA; 5xguA; 5xh2A; 5xhbA; 5xi8A; 5xj1A; 5xj5A; 5xk6A; 5xkrA; 5xktA; 5xkxA; 5x1jA; 5x1yB; 5xm5A; 5xmzA; 5xn3A; 5xnhA; 5xopA; 5xpcA; 5xtsA; 5xunA; 5xw4A; 5xx1A; 5y27A; 5y27B; 5y3cA; 5y4fA; 5y4zA; 5y69A; 5y9aA; 5y9qA; 5y9wA; 5y9wC; 5ya1A; 5yayA; 5yayB; 5ydeA; 5yfcA; 5yhyA; 5yj6A; 5yobA; 5yqjA; 5yxcA; 5z02A; 6amgA; 6an0A; 6anzA; 6ao1A; 6ao7A; 6ao8A; 6ao9A; 6aokA; 6appB; 6as4A; 6at0A; 6au8A; 6au8C; 6avjA; 6avxA; 6az6A; 6azhA; 6aziA; 6b00A; 6b0gE; 6b12B; 6b12A; 6b1zA; 6b26A; 6b29A; 6b2yA; 6b3aA; 6b3yA; 6b4aA; 6b57A; 6b61A; 6b6uA; 6b8wA; 6b9fA; 6b9rA; 6b9xB; 6b9xC; 6b9xD; 6b9xE; 6b9xA; 6bcbA; 6bevA; 6bgdA; 6bhdA; 6bk0A; 6b1kA; 6b1mA; 6bmeA; 6bo0A; 6bphA; 6bu6A; 6bus1; 6bvcA; 6bweA; 6bxgA; 6c0cA; 6c4qA; 6c4vA; 6ehiA; 6ekbA; 6ektA; 6e1mA; 6ensA; 6eofA; 6eonA; 6eroA; 6es9A; 6euwA; 6fDpA; 6f72A; 6f8 pA; 6ff1A; 6fg8A; 7fd1A;

### REFERENCES

1. C. B. Anfinsen, Principles that govern the folding of protein chains. Science 181, 223 (1973).
2. I. V. Korendovych, W. F. DeGrado, De novo protein design, a retrospective. Q. Rev. Biophys. 53, e3 (2020).
3. B. Kuhlman, P. Bradley, Advances in protein structure prediction and design. Nat. Rev. Mol. Cell Biol. 20, 681-697 (2019).
4. J. Dou, L. Doyle, P. Jr. Greisen, A. Schena, H. Park, K. Johnsson, B. L. Stoddard, D. Baker, Sampling and energy evaluation challenges in ligand binding protein design. Protein Sci. 26, 2426-2437 (2017).
5. E. Marcos, B. Basanta, T. M. Chidyausiku, Y. Tang, G. Oberdorfer, G. Liu, G. V. T. Swapna, R. Guan, D.-A. Silva, J. Dou, J. H. Pereira, R. Xiao, B. Sankaran, P. H. Zwart, G. T. Montelione, D. Baker, Principles for designing proteins with cavities formed by curved β sheets. Science 355, 201 (2017).
6. C. E. Tinberg, S. D. Khare, J. Dou, L. Doyle, J. W. Nelson, A. Schena, W. Jankowski, C. G. Kalodimos, K. Johnsson, B. L. Stoddard, D. Baker, Computational design of ligand-binding proteins with high affinity and selectivity. Nature 501, 212-216 (2013).
7. A. L. Day, P. Greisen, L. Doyle, A. Schena, N. Stella, K. Johnsson, D. Baker, B. Stoddard, Unintended specificity of an engineered ligand-binding protein facilitated by unpredicted plasticity of the protein fold. Protein Eng. Des. Sel 31, 375-387 (2018).
8. J. Dou, A. A. Vorobieva, W. Sheffler, L. A. Doyle, H. Park, M. J. Bick, B. Mao, G. W. Foight, M. Y. Lee, L. A. Gagnon, L. Carter, B. Sankaran, S. Ovchinnikov, E. Marcos, P.-S. Huang, J. C. Vaughan, B. L. Stoddard, D. Baker, De novo design of a fluorescence-activating (3-barrel. Nature 561, 485-491 (2018).
9. E. P. Barros, J. M. Schiffer, A. Vorobieva, J. Dou, D. Baker, R. E. Amaro, Improving the efficiency of ligand-binding protein design with molecular dynamics simulations. J. Chem. Theory Comput. 15, 5703-5715 (2019).
10. G. Grigoryan, W. F. DeGrado, Probing designability via a generalized model of helical bundle geometry. J. Mol. Biol. 405, 1079-1100 (2011).
11. P.-S. Huang, G. Oberdorfer, C. Xu, X. Y. Pei, B. L. Nannenga, J. M. Rogers, F. DiMaio, T. Gonen, B. Luisi, D. Baker, High thermodynamic stability of parametrically designed helical bundles. Science 346, 481 (2014).
12. K. Szczepaniak, G. Lach, J. M. Bujnicki, S. Dunin-Horkawicz, Designability landscape reveals sequence features that define axial helix rotation in four-helical homo-oligomeric antiparallel coiled-coil structures. J. Struct. Biol. 188, 123-133 (2014).
13. N. F. Polizzi, Y. Wu, T. Lemmin, A. M. Maxwell, S.-Q. Zhang, J. Rawson, D. N. Beratan, M. J. Therien, W. F. DeGrado, De novo design of a hyperstable non-natural protein-ligand complex with sub-a accuracy. Nat. Chem. 9, 1157-1164 (2017).
14. G. G. Rhys, C. W. Wood, J. L. Beesley, N. R. Zaccai, A. J. Burton, R. L. Brady, A. R. Thomson, D. N. Woolfson, Navigating the structural landscape of de novo α-helical bundles. J. Am. Chem. Soc. 141, 8787-8797 (2019).
15. A. J. Reig, M. M. Pires, R. A. Snyder, Y. Wu, H. Jo, D. W. Kulp, S. E. Butch, J. R. Calhoun, T. Szyperski, E. I. Solomon, W. F. DeGrado, Alteration of the oxygen-dependent reactivity of de novo due fern proteins. Nat. Chem. 4, 900-906 (2012).
16. A. N. Lupas, J. Bassler, S. Dunin-Horkawicz, in Fibrous proteins: Structures and mechanisms, D. A. D. Parry, J. M. Squire, Eds. (Springer International Publishing, Cham, 2017), pp. 95-129.
17. A. Lombardi, F. Pirro, O. Maglio, M. Chino, W. F. DeGrado, De novo design of four-helix bundle metalloproteins: One scaffold, diverse reactivities. Acc. Chem. Res. 52, 1148-1159 (2019).
18. J. R. Desjarlais, T. M. Handel, De novo design of the hydrophobic cores of proteins. Protein Sci. 4, 2006-2018 (1995).
19. J. Janin, S. Wodak, M. Levitt, B. Maigret, Conformation of amino acid side-chains in proteins. J. Mol. Biol. 125, 357-386 (1978).
20. M. J. McGregor, S. A. Islam, M. J. E. Sternberg, Analysis of the relationship between side-chain conformation and secondary structure in globular proteins. J. Mol. Biol. 198, 295-310 (1987).
21. J. W. Ponder, F. M. Richards, Tertiary templates for proteins: Use of packing criteria in the enumeration of allowed sequences for different structural classes. J. Mol. Biol. 193, 775-791 (1987).
22. B. I. Dahiyat, S. L. Mayo, Protein design automation. Protein Sci. 5, 895-903 (1996).
23. J. K. Lassila, H. K. Privett, B. D. Allen, S. L. Mayo, Combinatorial methods for small-molecule placement in computational enzyme design. Proc. Natl. Acad. Sci. USA 103, 16710 (2006).
24. J. Singh, J. M. Thornton, Atlas of protein side-chain interactions. (IRL Press at Oxford University Press, Oxford; New York, 1992).
25. A. Zanghellini, L. Jiang, A. M. Wollacott, G. Cheng, J. Meiler, E. A. Althoff, D. Röthlisberger, D. Baker, New algorithms and an in silico benchmark for computational enzyme design. Protein Sci. 15, 2785-2794 (2006).
26. K. W. Kaufmann, G. H. Lemmon, S. L. DeLuca, J. H. Sheehan, J. Meiler, Practically useful: What the Rosetta protein modeling suite can do for you. Biochemistry 49, 2987-2998 (2010).
27. R. Ferreira de Freitas, M. Schapira, A systematic analysis of atomic protein-ligand interactions in the PDB. MedChemComm 8, 1970-1981 (2017).
28. B. North, C. M. Summa, G. Ghirlanda, W. F. DeGrado, Dn-symmetrical tertiary templates for the design of tubular proteins. J. Mol. Biol. 311, 1081-1090 (2001).
29. D. H. Williams, E. Stephens, D. P. O'Brien, M. Zhou, Understanding noncovalent interactions: Ligand binding energy and catalytic efficiency from ligand-induced reductions in motion within receptors and enzymes. Angew. Chem. Int. Ed. 43, 6596-6616 (2004).
30. S. K. Tan, K. P. Fong, N. F. Polizzi, A. Stemisha, J. S. G. Slusky, K. Yoon, W. F. DeGrado, J. S. Bennett, Modulating integrin αIIbβ3 activity through mutagenesis of allosterically regulated intersubunit contacts. Biochemistry 58, 3251-3259 (2019).
31. F. Thomas, W. M. Dawson, E. J. M. Lang, A. J. Burton, G. J. Bartlett, G. G. Rhys, A. J. Mulholland, D. N. Woolfson, De novo-designed α-helical barrels as receptors for small molecules. ACS Synthetic Biology 7, 1808-1816 (2018).
32. J. Park, B. Selvaraj, A. C. McShan, S. E. Boyken, K. Y. Wei, G. Oberdorfer, W. DeGrado, N. G. Sgourakis, M. J. Cuneo, D. A. A. Myles, D. Baker, De novo design of a homo-trimeric amantadine-binding protein. eLife 8, e47839 (2019).
33. A. A. Glasgow, Y.-M. Huang, D. J. Mandell, M. Thompson, R. Ritterson, A. L. Loshbaugh, J. Pellegrino, C. Krivacic, R. A. Pache, K. A. Barlow, N. Ollikainen, D. Jeon, M. J. S. Kelly, J. S. Fraser, T. Kortemme, Computational design of a modular protein sense-response system. Science 366, 1024 (2019).
34. N. Tokuriki, D. S. Tawfik, Protein dynamism and evolvability. Science 324, 203 (2009).
35. T. J. Stout, C. R. Sage, R. M. Stroud, The additivity of substrate fragments in enzyme-ligand binding. Structure 6, 839-848 (1998).
36. D. A. Keedy, Z. B. Hill, J. T. Biel, E. Kang, T. J. Rettenmaier, J. Brandão-Neto, N. M. Pearce, F. von Delft, J. A. Wells, J. S. Fraser, An expanded allosteric network in PTP1B by multitemperature crystallography, fragment screening, and covalent tethering. eLife 7, e36307 (2018).
37. J. M. Word, S. C. Lovell, J. S. Richardson, D. C. Richardson, Asparagine and glutamine: Using hydrogen atom contacts in the choice of side-chain amide orientation. J. Mol. Biol. 285, 1735-1747 (1999).
38. V. B. Chen, W. B. Arendall, III, J. J. Headd, D. A. Keedy, R. M. Immormino, G. J. Kapral, L. W. Murray, J. S. Richardson, D. C. Richardson, Molprobity: All-atom structure validation for macromolecular crystallography. Acta Cryst. D 66, 12-21 (2010).
39. A. Bakan, L. M. Meireles, I. Bahar, Prody: Protein dynamics inferred from theory and experiments. Bioinformatics 27, 1575-1577 (2011).
40. J. M. Word, S. C. Lovell, T. H. LaBean, H. C. Taylor, M. E. Zalis, B. K. Presley, J. S. Richardson, D. C. Richardson, Visualizing and quantifying molecular goodness-of-fit: Small-probe contact dots with explicit hydrogen atoms. J. Mol. Biol. 285, 1711-1733 (1999).
41. J. Zhou, G. Grigoryan, Rapid search for tertiary fragments reveals protein sequence-structure relationships. Protein Sci. 24, 508-524 (2015).
42. A. Lombardi, C. M. Summa, S. Geremia, L. Randaccio, V. Pavone, W. F. DeGrado, Retrostructural analysis of metalloproteins: Application to the design of a minimal model for diiron proteins. Proc. Natl. Acad. Sci. USA 97, 6298 (2000).
43. J. M. Dunce, O. M. Dunne, M. Ratcliff, C. Millán, S. Madgwick, I. Usón, O. R. Davies, Structural basis of meiotic chromosome synapsis through SYCP1 self-assembly. Nat. Struct. Mol. Biol. 25, 557-569 (2018).
44. C. A. K. Lundgren, D. Sjöstrand, O. Biner, M. Bennett, A. Rudling, A.-L. Johansson, P. Brzezinski, J. Carlsson, C. von Ballmoos, M. Hogbom, Scavenging of superoxide by a membrane-bound superoxide oxidase. Nat. Chem. Biol. 14, 788-793 (2018).
45. S. E. Boyken, Z. Chen, B. Groves, R. A. Langan, G. Oberdorfer, A. Ford, J. M. Gilmore, C. Xu, F. DiMaio, J. H. Pereira, B. Sankaran, G. Seelig, P. H. Zwart, D. Baker, De novo design of protein homo-oligomers with modular hydrogen-bond network-mediated specificity. Science 352, 680 (2016).
46. Y. Hong, Z. Huang, L. Guo, B. Ni, C.-Y. Jiang, X.-J. Li, Y.-J. Hou, W.-S. Yang, D.-C. Wang, I. B. Zhulin, S.-J. Liu, D.-F. Li, The ligand-binding domain of a chemoreceptor from Comamonas testosteroni has a previously unknown homotrimeric structure. Mol. Microbiol. 112, 906-917 (2019).
47. M. Valiev, E. J. Bylaska, N. Govind, K. Kowalski, T. P. Straatsma, H. J. J. Van Dam, D. Wang, J. Nieplocha, E. Apra, T. L. Windus, W. A. de Jong, NWchem: A comprehensive and scalable open-source solution for large scale molecular simulations. Comput. Phys. Commun. 181, 1477-1489 (2010).
48. J. Liang, K. A. Dill, Are proteins well-packed? Biophys. J. 81, 751-766 (2001).
49. N. D. Clarke, S.-M. Yuan, Metal search: A computer program that helps design tetrahedral metal-binding sites. Proteins: Struct. Funct. Bioinform. 23, 256-263 (1995).
50. M. Lee, T. Wang, O. V. Makhlynets, Y. Wu, N. F. Polizzi, H. Wu, P. M. Gosavi, J. Stohr, I. V. Korendovych, W. F. DeGrado, M. Hong, Zinc-binding structure of a catalytic amyloid from solid-state nmr. Proc. Natl. Acad. Sci. USA 114, 6191 (2017).
51. S. J. Lahr, D. E. Engel, S. E. Stayrook, O. Maglio, B. North, S. Geremia, A. Lombardi, W. F. DeGrado, Analysis and design of turns in α-helical hairpins. J. Mol. Biol. 346, 1441-1454 (2005).
52. C. M. Summa, M. M. Rosenblatt, J.-K. Hong, J. D. Lear, W. F. DeGrado, Computational de novo design and characterization of an A2B2 diiron protein. J. Mol. Biol. 321, 923-938 (2002).
53. P. Bradley, K. M. S. Misura, D. Baker, Toward high-resolution de novo structure prediction for small proteins. Science 309, 1868 (2005).
54. S. L. Reid, D. Parry, H.-H. Liu, B. A. Connolly, Binding and recognition of GATATC target sequences by the EcoRV restriction endonuclease: A study using fluorescent oligonucleotides and fluorescence polarization. Biochemistry 40, 2484-2494 (2001).
55. A. M. Rossi, C. W. Taylor, Analysis of protein-ligand interactions by fluorescence polarization. Nat. Protoc. 6, 365-387 (2011).

What is claimed is:

1. A system, comprising:
at least one data processor; and
at least one memory storing instructions, which when executed by the at least one data processor, result in operations comprising:
querying a van der Mer (vdM) database to identify a first van der Mer known to interact with a first portion of a first compound, the first van der Mer corresponding to an in silico unit of protein structure that defines, based at least on a statistically preferred orientation of the first portion of the first compound relative to a backbone structure of a protein, a binding site for the first compound relative to the backbone structure of the protein; and
generating, based at least on the first van der Mer, a sequence for the protein such that the protein exhibits a binding affinity for the first compound.

2. The system of claim 1, wherein the van der Mer database includes a plurality of van der Mers, and wherein each of the plurality of van der Mers is associated with a portion of a compound and a backbone structure.

3. The system of claim 2, wherein the plurality of van der Mers are organized into one or more clusters of van der Mers exhibiting a same or similar interaction with the portion of the compound.

4. The system of any one of claims 2 to 3, wherein the plurality of van der Mers are clustered based at least on a first set of atomic protein coordinates associated with the portion of the compound and a second set of atomic protein coordinates associated with the backbone structure.

5. The system of any one of claims 2 to 4, wherein the plurality of van der Mers included in the van der Mer database are identified by searching a database of known protein structures for one or more units of protein structure exhibiting a van der Waals (vdW) contact with the portion of the compound.

6. The system of claim 5, wherein the one or more units of protein structure exhibiting the van der Waals (vdW) contact with the portion of the compound are identified as van der Mers based at least on a nature of contact with the portion of the compound.

7. The system of claim 6, wherein the nature of contact comprises one of a hydrogen bond, a close van der Waals contact, and a wide van der Waals contact.

8. The system of any one of claims 1 to 7, further comprising:
generating a first set of coordinates corresponding to the backbone structure of the protein;
generating a second set of coordinates corresponding to the compound or the portion of the compound; and
querying, based at least on the first set of coordinates and the second set of coordinates, the van der Mer database.

9. The system of any one of claims 1 to 8, further comprising:
querying the van der Mer database to identify a second van der Mer known to interact with the first portion of the first compound or a second portion of the first compound; and
generating, based at least on the second van der Mer, the sequence for the protein.

10. The system of any one of claims 1 to 9, wherein the backbone structure of the protein comprises one of a plurality of backbone structures with a geometry consistent with a known plasticity of a selected protein fold.

11. The system of any one of claims 1 to 10, wherein the sequence of the protein is further generated by packing additional residues in the binding site.

12. The system of any one of claims 1 to 11, wherein the sequence of the protein is further generated by packing a core of the protein.

13. The system of any one of claims 1 to 12, wherein the portion of the compound comprises a chemical group.

14. The system of any one of claims 1 to 13, wherein the compound comprises a ligand.

15. The system of claim 14, wherein the ligand comprises a peptide, a protein, a small molecule, or a small molecule-metal-ion complex.

16. The system of any one of claims 1 to 15, wherein the first van der Mer is selected instead of a second van der Mer based at least on the first van der Mer being observed in more experimentally determined protein structures than the second van der Mer.

17. The system of any one of claims 1 to 16, further comprising:
optimizing the sequence of the protein by at least identifying a location of the binding site relative to the backbone structure of the protein associated with a minimum energy function.

18. The system of claim 17, wherein the optimizing is performed by applying one or more of an interative algorithm, a heurisitic algorithm, a Monte Carlo sampling algorithm, a dead-end elimination algorithm, a branch and bound algorithm, a pruning algorithm, a simplex algorithm, a memetic algorithm, a differential evolution algorithm, an evolutionary algorithm, a genetic algorithm, a tabu algorithm, a particle swarm algorithm, and a simulated annealing algorithm.

19. The system of any one of claims 17 to 18, wherein the energy function comprises a molecular mechanics function, a structural bioinformatics function, an amino acid sidechain packing function, and/or protein radius of gyration function.

20. The system of any one of claims 1 to 19, wherein the sequence of the protein is further generated such that the protein exhibits a desired tertiary structure including one or more folds.

21. A computer-implemented method, comprising:
querying a van der Mer (vdM) database to identify a first van der Mer known to interact with a first portion of a first compound, the first van der Mer corresponding to an in silico unit of protein structure that defines, based at least on a statistically preferred orientation of the first portion of the first compound relative to a backbone structure of a protein, a binding site for the first compound relative to the backbone structure of the protein; and
generating, based at least on the first van der Mer, a sequence for the protein such that the protein exhibits a binding affinity for the first compound.

22. The method of claim 21, wherein the van der Mer database includes a plurality of van der Mers, and wherein each of the plurality of van der Mers is associated with a portion of a compound and a backbone structure.

23. The method of claim 22, wherein the plurality of van der Mers are organized into one or more clusters of van der Mers exhibiting a same or similar interaction with the portion of the compound.

24. The method of any one of claims 22 to 23, wherein the plurality of van der Mers are clustered based at least on a first set of atomic protein coordinates associated with the portion of the compound and a second set of atomic protein coordinates associated with the backbone structure.

25. The method of any one of claims 22 to 24, wherein the plurality of van der Mers included in the van der Mer database are identified by searching a database of known protein structures for one or more units of protein structure exhibiting a van der Waals (vdW) contact with the portion of the compound.

26. The method of claim 25, wherein the one or more units of protein structure exhibiting the van der Waals (vdW) contact with the portion of the compound are identified as van der Mers based at least on a nature of contact with the portion of the compound.

27. The method of claim 26, wherein the nature of contact comprises one of a hydrogen bond, a close van der Waals contact, and a wide van der Waals contact.

28. The method of any one of claims 21 to 27, further comprising:
generating a first set of coordinates corresponding to the backbone structure of the protein;
generating a second set of coordinates corresponding to the compound or the portion of the compound; and
querying, based at least on the first set of coordinates and the second set of coordinates, the van der Mer database.

29. The method of any one of claims 21 to 28, further comprising:
querying the van der Mer database to identify a second van der Mer known to interact with the first portion of the first compound or a second portion of the first compound;
and generating, based at least on the second van der Mer, the sequence for the protein.

30. The method of any one of claims 21 to 29, wherein the backbone structure of the protein comprises one of a plurality of backbone structures with a geometry consistent with a known plasticity of a selected protein fold.

31. The method of any one of claims 21 to 30, wherein the sequence of the protein is further generated by packing additional residues in the binding site.

32. The method of any one of claims 21 to 31, wherein the sequence of the protein is further generated by packing a core of the protein.

33. The method of any one of claims 21 to 32, wherein the portion of the compound comprises a chemical group.

34. The method of any one of claims 21 to 33, wherein the compound comprises a ligand.

35. The method of claim 34, wherein the ligand comprises a peptide, a protein, a small molecule, or a small molecule-metal-ion complex.

36. The method of any one of claims 21 to 35, wherein the first van der Mer is selected instead of a second van der Mer based at least on the first van der Mer being observed in more experimentally determined protein structures than the second van der Mer.

37. The method of any one of claims 21 to 36, further comprising:
optimizing the sequence of the protein by at least identifying a location of the binding site relative to the backbone structure of the protein associated with a minimum energy function.

38. The method of claim 37, wherein the optimizing is performed by applying one or more of an interative algorithm, a heurisitic algorithm, a Monte Carlo sampling algorithm, a dead-end elimination algorithm, a branch and bound algorithm, a pruning algorithm, a simplex algorithm, a memetic algorithm, a differential evolution algorithm, an evolutionary algorithm, a genetic algorithm, a tabu algorithm, a particle swarm algorithm, and a simulated annealing algorithm.

39. The method of any one of claims 37 to 38, wherein the energy function comprises a molecular mechanics function, a structural bioinformatics function, an amino acid sidechain packing function, and/or protein radius of gyration function.

40. The method of any one of claims 21 to 39, wherein the sequence of the protein is further generated such that the protein exhibits a desired tertiary structure including one or more folds.

41. A computer-implemented method for identifying a protein capable of binding a compound, comprising:
(a) generating a first set of atomic protein coordinates representing a backbone structure of the protein;
(b) identifying a first van der Mer from a van der Mer database comprising a first set of atomic van der Mer coordinates representing a first chemical group of the compound, a first amino acid side chain and a first portion of a protein backbone bound to said first amino acid side chain, wherein said first chemical group interacts in silico with said first portion of a protein backbone or said first amino acid side chain;
(c) identifying portions of the backbone structure of the protein having atomic protein coordinates that are capable of overlapping with the atomic van der Mer coordinates of the first portion of a protein backbone of the first van der Mer;
(d) repeating steps (b) and (c) for at least one additional van der Mer independently representing an optionally different chemical group of the compound, an additional independent amino acid side chain and an additional independent portion of a protein backbone bound to said independent additional amino acid side chain;
(e) generating a set of atomic chemical coordinates representing the compound;
(f) generating at least one set of atomic coordinates of an in silico complex of said protein capable of binding said compound bound to said compound;
wherein said in silico complex optimizes the overlap between the atomic chemical coordinates of the compound chemical groups and the atomic van der Mer coordinates representing the independent chemical groups of the compound identified in steps (b) to (d);
(g) generating a set of atomic amino acid coordinates for amino acid side chains and portions of protein backbone independently representing those portions of the backbone structure of the protein that are not overlapping with atomic van der Mer coordinates of the van der Mer comprising atomic van der Mer coordinates that are overlapping with the atomic chemical coordinates representing the compound of the in silico complex of step (f);
(h) based at least in part on steps (a) to (g), optimizing atomic coordinates of the compound and protein thereby identifying a protein capable of binding said compound.

42. The method of claim 41, comprising generating a plurality of sets of atomic coordinates of an in silico complex of said protein capable of binding said compound bound to said compound in step (f).

43. The method of claim 42, wherein said plurality of sets of atomic coordinates of an in silico complex of said protein capable of binding said compound bound to said compound are independently different from each other and optimization of the overlap between the atomic chemical coordinates of the compound chemical groups and the atomic van der Mer coordinates representing the independent chemical groups of the compound identified in steps (b) to (d) is performed without duplication of said sets.

44. The method of one of claims 42 to 43, wherein said plurality of independent sets of atomic coordinates of an in silico complex of said protein capable of binding said compound bound to said compound are independently different from each other and are scored.

45. The method of claim 44, wherein said scoring comprises calculating a cluster score for each of said plurality of sets of atomic coordinates of an in silico complex of said protein capable of binding said compound bound to said compound.

46. The method of claim 45, wherein the cluster score is the natural logarithm of the ratio of 1) the number of members in an independent set of geometrically overlapping van der Mer of one chemical group and one amino acid to 2) the average number of members in all independent sets van der Mer of said chemical group and said amino acid.

47. The method of claim 46, wherein the members in an independent set of geometrically overlapping compound van der Mer of one chemical group are identified by having an RMSD below a threshold, wherein the RMSD is calculated using the atomic coordinates of the chemical group and the backbone atoms of the amino acid residue of a van der Mer.

48. The method of claim 47, wherein the RMSD threshold is 0.5 angstrom.

49. The method of any one of claims 41 to 48, wherein step (a) comprises generating a plurality of independent sets of atomic protein coordinates representing independent backbone structures of the protein.

50. The method of claim 49, wherein the plurality of independent backbone structures of the protein have a similar overall three dimensional fold.

51. The method of any one of claims 49 to 50, wherein the plurality of independent backbone structures of the protein have an RMSD of less than 3 angstrom.

52. The method of any one of claims 41 to 51, wherein the compound chemical groups and van der Mer chemical groups are polar groups.

53. The method of any one of claims 41 to 52, wherein steps (g) and (h) comprise use of a method described in international application no. WO2019/023644.

54. The method of any one of claims 41 to 53, wherein step (c) comprises identifying all portions of the backbone structure of the protein having atomic protein coordinates that are capable of overlapping with the atomic van der Mer coordinates of the first portion of a protein backbone of the first van der Mer.

55. The method of any one of claims 41 to 54, wherein step (d) comprises repeating steps (b) and (c) for all van der Mer in the van der Mer database independently representing all chemical groups of the compound, an additional independent amino acid side chain and an additional independent portion of a protein backbone bound to said independent additional amino acid side chain.

56. A computer-implemented method for identifying a complex of a protein bound to a compound, comprising:
(a) generating a first set of atomic protein coordinates representing the side chain and backbone structure of the protein;
(b) identifying a first van der Mer from a van der Mer database comprising a first set of atomic van der Mer coordinates representing a first chemical group of the compound, a first amino acid side chain and a first portion of a protein backbone bound to said first amino acid side chain, wherein said first chemical group interacts in silico with said first portion of a protein backbone or said first amino acid side chain;
(c) identifying portions of the backbone structure of the protein having atomic protein coordinates that are capable of overlapping with the atomic van der Mer coordinates of the first portion of a protein backbone of the first van der Mer wherein the amino acid side chain of the van der Mer and the amino acid side chain directly attached to the overlapping portions of the backbone structure of the protein are the same side chain;
(d) repeating steps (b) and (c) for at least one additional van der Mer independently representing an optionally different chemical group of the compound, an additional independent amino acid side chain and an additional independent portion of a protein backbone bound to said independent additional amino acid side chain;
(e) generating a set of atomic chemical coordinates representing the compound;
(f) generating at least one set of atomic coordinates of an in silico complex of said protein capable of binding said compound bound to said compound; wherein said in silico complex optimizes the overlap between the atomic chemical coordinates of the compound chemical groups and the atomic van der Mer coordinates representing the independent chemical groups of the compound identified in steps (b) to (d);
(g) based at least in part on steps (a) to (f), optimizing atomic coordinates of the compound and protein thereby identifying a complex of a protein bound to a compound.

57. A computer-implemented method for identifying a protein capable of binding a compound, comprising:
(a) generating a first set of atomic protein coordinates representing a protein backbone structure;
(b) generating a first set of atomic chemical coordinates representing a first chemical group of the compound;
(c) identifying a first van der Mer from a van der Mer database comprising a first set of atomic van der Mer coordinates representing said first chemical group of the compound, a first amino acid side chain and a first portion of a protein backbone bound to said first amino acid side chain, wherein said first chemical group interacts in silico with said first portion of a protein backbone or said first amino acid side chain;
(d) generating a second set of atomic chemical coordinates representing a second chemical group of the compound;
(e) identifying a second van der Mer from said van der Mer database comprising a second set of atomic van der Mer coordinates representing said second chemical group of the compound, a second amino acid side chain and a second portion of a protein backbone bound to said second amino acid side chain, wherein said second chemical group interacts in silico with said second portion of a protein backbone or said second amino acid side chain;
(f) calculating an energetic stability of said protein backbone structure bound to said compound using said first set of atomic van der Mer coordinates and said second set of atomic van der Mer coordinates in silico;
(g) repeating steps (a) to (f) for additional van der Mers representing said first chemical group of the compound, a first amino acid side chain and a first portion of a protein backbone bound to said first amino acid side chain and additional van der Mers representing said second chemical group of the compound, a second amino acid side chain and a second portion of a protein backbone bound to said second amino acid side chain;
(h) generating a set of atomic amino acid coordinates for amino acid side chains and portions of protein backbone independently representing those portions of the protein backbone structure not represented by a van der Mer of steps (a) to (g);
(i) based at least in part on steps (a) to (h), optimizing atomic coordinates of the compound and protein thereby identifying a protein capable of binding said compound.

58. A computer-implemented method for identifying a protein capable of binding a compound, comprising:
(a) identifying a first van der Mer from a van der Mer database comprising atomic van der Mer coordinates of a chemical group of the compound, wherein the atomic van der Mer coordinates of the chemical group in the first van der Mer overlap with the atomic chemical coordinates of the chemical group of the compound;
(b) identifying a protein backbone for the protein wherein the atoms of the protein backbone are associated with a set of atomic protein coordinates;
(c) identifying an overlap between the atomic van der Mer coordinates of the amino acid backbone of the first van der Mer identified in step (a) and the atomic protein coordinates of an amino acid residue of the protein backbone identified in step (b);
(d) optionally repeating steps (a) to (c) for a different chemical group of the compound;
(e) identifying independent sets of van der Mer identified in steps (a) to (d) wherein all van der Mer of each independent set include atomic van der Mer coordinates that collectively simultaneously overlap atomic protein coordinates of the protein backbone identified in step (b);
(f) identifying at least one independent set of van der Mer identified in step (e) with a cluster score above a threshold;
(g) identifying an amino acid residue for each amino acid of the protein backbone identified in step (b) having atomic amino acid coordinates that are not overlapping with the atomic van der Mer coordinates of the set of van der Mer identified in step (f);
(h) optimizing atomic coordinates of the compound and protein;
wherein the optimization is performed using at least an energy minimization calculation, and wherein the optimization is performed to energetically stabilize a complex of the compound and protein.

59. A computer-implemented method for identifying a protein capable of binding a compound, comprising:
(a) identifying covalently bonded amino acid backbone residues of the protein wherein each amino acid backbone residue atom is associated with a set of atomic protein coordinates;
(b) identifying an independent set of van der Mer associated with an amino acid backbone residue and a chemical group of the compound, wherein each van der Mer is associated with a set of atomic van der Mer coordinates for an amino acid and chemical group of the compound and the atomic van der Mer coordinates for the van der Mer amino acid backbone atoms of each independent set of van der Mer overlap with amino acid backbone residue atomic protein coordinates of the protein;
(c) identifying and removing from each independent set of van der Mer, any van der Mer wherein atomic van der Mer coordinates of a sidechain or chemical group of the van der Mer overlap with atomic protein coordinates of the covalently bonded amino acid backbone residues of the protein;
(d) identifying and removing any van der Mer wherein atomic van der Mer coordinates of the chemical group of the van der Mer is characterized as exposed to bulk solvent;
(e) identifying independent sets of atomic chemical coordinates of the compound wherein, the atomic chemical coordinates of the compound chemical group atoms of each independent set overlap with atomic van der Mer coordinates of chemical group atoms of van der Mer identified in steps (b) to (d) and atomic van der Mer coordinates of said van der Mer further include atomic van der Mer coordinates for amino acid backbone atoms that overlap with atomic protein coordinates of amino acid backbone atoms of the protein;
(f) identifying and sorting independent sets of atomic chemical coordinates of the compound of step (e) based on the value of the compound van der Mer cluster score;
(g) identifying a preferred amino acid for an amino acid residue position of the protein when the amino acid residue position of the protein has amino acid backbone atom atomic protein coordinates that overlap with the amino acid backbone atomic van der Mer coordinates of a van der Mer identified in step (f) and the preferred amino acid is the amino acid associated with said van der Mer;
(h) optimizing atomic coordinates of the compound and amino acid residues of the protein;
wherein the optimization is performed using at least an energy minimization calculation, and wherein the optimization is performed to energetically stabilize said protein.

60. The method of any one of claims 41 to 59, wherein the optimizing comprises an iterative or heuristic algorithm.

61. The method of any one of claims 41 to 59, wherein the optimizing comprises a simplex algorithm, memetic algorithm, differential evolution algorithm, evolutionary algorithm, genetic algorithm, tabu algorithm, particle swarm algorithm, or stimulated annealing algorithm.

62. The method of any one of claims 41 to 59, wherein the optimizing comprises a Monte Carlo sampling algorithm, dead-end elimination algorithm, branch and bound algorithm, or a pruning algorithm.

63. The method of one of claims 41 to 59, wherein the energy minimization calculation comprises a molecular mechanics function, a structural bioinformatics function, an amino acid sidechain packing function, a protein radius of gyration function, or a combination thereof.

64. The method of one of claims 41 to 63, wherein identifying atomic van der Mer coordinates of a chemical group of a van der Mer as exposed to bulk solvent is performed using a convex hull algorithm.

65. The method of one of claims 57 to 64, wherein the cluster score is the natural logarithm of the ratio of 1) the number of members in an independent set of geometrically overlapping van der Mer of one chemical group and one amino acid to 2) the average number of members in all independent sets van der Mer of said chemical group and said amino acid.

66. The method of claim 65, wherein the members in an independent set of geometrically overlapping compound van der Mer of one chemical group are identified by having an RMSD below a threshold, wherein the RMSD is calculated using the atomic coordinates of the chemical group and the backbone atoms of the amino acid residue of a van der Mer.

67. The method of claim 66, wherein the RMSD threshold is 0.5 angstrom.

68. The method of one of claims 59 to 67, wherein the preferred amino acid of step (g) is an amino acid in a van der Mer having a cluster score greater than 2.

69. The method of any one of claims 41 to 68, wherein identifying an amino acid residue for each protein backbone residue having atomic amino acid coordinates that are not overlapping with the atomic van der Mer coordinates of a van der Mer is performed using The Rosetta Software.

70. The method of any one of claims 41 to 69, wherein the van der Mer database is a collection of independent van der Mer each comprising a unique set of atomic van der Mer coordinates describing the three dimensional positions of a chemical group interacting in silico with an amino acid residue, further wherein said interacting was identified in an empirically determined protein and chemical group complex.

71. The method of any one of claims 41 to 70, wherein the protein is a 4-helix bundle protein.

72. The method of any one of claims 41 to 71, wherein the compound comprises a charged chemical group at physiological pH.

73. The method of any one of claims 41 to 72, wherein the compound comprises a polar chemical group at physiological pH.

74. The method of any one of claims 41 to 73, further comprising making the protein.

75. The method of any one of claims 57 to 74, comprising use of a method described in international application no. WO2019/023644.

76. A non-transitory computer readable medium storing instructions, which when executed by at least one data processor, result in operations comprising:
querying a van der Mer (vdM) database to identify a first van der Mer known to interact with a first portion of a first compound, the first van der Mer corresponding to an in silico unit of protein structure that defines, based at least on a statistically preferred orientation of the first portion of the first compound relative to a backbone structure of a protein, a binding site for the first compound relative to the backbone structure of the protein; and
generating, based at least on the first van der Mer, a sequence for the protein such that the protein exhibits a binding affinity for the first compound.

77. An apparatus, comprising:
means for querying a van der Mer (vdM) database to identify a first van der Mer known to interact with a first portion of a first compound, the first van der Mer corresponding to an in silico unit of protein structure that defines, based at least on a statistically preferred orientation of the first portion of the first compound relative to a backbone structure of a protein, a binding site for the first compound relative to the backbone structure of the protein; and
means for generating, based at least on the first van der Mer, a sequence for the protein such that the protein exhibits a binding affinity for the first compound.

78. The apparatus of claim 77, further comprising means for performing the method of any one of claims 21-40.
